(19) United States
(12) Patent Application Publication
(10) Pub. No.: US 2024/0102052 A1
(43) Pub. Date: Mar. 28, 2024

(54) PNMA2-BASED CAPSIDS AND USES THEREOF

(71) Applicants: VNV NEWCO INC., New York, NY (US); University Of Utah Research Foundation, Salt Lake City, UT (US)

(72) Inventors: Colin MALONE, Cambridge, MA (US); Oleg LAPTENKO, Cambridge, MA (US); Adam FRAITES, Cambridge, MA (US); Jessica CRISP, Cambridge, MA (US); Andrey PISAREV, Cambridge, MA (US); Anil ANGAD, Cambridge, MA (US); Heley ONG, Cambridge, MA (US); Zachary GILBERT, Cambridge, MA (US); Jason D. SHEPHERD, Salt Lake City, UT (US)

(21) Appl. No.: 18/262,522

(22) PCT Filed: Jan. 26, 2022
(86) PCT No.: PCT/US2022/013954

§ 371 Date: Jul. 21, 2023

Related U.S. Application Data
(60) Provisional application No. 63/142,441, filed on Jan. 27, 2021.
(60) Provisional application No. 63/152,653, filed on Feb. 23, 2021.

(51) Int. Cl. C12N 15/88; C07K 14/005; C12N 9/22; C12N 15/113; C12N 15/86

(52) U.S. Cl. CPC C12N 15/88; C07K 14/005; C12N 9/22; C12N 15/113; C12N 15/86; C12N 2310/20; C12N 2740/10022; C12N 2740/10042

(57) ABSTRACT

Disclosed herein, in certain embodiments, are recombinant PNMA2 and endogenous Gag polypeptides, capsids comprising the recombinant PNMA2 or endogenous Gag polypeptides, and methods of making and using recombinant PNMA2 and endogenous Gag polypeptides.

### CROSS REFERENCE

This application claims the benefit of U.S. Provisional Application No. 63/142,441, filed Jan. 27, 2021, and U.S. Provisional Application No. 63/152,653, filed Feb. 23, 2021, each of which is incorporated herein by reference in its entirety.

### SEQUENCE LISTING STATEMENT

The instant application contains a Sequence Listing which has been submitted electronically in ASCII format and is hereby incorporated by reference in its entirety. Said ASCII copy, created on Jan. 26, 2022, is named 54838_709_601_SL.txt and is 94,168 bytes in size.

### SUMMARY OF THE DISCLOSURE

Administering diagnostic or therapeutic agents to a site of interest with precision has presented an ongoing challenge. Available methods of delivering nucleic acids to cells suffer from a number of limitations. For example, AAV viral vectors often used for gene therapy are immunogenic, have a limited payload capacity, suffer from poor bio-distribution, can only be administered by direct injection, and pose a risk of disrupting host genes by integration. Some studies have suggested that a significant proportion (e.g., ≥50%) of the population have pre-existing immunity to viral vectors such as AAV, which could severely limit their effectiveness in therapeutic applications. Thus, there is a need for new and improved compositions and methods for delivering therapeutic payloads. Capsids (or virus-like particles, VLPs) disclosed herein have the potential to address many of these shortcomings. Disclosed herein, in certain embodiments, are endogenous retroviral capsid polypeptides. In some embodiments, endogenous Gag (endo-Gag) polypeptides of the disclosure, such as PNMA2 polypeptides, assemble into capsids for delivery of a cargo of interest. Endo-Gag polypeptides of the disclosure exhibit surprising and unexpected advantages over existing and alternate capsid-forming polypeptides, such as improved efficiency in capsid assembly, capsid disassembly, and/or capsid reassembly, e.g., reassembly with a heterologous cargo. In additional embodiments, described herein are capsids, e.g., PNMA2-based or endo-Gag-based capsids, for delivery of a cargo of interest. Also disclosed herein are engineered endo-Gag polypeptides, such as PNMA2 polypeptides, with additional modifications of particular advantage and utility.

Disclosed herein, in some aspects, is a capsid comprising an engineered endogenous retroviral capsid polypeptide, wherein the capsid is capable of disassembling into a non-capsid state with an efficiency of at least 1%, and reassembling into a capsid state with an efficiency of at least 1%.

In some embodiments, the efficiency of the disassembling is as determined by quantifying an amount of the endogenous retroviral capsid polypeptide in solution after treating purified capsids with a disassembly buffer. determined by In some embodiments, the efficiency of the reassembling is as determined by comparing an amount of engineered endogenous retroviral capsid polypeptide in a capsid peak from an amount of engineered endogenous retroviral capsid polypeptide in a monomer peak, and wherein the capsid peak and the monomer peak are identified by multi-angle dynamic light scattering or by size exclusion chromatography. In some embodiments, the efficiency of the reassembling is as determined by quantifying an amount of the retroviral capsid polypeptide in the non-capsid state prior to the reassembling and quantifying an amount of the retroviral capsid polypeptide in the capsid state after the reassembling by size exclusion chromatography. In some embodiments, the capsid is capable of disassembling into the non-capsid state with an efficiency of at least about 20%. In some embodiments, the capsid is capable of reassembling into the capsid state with an efficiency of at least about 20%. In some embodiments, the engineered endogenous retroviral capsid polypeptide is an engineered endo-Gag polypeptide. In some embodiments, the engineered endogenous retroviral capsid polypeptide comprises a sequence of an endo-Gag polypeptide. In some embodiments, the engineered endogenous retroviral capsid polypeptide is an engineered PNMA protein. In some embodiments, the engineered endogenous retroviral capsid polypeptide is an engineered PNMA2 protein. In some embodiments, the reassembling is in a buffer containing less than 500 mOsm/kg of salt. In some embodiments, the capsid further comprises a heterologous cargo that is not associated the endogenous retroviral capsid polypeptide in nature. In some embodiments, the heterologous cargo comprises a nucleic acid. In some embodiments, the heterologous cargo comprises an RNA. In some embodiments, the heterologous cargo comprises a DNA. In some embodiments, the heterologous cargo comprises or encodes a gene editing system or a component thereof. In some embodiments, the heterologous cargo comprises or encodes a CRISPR/Cas system or a component thereof. In some embodiments, the heterologous cargo comprises or encodes a zinc finger nuclease system or a component thereof. In some embodiments, the heterologous cargo comprises or encodes a TALEN system or a component thereof. In some embodiments, the heterologous cargo comprises a therapeutic agent. In some embodiments, the heterologous cargo comprises a polypeptide. In some embodiments, the heterologous cargo comprises an antibody or antigen-binding fragment thereof, a peptidomimetic, a nucleotidomimetic, a drug, a diagnostic tool, an imaging tool, a small molecule, or a combination thereof. In some embodiments, at least 50% of the heterologous cargo is in an interior of the capsids. In some embodiments, at least 50% of the heterologous cargo is on an exterior of the capsids. In some embodiments, the heterologous cargo is in an interior of at least 50% of the capsids. In some embodiments, the heterologous cargo is on an exterior of at least 1% of the capsids. In some embodiments, the engineered PNMA2 polypeptide comprises an amino acid sequence of a mammalian PNMA2. In some embodiments, the engineered PNMA2 polypeptide comprises an amino acid sequence of a human PNMA2. In some embodiments, the engineered PNMA2 polypeptide comprises an amino acid sequence with at least 90% sequence identity to a sequence comprising at least 100 consecutive amino acids of SEQ ID NO: 7, SEQ ID NO: 8, or SEQ ID NO: 1. In some embodiments, the PNMA2 polypeptide comprises an amino acid sequence with at least 90% sequence identity to a sequence comprising at least 250 consecutive amino acids of SEQ ID NO: 7, SEQ ID NO: 8, or SEQ ID NO: 1. In some embodiments, the engineered PNMA2 polypeptide comprises an amino acid sequence with at least 90% sequence identity to SEQ ID NO: 7, SEQ ID NO: 8, or SEQ ID NO: 1. In some embodiments, the engineered PNMA2 polypeptide comprises an amino acid sequence that is SEQ ID NO: 7, SEQ ID NO: 8, or SEQ ID NO: 1.

Disclosed herein, in some aspects is an isolated engineered endogenous retroviral capsid polypeptide that is capable of assembling to form a capsid with an efficiency of at least 5%.

In some embodiments, the efficiency of the assembling is as determined by size exclusion chromatography assay to quantify the percentage of endogenous retroviral capsid polypeptide present in a capsid state. In some embodiments, upon isolation the engineered endogenous retroviral capsid polypeptide is capable of assembling to form a capsid with an efficiency of at least about 20%. In some embodiments, upon isolation the engineered endogenous retroviral capsid polypeptide is capable of assembling to form a capsid with an efficiency of at least about 50%. In some embodiments, the assembling is in a buffer containing less than 500 mOsm/kg of salt. In some embodiments, the engineered endogenous retroviral capsid polypeptide is an endo-Gag polypeptide. In some embodiments, the engineered endogenous retroviral capsid polypeptide is an engineered PNMA protein. In some embodiments, the engineered endogenous retroviral capsid polypeptide is an engineered PNMA2 protein. In some embodiments, the engineered PNMA2 protein comprises a sequence modification relative to SEQ ID NO: 1 or SEQ ID NO: 7. In some embodiments, the sequence modification comprises an amino acid insertion. In some embodiments, the sequence modification comprises an amino acid deletion. In some embodiments, the sequence modification comprises an amino acid substitution In some embodiments, the sequence modification comprises a cargo binding domain. In some embodiments, the sequence modification comprises a nucleic acid binding domain. In some embodiments, the sequence modification comprises an RNA binding domain. In some embodiments, the sequence modification comprises a DNA binding domain. In some embodiments, the sequence modification comprises a zinc finger domain. In some embodiments, the sequence modification comprises a sub-cellular localization signal. In some embodiments, the sequence modification comprises a nuclear localization signal (NLS). In some embodiments, the sequence modification comprises an antibody or an antigen-binding fragment thereof. In some embodiments, the antibody or antigen-binding fragment comprises a single chain variable fragment (scFv) or a single domain antibody. In some embodiments, the sequence modification comprises a domain that binds to a cell surface molecule. In some embodiments, the sequence modification comprises an arginine-rich domain. In some embodiments, the sequence modification is at an N-terminus of the engineered PNMA2 polypeptide. In some embodiments, the sequence modification is at a C-terminus of the engineered PNMA2 polypeptide. In some embodiments, the sequence modification is within the PNMA2 polypeptide. In some embodiments, the sequence modification comprises addition of a cysteine residue that is not present in native PNMA2 or elimination of a cysteine residue that is present in native PNMA2.

Disclosed herein, in some aspects, is a method of making a capsid, the method comprising: (a) expressing an endogenous retroviral capsid polypeptide in a host cell or a cell-free expression system; and (b) isolating the endogenous retroviral capsid polypeptide; wherein at least about 5% of the isolated endogenous retroviral capsid polypeptide assembles to form the capsid.

Disclosed herein, in some aspects, is a method of making a capsid comprising a cargo, the method comprising: (a) expressing an endogenous retroviral capsid polypeptide in a host cell or a cell-free expression system; (b) isolating a capsid comprising the endogenous retroviral capsid polypeptide; (c) disassembling the capsid, thereby generating endogenous retroviral capsid polypeptide in a disassembled state; (d) contacting the disassembled endogenous retroviral capsid polypeptide to a cargo; (e) reassembling the disassembled endogenous retroviral capsid polypeptide, thereby generating the capsid comprising the cargo.

In some embodiments, the percentage of the isolated endogenous retroviral capsid that assembles to form the capsid is as determined by size exclusion chromatography. In some embodiments, at least 20% of the isolated endogenous retroviral capsid polypeptide assembles to form the capsid.

Disclosed herein, in some aspects, is a method of making a capsid comprising a heterologous cargo that is not associated with the capsid in nature, the method comprising: (a) expressing an endogenous retroviral capsid polypeptide in a host cell or a cell-free expression system; (b) isolating endogenous retroviral capsid polypeptide; (c) treating the isolated endogenous retroviral capsid polypeptide with a disassembly buffer, thereby generating endogenous retroviral capsid polypeptide in a disassembled state; (d) reassembling the disassembled endogenous retroviral capsid polypeptide in a reassembly buffer with the heterologous cargo present; thereby generating the capsid comprising the heterologous cargo.

In some embodiments, the isolated PNMA2 polypeptide is at least partially present in a capsid form prior to step (c). In some embodiments, at least about 5% of the PNMA2 polypeptide that is present in capsid form before step (c) is disassembled after step (c). In some embodiments, at least about 5% of the PNMA2 polypeptide that is present in disassembled form after step (c) is present in capsid form after step (d).

Disclosed herein, in some aspects, is a method of loading endogenous retroviral capsids, the method comprising: (a) disassembling endogenous retroviral capsids into a composition comprising endogenous retroviral capsid monomers; (b) contacting a cargo with the composition comprising endogenous retroviral capsid monomers; and (c) reassembling the composition comprising endogenous retroviral capsid monomers into endogenous retroviral capsids; thereby loading the cargo into an interior of the endogenous retroviral capsids.

In some embodiments, the endogenous retroviral capsid polypeptide is a native endo-Gag polypeptide or an engineered endo-Gag polypeptide. In some embodiments, the endogenous retroviral capsid polypeptide is a native PNMA protein or an engineered PNMA protein. In some embodiments, the endogenous retroviral capsid polypeptide is a native PNMA2 protein or an engineered PNMA2 protein. In some embodiments, the method produces assembled capsids of at least about 50% purity as determined by SDS-PAGE. In some embodiments, the method produces assembled capsids with at least about 50% particle homogeneity as determined by multi-angle dynamic light scattering. In some embodiments, the disassembling occurs in a disassembly buffer comprising a reducing agent. In some embodiments, the disassembly buffer comprises a reducing agent. In some embodiments, the reducing agent comprises reduced glutathione (GSH), beta mercaptoethanol ((3-ME), Dithiothreitol (DTT), or tris(2-carboxyethyl)phosphine (TCEP). In some embodiments, the disassembly buffer comprises a non-denaturing detergent. In some embodiments, the disassembling occurs in a disassembly buffer comprising a non-denaturing detergent. In some embodiments, the non-denaturing detergent is CHAPS. In some embodiments, the reassembling occurs in a reassembly buffer containing less than 500 mOsm/kg of salt. In some embodiments, the reassembling occurs in a reassembly buffer containing about 270-330 mOsm/kg of salt.

Disclosed herein, in some aspects, is an engineered PNMA2 polypeptide that comprises a sequence modification relative to SEQ ID NO: 1 or SEQ ID NO: 7.

In some embodiments, the sequence modification comprises an amino acid insertion. In some embodiments, the sequence modification comprises an amino acid deletion. In some embodiments, the sequence modification comprises an amino acid substitution In some embodiments, the sequence modification comprises a cargo binding domain. In some embodiments, the sequence modification comprises a nucleic acid binding domain. In some embodiments, the sequence modification comprises an RNA binding domain. In some embodiments, the sequence modification comprises a DNA binding domain. In some embodiments, the sequence modification comprises a zinc finger domain. In some embodiments, the sequence modification comprises a sub-cellular localization signal. In some embodiments, the sequence modification comprises a nuclear localization signal (NLS). In some embodiments, the sequence modification comprises an antibody or an antigen-binding fragment thereof. In some embodiments, the antibody or antigen-binding fragment comprises a single chain variable fragment (scFv) or a single domain antibody. In some embodiments, the sequence modification comprises a domain that binds to a cell surface molecule. In some embodiments, the sequence modification comprises an arginine-rich domain. In some embodiments, the sequence modification is at an N-terminus of the engineered PNMA2 polypeptide. In some embodiments, the sequence modification is at a C-terminus of the engineered PNMA2 polypeptide. In some embodiments, the sequence modification is within the PNMA2 polypeptide. In some embodiments, the sequence modification comprises a cysteine residue that is not present in native PNMA2.

Disclosed herein, in some aspects, is a capsid comprising a PNMA2 polypeptide and a heterologous cargo that is not associated with a capsid comprising PNMA2 in nature, wherein the PNMA2 polypeptide comprises an amino acid sequence having at least 90% sequence identity to SEQ ID NO: 7, SEQ ID NO: 8, or SEQ ID NO: 1.

Disclosed herein, in some aspects, is a capsid comprising the engineered PNMA2 polypeptide of any one of the preceding embodiments.

A capsid comprising: (a) a first endogenous retroviral capsid polypeptide that comprises an amino acid sequence of a native PNMA2 polypeptide or an engineered PNMA2 polypeptide; and (b) a second endogenous retroviral capsid polypeptide; wherein the amino acid sequence of the first endogenous retroviral capsid polypeptide is not identical to the amino acid sequence of the second endogenous retroviral capsid polypeptide.

Disclosed herein, in some aspects, is a capsid comprising: (a) a first endogenous retroviral capsid polypeptide that is the engineered PNMA2 polypeptide of any one of the preceding embodiments; and (b) a second endogenous retroviral capsid polypeptide; wherein the amino acid sequence of the first endogenous retroviral capsid polypeptide is not identical to the amino acid sequence of the second endogenous retroviral capsid polypeptide.

In some embodiments, the second endogenous retroviral capsid polypeptide comprises an amino acid sequence of a native PNMA polypeptide or an engineered PNMA polypeptide. In some embodiments, the second endogenous retroviral capsid polypeptide comprises an amino acid sequence of a native PNMA2 polypeptide or an engineered PNMA2 polypeptide. In some embodiments, the second endogenous retroviral capsid polypeptide is not a PNMA2 polypeptide. In some embodiments, the capsid further comprises a heterologous cargo that is not associated with a capsid comprising PNMA2 in nature. In some embodiments, the heterologous cargo comprises a nucleic acid. In some embodiments, the heterologous cargo comprises an RNA. In some embodiments, the heterologous cargo comprises a DNA. In some embodiments, the heterologous cargo comprises or encodes a gene editing system or a component thereof. In some embodiments, the heterologous cargo comprises or encodes a CRISPR/Cas system or a component thereof. In some embodiments, the heterologous cargo comprises or encodes a zinc finger nuclease system or a component thereof. In some embodiments, the heterologous cargo comprises or encodes a TALEN system or a component thereof. In some embodiments, the heterologous cargo comprises a therapeutic agent. In some embodiments, the heterologous cargo comprises a polypeptide. In some embodiments, the heterologous cargo comprises an antibody or antigen-binding fragment thereof, a peptidomimetic, a nucleotidomimetic, a drug, a diagnostic tool, an imaging tool, a small molecule, or a combination thereof. In some embodiments, at least 50% of the heterologous cargo is in an interior of the capsids. In some embodiments, at least 50% of the heterologous cargo is on an exterior of the capsids. In some embodiments, the heterologous cargo is in an interior of at least 50% of the capsids. In some embodiments, the heterologous cargo is on an exterior of at least 1% of the capsids. In some embodiments, the PNMA2 polypeptide comprises an amino acid sequence of a mammalian PNMA2. In some embodiments, the PNMA2 polypeptide comprises an amino acid sequence of a human PNMA2. In some embodiments, the PNMA2 polypeptide comprises an amino acid sequence with at least 90% sequence identity to a sequence comprising at least 100 consecutive amino acids of SEQ ID NO: 7, SEQ ID NO: 8, or SEQ ID NO: 1. In some embodiments, the PNMA2 polypeptide comprises an amino acid sequence with at least 90% sequence identity to a sequence comprising at least 250 consecutive amino acids of SEQ ID NO: 7, SEQ ID NO: 8, or SEQ ID NO: 1. In some embodiments, the PNMA2 polypeptide comprises an amino acid sequence with at least 90% sequence identity to SEQ ID NO: 7, SEQ ID NO: 8, or SEQ ID NO: 1. In some embodiments, the PNMA2 polypeptide comprises an amino acid sequence that is SEQ ID NO: 7, SEQ ID NO: 8, or SEQ ID NO: 1. In some embodiments, the capsid comprises a disulfide bond.

Disclosed herein, in some aspects, is a composition comprising a plurality of capsids that comprise a PNMA2 polypeptide, wherein the capsids are at least 50% pure as determined by SDS-PAGE.

Disclosed herein, in some aspects, is a composition comprising a plurality of the capsid of any one of the preceding embodiments, wherein the capsids are at least 50% pure as determined by SDS-PAGE.

Disclosed herein, in some aspects, is a composition comprising a plurality of capsids that comprise a PNMA2 polypeptide, wherein the capsids comprise at least 50% particle homogeneity as determined by multi-angle dynamic light scattering.

Disclosed herein, in some aspects, is a composition comprising a plurality of capsids that comprise a PNMA2 polypeptide, wherein the composition comprises at least 50% particle homogeneity as determined by multi-angle dynamic light scattering.

Disclosed herein, in some aspects, is a composition comprising a plurality of capsids of any one of the preceding embodiments, wherein the capsids comprise at least 50% particle homogeneity as determined by multi-angle dynamic light scattering.

Disclosed herein, in some aspects, is a composition comprising a plurality of capsids of any one of the preceding embodiments, wherein the composition comprises at least 50% particle homogeneity as determined by multi-angle dynamic light scattering.

In some embodiments, the composition further comprises a delivery component. In some embodiments, the delivery component comprises a lipid. In some embodiments, the delivery component comprises a polypeptide. In some embodiments, the delivery component comprises a polymer. In some embodiments, the delivery component comprises a cationic lipid. In some embodiments, the delivery component comprises a cationic peptide. In some embodiments, the delivery component comprises a cationic polymer. In some embodiments, the delivery component comprises a cell-penetrating peptide. In some embodiments, the delivery component comprises a fusogenic protein. In some embodiments, the delivery component comprises an endogenous retroviral envelope protein. In some embodiments, the delivery component comprises a liposome. Disclosed herein, in some aspects, is a nucleic acid encoding the engineered PNMA2 polypeptide of any one of the preceding embodiments. Disclosed herein, in some aspects, is a vector comprising the nucleic acid. Disclosed herein, in some aspects, is a cell comprising the nucleic acid.

Disclosed herein, in some aspects, is a method of delivering a heterologous cargo to a cell, comprising contacting the cell with the composition or the capsid of any one of the preceding embodiments.

Disclosed herein, in some aspects, is a method of delivering a cargo to a cell, the method comprising contacting the cell with a composition comprising a plurality of isolated capsids, wherein each capsid of the plurality of isolated capsids comprises a polypeptide comprising a sequence of PNMA2 and the cargo.

Disclosed herein, in some aspects, is a method of delivering a cargo to a cell, the method comprising contacting the cell with at least about 0.001 pg/mL of a capsid that comprises a capsid polypeptide comprising at least 100 consecutive amino acids of SEQ ID NO: 1 and a heterologous cargo that is not associated with the polypeptide comprising the at least 100 consecutive amino acids of SEQ ID NO: 1 in nature.

Disclosed herein, in some aspects, is a method of delivering a cargo to a cell, the method comprising contacting the cell with at least about 0.001 pg/mL of a capsid that comprises a native PNMA2 polypeptide or an engineered PNMA2 polypeptide and a heterologous cargo that is not associated the endogenous retroviral capsid polypeptide in nature.

In some embodiments, the cell is a mammalian cell. In some embodiments, the cell is a human cell. In some embodiments, the cell is contacted with the capsid at a concentration of at least about at least about 0.01 pg/mL, at least about 0.1 pg/mL, at least about 1 pg/mL, at least about 10 pg/mL, at least about 100 pg/mL, at least about 1 ng/mL, at least about 10 ng/mL, at least about 100 ng/mL, at least about 1 μg/mL, at least about 10 pg/mL, at least about 100 pg/mL, at least about 1 mg/mL, at least about 10 mg/mL, or at least about 100 mg/mL. In some embodiments, the contacting is in vivo. In some embodiments, the contacting is in vitro or ex vivo. In some embodiments, the heterologous cargo comprises a nucleic acid, wherein the cell expresses a gene encoded by the nucleic acid after the delivering. In some embodiments, the heterologous cargo comprises a protein, a peptide, or an antibody or binding fragment thereof, a peptidomimetic, a nucleotidomimetic, a drug, a diagnostic tool, an imaging tool, a small molecule, or a combination thereof. In some embodiments, the heterologous cargo comprises or encodes a gene editing system or a component thereof. In some embodiments, the heterologous cargo comprises a CRISPR/Cas system or a component thereof. In some embodiments, the heterologous cargo comprises a zinc finger nuclease system or a component thereof. In some embodiments, the heterologous cargo comprises a TALEN system or a component thereof.

Disclosed herein, in some aspects, is a capsid comprising a recombinant PNMA2 polypeptide and a heterologous cargo.

In some embodiments, the heterologous cargo is a nucleic acid. In some embodiments, the heterologous cargo is a DNA. In some embodiments, the heterologous cargo is an RNA. In some embodiments, the heterologous cargo is a therapeutic agent. In some embodiments, the heterologous cargo is a protein, a peptide, or an antibody or binding fragment thereof, a peptidomimetic, a nucleotidomimetic, a drug, a diagnostic tool, an imaging tool, or a small molecule. In some embodiments, the recombinant PNMA2 polypeptide is a mammalian PNMA2. In some embodiments, the recombinant PNMA2 polypeptide is a human PNMA2. In some embodiments, the recombinant PNMA2 polypeptide comprises an amino acid sequence with at least 90% sequence identity to SEQ ID NO: 7, SEQ ID NO: 8, or SEQ ID NO: 1. In some embodiments, the PNMA2 polypeptide comprises an amino acid sequence with at least 91%, at least 92%, at least 93%, at least 94%, at least 95%, at least 96%, at least 97%, at least 98%, at least 99%, or at least 99.5% sequence identity to SEQ ID NO: 7, SEQ ID NO: 8, or SEQ ID NO: 1. In some embodiments, the PNMA2 polypeptide comprises an amino acid sequence that is SEQ ID NO: 7, SEQ ID NO: 8, or SEQ ID NO: 1. In some embodiments, the recombinant PNMA2 polypeptide comprises a sequence modification relative to SEQ ID NO: 1. In some embodiments, the capsid comprises a disulfide bond. In some embodiments, the capsid comprises a disulfide bond that comprises a Cysteine at position 10, 136, 233, or 310 of the recombinant PNMA2 polypeptide relative to SEQ ID NO: 1. In some embodiments, the capsid comprises a disulfide bond that comprises a Cysteine at position 136 of the recombinant PNMA2 polypeptide relative to SEQ ID NO: 1. In some embodiments, the capsid comprises a disulfide bond that comprises a Cysteine at position 310 of the recombinant PNMA2 polypeptide relative to SEQ ID NO: 1. In some aspects, the disclosure provides a composition comprising a plurality of the capsid of any one of the preceding embodiments, wherein the capsids are at least about 5%, at least about 10%, at least about 20%, at least about 30%, at least about 40%, at least about 50%, at least about 60%, at least about 70%, at least about 80%, or at least about 90% pure as determined by SDS-PAGE. In some aspects, the disclosure provides a composition comprising a plurality of the capsid of any one of the preceding embodiments, wherein the capsids comprise at least about 5%, at least about 10%, at least about 20%, at least about 30%, at least about 40%, at least about 50%, at least about 60%, at least about 70%, at least about 80%, or at least about 90% particle homogeneity as determined by multi-angle dynamic light scattering.

Disclosed herein, in some aspects, is an engineered PNMA2 polypeptide comprising a sequence modification relative to SEQ ID NO: 1.

In some embodiments, the sequence modification comprises an amino acid deletion. In some embodiments, the sequence modification comprises an amino acid insertion. In some embodiments, the sequence modification comprises an amino acid substitution. In some embodiments, the sequence modification comprises a cargo binding domain. In some embodiments, the sequence modification comprises a nucleic acid binding domain. In some embodiments, the sequence modification comprises a DNA binding domain. In some embodiments, the sequence modification comprises an RNA binding domain. In some embodiments, the sequence modification comprises a zinc finger domain. In some embodiments, the sequence modification comprises a sub-cellular localization signal. In some embodiments, the sequence modification comprises a nuclear localization signal (NLS). In some embodiments, the sequence modification comprises an arginine-rich domain. In some embodiments, the sequence modification is at an N-terminus of SEQ ID NO: 1. In some embodiments, the sequence modification is at a C-terminus of SEQ ID NO: 1. In some embodiments, the sequence modification is within SEQ ID NO: 1. In some aspects, the disclosure provides a nucleic acid encoding the PNMA2 polypeptide of any one of the preceding embodiments. In some embodiments, the disclosure provides a vector comprising the nucleic acid. In some embodiments, the disclosure provides a cell comprising the nucleic acid.

Disclosed herein, in some aspects, is a method of delivering a heterologous cargo to a cell, comprising contacting the cell with the capsid of any one of the preceding embodiments.

In some embodiments, the cell is a eukaryotic cell. In some embodiments, the cell is a vertebrate cell. In some embodiments, the cell is a mammalian cell. In some embodiments, the cell is a human cell. In some embodiments, the cell is contacted with the capsid at a concentration of at least about 0.001 pg/mL, at least about 0.01 pg/mL, at least about 0.1 pg/mL, at least about 1 pg/mL, at least about 10 pg/mL, at least about 100 pg/mL, at least about 1 ng/mL, at least about 10 ng/mL, at least about 100 ng/mL, at least about 1 μg/mL, at least about 10 μg/mL, at least about 100 μg/mL, at least about 1 mg/mL, at least about 10 mg/mL, or at least about 100 mg/mL. In some embodiments, the contacting is in vivo. In some embodiments, the contacting is in vitro or ex vivo. In some embodiments, the heterologous cargo comprises a nucleic acid, wherein the cell expresses a gene encoded by the nucleic acid after the delivering. In some embodiments, the heterologous cargo is a protein, a peptide, or an antibody or binding fragment thereof, a peptidomimetic, a nucleotidomimetic, a drug, a diagnostic tool, an imaging tool, or a small molecule.

Disclosed herein, in some aspects, is an engineered endogenous retroviral capsid polypeptide comprising a cysteine residue, wherein upon isolation the engineered endogenous retroviral capsid polypeptide is capable of assembling to form a capsid with an efficiency of at least 5%.

In some embodiments, upon isolation the engineered endogenous retroviral capsid polypeptide is capable of assembling to form a capsid with an efficiency of at least about 10%, at least about 20%, at least about 30%, at least about 40%, at least about 50%, at least about 60%, at least about 70%, at least about 80%, or at least about 90%. In some embodiments, the engineered endogenous retroviral capsid polypeptide is an endo-Gag polypeptide. In some embodiments, the engineered endogenous retroviral capsid polypeptide is an engineered PNMA protein. In some embodiments, the engineered endogenous retroviral capsid polypeptide is an engineered PNMA2 protein. In some embodiments, the assembling is in a buffer containing less than 2000, less than 1000, less than 900, less than 800, less than 700, less than 600, less than 500, less than 400, less than 300, or less than 200 mOsm/kg of salt.

Disclosed herein, in some aspects, is a capsid comprising an engineered endogenous retroviral capsid polypeptide, wherein the capsid is capable of being disassembled into a non-capsid state with an efficiency of at least 1%, and re-assembled into a capsid state with an efficiency of at least 1%.

In some embodiments, the capsid is capable of being disassembled into a non-capsid state with an efficiency of at least about 5%, at least about 10%, at least about 20%, at least about 30%, at least about 40%, at least about 50%, at least about 60%, at least about 70%, at least about 80%, or at least about 90%. In some embodiments, when in the disassembled non-capsid state, the capsid is capable of being reassembled into the capsid state with an efficiency of at least about 5%, at least about 10%, at least about 20%, at least about 30%, at least about 40%, at least about 50%, at least about 60%, at least about 70%, at least about 80%, or at least about 90%. In some embodiments, the engineered endogenous retroviral capsid polypeptide is an endo-Gag polypeptide. In some embodiments, the engineered endogenous retroviral capsid polypeptide is an engineered PNMA protein. In some embodiments, the engineered endogenous retroviral capsid polypeptide is an engineered PNMA2 protein. In some embodiments, the reassembly efficiency is in determined in a buffer containing less than 2000, less than 1000, less than 900, less than 800, less than 700, less than 600, less than 500, less than 400, less than 300, or less than 200 mOsm/kg of salt.

Disclosed herein, in some aspects, is a method of making a capsid, the method comprising: (a) expressing a PNMA2 polypeptide in a host cell or a cell-free expression system; and (b) isolating the PNMA2 polypeptide; wherein the isolated PNMA2 polypeptide assembles to form the capsid.

In some embodiments, at least about 5% of the isolated PNMA2 polypeptide assembles to form the capsid. In some embodiments, at least about 5%, at least about 10%, at least about 20%, at least about 30%, at least about 40%, at least about 50%, at least about 60%, at least about 70%, at least about 80%, at least about 90%, or at least about 95% of the isolated PNMA2 polypeptide assembles to form the capsid. In some embodiments, the percent of the isolated PNMA2 polypeptide present in capsid form is determined by a multi-angle dynamic light scattering assay. In some embodiments, the method produces assembled capsids of at least about 5%, at least about 10%, at least about 20%, at least about 30%, at least about 40%, at least about 50%, at least about 60%, at least about 70%, at least about 80%, or at least about 90% purity as determined by SDS-PAGE. In some embodiments, the method produces assembled capsids with at least about 5%, at least about 10%, at least about 20%, at least about 30%, at least about 40%, at least about 50%, at least about 60%, at least about 70%, at least about 80%, or at least about 90% particle homogeneity as determined by multi-angle dynamic light scattering. In some embodiments, the isolated PNMA2 polypeptide is assembled in a buffer containing less than 2000, less than 1000, less than 900, less than 800, less than 700, less than 600, less than 500, less than 400, less than 300, or less than 200 mOsm/kg of salt.

Disclosed herein, in some aspects, is a method of making the capsid of any one of the preceding embodiments, the method comprising: (a) expressing a PNMA2 polypeptide in a host cell or a cell-free expression system; (b) isolating the PNMA2 polypeptide; (c) treating the isolated PNMA2 polypeptide with a disassembly buffer, thereby generating PNMA2 polypeptide in a disassembled form; (d) incubating the disassembled PNMA2 polypeptide in a reassembly buffer with the heterologous cargo present; thereby generating the capsid comprising the heterologous cargo.

In some embodiments, the isolated PNMA2 polypeptide is at least partially present in a capsid form prior to step (c). In some embodiments, the PNMA2 polypeptide is the recombinant PNMA2 polypeptide of any one of the preceding embodiments. In some embodiments, the PNMA2 polypeptide is the engineered PNMA2 polypeptide of any one of the preceding embodiments. In some embodiments, at least about 5% of the PNMA2 polypeptide that is present in capsid form before step (c) is disassembled after step (c). In some embodiments, at least about 5% of the PNMA2 polypeptide that is present in disassembled form after step (c) is present in capsid form after step (d). In some embodiments, the percent of the PNMA2 polypeptide that is present in a capsid form or a disassembled form is determined by a multi-angle dynamic light scattering assay. In some embodiments, the disassembly buffer comprises a reducing agent. In some embodiments, the reducing agent is GSH or TCEP. In some embodiments, the disassembly buffer comprises a non-denaturing detergent. In some embodiments, the nondenaturing detergent is CHAPS. In some embodiments, the reassembly buffer contains less than 2000, less than 1000, less than 900, less than 800, less than 700, less than 600, less than 500, less than 400, less than 300, or less than 200 mOsm/kg of salt.

Disclosed herein, in some aspects, is a method of loading PNMA capsids comprising: disassembling PNMA capsids into a composition comprising PNMA monomers; contacting a cargo with the composition comprising PNMA monomers; and reassembling the composition comprising PNMA monomers into PNMA capsids; thereby loading the cargo into an interior of the PNMA capsids.

In some embodiments, the disassembling comprises reducing a disulfide bond. In some embodiments, the reassembling comprises incubating the cargo and the composition comprising PNMA monomers in a physiological buffer. In some embodiments, the reassembling comprises incubating the cargo and the composition comprising PNMA monomers in a buffer that comprises about 270-330 mOsm/kg of salt.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below.

FIG. 1A provides an illustrative alignment of the N-terminal 237 amino acids of PNMA2 to other PNMA family members, with select cysteines and a Lysine and Arginine-rich KRs region shown with boxes.

FIG. 1B provides an illustrative alignment of amino acids 238-364 of PNMA2 to other PNMA family members, with the cysteine at position 310 and a poly-glutamic acid rich region (PolyE) of PNMA2 shown with boxes.

FIG. 2A provides a schematic of an illustrative PNMA2 polypeptide construct that comprises a 6×His tag (SEQ ID NO: 27), TEV cleavage site, linkers, and PNMA2 sequence. FIG. 2A discloses “GSGSGS” as SEQ ID NO: 58.

FIG. 2B provides an outline of the purification procedure of recombinant 6×His-tagged PNMA2 (“6×His tag” disclosed as SEQ ID NO: 27).

FIG. 2C provides an illustrative polyacrylamide gel demonstrating the purity of recombinant PNMA2.

FIG. 2D provides an illustrative electron microscopy image showing concentrated PNMA2 capsids.

FIG. 2E provides a multi-angle dynamic light scattering (MADLS) profile of purified PNMA2 capsids.

FIG. 3A provides a schematic of an illustrative untagged PNMA2 polypeptide construct that comprises PNMA2 sequence.

FIG. 3B provides an outline of the purification procedure of recombinant untagged PNMA2.

FIG. 3C provides an illustrative polyacrylamide gel demonstrating the purity of recombinant untagged PNMA2.

FIG. 3D provides an illustrative electron microscopy image showing concentrated PNMA2 capsids comprising untagged PNMA2 polypeptides.

FIG. 3E provides a multi-angle dynamic light scattering (MADLS) profile of purified PNMA2 capsids comprising untagged PNMA2 polypeptides.

FIG. 4 provides a schematic of an illustrative engineered PNMA2 polypeptide construct with functional elements on the N- or C-terminus of the polypeptide.

FIG. 5A provides a schematic of an illustrative engineered PNMA2 polypeptide construct that comprises a 6×His tag (SEQ ID NO: 27), TEV cleavage site, linkers, an engineered RNA binding domain, and PNMA2 sequence. FIG. 5A discloses “GSGSGS” as SEQ ID NO: 58.

FIG. 5B provides an outline of the purification procedure of recombinant engineered 6×His-tagged PNMA2 (“6×His tag” disclosed as SEQ ID NO: 27) with an RNA binding domain.

FIG. 5C provides an illustrative polyacrylamide gel demonstrating the purity of the recombinant engineered 6×His-tagged PNMA2 (“6×His tag” disclosed as SEQ ID NO: 27) with an RNA binding domain.

FIG. 6A provides a schematic of an illustrative engineered untagged PNMA2 polypeptide construct that comprises an engineered RNA binding domain and PNMA2 sequence.

FIG. 6B provides an outline of the purification procedure of recombinant engineered untagged PNMA2 with an RNA binding domain.

FIG. 6C provides an illustrative polyacrylamide gel demonstrating the purity of the recombinant engineered untagged PNMA2 with an RNA binding domain.

FIG. 7A shows an alignment of the cysteine residues present in PNMA2 to equivalent residues in PNMA1, PNMA3, PNMA4, PNMA5, and PNMA6A, showing that some cysteines are conserved between PNMA family members (e.g., C10, C233), while other are not (e.g., C136, C310).

FIG. 7B shows the results of an SDS-PAGE and western blot assay, demonstrating that dimers, trimers, tetramers, and many higher-order interactions were observed for wild type PNMA2, and that the PNMA2 multimers were disrupted when disulfide bonds were reduced by TCEP treatment. C10S, C136S, C233S, and C310S mutant forms of PNMA2 exhibited reduced multimerization.

FIG. 8A shows an illustrative transmission electron microscopy image (top of the panel) and multi-angle dynamic light scattering (MADLS) profile (bottom of the panel) of initially purified PNMA2 capsids.

FIG. 8B shows an illustrative transmission electron microscopy image (top of the panel) and multi-angle dynamic light scattering (MADLS) profile (bottom of the panel) of chemically disassembled PNMA2 capsids.

FIG. 8C shows an illustrative transmission electron microscopy image (top of the panel) and multi-angle dynamic light scattering (MADLS) profile (bottom of the panel) of reassembled PNMA2 capsids.

FIG. 9A is a Mono Q chromatograph showing the association of RNA with PNMA2 capsids.

FIG. 9B quantifies RNA extracted from the PNMA2 capsid peak of the Mono Q chromatograph presented in FIG. 9A, demonstrating separation of PNMA2 capsids from the bulk of RNA while an RNA of approximately 116 nt tightly associated with the capsids.

FIG. 10A is a schematic illustrating packaging of an RNA cargo in PNMA2 capsids during reassembly.

FIG. 10B shows results of a gel-shift assay demonstrating association of a 67 nt RNA with reassembled PNMA2 capsids. The upshifted is associated with the RNA the reassembled PNMA2 capsids.

FIG. 11A provides a schematic of reassembly process of PNMA2 constructs with different engineered RNA binding domains into the corresponding RNA-loaded capsids.

FIG. 11B provides illustrative transmission electron microscopy images of the corresponding reassembled RNA or DNA-loaded PNMA2 capsids.

FIG. 11C provides representative images of gel-shift assays demonstrating association of various RNA or DNA cargos with Mono Q purified (nucleic acid free) PNMA2 polypeptides comprising various RNA-binding domains in reassembled capsids.

FIG. 12A provides a representative image of a gel shift assay demonstrating protection of various amounts of RNA cargo packaged in reassembled PNMA2 capsids from degradation by benzonase.

FIG. 12B provides a representative image of a gel shift assay demonstrating protection of various amounts of RNA cargo packaged in reassembled PNMA2 capsids from degradation by RNaseA.

FIG. 13A provides a schematic of two PNMA2 constructs with and without the N-terminal 6×His tag (SEQ ID NO: 27).

FIG. 13B schematically outlines the process of reassembly of hybrid capsids containing a mixture of untagged and 6×His tagged PNMA2 subunits (“6×His tag” disclosed as SEQ ID NO: 27).

FIG. 13C provides a representative transmission electron microscopy image of reassembled hybrid PNMA2 capsids.

FIG. 13D provides an illustrative image of a Coomassie R-250-stained denaturing polyacrylamide gel demonstrating the subunit composition of hybrid PNMA2 capsids comprising various amounts of untagged and 6×His-tagged PNMA2 polypeptides following reassembly and affinity purification on Ni-NTA agarose (“6×His tag” disclosed as SEQ ID NO: 27).

FIG. 14A shows a epifluorescence microscopy image of mock-treated neurons (incubated without PNMA2), then stained with anti-PNMA2 antibodies. The dotted line encloses the location of the nucleus.

FIG. 14B shows a fluorescence microscopy image of neurons that were incubated with PNMA2 capsids, washed, and then stained with anti-PNMA2 antibodies. A punctate staining pattern is observed, suggesting that the PNMA2 capsids were internalized by the neurons. The dotted line encloses the location of the nucleus.

FIG. 14C and FIG. 14D show epifluorescence microscopy images of tissue from a C57BL6 mouse that was injected intramuscularly in the hind quarter with 10 μg of PNMA2 capsids.

FIG. 14D is an image of stained muscle tissue from the injected side, while FIG. 14C is an image of stained muscle tissue from the contralateral (uninjected) hind quarter.

FIG. 15A, top panel, provides schematics of the wild type and ΔCYS PNMA2 proteins. Positions of the original cysteine residues within the wild type PNMA2 polypeptide are indicated. FIG. 15A, bottom panel provides a transmission electron microscopy image of PNMA2 ΔCYS capsids demonstrating that the cysteines are not essential for capsid assembly.

FIG. 15B, upper panel, provides schematic of a PNMA2 ΔCYS+6×GS-CYS construct with Alexa 647 dye conjugated to the single cysteine C-terminal residue via maleimide chemistry (“6×GS-CYS” disclosed as SEQ ID NO: 59). FIG. 15B, bottom panel, shows PNMA2 ΔCYS+6×GS-CYS coupled to Alexa 647 run on a denaturing SDS-polyacrylamide gel and either stained with Coomassie R-250 or visualized by Alexa 657 fluorescence.

FIG. 15C demonstrates intracellular detection of Alexa 647-labeled PNMA2 capsids by fluorescent microscopy in HCT116 colon carcinoma cells.

FIG. 16A provides an illustration of potential modifications to the PNMA2 protein to facilitate cellular targeting.

FIG. 16B is an illustration of a camelid heavy chain-only antibody (HCAb).

FIG. 16C depicts the addition of a single domain camelid antibody heavy chain variable region fragment (VH) to the N terminus of a PNMA2 construct (PNMA2-VH).

FIG. 16D demonstrates cellular targeting of PNMA2 capsids in A431 cancer cells, an epidermoid carcinoma with elevated expression of EGFR. Left two images, cells were treated with PNMA2 capsids, right two images, cells were treated with capsids that comprised 10% PNMA2-VH (anti-EGFR). Top two images show staining pattern from anti-PNMA2 primary antibody, bottom two images show fluorescence signal from anti-EGFR primary antibody.

FIG. 17A left panel is an illustration of unmodified PNMA2 protein structure, right panel shows immunofluorescence staining of PNMA2 after treatment of HeLa cells with the unmodified PNMA2.

FIG. 17B left panel is an illustration depicting the mixing of PNMA2 capsids and a positivity charged peptide comprised of 9 arginines (R9) (SEQ ID NO: 56). Right panel shows immunofluorescence staining of PNMA2 after treatment of HeLa cells with the PNMA2 capsid/peptide mixture.

FIG. 17C left panel depicts the addition of a cellular penetrating peptide (CPP) to the N terminus of the PNMA2 protein while the right panel shows immunofluorescence staining of PNMA2 after treatment of HeLa cells with the PNMA2-CPP.

FIG. 18A illustrates an experimental set up for administration of Cre mRNA loaded PNMA2 capsids onto U87 MG cells that constitutively express a LoxP-GFP-stop-LoxP-RFP (GFP/RFP switch) cassette.

FIG. 18B shows RFP expression in cells after incubation with Cre mRNA loaded PNMA2 capsids or unpackaged Cre mRNA (post nuclease treatment).

FIG. 19A illustrates an experimental set up for treatment of GFP/RFP switch cells with Cre mRNA loaded PNMA2-RBD capsids.

FIG. 19B shows RFP expression in GFP/RFP switch cells after treatment with Cre mRNA loaded PNMA2-RBD capsids (top panel) or Cre mRNA alone (bottom panel).

FIG. 20A illustrates an experimental set up for treatment of GFP/RFP switch cells with Cre mRNA loaded PNMA2-RBD capsids with the addition of a cationic lipid.

FIG. 20B shows RFP expression in cells after incubation with Cre mRNA loaded PNMA2-RBD capsids (top panel) or Cre mRNA alone (bottom panel) post nuclease treatment and the addition of cationic lipid.

FIG. 20C is a graph of the percent of “GFP+” pixels that also are “RFP+”, this serves as a surrogate readout of the percent of cells where Cre mRNA was successfully delivered and induced RFP expression.

FIG. 21A illustrates an experimental set up for treatment of GFP/RFP switch cells with Cre mRNA loaded PNMA2-RBD capsids with the addition of a cationic peptide.

FIG. 21B shows RFP expression in cells after incubation with Cre mRNA loaded PNMA2-RBD capsids (top panel) or Cre mRNA alone (bottom panel) post nuclease treatment and the addition of cationic peptide.

FIG. 22A illustrates an experimental set up for treatment of THP-1 reporter cells with hairpin RNA (hpRNA) loaded PNMA2-RBD capsids with the optional addition of transduction enhancers.

FIG. 22B depicts the cell-based assay for detecting delivery of hpRNA into THP-1 cells. THP-1 monocytes will express a secreted form of luciferase upon hpRNA induced interferon (INF) stimulation.

FIG. 22C is a plot of luminescence from culture media of THP-1 cells that were treated with either hpRNA loaded PNMA2-RBD capsids alone, loaded capsids with the addition of cationic polymer, or loaded capsids with the addition of cationic lipid.

FIG. 23A illustrates the experimental set up for treatment of GFP/RFP switch cells with pre-formed RNMA2-RBD capsids that are then incubated with Cre mRNA prior to nuclease treatment and the addition of a cationic lipid.

FIG. 23B shows RFP expression in cells after incubation with pre-formed PNMA2-RBD capsids mixed with Cre mRNA, either with (top panel) or without (bottom panel) the addition of nuclease.

FIG. 24A details the experimental set up for the administration of Cre mRNA loaded PNMA2-RBD capsids into Ai14 mice that are genetically modified to express robust tdTomato fluorescence following Cre-mediated recombination.

FIG. 24B shows tdTomato expression in cells associated with skeletal muscle after intramuscular (IM) injection of Cre mRNA loaded PNMA2-RBD capsids. FIG. 25A shows a multi-angle dynamic light scattering (MADLS) profile of disassembled PNMA2 capsids.

FIG. 25B provides an illustrative electron microscopy image showing disassembled PNMA2 capsids.

FIG. 25C shows a multi-angle dynamic light scattering (MADLS) profile of reassembled PNMA2 capsids.

FIG. 25D provides an illustrative electron microscopy image showing reassembled PNMA2 capsids.

FIG. 26 depicts a size exclusion chromatography trace demonstrating separation of assembled PNMA2 capsids from disassembled monomers. The vertical axis is mAU of 280.

### DETAILED DESCRIPTION OF THE DISCLOSURE

Administering diagnostic or therapeutic agents to a site of interest with precision has presented an ongoing challenge. Available methods of delivering nucleic acids to cells suffer from a number of limitations. For example, AAV viral vectors often used for gene therapy are immunogenic, have a limited payload capacity, suffer from poor bio-distribution, can only be administered by direct injection, and pose a risk of disrupting host genes by integration. Some studies have suggested that a significant proportion (e.g., ≥50%) of the population have pre-existing immunity to viral vectors such as AAV, which could severely limit their effectiveness in therapeutic applications. The utility of existing non-viral methods is also restricted by a number of shortcomings. Liposomes can be primarily delivered to the liver. Extracellular vesicles can have a limited payload capacity, limited scalability, and be subject to purification difficulties. Thus, there is a need for new and improved compositions and methods for delivering therapeutic payloads. Capsids (or virus-like particles, VLPs) disclosed herein have the potential to address many of these shortcomings.

Disclosed herein, in certain embodiments, are endogenous retroviral capsid polypeptides, such as endogenous Gag (endo-Gag) polypeptides. In some embodiments, endo-Gag polypeptides of the disclosure, such as PNMA2 polypeptides, assemble into capsids for delivery of a cargo of interest. Endo-Gag polypeptides of the disclosure exhibit surprising and unexpected advantages over existing and alternate capsid-forming polypeptides, such as improved efficiency in capsid assembly, capsid disassembly, and/or capsid reassembly, e.g., reassembly with a heterologous cargo. In additional embodiments, described herein are capsids, e.g., PNMA2-based or endo-Gag-based capsids, for delivery of a cargo of interest. Also disclosed herein are engineered endo-Gag polypeptides, such as PNMA2 polypeptides, with additional modifications of particular advantage and utility.

### Endogenous Gag Polypeptides and PNMA2 Polypeptides

Endogenous Gag (endo-Gag) proteins are eukaryotic proteins that have predicted and annotated similarity to viral Gag proteins. As described herein, in some embodiments an endo-Gag protein is capable of assembling into a capsid (VLP) state or form. Endo-Gag proteins of the disclosure can be useful, for example, for packaging and delivering cargos (e.g., heterologous cargos) to cells.

The Paraneoplastic Ma (PNMA) family of endo-Gag proteins comprises 15 members, PNMA1, PNMA2, PNMA3, PNMA4 (MOAP1), PNMA5, PNMA6A, PNMA6B/6D, PNMA6E, PNMA6F, PNMA7A, PNMA7B, PNMA8A, PNMA8B, PNMA8C, and CCDC8. PNMA family members share sequence homology to the Gag protein of Long Terminal Repeat (LTR) retrotransposons. PNMA2 proteins are discussed in Pang et al., (2018) PNMA family: Protein interaction network and cell signalling pathways implicated in cancer and apoptosis. Cellular signalling, 45, 54-62, which is incorporated herein by reference for such disclosure.

PNMA polypeptides comprise several domains. Most PNMA family members share high sequence homology at the N-terminal conserved domain (NCD), and the central conserved domain (CCD). In some family members, a unique protein sequence or domain (UPD) is situated between NCD and CCD domains. A region rich in Lysine and Arginine basic residues, designated KRs is also found in the protein sequences of most members of PNMA family. Many PNMA family members share sequence homology at a poly-glutamic acid rich region (PolyE) near the C-terminus. Beyond the polyglutamic acid rich region is the C-terminus with varying length and low sequence homology (variable C-terminal sequence, VCS) that can be identified in the protein sequences among the PNMA family members. Homologues of PNMA proteins exist in other mammalian species, for example, chimpanzee, monkey, rat, and mouse. In some family members, electrostatic interaction between the KRs and PolyE contribute to protein conformation.

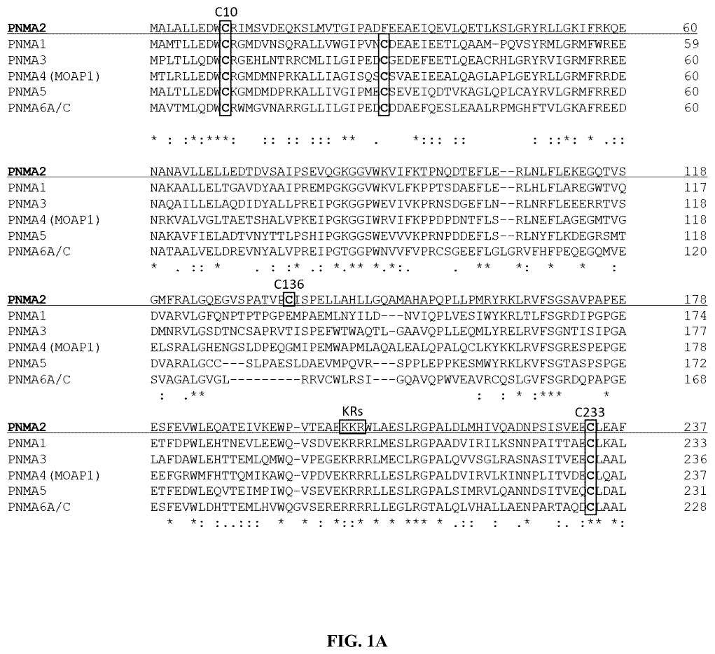

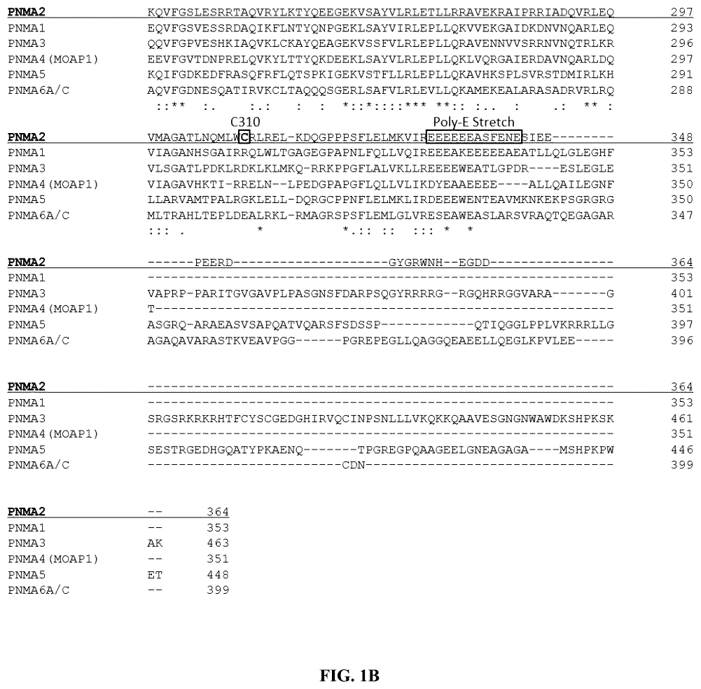

PNMA2 is one member of the PNMA family. An illustrative PNMA2 is human PNMA2, which can comprise 364 amino acids, e.g., as provided in SEQ ID NO: 1. PNMA2 comprises a central conserved domain (CCD), for example, at residues 202-204 of SEQ ID NO: 1, and a poly-glutamic acid rich region (PolyE) near the C-terminus, for example, at residues 333-338 of SEQ ID NO: 1. PNMA2 can form heterodimers with PNMA1 and PNMA4 (MOAP1), for example to modulate (e.g., inhibit) apoptotic signaling. PNMA2 can also comprise cysteine residues that have the potential to for disulfide bonds, for example, at positions 10, 136, 233, and 310 of SEQ ID NO: 1. An illustrative alignment of PNMA2 to other PNMA family members is provided in FIGS. 1A and 1B, with full sequences of illustrative PNMA family members provided in TABLE 1.

TABLE 1

| SEQ | | |
|---|---|---|
| ID | | |
| NO: | Description | Sequence |
| 1 | PNMA2 | MALALLEDWCRIMSVDEQKSLMVTGIPADFEEAEIQEVLQETLKSLG |
| | amino acid | RYRLLGKIFRKQENANAVLLELLEDTDVSAIPSEVQGKGGVWKVIFK |
| | sequence | TPNQDTEFLERLNLFLEKEGQTVSGMFRALGQEGVSPATVPCISPELL |
| | | AHLLGQAMAHAPQPLLPMRYRKLRVFSGSAVPAPEEESFEVWLEQA |
| | | TEIVKEWPVTEAEKKRWLAESLRGPALDLMHIVQADNPSISVEECLE |
| | | AFKQVFGSLESRRTAQVRYLKTYQEEGEKVSAYVLRLETLLRRAVE |
| | | KRAIPRRIADQVRLEQVMAGATLNQMLWCRLRELKDQGPPPSFLEL |
| | | MKVIREEEEEEASFENESIEEPEERDGYGRWNHEGDD |
| 2 | PNMA1 | MAMTLLEDWCRGMDVNSQRALLVWGIPVNCDEAEIEETLQAAMPQ |
| | amino acid | VSYRMLGRMFWREENAKAALLELTGAVDYAAIPREMPGKGGVWK |
| | sequence | VLFKPPTSDAEFLERLHLFLAREGWTVQDVARVLGFQNPTPTPGPEM |
| | | PAEMLNYILDNVIQPLVESIWYKRLTLFSGRDIPGPGEETFDPWLEHT |
| | | NEVLEEWQVSDVEKRRRLMESLRGPAADVIRILKSNNPAITTAECLK |
| | | ALEQVFGSVESSRDAQIKFLNTYQNPGEKLSAYVIRLEPLLQKVVEK |
| | | GAIDKDNVNQARLEQVIAGANHSGAIRRQLWLTGAGEGPAPNLFQL |
| | | LVQIREEEAKEEEEEAEATLLQLGLEGHF |
| 3 | PNMA3 | MPLTLLQDWCRGEHLNTRRCMLILGIPEDCGEDEFEETLQEACRHLG |
| | amino acid | RYRVIGRMFRREENAQAILLELAQDIDYALLPREIPGKGGPWEVIVKP |
| | sequence | RNSDGEFLNRLNRFLEEERRTVSDMNRVLGSDTNCSAPRVTISPEFW |
| | | TWAQTLGAAVQPLLEQMLYRELRVFSGNTISIPGALAFDAWLEHTTE |
| | | MLQMWQVPEGEKRRRLMECLRGPALQVVSGLRASNASITVEECLAA |
| | | LQQVFGPVESHKIAQVKLCKAYQEAGEKVSSFVLRLEPLLQRAVENN |
| | | VVSRRNVNQTRLKRVLSGATLPDKLRDKLKLMKQRRKPPGFLALVK |
| | | LLREEEEWEATLGPDRESLEGLEVAPRPPARITGVGAVPLPASGNSFD |
| | | ARPSQGYRRRRGRGQHRRGGVARAGSRGSRKRKRHTFCYSCGEDG |
| | | HIRVQCINPSNLLLVKQKKQAAVESGNGNWAWDKSHPKSKAK |
| 4 | PNMA4 | MTLRLLEDWCRGMDMNPRKALLIAGISQSCSVAEIEEALQAGLAPLG |
| | (MOAP1) | EYRLLGRMFRRDENRKVALVGLTAETSHALVPKEIPGKGGIWRVIFK |
| | amino acid | PPDPDNTFLSRLNEFLAGEGMTVGELSRALGHENGSLDPEQGMIPEM |
| | sequence | WAPMLAQALEALQPALQCLKYKKLRVFSGRESPEPGEEEFGRWMFH |
| | | TTQMIKAWQVPDVEKRRRLLESLRGPALDVIRVLKINNPLITVDECL |
| | | QALEEVFGVTDNPRELQVKYLTTYQKDEEKLSAYVLRLEPLLQKLV |
| | | QRGAIERDAVNQARLDQVIAGAVHKTIRRELNLPEDGPAPGFLQLLV |
| | | LIKDYEAAEEEEALLQAILEGNFT |
| 5 | PNMA5 | MALTLLEDWCKGMDMDPRKALLIVGIPMECSEVEIQDTVKAGLQPL |
| | amino acid | CAYRVLGRMFRREDNAKAVFIELADTVNYTTLPSHIPGKGGSWEVV |
| | sequence | VKPRNPDDEFLSRLNYFLKDEGRSMTDVARALGCCSLPAESLDAEV |
| | | MPQVRSPPLEPPKESMWYRKLKVFSGTASPSPGEETFEDWLEQVTEI |
| | | MPIWQVSEVEKRRRLLESLRGPALSIMRVLQANNDSITVEQCLDALK |
| | | QIFGDKEDFRASQFRFLQTSPKIGEKVSTFLLRLEPLLQKAVHKSPLSV |
| | | RSTDMIRLKHLLARVAMTPALRGKLELLDQRGCPPNFLELMKLIRDE |
| | | EEWENTEAVMKNKEKPSGRGRGASGRQARAEASVSAPQATVQARS |
| | | FSDSSPQTIQGGLPPLVKRRRLLGSESTRGEDHGQATYPKAENQTPGR |
| | | EGPQAAGEELGNEAGAGAMSHPKPWET |
| 6 | PNMA6A | MAVTMLQDWCRWMGVNARRGLLILGIPEDCDDAEFQESLEAALRP |
| | amino acid | MGHFTVLGKAFREEDNATAALVELDREVNYALVPREIPGTGGPWNV |
| | sequence | VFVPRCSGEEFLGLGRVFHFPEQEGQMVESVAGALGVGLRRVCWLR |
| | | SIGQAVQPWVEAVRCQSLGVFSGRDQPAPGEESFEVWLDHTTEMLH |
| | | VWQGVSERERRRRLLEGLRGTALQLVHALLAENPARTAQDCLAALA |
| | | QVFGDNESQATIRVKCLTAQQQSGERLSAFVLRLEVLLQKAMEKEA |
| | | LARASADRVRLRQMLTRAHLTEPLDEALRKLRMAGRSPSFLEMLGL |
| | | VRESEAWEASLARSVRAQTQEGAGARAGAQAVARASTKVEAVPGG |
| | | PGREPEGLLQAGGQEAEELLQEGLKPVLEECDN |

Additional examples of endo-Gag proteins are disclosed in Campillos et al., (2006) Computational characterization of multiple Gag-like human proteins, Trends Genet 22(11):585-9, which is incorporated herein by reference for such disclosure. Arc (activity-regulated cytoskeleton-associated protein) is another example of an endo-Gag protein. Arc regulates the endocytic trafficking of α-amino-3-hydroxy-5-methylisoxazole-4-propionic acid (AMPA) type glutamate receptors. Arc activities have been linked to synaptic strength and neuronal plasticity. Phenotypes of loss of Arc in experimental murine model included defective formation of long-term memory and reduced neuronal activity and plasticity.

In certain embodiments, disclosed herein is an endo-Gag polypeptide. It should be understood that in some embodiments, endo-Gag sequences are optional substitutes for PNMA2 sequences to form engineered PNMA2 polypeptides disclosed herein. For example, in some embodiments, a sequence from a PNMA family member other than PNMA2, or from another endo-Gag polypeptide, can be substituted for a PNMA2 sequence to form an engineered PNMA2 polypeptide of the disclosure.

In some embodiments, an endo-Gag polypeptide or PNMA polypeptide disclosed herein is not Arc or does not comprise an amino acid sequence from Arc. In some embodiments, an endo-Gag polypeptide or PNMA polypeptide disclosed herein is not PNMA5 or does not comprise an amino acid sequence from PNMA5. In some embodiments, an endo-Gag polypeptide or PNMA polypeptide disclosed herein is not PNMA6 or does not comprise an amino acid sequence from PNMA6. In some embodiments, an endo-Gag polypeptide or PNMA polypeptide disclosed herein is not PNMA6A or does not comprise an amino acid sequence from PNMA6A. In some embodiments, an endo-Gag polypeptide or PNMA polypeptide disclosed herein is not PNMA6B or does not comprise an amino acid sequence from PNMA6B. In some embodiments, an endo-Gag polypeptide or PNMA polypeptide disclosed herein is not a protein from the retrotransposon Gag-like family or does not comprise an amino acid sequence from a protein from the retrotransposon Gag-like family. In some embodiments, an endo-Gag polypeptide or PNMA polypeptide disclosed herein is not PEG10 (RTL2) or does not comprise an amino acid sequence from PEG10 (RTL2). In some embodiments, an endo-Gag polypeptide or PNMA polypeptide disclosed herein is not RTL1 or does not comprise an amino acid sequence from RTL1. In some embodiments, an endo-Gag polypeptide or PNMA polypeptide disclosed herein is not RTL3 or does not comprise an amino acid sequence from RTL3. In some embodiments, an endo-Gag polypeptide or PNMA polypeptide disclosed herein is not RTL6 or does not comprise an amino acid sequence from RTL6. In some embodiments, an endo-Gag polypeptide or PNMA polypeptide disclosed herein is not RTL8A or does not comprise an amino acid sequence from RTL8A. In some embodiments, an endo-Gag polypeptide or PNMA polypeptide disclosed herein is not RTL8B or does not comprise an amino acid sequence from RTL8B. In some embodiments, an endo-Gag polypeptide or PNMA polypeptide disclosed herein is not BOP (RTL10) or does not comprise an amino acid sequence from BOP (RTL10). In some embodiments, an endo-Gag polypeptide or PNMA polypeptide disclosed herein is not LDOC1 (RTL7) or does not comprise an amino acid sequence from LDOC1 (RTL7). In some embodiments, an endo-Gag polypeptide or PNMA polypeptide disclosed herein is not MOAP1 (PNMA4) or does not comprise an amino acid sequence from MOAP1 (PNMA4). In some embodiments, an endo-Gag polypeptide or PNMA polypeptide disclosed herein is not ZNF18 or does not comprise an amino acid sequence from ZNF18. In some embodiments, an endo-Gag polypeptide or PNMA polypeptide disclosed herein is not Asprv1 or does not comprise an amino acid sequence from Asprv1. In some embodiments, an endo-Gag polypeptide or PNMA polypeptide disclosed herein is not CCDC8 or does not comprise an amino acid sequence from CCDC8. In some embodiments, an endo-Gag polypeptide or PNMA polypeptide disclosed herein is not PGBD1 or does not comprise an amino acid sequence from PGBD1. In some embodiments, an endo-Gag polypeptide or PNMA polypeptide disclosed herein is not PNMA1 or does not comprise an amino acid sequence from PNMA1. In some embodiments, an endo-Gag polypeptide or PNMA polypeptide disclosed herein is not PNMA3 or does not comprise an amino acid sequence from PNMA3. In some embodiments, an endo-Gag polypeptide or PNMA polypeptide disclosed herein is not PNMA5 or does not comprise an amino acid sequence from PNMA5. In some embodiments, an endo-Gag polypeptide or PNMA polypeptide disclosed herein is not PNMA6B/6D or does not comprise an amino acid sequence from PNMA6B/6D. In some embodiments, an endo-Gag polypeptide or PNMA polypeptide disclosed herein is not PNMA6E or does not comprise an amino acid sequence from PNMA6E. In some embodiments, an endo-Gag polypeptide or PNMA polypeptide disclosed herein is not PNMA6F or does not comprise an amino acid sequence from PNMA6F. In some embodiments, an endo-Gag polypeptide or PNMA polypeptide disclosed herein is not PNMA7A or does not comprise an amino acid sequence from PNMA7A. In some embodiments, an endo-Gag polypeptide or PNMA polypeptide disclosed herein is not PNMA7B or does not comprise an amino acid sequence from PNMA7B. In some embodiments, an endo-Gag polypeptide or PNMA polypeptide disclosed herein is not PNMA8A or does not comprise an amino acid sequence from PNMA8A. In some embodiments, an endo-Gag polypeptide or PNMA polypeptide disclosed herein is not PNMA8B or does not comprise an amino acid sequence from PNMA8B. In some embodiments, an endo-Gag polypeptide or PNMA polypeptide disclosed herein is not PNMA8C or does not comprise an amino acid sequence from PNMA8C. In some embodiments, an endo-Gag polypeptide or PNMA polypeptide disclosed herein is not SCAN or does not comprise an amino acid sequence from SCAN. In some embodiments, an endo-Gag polypeptide or PNMA polypeptide disclosed herein is not ZCCHC12 or does not comprise an amino acid sequence from ZCCHC12. In some embodiments, an endo-Gag polypeptide or PNMA polypeptide disclosed herein is not ZCCHC18 or does not comprise an amino acid sequence from ZCCHC18. In some embodiments, an endo-Gag polypeptide or PNMA polypeptide disclosed herein is not ZNF274 or does not comprise an amino acid sequence from ZNF274. In some embodiments, an endo-Gag polypeptide or PNMA polypeptide disclosed herein is not a SUSHI family member or does not comprise an amino acid sequence from a SUSHI family member. In some embodiments, an endo-Gag polypeptide or PNMA polypeptide disclosed herein is not a SCAN family member or does not comprise an amino acid sequence from a SCAN family member. In some embodiments, an endo-Gag polypeptide or PNMA polypeptide disclosed herein is not PNMA2 or does not comprise an amino acid sequence from PNMA2.

In some instances, the PNMA2 polypeptide comprises a full-length PNMA2 polypeptide. In other instances, the PNMA2 polypeptide comprises a fragment of PNMA2, such as a truncated PNMA2 polypeptide, that participates in the formation of a capsid. In further instances, the PNMA2 polypeptide is a recombinant PNMA2 polypeptide.

In some instances, the endo-Gag polypeptide comprises a full-length endo-Gag protein. In other instances, the endo-Gag polypeptide comprises a fragment of an endo-Gag protein, such as a truncated endo-Gag polypeptide, that can participate in the formation of a capsid. In further instances, the endo-Gag polypeptide is a recombinant endo-Gag polypeptide.

In some instances, the PNMA2 or endo-Gag polypeptide comprises from N-terminus to C-terminus the following domains: NCD, UPD, CCD, KRs, PolyE, and VCS.

In some embodiments, one or more non-essential regions which are not involved in capsid formation are removed from a PNMA2 or endo-Gag protein to generate an engineered PNMA2 or endo-Gag polypeptide. In such instances, one or more non-essential regions, e.g., an N-terminal region (e.g., up to 10 amino acids, up to 15 amino acids, up to 20 amino acids, up to 25 amino acids, up to 30 amino acids, up to 40 amino acids, or up to 50 amino acids), a C-terminal region (e.g., up to 10 amino acids, up to 15 amino acids, up to 20 amino acids, up to 25 amino acids, up to 30 amino acids, up to 40 amino acids, or up to 50 amino acids), or a combination thereof, are deleted from a PNMA2 or endo-Gag protein (e.g., a native or wild type PNMA2 or endo-Gag protein) to generate an engineered PNMA2 or endo-Gag polypeptide.

In some embodiments, at least 5, at least 10, at least 15, at least 20, at least 25, at least 30, at least 35, at least 40, at least 45, or at least 50 amino acids are removed from an N-terminus of a PNMA2 or endo-Gag protein (e.g., a native or wild type PNMA2 or endo-Gag protein) to generate an engineered PNMA2 or endo-Gag polypeptide. In some embodiments, at most 5, at most 10, at most 15, at most 20, at most 25, at most 30, at most 35, at most 40, at most 45, or at most 50 amino acids are removed from an N-terminus of a PNMA2 or endo-Gag protein (e.g., a native or wild type PNMA2 or endo-Gag protein) to generate an engineered PNMA2 or endo-Gag polypeptide. In some embodiments, about 5, about 10, about 15, about 20, about 25, about 30, about 35, about 40, about 45, or about 50, amino acids are removed from an N-terminus of a PNMA2 or endo-Gag protein (e.g., a native or wild type PNMA2 or endo-Gag protein) to generate an engineered PNMA2 or endo-Gag polypeptide. In some embodiments, about 1 to about 50, about 1 to about 40, about 1 to about 35, about 1 to about 30, about 1 to about 27, about 1 to about 25, about 1 to about 23, about 1 to about 20, about 1 to about 15, about 1 to about 10, about 1 to about 5, about 5 to 50, about 5 to 40, about 5 to 35, about 5 to 30, about 5 to 27, about 5 to 25, about 5 to 23, about 5 to 20, about 5 to 15, about 5 to 10, about 10 to 50, about 10 to 40, about 10 to 35, about 10 to 30, about 10 to 27, about 10 to 25, about 10 to 23, about 10 to 20, about 10 to 15, about 15 to 50, about 15 to 40, about 15 to 35, about 15 to 30, about 15 to 27, about 15 to 25, about 15 to 23, about 15 to 20, about 20 to 50, about 20 to 40, about 20 to 35, about 20 to 30, about 20 to 27, about 20 to 25, or about 20 to 23 amino acids are removed from an N-terminus of a PNMA2 or endo-Gag protein (e.g., a native or wild type PNMA2 or endo-Gag protein) to generate an engineered PNMA2 or endo-Gag polypeptide.

In some embodiments, at least 5, at least 10, at least 15, at least 20, at least 25, at least 30, at least 35, at least 40, at least 45, or at least 50 amino acids are removed from a C-terminus of a PNMA2 or endo-Gag protein (e.g., a native or wild type PNMA2 or endo-Gag protein) to generate an engineered PNMA2 or endo-Gag polypeptide. In some embodiments, at most 5, at most 10, at most 15, at most 20, at most 25, at most 30, at most 35, at most 40, at most 45, or at most 50 amino acids are removed from a C-terminus of a PNMA2 or endo-Gag protein (e.g., a native or wild type PNMA2 or endo-Gag protein) to generate an engineered PNMA2 or endo-Gag polypeptide. In some embodiments, about 5, about 10, about 15, about 20, about 25, about 30, about 35, about 40, about 45, or about 50, amino acids are removed from a C-terminus of a PNMA2 or endo-Gag protein (e.g., a native or wild type PNMA2 or endo-Gag protein) to generate an engineered PNMA2 or endo-Gag polypeptide. In some embodiments, about 1 to about 50, about 1 to about 40, about 1 to about 35, about 1 to about 30, about 1 to about 27, about 1 to about 25, about 1 to about 23, about 1 to about 20, about 1 to about 15, about 1 to about 10, about 1 to about 5, about 5 to 50, about 5 to 40, about 5 to 35, about 5 to 30, about 5 to 27, about 5 to 25, about 5 to 23, about 5 to 20, about 5 to 15, about 5 to 10, about 10 to 50, about 10 to 40, about 10 to 35, about 10 to 30, about 10 to 27, about 10 to 25, about 10 to 23, about 10 to 20, about 10 to 15, about 15 to 50, about 15 to 40, about 15 to 35, about 15 to 30, about 15 to 27, about 15 to 25, about 15 to 23, about 15 to 20, about 20 to 50, about 20 to 40, about 20 to 35, about 20 to 30, about 20 to 27, about 20 to 25, or about 20 to 23 amino acids are removed from a C-terminus of a PNMA2 or endo-Gag protein (e.g., a native or wild type PNMA2 or endo-Gag protein) to generate an engineered PNMA2 or endo-Gag polypeptide.

In some embodiments, at least 15 amino acids are removed from a native or wild type PNMA2 to generate an engineered PNMA2 polypeptide. In some embodiments, at most 25 amino acids are removed from a C-terminus of a native or wild type PNMA2 to generate an engineered PNMA2 polypeptide. In some embodiments, about 25 amino acids are removed from a native or wild type PNMA2 to generate an engineered PNMA2 polypeptide. In some embodiments, about 10 to 30 amino acids are removed from a native or wild type PNMA2 to generate an engineered PNMA2 polypeptide. In some embodiments, about 20 to 27 amino acids are removed from a native or wild type PNMA2 to generate an engineered PNMA2 polypeptide. In some embodiments, about 24 to 26 amino acids are removed from a native or wild type PNMA2 to generate an engineered PNMA2 polypeptide.

In some cases, only the essential regions involved in capsid assembly/forming and cargo binding remain in a PNMA2 or endo-Gag polypeptide. In additional cases, only the essential regions involved in capsid assembly/forming remain in a PNMA2 polypeptide. In some embodiments, a PNMA2 or endo-Gag polypeptide comprises truncations or modifications of domains involved in capsid forming, nucleic acid binding, or delivery.

In some instances, the PNMA2 or endo-Gag polypeptide comprises from N-terminus to C-terminus the following domains: UPD, CCD, KRs, PolyE, and VCS. In some instances, the PNMA2 or endo-Gag polypeptide comprises from N-terminus to C-terminus the following domains: NCD, CCD, KRs, PolyE, and VCS. In some instances, the PNMA2 or endo-Gag polypeptide comprises from N-terminus to C-terminus the following domains: NCD, UPD, KRs, PolyE, and VCS. In some instances, the PNMA2 or endo-Gag polypeptide comprises from N-terminus to C-terminus the following domains: NCD, UPD, CCD, PolyE, and VCS. In some instances, the PNMA2 or endo-Gag polypeptide comprises from N-terminus to C-terminus the following domains: NCD, UPD, CCD, KRs, and VCS. In some instances, the PNMA2 or endo-Gag polypeptide comprises from N-terminus to C-terminus the following domains: NCD, UPD, CCD, KRs, and PolyE. In some instances, each of the domains is either directly or indirectly fused to the respective two flanking domains. In some embodiments a PNMA2 polypeptide or an endo-Gag polypeptide disclosed herein comprises a tag, for example, an epitope tag or an affinity tag, such as a polyhistidine tag. In some embodiments a PNMA2 polypeptide or an endo-Gag polypeptide disclosed herein lacks a tag, for example, does not contain an epitope tag, does not contain an affinity tag, or does not contain a polyhistidine tag. In some embodiments a tag is initially present but is removed, e.g., by enzymatic digestion. In some embodiments a PNMA2 polypeptide or an endo-Gag polypeptide disclosed herein comprises an N-terminal methionine. In some embodiments a PNMA2 polypeptide or an endo-Gag polypeptide disclosed herein lacks an N-terminal methionine. In some embodiments an N-terminal methionine is removed, co-translationally and/or via enzymatic digestion.

In some instances, the domains are arranged in an order that does not impede capsid assembly and cargo binding. In some instances, each of the domains is either directly or indirectly fused to the respective two flanking domains.

In some instances, the PNMA2 or endo-Gag polypeptide is an engineered PNMA2 or endo-Gag polypeptide. As used herein, an engineered polypeptide is a recombinant polypeptide that is not identical in sequence to a full length, wild-type polypeptide. An engineered PNMA2 or endo-Gag polypeptide can be functionally distinct from a native PNMA2 or endo-Gag polypeptide, for example, comprises an additional function than the native polypeptide or lacks a function that the native polypeptide has. In some instances, the PNMA2 or endo-Gag polypeptide is a native PNMA2 or endo-Gag polypeptide, for example, comprising, consisting essentially of, or consisting of a wild type amino acid sequence.

In some instances, the PNMA2 or endo-Gag polypeptide comprises a cargo binding domain. In some embodiments, a PNMA2 or endo-Gag polypeptide is engineered to comprise a cargo binding domain, for example, through insertion of a cargo binding domain, or modification of a cargo binding domain that is endogenous to the PNMA2 or endo-Gag polypeptide. In some embodiments, the cargo binding domain is endogenous to the PNMA2 or endo-Gag polypeptide.

A cargo binding domain can bind a cargo covalently or non-covalently, e.g., via a linker disclosed herein. A linker can be a peptide linker, for example, a peptide linker that binds the cargo binding domain to a peptide or polypeptide cargo. A linker can be a non-peptide linker disclosed herein that binds to the cargo via a covalent bond or a non-covalent bond as disclosed herein. A Cargo binding domain can be joined to a PNMA2 or endo-Gag polypeptide covalently or non-covalently, e.g., via a linker disclosed herein.

In some embodiments a cargo binding domain binds a heterologous cargo, for example, a cargo that is not native to capsids formed from the PNMA2 or endo-Gag polypeptide in nature.

In some cases, the cargo binding domain comprises a nucleic acid binding domain, an RNA binding domain, a DNA binding domain, a protein binding domain, a peptide binding domain, an antibody binding domain, a small molecule binding domain, or a peptidomimetic/nucleotidomimetic binding domain. Exemplary cargo binding domains include, but are not limited to, synthetic nucleic acid (e.g., RNA and/or DNA) binding domains, zinc finger domains, arginine-rich domains, domains from GPCRs, antibodies or binding fragments thereof, lipoproteins, integrins, tyrosine kinases, DNA-binding proteins, RNA-binding proteins, nucleases, ligases, proteases, integrases, isomerases, phosphatases, GTPases, aromatases, esterases, adaptor proteins, G-proteins, GEFs, cytokines, interleukins, interleukin receptors, interferons, interferon receptors, caspases, transcription factors, neurotrophic factors and their receptors, growth factors and their receptors, signal recognition particle and receptor components, extracellular matrix proteins, integral components of membrane, ribosomal proteins, translation elongation factors, translation initiation factors, GPI-anchored proteins, tissue factors, dystrophin, utrophin, dystrobrevin, any fusions, combinations, subunits, derivatives, or domains thereof. In some embodiments, a cargo binding domain comprises a domain from an endogenous Gag polypeptide or a PNMA polypeptide that is heterologous with respect to the remainder of the protein or the part of the protein that induces capsid formation. For example, a PNMA2 polypeptide can be engineered to comprise a cargo-binding domain that is from or derived from a heterologous endo-Gag polypeptide, such as a heterologous PNMA protein, e.g., PNMA3 or another endo-Gag protein.

In some embodiments, a cargo biding domain or a nucleic acid binding domain is from or derived from a viral protein, for example, TAT protein of HIV. In some embodiments, a cargo biding domain is inverted, for example, compared to a wild type version of the cargo binding domain.

A Cargo binding domain can be a synthetic cargo binding domain, for example, a synthetic nucleic acid binding domain. In some embodiments, a synthetic cargo binding domain is designed such that it comprises an extension of a C-terminal alpha helix of a PNMA2 polypeptide or endo-Gag polypeptide. In some embodiments, a synthetic cargo binding domain is designed such that the cargo binding domain is oriented to the interior of an assembled PNMA2 or endo-Gag capsid.

A nucleic acid binding domain can be from or derived from an RNA binding protein. A nucleic acid binding domain can be from or derived from a DNA binding protein.

In some embodiments, a cargo binding domain is lysine-rich, for example, comprising at least 5%, at least 10%, at least 15%, at least 20%, at least 25%, at least 30%, at least 35%, or at least 40% lysine residues. In some embodiments, a cargo binding domain is arginine-rich, for example, comprising at least 5%, at least 10%, at least 15%, at least 20%, at least 25%, at least 30%, at least 35%, or at least 40% arginine residues. In some embodiments, a cargo binding domain comprises one or more Lys/Arg (RK) repeats, for example, at least 1, at least 2, at least 3, at least 4, at least 5, at most 2, at most 3, at most 4, at most 5, about 1, about 2, about 3, about 4, or about 5 Lys/Arg repeats.

Illustrative, non-limiting examples of cargo binding domains that are nucleic acid-binding domains are the amino acid sequences SEQ ID NO: 39, SEQ ID NO: 42, and SEQ ID NO: 45, and amino acid sequences that comprise at least 70%, at least 80%, at least 85%, at least 90%, or at least 95% sequence identity to any one of SEQ ID NOs: 39, 72, and 45.

In some cases, the cargo binding domain binds to an RNA, for example, an mRNA, hairpin RNA, guide RNA, shRNA, siRNA, an mRNA, a tRNA, an rRNA, a snRNA, a microRNA, or a non-coding RNA. In some cases, the cargo binding domain binds to a DNA, for example, a ssDNA, dsDNA, or oligonucleotide. In some embodiments, a cargo binding domain binds to RNA and DNA. In some embodiments, a cargo binding domain preferentially binds RNA over DNA. In some embodiments, a cargo binding domain preferentially binds DNA over RNA. In some embodiments, a cargo binding domain preferentially binds ssDNA. In some embodiments, a cargo binding domain specifically or preferentially binds a particular nucleic acid structural motif, for example, a hairpin, such as a bulge hairpin. In some embodiments, a cargo binding domain non-specifically binds nucleic acids, RNA, and/or DNA, such as ssDNA.

In some embodiments, the PNMA2 is an engineered PNMA2 polypeptide with at least an RNA binding domain inserted and/or modified to bind to a heterologous cargo that is not native to capsids formed from the PNMA2 polypeptide in nature. In some instances, the PNMA2 polypeptide comprises a full-length PNMA2 polypeptide with at least an RNA binding domain inserted and/or modified to bind to a heterologous cargo that is not native to capsids formed from the PNMA2 protein in nature. In additional instances, the engineered PNMA2 polypeptide comprises one or more domains of a PNMA2 polypeptide, in which at least one of the domains participates in the formation of a capsid and in which an RNA binding domain is inserted and/or modified to bind to a heterologous cargo that the native PNMA2 protein does not bind to.

In some embodiments, the endo-Gag (e.g., PNMA family member) is an engineered Endo-Gag polypeptide with at least an RNA binding domain inserted and/or modified to bind to a heterologous cargo that is not native to capsids formed from the endo-Gag protein in nature. In some instances, the endo-Gag polypeptide comprises a full-length endo-Gag polypeptide with at least its RNA binding domain modified to bind to a heterologous cargo that is not native to capsids formed from the endo-Gag protein in nature. In other instances, the endo-Gag polypeptide comprises an engineered endo-Gag fragment comprising modification(s) in at least its RNA binding domain to bind to a heterologous cargo that a native endo-Gag protein does not bind to. In additional instances, the endo-Gag polypeptide comprises one or more domains of an engineered endo-Gag polypeptide, in which at least one of the domains participates in the formation of a capsid and in which the RNA binding domain is modified to bind to a heterologous cargo that is not native to capsids formed from the endo-Gag protein in nature.

In some embodiments, a cargo binding domain is at a C-terminus of an endo-Gag polypeptide or PNMA2 polypeptide disclosed herein. In some embodiments, a cargo binding domain is at an N-terminus of an endo-Gag polypeptide or PNMA2 polypeptide disclosed herein.

In some embodiments, a cargo binding domain can be inserted adjacent to a domain of a PNMA2 or endo-Gag polypeptide. For example, in some embodiments, a cargo binding domain can be inserted adjacent to (e.g., N-terminal or C-terminal to) one or more of an NCD, CCD, UPD, KRs, PolyE, or VCS domain.

In some instances, the PNMA2 or endo-Gag polypeptide comprises from N-terminus to C-terminus the following domains: cargo binding domain, and PolyE. In some instances, the PNMA2 or endo-Gag polypeptide comprises from N-terminus to C-terminus the following domains: PolyE, and cargo binding domain.

In some instances, the PNMA2 or endo-Gag polypeptide comprises from N-terminus to C-terminus the following domains: cargo binding domain, and KRs. In some instances, the PNMA2 or endo-Gag polypeptide comprises from N-terminus to C-terminus the following domains: KRs, and cargo binding domain.

In some instances, the PNMA2 or endo-Gag polypeptide comprises from N-terminus to C-terminus the following domains: cargo binding domain, KRs, and PolyE. In some instances, the PNMA2 or endo-Gag polypeptide comprises from N-terminus to C-terminus the following domains: KRs, cargo binding domain, and PolyE. In some instances, the PNMA2 or endo-Gag polypeptide comprises from N-terminus to C-terminus the following domains: KRs, PolyE, and cargo binding domain. In some instances, each of the domains is either directly or indirectly fused to the respective two flanking domains.

In some instances, the PNMA2 or endo-Gag polypeptide comprises from N-terminus to C-terminus the following domains: cargo binding domain, NCD, UPD, CCD, KRs, PolyE, and VCS. In some instances, the PNMA2 or endo-Gag polypeptide comprises from N-terminus to C-terminus the following domains: NCD, cargo binding domain, UPD, CCD, KRs, PolyE, and VCS. In some instances, the PNMA2 or endo-Gag polypeptide comprises from N-terminus to C-terminus the following domains: NCD, UPD, cargo binding domain, CCD, KRs, PolyE, and VCS. In some instances, the PNMA2 or endo-Gag polypeptide comprises from N-terminus to C-terminus the following domains: NCD, UPD, CCD, cargo binding domain, KRs, PolyE, and VCS. In some instances, the PNMA2 or endo-Gag polypeptide comprises from N-terminus to C-terminus the following domains: NCD, UPD, CCD, KRs, cargo binding domain, PolyE, and VCS. In some instances, the PNMA2 or endo-Gag polypeptide comprises from N-terminus to C-terminus the following domains: NCD, UPD, CCD, KRs, PolyE, cargo binding domain, and VCS. In some instances, the PNMA2 or endo-Gag polypeptide comprises from N-terminus to C-terminus the following domains: NCD, UPD, CCD, KRs, PolyE, VCS, and cargo binding domain. In some instances, each of the domains is either directly or indirectly fused to the respective two flanking domains.

In certain embodiments, a domain of a PNMA2 or endo-Gag polypeptide is replaced, partially replaced, or acts as an insertion site for a cargo binding domain. For example, in some embodiments, one or more of an NCD, CCD, UPD, KRs, PolyE, or VCS domain can be replaced, partially replaced, or act as an insertion site for a cargo binding domain.

In some embodiments, an engineered PNMA2 or endo-Gag polypeptide is modified to comprise an exogenous polypeptide sequence, e.g., in addition to the cargo-binding domain. The exogenous polypeptide sequence can be fused to the PNMA2 or endo-Gag polypeptide. The exogenous polypeptide sequence can be covalently or non-covalently attached to the PNMA2 or endo-Gag polypeptide, e.g., via a linker disclosed herein.

In some embodiments, the exogenous polypeptide sequence can comprise a sub-cellular localization signal, for example, a nuclear localization signal (NLS), or a sequence that targets the polypeptide to a membrane (e.g., an Arginine-rich domain). In some embodiments, the exogenous polypeptide sequence comprises a domain that binds to a cell surface molecule, for example, an antigen, a polypeptide, a receptor, a lipid (e.g., phospholipid), a lipoprotein, a glycoprotein, or the like. In some embodiments, an exogenous polypeptide recognizes and binds to receptors displayed on the surface of targeted cells. Upon reaching a cell of interest, the cargo is optionally further delivered to an intracellular target. For example, a therapeutic RNA can be translated to a protein if it comes into contact with a ribosome in the cytoplasm of the cell.

In some embodiments, the cell surface molecule is a cell surface protein. In some instances, the cell surface molecule is an antigen expressed by a cancerous cell. In some instances, the cell surface molecule is a neoepitope. In some instances, the cell surface molecule comprises one or more mutations compared to a wild-type protein. Illustrative cancer antigens that can be bound by an exogenous polypeptide sequence include, but are not limited to, alpha fetoprotein, ASLG659, B7-H3, BAFF-R, Brevican, CA125 (MUC16), CA15-3, CA19-9, carcinoembryonic antigen (CEA), CA242, CRIPTO (CR, CR1, CRGF, CRIPTO, TDGF1, teratocarcinoma-derived growth factor), CTLA-4, CXCR5, E16 (LAT1, SLC7A5), FcRH2 (IFGP4, IRTA4, SPAP1A (SH2 domain containing phosphatase anchor protein 1a), SPAP1B, SPAP1C), epidermal growth factor, ETBR, Fc receptor-like protein 1 (FCRH1), GEDA, HLA-DOB (Beta subunit of MHC class II molecule (Ia antigen), human chorionic gonadotropin, ICOS, IL-2 receptor, IL20Rα, Immunoglobulin superfamily receptor translocation associated 2 (IRTA2), L6, Lewis Y, Lewis X, MAGE-1, MAGE-2, MAGE-3, MAGE 4, MART1, mesothelin, MDP, MPF (SMR, MSLN), MCP1 (CCL2), macrophage inhibitory factor (MIF), MPG, MSG783, mucin, MUC1-KLH, Napi3b (SLC34A2), nectin-4, Neu oncogene product, NCA, placental alkaline phosphatase, prostate specific membrane antigen (PMSA), prostatic acid phosphatase, PSCA hlg, anti-transferrin receptor, p97, Purinergic receptor P2X ligand-gated ion channel 5 (P2X5), LY64 (Lymphocyte antigen 64 (RP105), gp100, P21, six transmembrane epithelial antigen of prostate (STEAP1), STEAP2, Sema 5b, tumor-associated glycoprotein 72 (TAG-72), TrpM4 (BR22450, F1120041, TRPM4, TRPM4B, transient receptor potential cation channel, subfamily M, member 4) and the like.

In some instances, the cell surface molecule bound by an exogenous polypeptide sequence comprises a cluster of differentiation (CD) cell surface marker, for example, CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD71, CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), or the like.

In some cases, the exogenous polypeptide sequence is or is derived from a protein (e.g., a human protein), an antibody or binding fragment thereof, a viral protein, a Gag-like protein (e.g., a human Gag-like protein), or a de novo engineered protein designed to bind to a target receptor of interest. In some instances, the antibody or binding fragment thereof comprises a humanized antibody or binding fragments thereof, a murine antibody or binding fragment thereof, a chimeric antibody or binding fragment thereof, a monoclonal antibody or binding fragment thereof, a multi-specific antibody or binding fragment thereof, a bispecific antibody or biding fragment thereof, a monovalent Fab′, a divalent Fab₂, F(ab)′₃ fragments, a single-chain variable fragment (scFv), a bis-scFv, an (scFv)₂, a diabody, a minibody, a nanobody, a triabody, a tetrabody, a disulfide stabilized Fv protein (dsFv), a single-domain antibody (sdAb), an Ig NAR, a camelid antibody or binding fragment thereof (e.g., VHH domain), or a chemically modified derivative thereof. In some instances, the exogenous polypeptide sequence guides the delivery of a capsid formed by the engineered PNMA2 polypeptide to a target site of interest.

An antibody or an antigen-binding fragment thereof disclosed herein (e.g., as an exogenous polypeptide sequence or a cargo) can comprise complementarity determining regions (CDRs). In some embodiments, the CDRs determine or substantially determine binding specificity and/or affinity of the antibody or antigen-binding fragment. For example, the CDRs can be grafted onto a different suitable framework, or the framework region can be altered (e.g., via amino acid substitutions, deletions, and/or insertions), and the antigen-binding fragment or domain can retain binding for the target, and the extracellular binding domain remains functional despite the alterations outside of the CDRs. CDRs can be identified by various methods, including but not limited to the Kabat method, the Chothia method, the IMGT method, the AHO method, and the Paratome method. One antigen binding site of an antibody with heavy and light chains or variable regions therefrom comprises six CDRs, three in the hypervariable regions of the light chain variable region, and three in the hypervariable regions of the heavy chain variable region. The CDRs in the light chain are designated L1, L2, and L3, while the CDRs in the heavy chain are designated H1, H2, and H3. CDRs can also be designated LCDR1, LCDR2, LCDR3, HCDR1, HCDR2, and HCDR3, respectively. Certain antibodies or antigen-binding domains contain less than six CDRs. For example, certain antibodies lack a light chain, and can be referred to as heavy chain only antibodies (HCAbs). HCAbs have three CDRs in a variable region referred to as VHH. A single domain antibody, or nanobody, can be generated from such a VHH region of a heavy chain only antibody.

Non-liming examples of exogenous polypeptide sequences include those that encode zinc finger domains, arginine-rich domains, domains from GPCRs, antibodies or binding fragments thereof, lipoproteins, integrins, tyrosine kinases, DNA-binding proteins, RNA-binding proteins, nucleases, ligases, proteases, integrases, isomerases, phosphatases, GTPases, aromatases, esterases, adaptor proteins, G-proteins, GEFs, cytokines, interleukins, interleukin receptors, interferons, interferon receptors, caspases, transcription factors, neurotrophic factors and their receptors, growth factors and their receptors, signal recognition particle and receptor components, extracellular matrix proteins, integral components of membrane, ribosomal proteins, translation elongation factors, translation initiation factors, GPI-anchored proteins, tissue factors, dystrophin, utrophin, dystrobrevin, cell penetrating peptides, fusogenic proteins, viral envelope proteins, endogenous retroviral envelope proteins, any fusions, combinations, subunits, derivatives, or domains thereof.

An exogenous polypeptide sequence can be or can comprise a cell penetrating peptide (CPP). Cell-penetrating peptides (CPPs) can facilitate uptake of macromolecules through cellular membranes and enhance the delivery of CPP-modified molecules to the inside of a cell. A cell-penetrating peptide can comprise or can be a cationic CPP (e.g., TAT, R8, DPV3, DPV6, Penetratin, R9-TAT), an amphipathic CPP (e.g., pVEC, ARF, MPG, MAP, transportan), or a hydrophobic CPP (e.g., Bip4, C105Y, Melttin, gH625). A CPP can be a protein-derived CPP, a synthetic CPP, or a chimeric CPP. CPPs can be comprise amphipathic helical peptides, such as transportan and MAP, where lysine residues are major contributors to the positive charge, and Arg-rich peptides, such as TATp, Antennapedia or penetratin. Other CPPs can include: the minimal protein transduction domain of Antennapedia, a Drosophila homeoprotein, called penetratin, which is a 16-mer peptide (residues 43-58) present in the third helix of the homeodomain; a 27-amino acid-long chimeric CPP, containing the peptide sequence from the amino terminus of the neuropeptide galanin bound via the Lys residue, mastoparan, a wasp venom peptide; VP22, a major structural component of HSV-1 facilitating intracellular transport, and transportan (18-mer) amphipathic model peptide that translocates plasma membranes of mast cells and endothelial cells by both energy-dependent and—independent mechanisms. In some embodiments, a lipid moiety is modified with CPP(s), for intracellular.

In some embodiments, the exogenous polypeptide sequence comprises a non-native cysteine residue, for example, a cysteine residue that is not present in a native form of the PNMA2 polypeptide or endo-Gag polypeptide. The exogenous cysteine residue can be used, for example, for conjugation to a binging partner via suitable chemical reactions, such as Maleimide chemistry. In some embodiments, the exogenous polypeptide sequence comprises a non-native cysteine residue and lacks one or more native cysteine residues, for example, the one or more native cysteine residues are deleted or substituted for non-cysteine residues. The non-native cysteine residue or an exogenous polypeptide sequence comprising the non-native cysteine residue can be fused directly or via a linker to a C-terminus, N-terminus, or within the PNMA2 polypeptide or endo Gag polypeptide, e.g., within or between domains of the PNMA2 polypeptide or endo Gag polypeptide as disclosed herein.

In some instances, the exogenous polypeptide sequence is fused directly, indirectly via a linker, or chemically to one or more of: a cargo binding domain, NCD, UPD, CCD, KRs, PolyE, or VCS, if present. In some instances, the exogenous polypeptide sequence is fused directly, indirectly via a linker, or chemically to a C-terminus of the PNMA2 or endo-Gag polypeptide. In some instances, the exogenous polypeptide sequence is fused directly, indirectly via a linker, or chemically to an N-terminus of the PNMA2 or endo-Gag polypeptide.

In some embodiments, an exogenous polypeptide sequence can be inserted adjacent to a domain of a PNMA2 or endo-Gag polypeptide. For example, in some embodiments, an exogenous polypeptide sequence can be inserted adjacent to (e.g., N-terminal or C-terminal to) one or more of an NCD, CCD, UPD, KRs, PolyE, or VCS domain.

In some instances, the PNMA2 or endo-Gag polypeptide comprises from N-terminus to C-terminus the following domains: exogenous polypeptide sequence, and PolyE. In some instances, the PNMA2 or endo-Gag polypeptide comprises from N-terminus to C-terminus the following domains: PolyE, and exogenous polypeptide sequence.

In some instances, the PNMA2 or endo-Gag polypeptide comprises from N-terminus to C-terminus the following domains: exogenous polypeptide sequence, and KRs. In some instances, the PNMA2 or endo-Gag polypeptide comprises from N-terminus to C-terminus the following domains: KRs, and exogenous polypeptide sequence.

In some instances, the PNMA2 or endo-Gag polypeptide comprises from N-terminus to C-terminus the following domains: exogenous polypeptide sequence, KRs, and PolyE. In some instances, the PNMA2 or endo-Gag polypeptide comprises from N-terminus to C-terminus the following domains: KRs, exogenous polypeptide sequence, and PolyE. In some instances, the PNMA2 or endo-Gag polypeptide comprises from N-terminus to C-terminus the following domains: KRs, PolyE, and exogenous polypeptide sequence. In some instances, each of the domains is either directly or indirectly fused to the respective two flanking domains.

In some instances, the PNMA2 or endo-Gag polypeptide comprises from N-terminus to C-terminus the following domains: exogenous polypeptide sequence, NCD, UPD, CCD, KRs, PolyE, and VCS. In some instances, the PNMA2 or endo-Gag polypeptide comprises from N-terminus to C-terminus the following domains: NCD, exogenous polypeptide sequence, UPD, CCD, KRs, PolyE, and VCS. In some instances, the PNMA2 or endo-Gag polypeptide comprises from N-terminus to C-terminus the following domains: NCD, UPD, exogenous polypeptide sequence, CCD, KRs, PolyE, and VCS. In some instances, the PNMA2 or endo-Gag polypeptide comprises from N-terminus to C-terminus the following domains: NCD, UPD, CCD, exogenous polypeptide sequence, KRs, PolyE, and VCS. In some instances, the PNMA2 or endo-Gag polypeptide comprises from N-terminus to C-terminus the following domains: NCD, UPD, CCD, KRs, exogenous polypeptide sequence, PolyE, and VCS. In some instances, the PNMA2 or endo-Gag polypeptide comprises from N-terminus to C-terminus the following domains: NCD, UPD, CCD, KRs, PolyE, exogenous polypeptide sequence, and VCS. In some instances, the PNMA2 or endo-Gag polypeptide comprises from N-terminus to C-terminus the following domains: NCD, UPD, CCD, KRs, PolyE, VCS, and exogenous polypeptide sequence. In some instances, each of the domains is either directly or indirectly fused to the respective two flanking domains.

In certain embodiments, a domain of a PNMA2 or endo-Gag polypeptide is replaced, partially replaced, or acts as an insertion site for an exogenous polypeptide sequence. For example, in some embodiments, one or more of an NCD, CCD, UPD, KRs, PolyE, or VCS domain can be replaced, partially replaced, or act as an insertion site for an exogenous polypeptide sequence.

In some instances, PNMA2 is a human PNMA2 polypeptide. In some instances, PNMA2 is a non-human PNMA2 polypeptide. In additional instances, the PNMA2 polypeptide comprises one or more domains of a human PNMA2 polypeptide, in which at least one of the domains participates in the formation of a capsid. In additional instances, the PNMA2 polypeptide comprises one or more domains of a non-human PNMA2 polypeptide, in which at least one of the domains participates in the formation of a capsid.

In some instances, an endo-Gag (e.g., a PNMA family member) is a human endo-Gag polypeptide. In some instances, an endo-Gag (e.g., a PNMA family member) is a non-human endo-Gag polypeptide. In additional instances, the endo-Gag polypeptide comprises one or more domains of a human endo-Gag polypeptide, in which at least one of the domains participates in the formation of a capsid. In additional instances, the endo-Gag polypeptide comprises one or more domains of a non-human endo-Gag polypeptide, in which at least one of the domains participates in the formation of a capsid.

In some instances, an engineered PNMA2 or endo-Gag polypeptide comprises a fragment of a PNMA2 or endo-Gag polypeptide from a first species and at least an additional fragment from a PNMA2 or endo-Gag polypeptide of a second species.

In some embodiments, an exemplary mammalian PNMA2 or endo-Gag protein for expression as a recombinant or engineered PNMA2 or endo-Gag polypeptide is from the species Homo sapiens. Additional exemplary species of primate PNMA2 or endo-Gag proteins for expression as a recombinant or engineered PNMA2 or endo-Gag polypeptide include: Gorilla, Pongo abelii, Pan paniscus, Macaca nemestrina, Chlorocebus sabaeus, Papio anubis, rhinopithecus roxellana, Macaca fascicularis, Nomascus leucogenys, Callithrix jacchus, Aotus nancymaae, Cebus capucinus imitator, Saimiri boliviensis boliviensis, Otolemur garnettii, Macaca mulatta, and Macaca fascicularis.

An exemplary species list of rodent PNMA2 or endo-Gag proteins for expression as a recombinant or engineered PNMA2 or endo-Gag polypeptide includes: fukomys damarensis, Microcebus murinus, Heterocephalus glaber, Propithecus coquereli, Marmota marmota marmota, Galeopterus variegatus, Cavia porcellus, Dipodomys ordii, Octodon degus, castor canadensis Nannospalax galili, Carlito syrichta, Chinchilla lanigera, Mus musculus, Ictidomys tridecemlineatus, Rattus norvegicus, Microtus ochrogaster, Otolemur garnettii, Meriones unguiculatus, Cricetulus griseus, Rattus norvegicus, Neotoma lepida, Jaculus jaculus, Mustela putorius furo, Mesocricetus auratus, Tupaia chinensis, Cricetulus griseus, Chrysochloris asiatica, Elephantulus edwardii, Erinaceus europaeus, Ochotona princeps, Sorex araneus, Monodelphis domestica, Echinops telfairi, and Condylura cristata.

In some embodiments, a nucleic acid sequence or amino acid sequence of the disclosure (for example, encoding a PNMA2 polypeptide or endo-Gag polypeptide, or a fragment or domain thereof) has at least 70% homology, at least 71% homology, at least 72% homology, at least 73% homology, at least 74% homology, at least 75% homology, at least 76% homology, at least 77% homology, at least 78% homology, at least 79% homology, at least 80% homology, at least 81% homology, at least 82% homology, at least 83% homology, at least 84% homology, at least 85% homology, at least 86% homology, at least 87% homology, at least 88% homology, at least 89% homology, at least 90% homology, at least 91% homology, at least 92% homology, at least 93% homology, at least 94% homology, at least 95% homology, at least 96% homology, at least 97% homology, at least 98% homology, at least 99% homology, at least 99.1% homology, at least 99.2% homology, at least 99.3% homology, at least 99.4% homology, at least 99.5% homology, at least 99.6% homology, at least 99.7% homology, at least 99.8% homology, at least 99.9% or at least 99.99% homology to a nucleic acid sequence or an amino acid sequence provided herein.

Various methods and software programs are used to determine the homology (e.g., identity or similarity) between two or sequences. For example, the degree of sequence identity or similarity between two sequences can be determined by comparing the two sequences using computer programs commonly employed for this purpose, such as global or local alignment algorithms. Non-limiting examples include NCBI BLAST, BLASTp, BLASTn, BLASTx, tBLASTn, Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, SSEARCH2SEQ, or another suitable method or algorithm, e.g., using default parameters. A Needleman and Wunsch global alignment algorithm can be used to align two sequences over their entire length, maximizing the number of matches and minimizes the number of gaps.

In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to SEQ ID NO: 7.

In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to at least 100 consecutive amino acids of SEQ ID NO: 7. In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to at least 200 consecutive amino acids of SEQ ID NO: 7. In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to at least 250 consecutive amino acids of SEQ ID NO: 7. In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to at least 300 consecutive amino acids of SEQ ID NO: 7.

In certain embodiments, an endo-Gag or PNMA2 polypeptide of the disclosure (e.g., a recombinant or engineered endo-Gag or PNMA2 polypeptide) comprises, consists essentially of, or consists of an amino acid sequence that is SEQ ID NO: 7.

In certain embodiments, an endo-Gag or PNMA2 polypeptide of the disclosure (e.g., a recombinant or engineered endo-Gag or PNMA2 polypeptide) comprises one or more amino acid substitutions, deletions or insertions, from the polypeptide having the amino acid sequence of SEQ ID NO: 7. In some embodiments, the endo-Gag or PNMA2 polypeptide comprises from 1, 2, 3, 4, 5, 6, 7, 8, 9, 10 or more up to about 100, 90, 80, 70, 60, 50, 45, 40, 35, 30, 25, 20, 15 amino acid substitutions, deletions or insertions relative to SEQ ID NO: 7.

In some embodiments, the endo-Gag or PNMA2 polypeptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least or at least 50 amino acid substitutions, deletions, or insertions relative to SEQ ID NO: 7.

In some embodiments, the endo-Gag or PNMA2 polypeptide comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions, deletions, or insertions relative to SEQ ID NO: 7.

In some embodiments, the endo-Gag or PNMA2 polypeptide comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-4, 3-5, 3-6, 3-7, 3-8, 3-9, 3-10, 3-15, 3-20, 3-30, 3-40, 4-5, 4-6, 4-7, 4-8, 4-9, 4-10, 4-15, 4-20, 4-30, 5-6, 5-7, 5-8, 5-9, 5-10, 5-15, 5-20, 5-30, 5-40, 10-15, 15-20, or 20-25 amino acid substitutions, deletions, or insertions relative to SEQ ID NO: 7.

In some embodiments, the endo-Gag or PNMA2 polypeptide comprises 1, 2, 3, 4, 5, 6, 7, 8, 9, 10, 11, 12, 13, 14, 15, 16, 17, 18, 19, or 20 amino acid substitutions, deletions, or insertions relative to SEQ ID NO: 7. An amino acid substitution can be a conservative or a non-conservative substitution. The one or more amino acid substitutions, deletions, or insertions can be at the N-terminus, the C-terminus, within the amino acid sequence, or a combination thereof. The amino acid substitutions, deletions, or insertions can be contiguous, non-contiguous, or a combination thereof.

In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to SEQ ID NO: 8. In certain embodiments, an endo-Gag or PNMA2 polypeptide of the disclosure (e.g., a recombinant or engineered endo-Gag or PNMA2 polypeptide) comprises, consists essentially of, or consists of an amino acid sequence that is SEQ ID NO: 8.

In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to at least 100 consecutive amino acids of SEQ ID NO: 8. In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to at least 200 consecutive amino acids of SEQ ID NO: 8. In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to at least 250 consecutive amino acids of SEQ ID NO: 8. In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to at least 300 consecutive amino acids of SEQ ID NO: 8.

In certain embodiments, an endo-Gag or PNMA2 polypeptide of the disclosure (e.g., a recombinant or engineered endo-Gag or PNMA2 polypeptide) comprises one or more amino acid substitutions, deletions or insertions, from the polypeptide having the amino acid sequence of SEQ ID NO: 8. In some embodiments, the endo-Gag or PNMA2 polypeptide comprises from 1, 2, 3, 4, 5, 6, 7, 8, 9, 10 or more up to about 100, 90, 80, 70, 60, 50, 45, 40, 35, 30, 25, 20, 15 amino acid substitutions, deletions or insertions relative to SEQ ID NO: 8.

In some embodiments, the endo-Gag or PNMA2 polypeptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least or at least 50 amino acid substitutions, deletions, or insertions relative to SEQ ID NO: 8.

In some embodiments, the endo-Gag or PNMA2 polypeptide comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions, deletions, or insertions relative to SEQ ID NO: 8.

In some embodiments, the endo-Gag or PNMA2 polypeptide comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-4, 3-5, 3-6, 3-7, 3-8, 3-9, 3-10, 3-15, 3-20, 3-30, 3-40, 4-5, 4-6, 4-7, 4-8, 4-9, 4-10, 4-15, 4-20, 4-30, 5-6, 5-7, 5-8, 5-9, 5-10, 5-15, 5-20, 5-30, 5-40, 10-15, 15-20, or 20-25 amino acid substitutions, deletions, or insertions relative to SEQ ID NO: 8.

In some embodiments, the endo-Gag or PNMA2 polypeptide comprises 1, 2, 3, 4, 5, 6, 7, 8, 9, 10, 11, 12, 13, 14, 15, 16, 17, 18, 19, or 20 amino acid substitutions, deletions, or insertions relative to SEQ ID NO: 8. An amino acid substitution can be a conservative or a non-conservative substitution. The one or more amino acid substitutions, deletions, or insertions can be at the N-terminus, the C-terminus, within the amino acid sequence, or a combination thereof. The amino acid substitutions, deletions, or insertions can be contiguous, non-contiguous, or a combination thereof.

In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to SEQ ID NO: 1. In certain embodiments, an endo-Gag or PNMA2 polypeptide of the disclosure (e.g., a recombinant or engineered endo-Gag or PNMA2 polypeptide) comprises, consists essentially of, or consists of an amino acid sequence that is SEQ ID NO: 1.

In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to at least 100 consecutive amino acids of SEQ ID NO: 1. In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to at least 200 consecutive amino acids of SEQ ID NO: 1. In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to at least 250 consecutive amino acids of SEQ ID NO: 1. In certain embodiments, an endo-Gag or PNMA2 polypeptide of the disclosure (e.g., a recombinant or engineered endo-Gag or PNMA2 polypeptide) comprises, consists essentially of, or consists of an amino acid sequence with at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity to at least 300 consecutive amino acids of SEQ ID NO: 1.

In certain embodiments, an endo-Gag or PNMA2 polypeptide of the disclosure (e.g., a recombinant or engineered endo-Gag or PNMA2 polypeptide) comprises one or more amino acid substitutions, deletions or insertions, from the polypeptide having the amino acid sequence of SEQ ID NO: 1. In some embodiments, the endo-Gag or PNMA2 polypeptide comprises from 1, 2, 3, 4, 5, 6, 7, 8, 9, 10 or more up to about 100, 90, 80, 70, 60, 50, 45, 40, 35, 30, 25, 20, 15 amino acid substitutions, deletions or insertions relative to SEQ ID NO: 1.

In some embodiments, the endo-Gag or PNMA2 polypeptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least or at least 50 amino acid substitutions, deletions, or insertions relative to SEQ ID NO: 1.

In some embodiments, the endo-Gag or PNMA2 polypeptide comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions, deletions, or insertions relative to SEQ ID NO: 1.

In some embodiments, the endo-Gag or PNMA2 polypeptide comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-4, 3-5, 3-6, 3-7, 3-8, 3-9, 3-10, 3-15, 3-20, 3-30, 3-40, 4-5, 4-6, 4-7, 4-8, 4-9, 4-10, 4-15, 4-20, 4-30, 5-6, 5-7, 5-8, 5-9, 5-10, 5-15, 5-20, 5-30, 5-40, 10-15, 15-20, or 20-25 amino acid substitutions, deletions, or insertions relative to SEQ ID NO: 1.

In some embodiments, the endo-Gag or PNMA2 polypeptide comprises 1, 2, 3, 4, 5, 6, 7, 8, 9, 10, 11, 12, 13, 14, 15, 16, 17, 18, 19, or 20 amino acid substitutions, deletions, or insertions relative to SEQ ID NO: 1. An amino acid substitution can be a conservative or a non-conservative substitution. The one or more amino acid substitutions, deletions, or insertions can be at the N-terminus, the C-terminus, within the amino acid sequence, or a combination thereof. The amino acid substitutions, deletions, or insertions can be contiguous, non-contiguous, or a combination thereof.

### Linkers

In certain embodiments, a polypeptide or capsid of the disclosure comprises a linker, for example, a peptide or non-peptide linker that joins two components covalently or non-covalently.

A linker can join a first component (e.g., domain, polypeptide, cargo binding domain, cargo, delivery component, or exogenous polypeptide sequence) to a second component (e.g., domain, polypeptide, cargo binding domain, cargo, delivery component, or exogenous polypeptide sequence).

A linker can join a cargo binding domain to a cargo (e.g., covalently or non-covalently). A linker can join a first cargo binding domain to a second cargo binding domain. A linker can join a cargo binding domain to a different domain. A linker can join a cargo binding domain to a polypeptide (e.g., a PNMA2 polypeptide or end-Gag polypeptide). A linker can join a cargo binding domain to an exogenous polypeptide sequence.

A linker can join a cargo to a cargo binding domain. A linker can join a first cargo to a second cargo. A linker can join a cargo to a domain. A linker can join a cargo to a polypeptide (e.g., a PNMA2 polypeptide or end-Gag polypeptide). A linker can join a cargo to an exogenous polypeptide sequence.

A linker can join an exogenous polypeptide sequence to a polypeptide (e.g., a PNMA2 polypeptide or end-Gag polypeptide). A linker can join a first exogenous polypeptide sequence to a second exogenous polypeptide sequence. A linker can join an exogenous polypeptide sequence to a domain. A linker can join an exogenous polypeptide sequence to a cargo binding domain. A linker can join an exogenous polypeptide sequence to a cargo.

A linker can join a first domain to a second domain (e.g., a cargo binding domain). A linker can join a domain to a polypeptide (e.g., a PNMA2 polypeptide or end-Gag polypeptide). A linker can join a domain to a cargo. A linker can join a domain to an exogenous polypeptide sequence.

A linker can join a first polypeptide (e.g., a PNMA2 polypeptide or end-Gag polypeptide) to a second polypeptide. A linker can join a polypeptide (e.g., a PNMA2 polypeptide or end-Gag polypeptide) to a domain. A linker can join a polypeptide (e.g., a PNMA2 polypeptide or end-Gag polypeptide) to a cargo binding domain. A linker can join a polypeptide (e.g., a PNMA2 polypeptide or end-Gag polypeptide) to a cargo. A linker can join a polypeptide (e.g., a PNMA2 polypeptide or end-Gag polypeptide) to an exogenous polypeptide sequence.

A linker can join any two components of a polypeptide, capsid, complex, or composition disclosed herein. For example, a linker can join a PNMA2 polypeptide to a cargo, an endo-Gag polypeptide to a cargo, a PNMA2 polypeptide to a cargo-binding domain, an endo-Gag polypeptide to a cargo-binding domain, a PNMA2 polypeptide to an exogenous polypeptide sequence, an endo Gag polypeptide to an exogenous polypeptide sequence, a PNMA2 polypeptide to a delivery component, an endo-Gag polypeptide to a delivery component, or any other two domains disclosed herein. A linker can join two domains within a PNMA polypeptide, two domains within a cargo, to domains within a cargo-binding domain, two domains within an exogenous polypeptide sequence, two domains within a delivery component, and the like. A polypeptide, capsid, complex, or composition can comprise multiple linkers. For example, a cargo, cargo-binding domain, or exogenous polypeptide sequence that comprises an antigen-binding fragment can comprise two variable regions joined by a linker, such as an scFv. A polypeptide or domain that comprises one or more linkers can be joined to another polypeptide or domains via another linker that is the same or different, for example, a cargo, cargo binding domain, or exogenous polypeptide sequence that is an scFv can comprise a linker between a VH and VL domain, and the scFv can in turn be joined to a PNMA2 or endo-Gag polypeptide by a second linker.

In some embodiments, the linker is a peptide linker. In some instances, the linker is a rigid linker. In other instances, the linker is a flexible linker. In some cases, the linker is a non-cleavable linker. In other cases, the linker is a cleavable linker. In additional cases, the linker comprises a linear structure, or a non-linear structure (e.g., a cyclic structure).

A flexible peptide linker can have a sequence containing stretches of glycine and serine residues. The small size of the glycine and serine residues provides flexibility and allows for mobility of the connected functional domains. The incorporation of serine or threonine can maintain the stability of the linker in aqueous solutions by forming hydrogen bonds with the water molecules, thereby reducing unfavorable interactions between the linker and protein moieties. Flexible linkers can also contain additional amino acids such as threonine and alanine to maintain flexibility, as well as polar amino acids such as lysine and glutamine to improve solubility. A rigid peptide linker can have, for example, an alpha helix-structure. An alpha-helical rigid linker can act as a spacer between certain protein domains. A peptide linker can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues in length. In some cases, a linker sequence can be, for example at least about 2, at least about 3, at least about 4, at least about 5, at least about 6, at least about 7, at least about 8, at least about 9, at least about 10, at least about 15, at least about 20, at least about 30, at least about 40, or at least about 50 amino acids in length. In some cases, a linker sequence can be, for example at most about 2, at most about 3, at most about 4, at most about 5, at most about 6, at most about 7, at most about 8, at most about 9, at most about 10, at most about 15, at most about 20, at most about 30, at most about 40, at most about 50, at most about 60, at most about 70, at most about 80, or at most about 100 amino acids in length. In some cases, a linker is 5-20 amino acids in length. In some cases, a linker is 10-20 amino acids in length. In some cases, a linker is 4-8 amino acids in length.

A linker can be a non-cleavable linker. A non-cleavable linker of the present disclosure can include a chemical linker that is stable. Examples of non-cleavable linkers that can be used in proteins of the present disclosure to link domains and/or polypeptides can include a thioether linker, an alkyl linker, a polymeric linker. A linker may be an SMCC linker or a PEG linker. In some embodiments, the linker may be a PEG linker. A non-cleavable linker can also include a non-proteolytically cleavable peptide linker. A non-proteolytically cleavable peptide can be inert to proteases present in a given sample, tissue, or organism. For example, a peptide can be inert or substantially inert to all or most human protease cleavage sequences, and thereby can comprise a high degree of stability within humans and human samples. Such a peptide can also comprise a secondary structure which renders a protease cleavage site inert or inaccessible to a protease. A non-cleavable linker of the present disclosure can comprise a half-life for cleavage of at least 1 hour, at least 2 hours, at least 4 hours, at least 8 hours, at least 12 hours, at least 16 hours, at least 1 day, at least 2 days, at least 3 days, at least 1 week, at least 2 weeks, or at least 1 month in the presence of human proteases at 25° C. in pH 7 buffer.

In certain embodiments, non-cleavable linkers comprise short peptides of varying lengths. Exemplary non-cleavable linkers include (EAAAK)n (SEQ ID NO: 9), or (EAAAR)n (SEQ ID NO: 10), where n is from 1 to 5, and up to 30 residues of glutamic acid-proline or lysine-proline repeats. In some embodiments, the non-cleavable linker comprises (GS)n, (SG)n, (GGGGS)n (SEQ ID NO: 11) or (GGGS)n (SEQ ID NO: 12), wherein n is 1 to 10 (e.g., 1, 2, 3, 4, 5, 6, 7, 8, 9, or 10); KESGSVSSEQLAQFRSLD (SEQ ID NO: 13); or EGKSSGSGSESKST (SEQ ID NO: 14). In some embodiments, the non-cleavable linker comprises a poly-Gly/Ala polymer.

In certain embodiments, the linker is a cleavable linker, e.g., an extracellular cleavable linker or an intracellular cleavable linker. In some instances, the linker is designed for cleavage in the presence of particular conditions or in a particular environment (e.g., under physiological conditions). For example, the design of a linker for cleavage by specific conditions, such as by a specific enzyme, allows the targeting of cellular uptake to a specific location.

In some embodiments, the linker is a pH-sensitive linker. In one instance, the linker is cleaved under basic pH conditions. In other instance, the linker is cleaved under acidic pH conditions.

In some embodiments, the linker is cleaved in vivo by endogenous enzymes (e.g., proteases) such as serine proteases including but not limited to thrombin, metalloproteases, furin, cathepsin B, necrotic enzymes (e.g., calpains), and the like. For example, a cleavable linker can be used to covalently link a cargo to a cargo binding domain, and linker can be cleaved in vivo to release the cargo. Exemplary cleavable linkers include, but are not limited to, GGAANLVRGG (SEQ ID NO: 15); SGRIGFLRTA (SEQ ID NO: 16); SGRSA (SEQ ID NO: 17); GFLG (SEQ ID NO: 18); ALAL (SEQ ID NO: 19); FK; PIC(Et)F-F (SEQ ID NO: 20), where C(Et) indicates S-ethylcysteine; PR(S/T)(L/I)(S/T) (SEQ ID NO: 21); DEVD (SEQ ID NO: 22); GWEHDG (SEQ ID NO: 23); RPLALWRS (SEQ ID NO: 24); or a combination thereof.

In certain embodiments, a cleavable linker comprises a 2A linker, which can be processed into separate polypeptides co-translationally or after translation. Inclusion of a 2A linker can increase the likelihood that an appropriate ratio of components are produced (e.g., a 1:1, 1:2, 1:3, 1:4, or 1:5 ratio of two components). In some cases, inclusion of a 2A linker can increase the likelihood that equal or close to equal levels of two components are produced (e.g., a capsid subunit and a cargo).

A linker can be a non-peptide linker. A linker can be a chemical linker. A linker can be a chemical bond, for example, a covalent bond or a non-covalent bond. A linker of the disclosure can include a chemical linker. For example, two a first component (e.g., domain, polypeptide, cargo binding domain, cargo, or exogenous polypeptide sequence) can be joined to a second component (e.g., domain, polypeptide, cargo binding domain, cargo, or exogenous polypeptide sequence) by a chemical linker. Each chemical linker of the disclosure can be alkylene, alkenylene, alkynylene, heteroalkylene, cycloalkylene, heterocycloalkylene, arylene, or heteroarylene, any of which is optionally substituted. In some embodiments, a chemical linker of the disclosure can be an ester, ether, amide, thioether, or polyethyleneglycol (PEG). In some embodiments, a linker can reverse the order of the amino acids sequence in a compound where two amino acid sequences are linked, for example, so that the amino acid sequences linked by the linked are head-to-head, rather than head-to-tail. Non-limiting examples of such linkers include diesters of dicarboxylic acids, such as oxalyl diester, malonyl diester, succinyl diester, glutaryl diester, adipyl diester, pimetyl diester, fumaryl diester, maleyl diester, phthalyl diester, isophthalyl diester, and terephthalyl diester. Non-limiting examples of such linkers include diamides of dicarboxylic acids, such as oxalyl diamide, malonyl diamide, succinyl diamide, glutaryl diamide, adipyl diamide, pimetyl diamide, fumaryl diamide, maleyl diamide, phthalyl diamide, isophthalyl diamide, and terephthalyl diamide. Non-limiting examples of such linkers include diamides of diamino linkers, such as ethylene diamine, 1,2-di(methylamino)ethane, 1,3-diaminopropane, 1,3-di(methylamino)propane, 1,4-di(methylamino)butane, 1,5-di(methylamino)pentane, 1,6-di(methylamino)hexane, and piperazine.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

In some embodiments, a non-peptide linker or a chemical linker is a cleavable linker, such as a self-immolative linker.

In some embodiments, a cleavable linker comprises a chemical trigger that controls cleavage (and, e.g., release of a cargo). A chemical trigger that can be used in a linker of the disclosure can be a dipeptide trigger, cathepsin-cleavable trigger, acid-cleavable trigger, GSH-cleavable trigger, Fe(II)-cleavable trigger, enzyme-cleavable trigger, photo-responsive-cleavable trigger, bioorthogonal cleavable trigger, glycosidase cleavable triggers, phosphatase cleavable trigger, Sulfatase cleavable trigger, Hydrazone trigger, Carbonate trigger, Silyl ether trigger, Disulfide trigger, 1,2,4-Trioxolane trigger, Dipeptide trigger, Triglycol (CX) trigger, cBu-Cit trigger, 0-Glucuronide trigger, 0-Galactoside trigger, Pyrophosphate trigger, Arylsulfate trigger, Heptamethine cyanine fluorophore trigger, O-Nitrobenzyl trigger, PC4AP trigger, or a dsProc trigger.

In some embodiments, a linker comprises a Maleimide attachment, Bis(vinylsulfonyl)piperazine attachment, N-methyl-N-phenylvinylsulfonamide attachment, Pt(II)-based attachment, carbamate attachment, carbonate attachment, quaternary ammonium attachment.

In some embodiments, a linker comprises a bond generated by carbonyl condensation, a Staudinger reaction, modified Staudinger ligation, traceless Staudinger ligation, Inverse-electron demand Diels-Alter cycloadditions, Copper-catalyzed azide-alkyne cycloaddition (CuAAC), strain-promoted azide-alkyne cycloaddition (SPAAC), Huisgen cycloaddition, click chemistry, or the like.

In some embodiments, a linker comprises a bond generated between cysteine and a cysteine reactive group, such as malemide or iodoacetamide. For example, cysteine-based conjugation can be used to link two components, such as a cargo binding domain to a cargo. One or more reactive cysteine residue(s) can be introduced at selected positions, for example, via insertion or substitution at a C-terminus of a PNMA2 or endo-Gag polypeptide, an N-terminus of a PNMA2 or endo-Gag polypeptide, or within the PNMA2 or endo-Gag polypeptide (e.g., in a domain disclosed herein or between to domains). The cysteine residue(s) introduced can be joined to another part of a polypeptide via a linker or spacer disclosed herein. The cysteine residue can be introduced such that it does not interfere with structure or function of the PNMA or endo-Gag polypeptide, e.g., the ability to assemble into a capsid state or form, disassemble into a non-capsid state or form, and/or re-assemble into a capsid state or form. Cysteine residues can optionally be deleted or substituted for non-reactive or less-reactive residues elsewhere to reduce or eliminate reactivity or conjugation at undesirable sites.

In some embodiments, a linker comprises a covalent attachment of two components. In some embodiments, a linker comprises a non-covalent attachment of two components. Two components present in a polypeptide, complex, or capsid can be non-covalently coupled, for example, by ionic bonds, hydrogen bonds, interactions mediated by oligomerization or dimerization domains, etc.

### Capsids

In some embodiments, disclosed herein is a capsid. In some instances, the capsid comprises an endo-Gag polypeptide, for example, a PNMA family polypeptide, such as a PNMA2 polypeptide. Additional illustrative endo-Gag polypeptides include PNMA1, PNMA2, PNMA3, PNMA4 (MOAP1), PNMA5, PNMA6A, PNMA6B/6D, PNMA6E, PNMA6F, PNMA7A, PNMA7B, PNMA8A, PNMA8B, PNMA8C, CCDC8, Arc, BOP, LDOC1, PEG10, RTL3, RTL6, RTL8A, RTL8B, and ZNF18. In some instances, the polypeptide is a functional fragment, e.g., that is capable of forming a subunit of a capsid. PNMA or end-Gag polypeptides of the disclosure can self-assemble to form capsids, also referred to as Virus-like particles (VLPs).

In some embodiments, the capsid comprises a PNMA2-based capsid. In some instances, the PNMA2-based capsid comprises a plurality of recombinant PNMA2 polypeptides of the disclosure. In some instances, the PNMA2-based capsid comprises a plurality of engineered PNMA2 polypeptides of the disclosure. In some embodiments, the PNMA2 polypeptides are recombinant and engineered.

In some embodiments, the capsid comprises an endo-Gag-based capsid. In some instances, the endo-Gag capsid comprises a plurality of recombinant endo-Gag polypeptides of the disclosure. In some instances, the endo-Gag capsid comprises a plurality of engineered endo-Gag polypeptides of the disclosure. In some embodiments, the endo-Gag polypeptides are recombinant and engineered.

In certain embodiments, the assembly of PNMA2 and/or endo-Gag-based capsids occurs ex vivo or in vitro. In some instances, the PNMA2 and/or endo-Gag-based capsid is assembled in vivo.

In some embodiments, the capsid comprises a disulfide bond. The PNAM2 or endo-Gag polypeptides that assemble to form the capsids can comprise one or more cysteine residues that form one or more disulfide bonds. Disulfide bonds can contribute to, for example, assembly, re-assembly, and/or stability of capsids disclosed herein. In some embodiments, disulfide bonds can be reduced in a target site, such as an intracellular (e.g., cytoplasmic) environment, facilitating capsid disassembly and cargo delivery.

In some embodiments, a first PNAM2 or endo-Gag polypeptide subunit of a capsid forms an intermolecular disulfide bond with a second PNMA2 or endo-Gag polypeptide. In some embodiments, a PNAM2 or endo-Gag polypeptide subunit of a capsid forms an intramolecular disulfide bond. In some embodiments, a PNAM2 or endo-Gag polypeptide subunit of a capsid forms an intramolecular disulfide bond and an intermolecular disulfide bond. In some embodiments, disulfide bonds contribute to the formation of PNMA2 or endo-Gag polypeptide dimers, trimers, tetramers, and/or or other higher-order multimers.

In some embodiments, a PNMA2 polypeptide or endo-Gag polypeptide comprises at least one, at least two, at least three, or at least four cysteines that form disulfide bonds, e.g., upon assembly to form a capsid. In some embodiments, a PNMA2 polypeptide or endo-Gag polypeptide comprises one cysteine that forms a disulfide bond. In some embodiments, a PNMA2 polypeptide or endo-Gag polypeptide comprises two cysteines that form disulfide bonds. In some embodiments, a PNMA2 polypeptide or endo-Gag polypeptide comprises three cysteines that form disulfide bonds. In some embodiments, a PNMA2 polypeptide or endo-Gag polypeptide comprises four cysteines that form disulfide bonds.

In some embodiments, a PNMA2 polypeptide or endo-Gag polypeptide comprises a cysteine residue, e.g., a disulfide-forming cysteine residue at a position corresponding to, for example, any one or more of positions 10, 136, 233, and 310 of SEQ ID NO: 1. In some embodiments, a PNMA2 polypeptide or endo-Gag polypeptide comprises a cysteine residue (e.g., a disulfide-forming cysteine residue) at a position corresponding to residue 10 of SEQ ID NO: 1. In some embodiments, a PNMA2 polypeptide or endo-Gag polypeptide comprises a cysteine residue (e.g., a disulfide-forming cysteine residue) at a position corresponding to residue 136 of SEQ ID NO: 1. In some embodiments, a PNMA2 polypeptide or endo-Gag polypeptide comprises a cysteine residue (e.g., a disulfide-forming cysteine residue) at a position corresponding to residue 233 of SEQ ID NO: 1. In some embodiments, a PNMA2 polypeptide or endo-Gag polypeptide comprises a cysteine residue (e.g., a disulfide-forming cysteine residue) at a position corresponding to residue 310 of SEQ ID NO: 1.

In some embodiments, the capsid has an average diameter of at least 1 nm, or more. In some instances, the capsid has an average diameter of at least 2 nm, at least 3 nm, at least 4 nm, at least 5 nm, at least 10 nm, at least 11 nm, at least 12 nm, at least 13 nm, at least 14 nm, at least 15 nm, at least 16 nm, at least 17 nm, at least 18 nm, at least 19 nm, at least 20 nm, at least 25 nm, at least 30 nm, at least 40 nm, at least 50 nm, at least 60 nm, at least 70 nm, at least 80 nm, at least 90 nm, at least 100 nm, at least 150 nm, at least 200 nm, at least 300 nm, at least 400 nm, at least 500 nm, at least 600 nm, or more. In some instances, the capsid has an average diameter of at least 5 nm, or more. In some instances, the capsid has an average diameter of at least 7 nm, or more. In some cases, the capsid has an average diameter of at least 10 nm, or more. In some instances, the capsid has an average diameter of at least 12 nm, or more. In some instances, the capsid has an average diameter of at least 15 nm, or more. In some instances, the capsid has an average diameter of at least 17 nm, or more. In some instances, the capsid has an average diameter of at least 20 nm, or more. In some cases, the capsid has an average diameter of at least 30 nm, or more. In some cases, the capsid has an average diameter of at least 40 nm, or more. In some cases, the capsid has an average diameter of at least 50 nm, or more. In some cases, the capsid has an average diameter of at least 80 nm, or more. In some cases, the capsid has an average diameter of at least 100 nm, or more. In some cases, the capsid has an average diameter of at least 200 nm, or more. In some cases, the capsid has an average diameter of at least 300 nm, or more. In some cases, the capsid has an average diameter of at least 400 nm, or more. In some cases, the capsid has an average diameter of at least 500 nm, or more. In some cases, the capsid has an average diameter of at least 600 nm, or more.

In some embodiments, the capsid has an average diameter of at most 100 nm, or less. In some instances, the capsid has an average diameter of at most 1 nm, at most 5 nm, at most 10 nm, at most 20 nm, at most 30 nm, at most 40 nm, at most 50 nm, at most 60 nm, at most 70 nm, at most 80 nm, at most 90 nm, at most 95 nm, at most 100 nm, at most 105 nm, at most 110 nm, at most 115 nm, at most 120 nm, at most 125 nm, at most 130 nm, at most 140 nm, at most 150 nm, at most 200 nm, at most 300 nm, at most 400 nm, at most 500 nm, at most 600 nm, or less. In some cases, the capsid has an average diameter of at most 50 nm, or less. In some cases, the capsid has an average diameter of at most 80 nm, or less.

In some cases, the capsid has an average diameter of at most 90 nm, or less. In some cases, the capsid has an average diameter of at most 95 nm, or less. In some cases, the capsid has an average diameter of at most 100 nm, or less. In some cases, the capsid has an average diameter of at most 110 nm, or less. In some cases, the capsid has an average diameter of at most 120 nm, or less. In some cases, the capsid has an average diameter of at most 150 nm, or less. In some cases, the capsid has an average diameter of at most 200 nm, or less. In some cases, the capsid has an average diameter of at most 300 nm, or less. In some cases, the capsid has an average diameter of at most 400 nm, or less. In some cases, the capsid has an average diameter of at most 500 nm, or less. In some cases, the capsid has an average diameter of at most 600 nm, or less.

In some embodiments, the capsid has an average diameter of about 1 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 120 nm, about 150 nm, about 200 nm, about 300 nm, about 400 nm, about 500 nm, or about 600 nm. In some instances, the capsid has an average diameter of about 20 nm. In some cases, the capsid has an average diameter of about 30 nm. In some cases, the capsid has an average diameter of about 40 nm. In some cases, the capsid has an average diameter of about 50 nm. In some cases, the capsid has an average diameter of about 60 nm. In some cases, the capsid has an average diameter of about 70 nm. In some cases, the capsid has an average diameter of about 80 nm. In some cases, the capsid has an average diameter of about 100 nm. In some cases, the capsid has an average diameter of about 200 nm.

In some embodiments, the capsid has an average diameter of from about 1 nm to about 600 nm. In some instances, the capsid has an average diameter of from about 5 nm to about 500 nm, from about 5 nm to about 400 nm, from about 5 nm to about 300 nm, from about 5 nm to about 200 nm, from about 5 nm to about 100 nm, from about 5 nm to about 50 nm, from about 5 nm to about 30 nm, 10 nm to about 500 nm, from about 10 nm to about 400 nm, from about 10 nm to about 300 nm, from about 10 nm to about 200 nm, from about 10 nm to about 150 nm, from about 10 nm to about 120 nm, from about 10 nm to about 100 nm, from about 10 nm to about 90 nm, from about 10 nm to about 50 nm, from about 10 nm to about 30 nm, 15 nm to about 500 nm, from about 15 nm to about 400 nm, from about 15 nm to about 300 nm, from about 15 nm to about 200 nm, from about 15 nm to about 150 nm, from about 15 nm to about 120 nm, from about 15 nm to about 100 nm, from about 15 nm to about 90 nm, from about 15 nm to about 50 nm, from about 20 nm to about 500 nm, from about 20 nm to about 400 nm, from about 20 nm to about 300 nm, from about 20 nm to about 200 nm, from about 20 nm to about 150 nm, from about 20 nm to about 120 nm, from about 20 nm to about 100 nm, from about 20 nm to about 90 nm, from about 20 nm to about 50 nm, from about 30 nm to about 500 nm, from about 30 nm to about 400 nm, from about 30 nm to about 300 nm, from about 30 nm to about 200 nm, from about 30 nm to about 100 nm, from about 30 nm to about 50 nm, from about 50 nm to about 300 nm, from about 50 nm to about 200 nm, or from about 50 nm to about 100 nm.

Capsids in a composition of the disclosure can be, for example, at least about 5%, at least about 10%, at least about 20%, at least about 30%, at least about 40%, at least about 50%, at least about 60%, at least about 70%, at least about 80%, at least about 90%, at least 92%, at least 94%, at least 96%, at least 98%, or at least 99% pure. Purity can be determined, for example, by SDS-PAGE.

Capsids in a composition of the disclosure can exhibit, for example, at least about 5%, at least about 10%, at least about 20%, at least about 30%, at least about 40%, at least about 50%, at least about 60%, at least about 70%, at least about 80%, or at least about 90% particle homogeneity. Particle homogeneity can be determined, for example, by multi-angle dynamic light scattering (MADLS), e.g., using a particle diameter size range disclosed herein.

In some instances, the PNMA2 and/or endo-Gag-based capsid is stable at room temperature. In some cases, the PNMA2 and/or endo-Gag-based capsid is empty. In other cases, the PNMA2 and/or endo-Gag-based capsid is loaded (for example, loaded with a heterologous cargo disclosed herein). In some embodiments, the heterologous cargo is in an interior of at least 1%, at least 5%, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or at least 99.5% of a plurality of capsids. In some embodiments, the heterologous cargo is in an interior of at least 50% of a plurality of the capsids. In some embodiments, the heterologous cargo is on an exterior of at least 1%, at least 3%, at least 5%, at least 7%, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or at least 99.5% of a plurality of capsids.

In some embodiments, at least 1%, at least 5%, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or at least 99.5% of the heterologous cargo is in an interior of a plurality of capsids. In some embodiments, at most 1%, at most 3%, at most 5%, at most 7%, at most 10%, at most 20%, at most 30%, at most 40%, at most 50%, at most 60%, at most 70%, at most 80%, at most 90%, at most 91%, at most 92%, at most 93%, at most 94%, at most 95%, at most 96%, at most 97%, at most 98%, at most 99%, or at most 99.5% of the heterologous cargo is on an exterior of a plurality of capsids. In some embodiments, at least 1%, at least 3%, at least 5%, at least 7%, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or at least 99.5% of the heterologous cargo is on an exterior of a plurality of capsids.

In some instances, the PNMA2 and/or endo-Gag-based capsid is stable at a temperature from about 2° C. to about 37° C. In some instances, the PNMA2 and/or endo-Gag-based capsid is stable at a temperature from about 2° C. to about 40° C., 2° C. to about 37° C., 2° C. to about 25° C., 2° C. to about 20° C., 4° C. to about 40° C., 4° C. to about 37° C., 4° C. to about 25° C., 4° C. to about 20° C., 2° C. to about 8° C., about 2° C. to about 4° C., about 20° C. to about 37° C., about 25° C. to about 37° C., about 20° C. to about 30° C., about 25° C. to about 30° C., or about 30° C. to about 37° C. In some cases, the PNMA2 and/or endo-Gag-based capsid is empty. In other cases, the PNMA2 and/or endo-Gag-based capsid is loaded (for example, loaded with a heterologous cargo and/or a therapeutic agent, e.g., a DNA or an RNA).

In some instances, the PNMA2 and/or endo-Gag-based capsid is stable for at least about 1 day, at least about 2 days, at least about 4 days, at least about 5 days, at least about 7 days, at least about 14 days, at least about 28 days, at least about 30 days, at least about 60 days, at least about 2 months, at least about 3 months, at least about 4 months, at least about 5 months, at least about 6 months, at least about 12 months, at least about 18 months, at least about 24 months, at least about 3 years, at least about 5 years, or longer. In some case, the PNMA2 and/or endo-Gag-based capsid exhibits low or minimal degradation, e.g., less than 10%, less than 5%, less than 4%, less than 3%, less than 2%, less than 1%, or less than 0.5% based on the total population of the PNMA2 and/or endo-Gag-based capsids that are degraded. In some cases, the PNMA2 and/or endo-Gag-based capsid is empty. In other cases, the PNMA2 and/or endo-Gag-based capsid is loaded (for example, loaded with a therapeutic agent, e.g., a DNA or an RNA).

In some embodiments, a capsid comprises a first endogenous retroviral capsid polypeptide and a second endogenous retroviral capsid polypeptide; wherein the amino acid sequence of the first endogenous retroviral capsid polypeptide is not identical to the amino acid sequence of the second endogenous retroviral capsid polypeptide.

In some embodiments, the first endogenous retroviral capsid polypeptide is an endo Gag polypeptide or comprises an amino acid sequence of an endo Gag polypeptide. In some embodiments, the first endogenous retroviral capsid polypeptide is a native endo Gag polypeptide or comprises an amino acid sequence of a native endo Gag polypeptide. In some embodiments, the first endogenous retroviral capsid polypeptide is an engineered endo Gag polypeptide or comprises an amino acid sequence of an engineered endo Gag polypeptide. In some embodiments, the first endogenous retroviral capsid polypeptide is an engineered endo Gag polypeptide that comprises an amino acid deletion relative to a corresponding native endo Gag polypeptide. In some embodiments, the first endogenous retroviral capsid polypeptide is an engineered endo Gag polypeptide that comprises an amino acid substitution relative to a corresponding native endo Gag polypeptide. In some embodiments, the first endogenous retroviral capsid polypeptide is an engineered endo Gag polypeptide that comprises an amino acid insertion relative to a corresponding native endo Gag polypeptide. In some embodiments, the first endogenous retroviral capsid polypeptide is an engineered endo Gag polypeptide that comprises a non-native cysteine that is not present in a corresponding native endo Gag polypeptide.

In some embodiments, the first endogenous retroviral capsid polypeptide is a PNMA2 polypeptide or comprises an amino acid sequence of a PNMA2 polypeptide. In some embodiments, the first endogenous retroviral capsid polypeptide is a native PNMA2 polypeptide or comprises an amino acid sequence of a native PNMA2 polypeptide. In some embodiments, the first endogenous retroviral capsid polypeptide is an engineered PNMA2 polypeptide or comprises an amino acid sequence of an engineered PNMA2 polypeptide. In some embodiments, the first endogenous retroviral capsid polypeptide is an engineered PNMA2 polypeptide that comprises an amino acid deletion relative to a corresponding native PNMA2 polypeptide. In some embodiments, the first endogenous retroviral capsid polypeptide is an engineered PNMA2 polypeptide that comprises an amino acid substitution relative to a corresponding native PNMA2 polypeptide. In some embodiments, the first endogenous retroviral capsid polypeptide is an engineered PNMA2 polypeptide that comprises an amino acid insertion relative to a corresponding native PNMA2 polypeptide. In some embodiments, the first endogenous retroviral capsid polypeptide is an engineered PNMA2 polypeptide that comprises a non-native cysteine that is not present in a corresponding native PNMA2 polypeptide.

In some embodiments, the first endogenous retroviral capsid polypeptide is not a PNMA2 polypeptide or does not contain an amino acid sequence of a PNMA2 polypeptide. In some embodiments, the first endogenous retroviral capsid polypeptide is not a native PNMA2 polypeptide or does not contain an amino acid sequence of a native PNMA2 polypeptide. In some embodiments, the first endogenous retroviral capsid polypeptide is not an engineered PNMA2 polypeptide or does not contain an amino acid sequence of an engineered PNMA2 polypeptide.

In some embodiments, the first endogenous retroviral capsid polypeptide comprises an exogenous polypeptide sequence disclosed herein. In some embodiments, the first endogenous retroviral capsid polypeptide comprises a cargo binding domain disclosed herein (e.g., an RNA, DNA, or protein-binding domain). In some embodiments, the first endogenous retroviral capsid polypeptide comprises a domain that binds to a cell surface molecule. In some embodiments, the first endogenous retroviral capsid polypeptide comprises an antibody or antigen-binding fragment thereof.

In some embodiments, the second endogenous retroviral capsid polypeptide is an endo Gag polypeptide or comprises an amino acid sequence of an endo Gag polypeptide. In some embodiments, the second endogenous retroviral capsid polypeptide is a native endo Gag polypeptide or comprises an amino acid sequence of a native endo Gag polypeptide. In some embodiments, the second endogenous retroviral capsid polypeptide is an engineered endo Gag polypeptide or comprises an amino acid sequence of an engineered endo Gag polypeptide. In some embodiments, the second endogenous retroviral capsid polypeptide is an engineered endo Gag polypeptide that comprises an amino acid deletion relative to a corresponding native endo Gag polypeptide. In some embodiments, the second endogenous retroviral capsid polypeptide is an engineered endo Gag polypeptide that comprises an amino acid substitution relative to a corresponding native endo Gag polypeptide. In some embodiments, the second endogenous retroviral capsid polypeptide is an engineered endo Gag polypeptide that comprises an amino acid insertion relative to a corresponding native endo Gag polypeptide. In some embodiments, the second endogenous retroviral capsid polypeptide is an engineered endo Gag polypeptide that comprises a non-native cysteine that is not present in a corresponding native endo Gag polypeptide.

In some embodiments, the second endogenous retroviral capsid polypeptide is a PNMA2 polypeptide or comprises an amino acid sequence of a PNMA2 polypeptide. In some embodiments, the second endogenous retroviral capsid polypeptide is a native PNMA2 polypeptide or comprises an amino acid sequence of a native PNMA2 polypeptide. In some embodiments, the second endogenous retroviral capsid polypeptide is an engineered PNMA2 polypeptide or comprises an amino acid sequence of an engineered PNMA2 polypeptide. In some embodiments, the second endogenous retroviral capsid polypeptide is an engineered PNMA2 polypeptide that comprises an amino acid deletion relative to a corresponding native PNMA2 polypeptide. In some embodiments, the second endogenous retroviral capsid polypeptide is an engineered PNMA2 polypeptide that comprises an amino acid substitution relative to a corresponding native PNMA2 polypeptide. In some embodiments, the second endogenous retroviral capsid polypeptide is an engineered PNMA2 polypeptide that comprises an amino acid insertion relative to a corresponding native PNMA2 polypeptide. In some embodiments, the second endogenous retroviral capsid polypeptide is an engineered PNMA2 polypeptide that comprises a non-native cysteine that is not present in a corresponding native PNMA2 polypeptide.

In some embodiments, the second endogenous retroviral capsid polypeptide comprises an exogenous polypeptide sequence disclosed herein. In some embodiments, the second endogenous retroviral capsid polypeptide comprises a cargo binding domain disclosed herein (e.g., an RNA, DNA, or protein-binding domain). In some embodiments, the second endogenous retroviral capsid polypeptide comprises a domain that binds to a cell surface molecule. In some embodiments, the second endogenous retroviral capsid polypeptide comprises an antibody or antigen-binding fragment thereof.

In some embodiments, the first endogenous retroviral capsid polypeptide and the second endogenous retroviral capsid polypeptide are present in the capsid at a ratio of about 1:1, 2:1, 3:1, 4:1, 5:1, 6:1, 7:1, 8:1, 9:1, 10:1, 20:1, 50:1, 100:1, 1:2, 1:3, 1:4, 1:5, 1:6, 1:7, 1:8, 1:9, 1:10, 1:20, or 1:50. In some instances, the ratio is the comparison in molar concentration. In some instances, the ratio is the comparison in the number of capsid forming subunits. In some instances, the ratio based on the mass (e.g., number of ng) of the capsid forming subunits present.

In some embodiments, the PNMA2-based capsid or endo-Gag-based capsid comprises a plurality of recombinant or engineered PNMA2 polypeptides and a plurality of non-PNMA2 proteins. Exemplary species of non-PNMA2 proteins include but are not limited to, PNMA1, PNMA3, PNMA4 (MOAP1), PNMA5, PNMA6A, PNMA6B/6D, PNMA6E, PNMA6F, PNMA7A, PNMA7B, PNMA8A, PNMA8B, PNMA8C, CCDC8, Arc, BOP, LDOC1, PEG10, RTL3, RTL6, RTL8A, RTL8B, ZNF18, Copia, ASPRV1, a protein or a combination of proteins chosen from the SCAN domain family, and a protein or a combination of proteins chosen from the retrotransposon Gag-like family.

In some instances, the ratio of the plurality of recombinant or engineered PNMA2 polypeptides to the plurality of non-PNMA2 proteins is 1:1, 2:1, 3:1, 4:1, 5:1, 6:1, 7:1, 8:1, 9:1, 10:1, 20:1, 50:1, 100:1, 1:2, 1:3, 1:4, 1:5, 1:6, 1:7, 1:8, 1:9, 1:10, 1:20, or 1:50. In some instances, the ratio is the comparison in molar concentration. In some instances, the ratio is the comparison in the number of capsid forming subunits.

### Cargos

In some embodiments, a capsid of the disclosure comprises a cargo. In some embodiments, the cargo is a heterologous cargo that is not native to the PNMA2 polypeptide. In some embodiments, the cargo is a therapeutic agent. In some embodiments, the cargo is a nucleic acid molecule, a small molecule, a protein, a peptide, an antibody or binding fragment thereof, a peptidomimetic, or a nucleotidomimetic. In some instances, the cargo is a therapeutic cargo, comprising e.g., one or more drugs. In some instances, the cargo comprises a diagnostic tool or component thereof, for profiling, e.g., one or more markers (such as markers associates with one or more disease phenotypes). In additional instances, the cargo comprises an imaging tool or component thereof.

In some instances, the cargo is a nucleic acid molecule. Examples of nucleic acid molecules include DNA, RNA, and mixtures of DNA and RNA. In some embodiments, the nucleic acid molecule comprises a hybrid of DNA and RNA.

In some instances, the nucleic acid molecule is a DNA polymer. In some cases, the DNA is a single stranded DNA polymer. In other cases, the DNA is a double stranded DNA polymer. In additional cases, the DNA is a hybrid of single and double stranded DNA polymers.

In some embodiments, the nucleic acid molecule is an RNA polymer, e.g., a single stranded RNA polymer, a double stranded RNA polymer, or a hybrid of single and double stranded RNA polymers. In some instances, the RNA comprises and/or encodes an antisense oligoribonucleotide, a siRNA, an mRNA, a tRNA, an rRNA, a snRNA, a shRNA, microRNA, or a non-coding RNA.

In some embodiments, the nucleic acid molecule is an antisense oligonucleotide, optionally comprising DNA, RNA, or a hybrid of DNA and RNA. In some instances, the nucleic acid molecule comprises and/or encodes an mRNA molecule. In some embodiments, the nucleic acid molecule comprises and/or encodes an RNAi molecule. In some cases, the RNAi molecule is a microRNA (miRNA) molecule. In other cases, the RNAi molecule is a siRNA molecule. The miRNA and/or siRNA are optionally double-stranded or as a hairpin, and further optionally encapsulated as precursor molecules.

In some embodiments, the nucleic acid molecule is for use in a nucleic acid-based therapy. In some instances, the nucleic acid molecule is for regulating gene expression (e.g., modulating mRNA translation or degradation), modulating RNA splicing, or RNA interference. In some cases, the nucleic acid molecule comprises and/or encodes an antisense oligonucleotide, microRNA molecule, siRNA molecule, mRNA molecule, for use in regulation of gene expression, modulating RNA splicing, or RNA interference.

In some instances, the nucleic acid molecule is for use in gene editing. Exemplary gene editing systems include, but are not limited to, CRISPR-Cas systems, zinc finger nuclease (ZFN) systems, and transcription activator-like effector nuclease (TALEN) systems. In some cases, the nucleic acid molecule comprises and/or encodes a component involved in the CRISPR-Cas systems, the ZFN systems, or the TALEN systems.

In some cases, the nucleic acid molecule is for use in antigen production for therapeutic and/or prophylactic vaccine production. For example, the nucleic acid molecule encodes an antigen that is expressed and elicits a desirable immune response (e.g., a pro-inflammatory immune response, an anti-inflammatory immune response, a tolerogenic immune response, an B cell response, an antibody response, a T cell response, a CD4+ T cell response, a CD8+ T cell response, a Th1 immune response, a Th2 immune response, a Th17 immune response, a Treg immune response, an M1 macrophage response, an M2 macrophage response, or a combination thereof).

In some cases, the nucleic acid molecule comprises a nucleic acid enzyme. Nucleic acid enzymes are RNA molecules (e.g., ribozymes) or DNA molecules (e.g., deoxyribozymes) that have catalytic activities. In some instances, the nucleic acid molecule is a ribozyme. In other instances, the nucleic acid molecule is a deoxyribozyme. In some cases, the nucleic acid molecule is a MNAzyme, which functions as a biosensor and/or a molecular switch (see, e.g., Mokany, et al., (2010) MNAzymes, a versatile new class of nucleic acid enzymes that can function as biosensors and molecular switches, JACS 132(2): 1051-1059).

In some instances, exemplary targets of the nucleic acid molecule include, but are not limited to, UL123 (human cytomegalovirus), APOB, AR (androgen receptor) gene, KRAS, PCSK9, CFTR, and SMN (e.g., SMN2).

In some embodiments, the nucleic acid molecule is at least 5 nucleotides or more in length. In some instances, the nucleic acid molecule is at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 50, at least 60, at least 70, at least 80, at least 90, at least 100, at least 150, at least 200, at least 250, at least 300, at least 400, at least 500, at least 1000, at least 1500, at least 2000, at least 3000, at least 4000, at least 5000, at least 6000, at least 7000, at least 8000, or at least 9000 nucleotides or more in length. In some instances, the nucleic acid molecule is at least 10 nucleotides or more in length. In some instances, the nucleic acid molecule is at least 15 nucleotides or more in length. In some instances, the nucleic acid molecule is at least 20 nucleotides or more in length. In some instances, the nucleic acid molecule is at least 30 nucleotides or more in length. In some instances, the nucleic acid molecule is at least 40 nucleotides or more in length. In some instances, the nucleic acid molecule is at least 50 nucleotides or more in length. In some instances, the nucleic acid molecule is at least 100 nucleotides or more in length. In some instances, the nucleic acid molecule is at least 200 nucleotides or more in length. In some instances, the nucleic acid molecule is at least 300 nucleotides or more in length. In some instances, the nucleic acid molecule is at least 500 nucleotides or more in length. In some instances, the nucleic acid molecule is at least 1000 nucleotides or more in length. In some instances, the nucleic acid molecule is at least 2000 nucleotides or more in length. In some instances, the nucleic acid molecule is at least 3000 nucleotides or more in length. In some instances, the nucleic acid molecule is at least 4000 nucleotides or more in length. In some instances, the nucleic acid molecule is at least 5000 nucleotides or more in length. In some instances, the nucleic acid molecule is at least 8000 nucleotides or more in length.

In some embodiments, the nucleic acid molecule is at most 10,000 nucleotides or less in length. In some instances, the nucleic acid molecule is at most 15, at most 20, at most 25, at most 30, at most 35, at most 40, at most 50, at most 60, at most 70, at most 80, at most 90, at most 100, at most 150, at most 200, at most 250, at most 300, at most 400, at most 500, at most 1000, at most 1500, at most 2000, at most 3000, at most 4000, at most 5000, at most 6000, at most 7000, at most 8000, at most 9000, or at most 10,000 nucleotides or less in length. In some instances, the nucleic acid molecule is at most 15 nucleotides or less in length. In some instances, the nucleic acid molecule is at most 20 nucleotides or less in length. In some instances, the nucleic acid molecule is at most 25 nucleotides or less in length. In some instances, the nucleic acid molecule is at most 30 nucleotides or less in length. In some instances, the nucleic acid molecule is at most 40 nucleotides or less in length. In some instances, the nucleic acid molecule is at most 50 nucleotides or less in length. In some instances, the nucleic acid molecule is at most 100 nucleotides or less in length. In some instances, the nucleic acid molecule is at most 200 nucleotides or less in length. In some instances, the nucleic acid molecule is at most 300 nucleotides or less in length. In some instances, the nucleic acid molecule is at most 500 nucleotides or less in length. In some instances, the nucleic acid molecule is at most 1000 nucleotides or less in length. In some instances, the nucleic acid molecule is at most 2000 nucleotides or less in length. In some instances, the nucleic acid molecule is at most 3000 nucleotides or less in length. In some instances, the nucleic acid molecule is at most 4000 nucleotides or less in length. In some instances, the nucleic acid molecule is at most 5000 nucleotides or less in length. In some instances, the nucleic acid molecule is at most 8000 nucleotides or less in length. In some instances, the nucleic acid molecule is at most 9000 nucleotides or less in length.

In some embodiments, the nucleic acid molecule is about 100 nucleotides in length. In some instances, the nucleic acid molecule is about 6, about 7, about 8, about 9, about 10, about 15, about 20, about 25, about 30, about 35, about 40, about 50, about 60, about 70, about 80, about 90, about 100, about 150, about 200, about 250, about 300, about 400, about 500, about 1000, about 1500, about 2000, about 3000, about 4000, about 5000, about 6000, about 7000, about 8000, about 9000, or about 10000 nucleotides in length. In some instances, the nucleic acid molecule is about 10 nucleotides in length. In some instances, the nucleic acid molecule is about 15 nucleotides in length. In some instances, the nucleic acid molecule is about 20 nucleotides in length. In some instances, the nucleic acid molecule is about 25 nucleotides in length. In some instances, the nucleic acid molecule is about 30 nucleotides in length. In some instances, the nucleic acid molecule is about 40 nucleotides in length. In some instances, the nucleic acid molecule is about 50 nucleotides in length. In some instances, the nucleic acid molecule is about 100 nucleotides in length. In some instances, the nucleic acid molecule is about 150 nucleotides in length. In some instances, the nucleic acid molecule is about 200 nucleotides in length. In some instances, the nucleic acid molecule is about 300 nucleotides in length. In some instances, the nucleic acid molecule is about 500 nucleotides in length. In some instances, the nucleic acid molecule is about 1000 nucleotides in length. In some instances, the nucleic acid molecule is about 2000 nucleotides in length. In some instances, the nucleic acid molecule is about 3000 nucleotides in length. In some instances, the nucleic acid molecule is about 4000 nucleotides in length. In some instances, the nucleic acid molecule is about 5000 nucleotides in length. In some instances, the nucleic acid molecule is about 8000 nucleotides in length. In some instances, the nucleic acid molecule is about 9000 nucleotides in length.

In some embodiments, the nucleic acid molecule is from about 5 to about 10,000 nucleotides in length. In some instances, the nucleic acid molecule is from about 5 to about 9000 nucleotides in length, from about 5 to about 8000 nucleotides in length, from about 5 to about 7000 nucleotides in length, from about 5 to about 6000 nucleotides in length, from about 5 to about 5000 nucleotides in length, from about 5 to about 4000 nucleotides in length, from about 5 to about 3000 nucleotides in length, from about 5 to about 2000 nucleotides in length, from about 5 to about 1000 nucleotides in length, from about 5 to about 500 nucleotides in length, from about 5 to about 100 nucleotides in length, from about 5 to about 50 nucleotides in length, from about 5 to about 40 nucleotides in length, from about 5 to about 30 nucleotides in length, from about 5 to about 25 nucleotides in length, from about 5 to about 20 nucleotides in length, from about 10 to about 10,000 nucleotides in length. In some instances, the nucleic acid molecule is from about 10 to about 9000 nucleotides in length, from about 10 to about 8000 nucleotides in length, from about 10 to about 7000 nucleotides in length, from about 10 to about 6000 nucleotides in length, from about 10 to about 5000 nucleotides in length, from about 10 to about 4000 nucleotides in length, from about 10 to about 3000 nucleotides in length, from about 10 to about 2000 nucleotides in length, from about 10 to about 1000 nucleotides in length, from about 10 to about 500 nucleotides in length, from about 10 to about 100 nucleotides in length, from about 10 to about 50 nucleotides in length, from about 10 to about 40 nucleotides in length, from about 10 to about 30 nucleotides in length, from about 10 to about 25 nucleotides in length, from about 10 to about 20 nucleotides in length, from about 50 to about 10,000 nucleotides in length. In some instances, the nucleic acid molecule is from about 50 to about 9000 nucleotides in length, from about 50 to about 8000 nucleotides in length, from about 50 to about 7000 nucleotides in length, from about 50 to about 6000 nucleotides in length, from about 50 to about 5000 nucleotides in length, from about 50 to about 4000 nucleotides in length, from about 50 to about 3000 nucleotides in length, from about 50 to about 2000 nucleotides in length, from about 50 to about 1000 nucleotides in length, from about 50 to about 500 nucleotides in length, from about 50 to about 100 nucleotides in length, from about 50 to about 50 nucleotides in length, from about 50 to about 40 nucleotides in length, from about 50 to about 30 nucleotides in length, from about 50 to about 25 nucleotides in length, from about 50 to about 20 nucleotides in length, from about 100 to about 10,000 nucleotides in length. In some instances, the nucleic acid molecule is from about 100 to about 9000 nucleotides in length, from about 100 to about 8000 nucleotides in length, from about 100 to about 7000 nucleotides in length, from about 100 to about 6000 nucleotides in length, from about 100 to about 5000 nucleotides in length, from about 100 to about 4000 nucleotides in length, from about 100 to about 3000 nucleotides in length, from about 100 to about 2000 nucleotides in length, from about 100 to about 1000 nucleotides in length, from about 100 to about 500 nucleotides in length, from about 100 to about 100 nucleotides in length, from about 100 to about 50 nucleotides in length, from about 100 to about 40 nucleotides in length, from about 100 to about 30 nucleotides in length, from about 100 to about 25 nucleotides in length, from about 100 to about 20 nucleotides in length, from about 500 to about 10,000 nucleotides in length. In some instances, the nucleic acid molecule is from about 500 to about 9000 nucleotides in length, from about 500 to about 8000 nucleotides in length, from about 500 to about 7000 nucleotides in length, from about 500 to about 6000 nucleotides in length, from about 500 to about 5000 nucleotides in length, from about 500 to about 4000 nucleotides in length, from about 500 to about 3000 nucleotides in length, from about 500 to about 2000 nucleotides in length, from about 500 to about 1000 nucleotides in length, from about 500 to about 500 nucleotides in length, from about 500 to about 100 nucleotides in length, from about 500 to about 50 nucleotides in length, from about 500 to about 40 nucleotides in length, from about 500 to about 30 nucleotides in length, from about 500 to about 25 nucleotides in length, or from about 500 to about 20 nucleotides in length.

In some embodiments, the nucleic acid molecule comprises natural, synthetic, or artificial nucleotide analogues or bases. In some cases, the nucleic acid molecule comprises combinations of DNA, RNA and/or nucleotide analogues. In some instances, the synthetic or artificial nucleotide analogues or bases comprise modifications at one or more of ribose moiety, phosphate moiety, nucleoside moiety, or a combination thereof.

In some embodiments, a nucleotide analogue or artificial nucleotide base described above comprises a nucleic acid with a modification at a 2′ hydroxyl group of the ribose moiety. In some instances, the modification includes an H, OR, R, halo, SH, SR, NH2, NHR, NR2, or CN, wherein R is an alkyl moiety. Exemplary alkyl moiety includes, but is not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′O-methoxyethyl modification adds a methoxyethyl group to the 2′ hydroxyl group of the ribose moiety.

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate-derived overall negative charge of the oligonucleotide molecule by introducing one positive charge from the amine group per sugar and thereby improves cellular uptake properties due to its zwitterionic properties.

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer.

In some embodiments, additional modifications at the 2′ hydroxyl group include 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA).

In some embodiments, a nucleotide analogue comprises a modified base such as, but not limited to, N1-methylpseudouridine, 5-propynyluridine, 5-propynylcytidine, 6-methyladenine, 6-methylguanine, N, N,-dimethyladenine, 2-propyladenine, 2 propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or are based on, mannoses, arabinoses, glucopyranoses, galactopyranoses, 4′-thioribose, and other sugars, heterocycles, or carbocycles. The term nucleotide also includes universal bases. By way of example, universal bases include but are not limited to 3-nitropyrrole, 5-nitroindole, or nebularine.

In some embodiments, a nucleotide analogue further comprises a morpholino, a peptide nucleic acid (PNA), a methylphosphonate nucleotide, a thiolphosphonate nucleotide, a 2′-fluoro N3-P5′-phosphoramidite, or a 1′,5′-anhydrohexitol nucleic acid (HNA). Morpholino or phosphorodiamidate morpholino oligo (PMO) comprises synthetic molecules whose structure mimics natural nucleic acid structure but deviates from the normal sugar and phosphate structures. In some instances, the five-member ribose ring is substituted with a six-member morpholino ring containing four carbons, one nitrogen, and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

In some embodiments, peptide nucleic acid (PNA) does not contain sugar ring or phosphate linkage and the bases are attached and appropriately spaced by oligoglycine-like molecules, therefore, eliminating a backbone charge.

In some embodiments, one or more modifications optionally occur at the internucleotide linkage. In some instances, modified internucleotide linkage includes, but is not limited to, phosphorothioates; phosphorodithioates; methylphosphonates; 5′-alkylenephosphonates; 5′-methylphosphonate; 3′-alkylene phosphonates; borontrifluoridates; borano phosphate esters and selenophosphates of 3′-5′ linkage or 2′-5′ linkage; phosphotriesters; thionoalkylphosphotriesters; hydrogen phosphonate linkages; alkyl phosphonates; alkylphosphonothioates; arylphosphonothioates; phosphoroselenoates; phosphorodiselenoates; phosphinates; phosphoramidates; 3′-alkylphosphoramidates; aminoalkylphosphoramidates; thionophosphoramidates; phosphoropiperazidates; phosphoroanilothioates; phosphoroanilidates; ketones; sulfones; sulfonamides; carbonates; carbamates; methylenehydrazos; methylenedimethylhydrazos; formacetals; thioformacetals; oximes; methyleneiminos; methylenemethyliminos; thioamidates; linkages with riboacetyl groups; aminoethyl glycine; silyl or siloxane linkages; alkyl or cycloalkyl linkages with or without heteroatoms of, for example, 1 to 10 carbons that are saturated or unsaturated and/or substituted and/or contain heteroatoms; linkages with morpholino structures, amides, or polyamides wherein the bases are attached to the aza nitrogens of the backbone directly or indirectly; and combinations thereof.

In some embodiments, one or more modifications comprise a modified phosphate backbone in which the modification generates a neutral or uncharged backbone. In some instances, the phosphate backbone is modified by alkylation to generate an uncharged or neutral phosphate backbone. As used herein, alkylation includes methylation, ethylation, and propylation. In some cases, an alkyl group, as used herein in the context of alkylation, refers to a linear or branched saturated hydrocarbon group containing from 1 to 6 carbon atoms. In some instances, exemplary alkyl groups include, but are not limited to, methyl, ethyl, n-propyl, isopropyl, n-butyl, isobutyl, sec-butyl, tert-butyl, n-pentyl, isopentyl, neopentyl, hexyl, isohexyl, 1,1-dimethylbutyl, 2,2-dimethylbutyl, 3.3-dimethylbutyl, and 2-ethylbutyl groups. In some cases, a modified phosphate is a phosphate group as described in U.S. Pat. No. 9,481,905.

In some embodiments, additional modified phosphate backbones comprise methylphosphonate, ethylphosphonate, methylthiophosphonate, or methoxyphosphonate. In some cases, the modified phosphate is methylphosphonate. In some cases, the modified phosphate is ethylphosphonate. In some cases, the modified phosphate is methylthiophosphonate. In some cases, the modified phosphate is methoxyphosphonate.

In some embodiments, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

In some embodiments, exemplary nucleic acid cargos include, but are not limited to, Fomivirsen, Mipomersen, AZD5312 (AstraZeneca), Nusinersen, and SB010 (Sterna Biologicals).

### Gene Editing Systems

In some embodiments, the cargo comprises or encodes a gene editing system or a component thereof. Non-limiting examples of gene editing tools and techniques include CRISPR, TALEN, zinc finger nuclease (ZFN), meganuclease, Mega-TAL, and transposon-based systems.

In some embodiments, the cargo comprises or encodes a CRISPR-associated polypeptide (Cas), zinc finger nuclease (ZFN), zinc finger associate gene regulation polypeptide, transcription activator-like effector nuclease (TALEN), transcription activator-like effector associated gene regulation polypeptides, meganuclease, natural master transcription factors, epigenetic modifying enzymes, recombinase, flippase, transposase, RNA-binding proteins (RBP), an Argonaute protein, any derivative thereof, any variant thereof, or any fragment thereof.

In some embodiments, the cargo comprises or encodes a CRISPR system or a component thereof. A CRISPR system can be utilized to facilitate insertion of a recombinant nucleic acid encoding a regulatable membrane protein or a component thereof into a cell genome. For example, a CRISPR system can introduce a double stranded break at a target site in a genome or a random site of a genome.

In some embodiments, the cargo comprises or encodes a Cas protein. Non-limiting examples of Cas proteins that can be used in the CRISPR systems include Cas1, Cas1B, Cas2, Cas3, Cas4, Cas5, Cash, Cas7, Cas8, Cas9 (also known as Csn1 or Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, and modified versions thereof. An unmodified CRISPR enzyme can have DNA cleavage activity, such as Cas9. A CRISPR enzyme can direct cleavage of one or both strands at a target sequence, such as within a target sequence and/or within a complement of a target sequence. For example, a CRISPR enzyme can direct cleavage of one or both strands within or within about 1, 2, 3, 4, 5, 6, 7, 8, 9, 10, 15, 20, 25, 50, 100, 200, 500, or more base pairs from the first or last nucleotide of a target sequence. A Cas protein can be a high fidelity Cas protein. Alternatives to S. pyogenes Cas9 may include RNA-guided endonucleases from the Cpf1 family that display cleavage activity in mammalian cells.

In some embodiments, the cargo comprises or encodes a guide RNA (gRNA), e.g., that complexes with a Cas protein.

In some embodiments, the cargo comprises or encodes a nucleotide sequence to be inserted in to a genome. In some embodiments, the cargo comprises or encodes a repair template, e.g., for homology-directed repair.

In some embodiments, the cargo comprises or encodes a dual nickase CRISPR system or a component thereof. A dual nickase approach may be used to introduce a double stranded break. Cas proteins can be mutated at certain amino acids within either nuclease domains, thereby deleting activity of one nuclease domain and generating a nickase Cas protein capable of generating a single strand break. A nickase along with two distinct guide RNAs targeting opposite strands may be utilized to generate a DSB within a target site (often referred to as a “double nick” or “dual nickase” CRISPR system).

In some embodiments, the cargo comprises or encodes a transposon based system or a component thereof. A transposon-based system can be utilized for insertion of a recombinant nucleic acid encoding a regulatable membrane protein of the disclosure or a component thereof into a genome. A transposon can comprise a recombinant nucleic acid that can be inserted into a DNA sequence. A class I transposon can be transcribed into an RNA intermediate, then reverse transcribed and inserted into a DNA sequence. A class II transposon can comprise a DNA sequence that is excised from one DNA sequence and/or inserted into another DNA sequence. A class II transposon system can comprise (i) a transposon vector that contains a sequence (e.g., comprising a transgene) flanked by inverted terminal repeats, and (ii) a source for the transposase enzyme.

In some embodiments, the cargo comprises or encodes a TALEN system or a component thereof. TALENs can refer to engineered transcription activator-like effector nucleases that generally contain a central domain of DNA-binding tandem repeats and a cleavage domain. TALENs can be produced by fusing a TAL effector DNA binding domain to a DNA cleavage domain. In some cases, a DNA-binding tandem repeat comprises 33-35 amino acids in length and contains two hypervariable amino acid residues at positions 12 and 13 that can recognize at least one specific DNA base pair. A transcription activator-like effector (TALE) protein can be fused to a nuclease such as a wild-type or mutated Fok1 endonuclease or the catalytic domain of Fok1.

In some embodiments, the cargo comprises or encodes a zinc finger nuclease (ZFN) or a variant, fragment, or derivative thereof. ZFN can refer to a fusion between a cleavage domain, such as a cleavage domain of Fok1, and at least one zinc finger motif (e.g., at least 2, at least 3, at least 4, or at least 5 zinc finger motifs) which can bind polynucleotides such as DNA and RNA.

In some embodiments, the cargo comprises or encodes a meganuclease. Meganucleases generally refer to rare-cutting endonucleases or homing endonucleases that can be highly sequence specific. Meganucleases can recognize DNA target sites ranging from at least 12 base pairs in length, e.g., from 12 to 40 base pairs, 12 to 50 base pairs, or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. A nuclease-active meganuclease can generate a double-stranded break. The meganuclease can be monomeric or dimeric. In some embodiments, the meganuclease is naturally-occurring (found in nature) or wild-type, and in other instances, the meganuclease is non-natural, artificial, engineered, synthetic, rationally designed, or man-made. In some embodiments, the meganuclease of the present disclosure includes an I-CreI meganuclease, I-CeuI meganuclease, I-Mso1 meganuclease, I-SceI meganuclease, variants thereof, derivatives thereof, and fragments thereof.

### Small Molecules

In some embodiments, the cargo is a small molecule. In some instances, the small molecule is an inhibitor (e.g., a pan inhibitor or a selective inhibitor). In other instances, the small molecule is an activator. In additional cases, the small molecule is an agonist, antagonist, a partial agonist, a mixed agonist/antagonist, or a competitive antagonist.

In some embodiments, the small molecule is a drug that falls under the class of analgesics, antianxiety drugs, antiarrhythmics, antibacterials, antibiotics, anticoagulants and thrombolytics, anticonvulsants, antidepressants, antidiarrheals, antiemetics, antifungals, antihistamines, antihypertensives, anti-inflammatories, antineoplastics, antipsychotics, antipyretics, antivirals, barbiturates, beta-blockers, bronchodilators, common cold treatments, corticosteroids, cough suppressants, cytotoxics, decongestants, diuretics, expectorant, hormones, hypoglycemics, immunosuppressives, laxatives, muscle relaxants, sex hormones, sleeping drugs, or tranquilizers.

In some embodiments, the small molecule is an inhibitor, e.g., an inhibitor of a kinase pathway such as the Tyrosine kinase pathway or a Serine/Threonine kinase pathway. In some cases, the small molecule is a dual protein kinase inhibitor. In some cases, the small molecule is a lipid kinase inhibitor.

In some cases, the small molecule is a neuraminidase inhibitor.

In some cases, the small molecule is a carbonic anhydrase inhibitor.

In some embodiments, exemplary targets of the small molecule include, but are not limited to, vascular endothelial growth factor receptor 1 (VEGFR1), vascular endothelial growth factor receptor 2 (VEGFR2), vascular endothelial growth factor receptor 3 (VEGFR3), fibroblast growth factor receptor 1 (FGFR1), fibroblast growth factor receptor 2 (FGFR2), fibroblast growth factor receptor 3 (FGFR3), fibroblast growth factor receptor 4 (FGFR4), cyclin-dependent kinase 4 (CDK4), cyclin-dependent kinase 6 (CDK6), a receptor tyrosine kinase, a phosphoinositide 3-kinase (PI3K) isoform (e.g., PI31δ, also known as p1106), Janus kinase 1 (JAK1), Janus kinase 3 (JAK3), a receptor from the family of platelet-derived growth factor receptors (PDFG-R), and carbonic anhydrase (e.g., carbonic anhydrase I).

In some embodiments, the small molecule targets a viral protein, e.g., a viral envelope protein. In some embodiments, the small molecule decreases viral adsorption to a host cell. In some embodiments, the small molecule decreases viral entry into a host cell. In some embodiments, the small molecule decreases viral replication in a host or a host cell. In some embodiments, the small molecule decreases viral assembly.

In some embodiments, exemplary small molecule cargos include, but are not limited to, lenvatinib, palbociclib, regorafenib, idelalisib, tofacitinib, nintedanib, zanamivir, ethoxzolamide, and artemisinin.

### Proteins

In some embodiments, the cargo is a protein. In some instances, the protein is a full-length protein. In other instances, the protein is a fragment, e.g., a functional fragment. In some cases, the protein is a naturally occurring protein. In additional cases, the protein is a de novo engineered protein. In further cases, the protein is a fusion protein. In further cases, the protein is a recombinant protein. Exemplary proteins include, but are not limited to, Fc fusion proteins, anticoagulants, blood factors, bone morphogenetic proteins, enzymes, growth factors, hormones, interferons, interleukins, and thrombolytics.

In some instances, the protein is for use in an enzyme replacement therapy.

In some cases, the protein is for use in antigen production for therapeutic and/or prophylactic vaccine production. For example, the protein comprises an antigen that elicits a desirable immune response (e.g., a pro-inflammatory immune response, an anti-inflammatory immune response, a tolerogenic immune response, an B cell response, an antibody response, a T cell response, a CD4+ T cell response, a CD8+ T cell response, a Th1 immune response, a Th2 immune response, a Th17 immune response, a Treg immune response, an M1 macrophage response, an M2 macrophage response, or a combination thereof).

In some instances, exemplary protein cargos include, but are not limited to, romiplostim, liraglutide, a human growth hormone (rHGH), human insulin (BHI), follicle-stimulating hormone (FSH), Factor VIII, erythropoietin (EPO), granulocyte colony-stimulating factor (G-CSF), alpha-galactosidase A, alpha-L-iduronidase, N-acetylgalactosamine-4-sulfatase, dornase alfa, tissue plasminogen activator (TPA), glucocerebrosidase, interferon-beta-1a, insulin-like growth factor 1 (IGF-1), or rasburicase.

### Peptides

In some embodiments, the cargo is a peptide. In some instances, the peptide is a naturally occurring peptide. In other instances, the peptide is an artificial engineered peptide or a recombinant peptide. In some cases, the peptide targets a G-protein coupled receptor, an ion channel, a microbe, an anti-microbial target, a catalytic or other Ig-family of receptors, an intracellular target, a membrane-anchored target, or an extracellular target.

In some cases, the peptide comprises at least 2 amino acids. In some cases, the peptide comprises at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, or at least 100 amino acids. In some cases, the peptide comprises at least 10 amino acids. In some cases, the peptide comprises at least 15 amino acids. In some cases, the peptide comprises at least 20 amino acids. In some cases, the peptide comprises at least 30 amino acids. In some cases, the peptide comprises at least 40 amino acids. In some cases, the peptide comprises at least 50 amino acids. In some cases, the peptide comprises at least 60 amino acids. In some cases, the peptide comprises at least 70 amino acids. In some cases, the peptide comprises at least 80 amino acids. In some cases, the peptide comprises at least 90 amino acids. In some cases, the peptide comprises at least 100 amino acids.

In some cases, the peptide comprises at most 3 amino acids. In some cases, the peptide comprises at most 4, at most 5, at most 6, at most 7, at most 8, at most 9, at most 10, at most 15, at most 20, at most 25, at most 30, at most 35, at most 40, at most 45, at most 50, at most 60, at most 70, at most 80, at most 90, or at most 100 amino acids. In some cases, the peptide comprises at most 10 amino acids. In some cases, the peptide comprises at most 15 amino acids. In some cases, the peptide comprises at most 20 amino acids. In some cases, the peptide comprises at most 30 amino acids. In some cases, the peptide comprises at most 40 amino acids. In some cases, the peptide comprises at most 50 amino acids. In some cases, the peptide comprises at most 60 amino acids. In some cases, the peptide comprises at most 70 amino acids. In some cases, the peptide comprises at most 80 amino acids. In some cases, the peptide comprises at most 90 amino acids. In some cases, the peptide comprises at most 100 amino acids.

In some cases, the peptide comprises from about 1 to about 10 kDa. In some cases, the peptide comprises from about 1 to about 9 kDa, about 1 to about 6 kDa, about 1 to about 5 kDa, about 1 to about 4 kDa, about 1 to about 3 kDa, about 2 to about 8 kDa, about 2 to about 6 kDa, about 2 to about 4 kDa, about 1.2 to about 2.8 kDa, about 1.5 to about 2.5 kDa, or about 1.5 to about 2 kDa.

In some embodiments, the peptide is a cyclic peptide. In some instances, the cyclic peptide is a macrocyclic peptide. In other instances, the cyclic peptide is a constrained peptide. The cyclic peptides are assembled with varied linkages, such as for example, head-to-tail, head-to-side-chain, side-chain-to-tail, and side-chain-to-side-chain linkages. In some instances, a cyclic peptide (e.g., a macrocyclic or a constrained peptide) has a molecular weight from about 500 Dalton to about 2000 Dalton. In other instances, a cyclic peptide (e.g., a macrocyclic or a constrained peptide) ranges from about 10 amino acids to about 100 amino acids, from about 10 amino acids to about 70 amino acids, or from about 10 amino acids to about 50 amino acids.

In some cases, the peptide is for use in antigen production for therapeutic and/or prophylactic vaccine production. For example, the peptide comprises an antigen that elicits a desirable immune response (e.g., a pro-inflammatory immune response, an anti-inflammatory immune response, a tolerogenic immune response, an B cell response, an antibody response, a T cell response, a CD4+ T cell response, a CD8+ T cell response, a Th1 immune response, a Th2 immune response, a Th17 immune response, a Treg immune response, an M1 macrophage response, an M2 macrophage response, or a combination thereof).

In some embodiments, the peptide comprises natural amino acids, unnatural amino acids, or a combination thereof. In some instances, an amino acid residue refers to a molecule containing both an amino group and a carboxyl group. Suitable amino acids include, without limitation, both the D- and L-isomers of the naturally occurring amino acids, as well as non-naturally occurring amino acids prepared by organic synthesis or other metabolic routes. The term amino acid, as used herein, includes, without limitation, α-amino acids, natural amino acids, non-natural amino acids, and amino acid analogs.

In some instances, α-amino acid refers to a molecule containing both an amino group and a carboxyl group bound to a carbon which is designated the α-carbon.

In some instances, β-amino acid refers to a molecule containing both an amino group and a carboxyl group in a β configuration.

In some embodiments, an amino acid analog is a racemic mixture. In some instances, the D isomer of the amino acid analog is used. In some cases, the L isomer of the amino acid analog is used. In some instances, the amino acid analog comprises chiral centers that are in the R or S configuration.

In some embodiments, exemplary peptide cargos include, but are not limited to, peginesatide, insulin, adrenocorticotropic hormone (ACTH), calcitonin, oxytocin, vasopressin, octreolide, and leuprorelin.

In some embodiments, exemplary peptide cargos include, but are not limited to, Telavancin, Dalbavancin, Oritavancin, Anidulafungin, Lanreotide, Pasireotide, Romidepsin, Linaclotide, and Peginesatide.

### Antibodies

In some embodiments, the cargo is an antibody or a binding fragment thereof. In some instances, the antibody or binding fragment thereof comprises a humanized antibody or binding fragment thereof, murine antibody or binding fragment thereof, chimeric antibody or binding fragment thereof, monoclonal antibody or binding fragment thereof, bispecific antibody or biding fragment thereof, monovalent Fab′, divalent Fab₂, F(ab)′₃ fragments, single-chain variable fragment (scFv), bis-scFv, (scFv)₂, diabody, minibody, nanobody, triabody, tetrabody, disulfide stabilized Fv protein (dsFv), single-domain antibody (sdAb), Ig NAR, camelid antibody or binding fragment thereof, or a chemically modified derivative thereof.

In some instances, the antibody or binding fragment thereof recognizes a cell surface protein. In some instances, the cell surface protein is an antigen expressed by a cancerous cell. In some instances, the cell surface protein is a neoepitope. In some instances, the cell surface protein comprises one or more mutations compared to a wild-type protein. Exemplary cancer antigens include, but are not limited to, alpha fetoprotein, ASLG659, B7-H3, BAFF-R, Brevican, CA125 (MUC16), CA15-3, CA19-9, carcinoembryonic antigen (CEA), CA242, CRIPTO (CR, CR1, CRGF, CRIPTO, TDGF1, teratocarcinoma-derived growth factor), CTLA-4, CXCR5, E16 (LAT1, SLC7A5), FcRH2 (IFGP4, IRTA4, SPAP1A (SH2 domain containing phosphatase anchor protein 1a), SPAP1B, SPAP1C), epidermal growth factor, ETBR, Fc receptor-like protein 1 (FCRH1), GEDA, HLA-DOB (Beta subunit of MHC class II molecule (Ia antigen), human chorionic gonadotropin, ICOS, IL-2 receptor, IL20Rα, Immunoglobulin superfamily receptor translocation associated 2 (IRTA2), L6, Lewis Y, Lewis X, MAGE-1, MAGE-2, MAGE-3, MAGE 4, MART1, mesothelin, MDP, MPF (SMR, MSLN), MCP1 (CCL2), macrophage inhibitory factor (MIF), MPG, MSG783, mucin, MUC1-KLH, Napi3b (SLC34A2), nectin-4, Neu oncogene product, NCA, placental alkaline phosphatase, prostate specific membrane antigen (PMSA), prostatic acid phosphatase, PSCA hlg, anti-transferrin receptor, p97, Purinergic receptor P2X ligand-gated ion channel 5 (P2X5), LY64 (Lymphocyte antigen 64 (RP105), gp100, P21, six transmembrane epithelial antigen of prostate (STEAP1), STEAP2, Sema 5b, tumor-associated glycoprotein 72 (TAG-72), TrpM4 (BR22450, F1120041, TRPM4, TRPM4B, transient receptor potential cation channel, subfamily M, member 4) and the like.

In some instances, the cell surface protein comprises clusters of differentiation (CD) cell surface markers. Exemplary CD cell surface markers include, but are not limited to, CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD71, CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), and the like.

In some embodiments, exemplary antibodies or binding fragments thereof include, but are not limited to, zalutumumab (HuMax-EFGr, Genmab), abagovomab (Menarini), abituzumab (Merck), adecatumumab (MT201), alacizumab pegol, alemtuzumab (Campath®, MabCampath, or Campath-1H; Leukosite), AlloMune (BioTransplant), amatuximab (Morphotek, Inc.), anti-VEGF (Genetech), anatumomab mafenatox, apolizumab (hu1D10), ascrinvacumab (Pfizer Inc.), atezolizumab (MPDL3280A; Genentech/Roche), B43.13 (OvaRex, AltaRex Corporation), basiliximab (Simulect®, Novartis), belimumab (Benlysta®, GlaxoSmithKline), bevacizumab (Avastin®, Genentech), blinatumomab (Blincyto, AMG103; Amgen), BEC2 (ImGlone Systems Inc.), carlumab (Janssen Biotech), catumaxomab (Removab, Trion Pharma), CEAcide (Immunomedics), Cetuximab (Erbitux®, ImClone), citatuzumab bogatox (VB6-845), cixutumumab (IMC-A12, ImClone Systems Inc.), conatumumab (AMG 655, Amgen), dacetuzumab (SGN-40, huS2C6; Seattle Genetics, Inc.), daratumumab (Darzalex®, Janssen Biotech), detumomab, drozitumab (Genentech), durvalumab (MedImmune), dusigitumab (MedImmune), edrecolomab (MAb17-1A, Panorex, Glaxo Wellcome), elotuzumab (Empliciti™, Bristol-Myers Squibb), emibetuzumab (Eli Lilly), enavatuzumab (Facet Biotech Corp.), enfortumab vedotin (Seattle Genetics, Inc.), enoblituzumab (MGA271, MacroGenics, Inc.), ensituxumab (Neogenix Oncology, Inc.), epratuzumab (LymphoCide, Immunomedics, Inc.), ertumaxomab (Rexomun®, Trion Pharma), etaracizumab (Abegrin, MedImmune), farletuzumab (MORAb-003, Morphotek, Inc), FBTA05 (Lymphomun, Trion Pharma), ficlatuzumab (AVEO Pharmaceuticals), figitumumab (CP-751871, Pfizer), flanvotumab (ImClone Systems), fresolimumab (GC1008, Aanofi-Aventis), futuximab, glaximab, ganitumab (Amgen), girentuximab (Rencarex®, Wilex AG), IMAB362 (Claudiximab, Ganymed Pharmaceuticals AG), imalumab (Baxalta), IMC-1C11 (ImClone Systems), IMC-C225 (Imclone Systems Inc.), imgatuzumab (Genentech/Roche), intetumumab (Centocor, Inc.), ipilimumab (Yervoy®, Bristol-Myers Squibb), iratumumab (Medarex, Inc.), isatuximab (SAR650984, Sanofi-Aventis), labetuzumab (CEA-CIDE, Immunomedics), lexatumumab (ETR2-ST01, Cambridge Antibody Technology), lintuzumab (SGN-33, Seattle Genetics), lucatumumab (Novartis), lumiliximab, mapatumumab (HGS-ETR1, Human Genome Sciences), matuzumab (EMD 72000, Merck), milatuzumab (hLL1, Immunomedics, Inc.), mitumomab (BEC-2, ImClone Systems), narnatumab (ImClone Systems), necitumumab (Portrazza™, Eli Lilly), nesvacumab (Regeneron Pharmaceuticals), nimotuzumab (h-R3, BIOMAb EGFR, TheraCIM, Theraloc, or CIMAher; Biotech Pharmaceutical Co.), nivolumab (Opdivo®, Bristol-Myers Squibb), obinutuzumab (Gazyva or Gazyvaro; Hoffmann-La Roche), ocaratuzumab (AME-133v, LY2469298; Mentrik Biotech, LLC), ofatumumab (Arzerra®, Genmab), onartuzumab (Genentech), Ontuxizumab (Morphotek, Inc.), oregovomab (OvaRex®, AltaRex Corp.), otlertuzumab (Emergent BioSolutions), panitumumab (ABX-EGF, Amgen), pankomab (Glycotope GMBH), parsatuzumab (Genentech), patritumab, pembrolizumab (Keytruda®, Merck), pemtumomab (Theragyn, Antisoma), pertuzumab (Perjeta, Genentech), pidilizumab (CT-011, Medivation), polatuzumab vedotin (Genentech/Roche), pritumumab, racotumomab (Vaxira®, Recombio), ramucirumab (Cyramza®, ImClone Systems Inc.), rituximab (Rituxan®, Genentech), robatumumab (Schering-Plough), Seribantumab (Sanofi/Merrimack Pharmaceuticals, Inc.), sibrotuzumab, siltuximab (Sylvant™, Janssen Biotech), Smart MI95 (Protein Design Labs, Inc.), Smart ID10 (Protein Design Labs, Inc.), tabalumab (LY2127399, Eli Lilly), taplitumomab paptox, tenatumomab, teprotumumab (Roche), tetulomab, TGN1412 (CD28-SuperMAB or TAB08), tigatuzumab (CD-1008, Daiichi Sankyo), tositumomab, trastuzumab (Herceptin®), tremelimumab (CP-672,206; Pfizer), tucotuzumab celmoleukin (EMD Pharmaceuticals), ublituximab, urelumab (BMS-663513, Bristol-Myers Squibb), volociximab (M200, Biogen Idec), and zatuximab.

In some instances, the antibody or binding fragments thereof is an antibody-drug conjugate (ADC). In some cases, the payload of the ADC comprises, for example, but is not limited to, an auristatin derivative, maytansine, a maytansinoid, a taxane, a calicheamicin, cemadotin, a duocarmycin, a pyrrolobenzodiazepine (PDB), or a tubulysin. In some instances, the payload comprises monomethyl auristatin E (MMAE) or monomethyl auristatin F (MMAF). In some instances, the payload comprises DM2 (mertansine) or DM4. In some instances, the payload comprises a pyrrolobenzodiazepine dimer.

### Additional Cargos

In some embodiments, the cargo is a peptidomimetic. A peptidomimetic is a small protein-like polymer designed to mimic a peptide. In some instances, the peptidomimetic comprises D-peptides. In other instances, the peptidomimetic comprises L-peptides. Exemplary peptidomimetics include peptoids and β-peptides.

In some embodiments, the cargo is a nucleotidomimetic.

### Vectors and Expression Systems

PNMA2 and endo-Gag polypeptides of the disclosure can be encoded by nucleic acids, for example, for expression in a host cell or using a cell-free expression system. In certain embodiments, the PNMA2 polypeptides, endo-Gag polypeptides, engineered PNMA2 and engineered endo-Gag polypeptides described herein are encoded by vectors, e.g., plasmid vectors. In some embodiments, vectors include any suitable vector derived from either a eukaryotic or prokaryotic source. In some cases, vectors are obtained from bacteria (e.g. E. coli), insects, yeast (e.g. Pichia pastoris), algae, or mammalian sources.

Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pA0815 Pichia vector, pFLD1 Pichia pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors include p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In certain embodiments, the PNMA2 polypeptides, endo-Gag polypeptides, engineered PNMA2 and engineered endo-Gag polypeptides described herein and/or heterologous cargos are expressed using a cell-free expression system. In some instances, a cell-free system is a mixture of cytoplasmic and/or nuclear components from a cell, or isolated transcription and/or translation machinery, and is used for in vitro RNA and/or protein expression. In some cases, a cell-free system utilizes either prokaryotic cell components or eukaryotic cell components, or transcription and/or translation machinery derived therefrom. In some embodiments, nucleic acid synthesis is obtained in a cell-free system based on for example Drosophila cell, Xenopus egg, or HeLa cells (ATCC® CCL-2™). Exemplary cell-free systems include, but are not limited to, E. coli S30 Extract system, E. coli T7 S30 system, or PURExpress®.

### Host Cells

In certain embodiments, the PNMA2 polypeptides, endo-Gag polypeptides, engineered PNMA2 and engineered endo-Gag polypeptides described herein, and/or heterologous cargos are expressed by a host cell. In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., a yeast cell), an animal cell, or a plant cell. In some cases, a prokaryotic cell is a bacterial cell. Examples of bacterial cell include gram-positive bacteria or gram-negative bacteria. In some embodiments the gram-negative bacteria are anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. In some embodiments, bacteria is Acidobacteria, Chloroflexi, Chrysiogenetes, Cyanobacteria, Deferribacteres, Dictyoglomi, Thermodesulfobacteria or Thermotogae. In some embodiments, a bacterial cell is Escherichia coli, Clostridium botulinum, or Coli bacilli.

Exemplary prokaryotic host cells include, but are not limited to, BL21, Mach1™ DH10B™, TOP10, DH5α, DH10Bac™, OmniMax™, MegaX™, DH12S™, INV110, TOP10F′, INVαF, TOP10/P3, ccdB Survival, PIR1, PIR2, Stb12™, Stb13™, or Stb14™.

In some instances, animal cells include a cell from a vertebrate or from an invertebrate. In some cases, an animal cell includes a cell from a marine invertebrate, fish, insects, amphibian, reptile, mammal, or human. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailiff, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a human, primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293H cells, CHO DG44 cells, CHO—S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™—CHO cell line, Flp-In™-CV-1 cell line, Flp-In™-Jurkat cell line, FreeStyle™ 293-F cells, FreeStyle™ CHO—S cells, GripTite™ 293 MSR cell line, GS-CHO cell line, HepaRG™ cells, T-REx™ Jurkat cell line, Per.C6 cells, T-REx™-293 cell line, T-REx™—CHO cell line, and T-REx™-HeLa cell line.

In some instances, a mammalian host cell is a primary cell. In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells. Exemplary insect cell lines include, but are not limited to, strains from Chlamydomonas reinhardtii 137c, or Synechococcus elongatus PPC 7942. In some instances, plant cells include cells from algae.

### Methods

Disclosed herein, in certain embodiments, are methods of preparing a capsid, e.g., a capsid which encapsulates a heterologous cargo.

PNMA2 and endo-Gag polypeptides of the disclosure exhibit favorable properties for capsid assembly, disassembly, and/or reassembly.

The presence of PNMA2 and endo-Gag polypeptides in a capsid form or non-capsid form can be determined using, for example, size exclusion chromatography, multi-angle dynamic light scattering (MADLS), transmission electron microscopy (TEM), or a combination thereof. Capsid assembly, disassembly, and re-assembly efficiency can be calculated, for example, by comparing the quantity of protein present in assembled capsids to the total quantity of protein, or to the quantity of protein present in particles of smaller size and/or larger size than the capsids. In some embodiments, when calculating reassembly, the amount of protein loss during disassembly and reassembly can be measured and accounted for in the calculations.

In some embodiments, measuring capsid assembly efficiency comprises quantifying the amount of purified protein that is present in a capsid state (e.g., as capsid particles), and quantifying the amount of purified protein that is present in non-capsid states (e.g., non-assembled and/or partially-assembled states). For example, size exclusion chromatography can be used to separate monomers and oligomers from assembled capsids, and to quantify the amount of protein that is present in a capsid state versus un-assembled or partially-assembled non-capsid states.

In some embodiments, measuring capsid disassembly efficiency comprises quantifying the amount of capsid-forming protein that remains in solution after disassembling from a capsid state to a non-capsid state (e.g., oligomers, capsomers, and/or monomers). In some embodiments, the efficiency of disassembly can be determined by quantifying the percent of total protein that is a monomer peak as measured by MADLS or SEC. In some embodiments, capsids are treated with a disassembly buffer, and optionally centrifuged to get precipitate (e.g., aggregates) and/or remaining capsids out of solution. The loss of capsid structures confirmed by electron microscopy (e.g., TEM) and/or dynamic light scattering (e.g., MADLS). The efficiency of disassembly can be determined by MADLS (e.g., via a capsid peak and a monomer peak) or size exclusion chromatography (SEC). The amount of protein that remains in solution can be quantified. The amount of monomer in solution can be quantified. Disassembly efficiency can be calculated. For example, a recovery of 90 ng of protein in solution from 100 ng of starting capsid material indicates a disassembly efficiency of 90%.

In some embodiments, measuring capsid reassembly efficiency comprises quantifying an amount of disassembled protein in solution that reassembles into a capsid state. In some embodiments, an amount of input protein in a non-capsid state in solution is quantified (e.g., after treating with a disassembly buffer, optionally centrifugation to remove precipitate and/or capsids, and confirming a lack of capsid structures via TEM and/or MADLS). In some embodiments, disassembled protein is treated with a reassembly buffer disclosed herein, e.g., that comprises high salt, does not contain a chaotropic agent, and/or does not contain a reducing agent. Aggregates can be removed by centrifugation, e.g., at 15,000×g for 5 min at +4° C. The efficiency of reassembly can be determined by comparing the amounts of endo-Gag polypeptides in the monomer and capsid peaks as assayed by MADLS or SEC. Capsid formation can be confirmed by TEM and/or MADLS. In some embodiments, size exclusion chromatography performed to quantify the amount of protein in a capsid state, and this can be compared to an amount of input protein. Reassembly efficiency is calculated. For example, 81 ng of protein detected in a capsid state by size exclusion chromatography after starting with 90 ng of protein in a non-capsid state in solution indicates a 90% reassembly efficiency. In some embodiments, reassembly efficiency is determined by comparing the amount of endo-Gag polypeptides in reassembled capsids without further purification to the input amount of endo-Gag monomer polypeptides. In some embodiments, reassembly efficiency is determined by comparing the amount of endo-Gag polypeptides in reassembled capsids with further purification (e.g., via SEC or IEC) to the input amount of endo-Gag monomer polypeptides

In some embodiments, MADLS is used to determine particle concentration and verify whether it is within the expected range for the amount of protein in solution. For example, for PNMA2, which has a molecular weight of 41509.37, the expected number of particles can be calculated from the amount of PNMA2 polypeptide based on an assumption of 60 PNMA2 monomers per capsid: 1 g Capsids=2.42×10¹⁷ Capsids.

In some embodiments, an observed concentration of a capsid disclosed herein is within about ±5%, within about ±10%, within about ±20%, within about ±30%, within about ±40%, within about ±50%, within about ±60%, within about ±70%, within about ±80%, within about ±90%, within about ±2-fold, within about ±5-fold, or within about ±10-fold of an expected particle concentration.

In some embodiments, further purification is performed (e.g., via SEC or ion-exchange chromatography) if capsid reassembly is less than 5%, less than 10%, less than 20%, less than 30%, less than 40%, less than 50%, less than 60%, less than 70%, less than less than 80%, less than 85%, less than 86%, less than 87%, less than 88%, less than 89%, less than 90%, less than 91%, less than 92%, less than 93%, less than 94%, less than 95%, less than 96%, less than 97%, less than 98%, less than 99%, or less than 99.5% efficient.

In some embodiments, a PNMA2 or endo-Gag polypeptide of the disclosure exhibits favorable assembly properties. In some embodiments, upon isolation, a PNMA2 or endo-Gag polypeptide of the disclosure assembles to form capsids with an efficiency of at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 80%, at least about 90%, at least about 95%, at least about 97%, or at least about 99%. In some embodiments, at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 80%, at least about 90%, at least about 95%, at least about 97%, or at least about 99% of the isolated PNMA2 or endo-Gag polypeptide assembles to form the capsid.

In some embodiments, a PNMA2 or endo-Gag polypeptide of the disclosure exhibits favorable disassembly properties. In some embodiments, after incubation in a disassembly buffer, PNMA2 or endo-Gag capsids disassemble (e.g., to monomers, or smaller subunits than the capsids) with an efficiency of at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 80%, at least about 90%, at least about 95%, at least about 97%, or at least about 99%. In some embodiments, at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 80%, at least about 90%, at least about 95%, at least about 97%, or at least about 99% of the PNMA2 or endo-Gag polypeptide present in capsids disassembles to a non-capsid state.

In some embodiments, a PNMA2 or endo-Gag polypeptide of the disclosure exhibits favorable reassembly properties. In some embodiments, after disassembly, upon incubation in a suitable reassembly buffer, a PNMA2 or endo-Gag polypeptide of the disclosure reassembles to form capsids with an efficiency of at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 80%, at least about 90%, at least about 95%, at least about 97%, or at least about 99%. In some embodiments, at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 80%, at least about 90%, at least about 95%, at least about 97%, or at least about 99% of the PNMA2 or endo-Gag polypeptide reassembles to form the capsid, e.g., as a percentage of the PNMA2 or endo-Gag protein that is present after disassembly, as a percentage of the PNMA2 or endo-Gag protein that was originally present in capsids, or as a percentage of the PNMA2 or endo-Gag protein that is present upon initial protein isolation.

In some embodiments, an assembly buffer comprises a physiological buffer. In some embodiments, an assembly buffer is or comprises a balanced salt solution. In some embodiments, an assembly buffer is or comprises a saline, e.g., a buffered saline. In some embodiments, an assembly buffer is or comprises PBS. In some embodiments, an assembly buffer is or comprises HBSS. In some embodiments, an assembly buffer comprises is or ringer's solution. In some embodiments, an assembly buffer comprises sodium phosphate.

In some embodiments, a PNMA2 or endo-Gag polypeptide of the disclosure exhibits favorable assembly properties in buffers with low or physiological levels of salt.

In some embodiments, an assembly buffer comprises less than 2000, less than 1000, less than 900, less than 800, less than 700, less than 600, less than 500, less than 400, less than 300, or less than 200 mOsm/kg of salt. In some embodiments, an assembly buffer is not a balanced salt solution. In some embodiments, an assembly buffer comprises more than 200 mOsm/kg of salt.

In some embodiments, an assembly buffer comprises glycerol. One example of an assembly buffer is 75 mM NaCl, 50 mM Tris pH 8.0, 10% Glycerol. An additional example of an assembly buffer is phosphate buffered saline (PBS).

In some embodiments, capsid assembly is conducted at a temperature of at least −20° C., at least −10° C., at least 0° C., at least 2° C., at least 4° C., at least 6° C., at least 8° C., at least 10° C., at least 12° C., at least 14° C., at least 16° C., at least 18° C., at least 20° C., at least 22° C., at least 25° C., at least 30° C., or at least 37° C. In some embodiments, capsid assembly is conducted at a temperature of at most 2° C., at most 4° C., at most 6° C., at most 8° C., at most 10° C., at most 12° C., at most 14° C., at most 16° C., at most 18° C., at most 20° C., at most 22° C., at most 25° C., at most 30° C., at most 37° C., at most 40° C. or at most 45° C. In some embodiments, capsid assembly is conducted at a temperature of about −20° C., about −10° C., about 0° C., about 2° C., about 4° C., about 6° C., about 8° C., about 10° C., about 12° C., about 14° C., about 16° C., about 18° C., about 20° C., about 22° C., about 25° C., about 30° C., or about 37° C. In some embodiments, capsid assembly is conducted at a temperature of about 2° C. to about 40° C., 2° C. to about 37° C., 2° C. to about 25° C., 2° C. to about 20° C., 4° C. to about 40° C., 4° C. to about 37° C., 4° C. to about 25° C., 4° C. to about 20° C., 2° C. to about 8° C., about 2° C. to about 4° C., about 20° C. to about 37° C., about 25° C. to about 37° C., about 20° C. to about 30° C., about 25° C. to about 30° C., or about 30° C. to about 37° C.

In some embodiments, capsid assembly comprises incubating in an assembly buffer for at least about 5 minutes, at least about 10 minutes, at least about 20 minutes, at least about 30 minutes, at least about 1 hour, at least about 2 hours, at least about 4 hours, at least about 6 hours, at least about 8 hours, at least about 10 hours, at least about 12 hours, at least about 16 hours, at least about 20 hours, at least about 24 hours, or more.

In some embodiments, an assembly buffer comprises a pH of about 3, about 4, about 5, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 10, or about 11. In some embodiments, an assembly buffer comprises a pH of at least about 3, at least about 4, at least about 5, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, or at least about 8. In some embodiments, an assembly buffer comprises a pH of at most about 7, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 10, or at most about 11. In some embodiments, an assembly buffer comprises a pH of about 5-9, 5-8, 5-7.8, 5-7.6, 5-7.5, 5-7.4, 6-8, 6-7.8, 6-7.6, 6-7.5, 6-7.4, 7-8, 7-7.8, 7-7.6, or 7-7.5.

A disassembly buffer can comprise one or more reducing agents, e.g., reduced glutathione (GSH), beta mercaptoethanol ((3-ME), Dithiothreitol (DTT), tris(2-carboxyethyl)phosphine (TCEP), or a combination thereof. A disassembly buffer can comprise one or more solubilizing agents, for example, a detergent, including a non-denaturing detergent, such as CHAPS, nP-40, n-OG, DDM, Triton X-100, LDAO, Tween 20, or a combination thereof. A disassembly buffer can comprise one or more non-detergent sulfobetaines, for example, NDSB-195, NDSB-201, NDSB-211, NDSB-221, NDSB-256, NDSB-256-4T, or a combination thereof. A disassembly buffer can comprise one or more chaotropic agent. A disassembly buffer can comprise n-butanol, ethanol, guanidinium chloride, lithium perchlorate, lithium acetate, magnesium chloride, phenol, 2-propanol, sodium dodecyl sulfate, thiourea, urea, mgc12. Cacl2, NaI, or a combination thereof.

A disassembly buffer can comprise Tris. A disassembly buffer can comprise NaCl. A disassembly buffer can comprise NaP. A disassembly buffer can comprise glycerol. In some embodiments, a disassembly buffer comprises MgCl₂. A disassembly buffer can comprise urea. A disassembly buffer can comprise DTT. A disassembly buffer can comprise urea and DTT. An illustrative example of a disassembly buffer is 10% CHAPS, 20 mM TCEP, 100 mM Tris pH 8.0. An additional example of a disassembly buffer is 10 mM TCEP, 10 mM MgCl₂, 50 mM Tris pH 8.0. A further example of a disassembly buffer is a buffer containing 12 mM GSH. Additional examples of disassembly buffers include: (i) 5.33 M Urea, 66.67 mM DTT, 500 mM NaCl, 20 mM NaP pH 7.4, 10% glycerol; (ii) 4 M Urea, 50 mM DTT, 500 mM NaCl, 20 mM NaP pH 7.4, 10% glycerol; and (iii) 4-6 M Urea, 30-50 mM DTT, 500 mM NaCl, 10% glycerol.

A disassembly buffer can comprise a component disclosed herein at a concentration of at least 1 μm, at least 10 pM, at least 100 pM, at least 1 nM, at least 10 nM, at least 100 nM, at least 1 μM, at least 10 μM, at least 100 μM, at least 1 mM, at least 10 mM, at least 100 mM, at least 1 M, or at least 5 M. A disassembly buffer can comprise a component disclosed herein at a concentration of at most 1 μm, at most 10 pM, at most 100 pM, at most 1 nM, at most 10 nM, at most 100 nM, at most 1 μM, at most 10 μM, at most 100 μM, at most 1 mM, at most 10 mM, at most 100 mM, at most 1 M, or at most 5 M.

In some embodiments, capsid disassembly is conducted at a temperature of at least −20° C., at least −10° C., at least 0° C., at least 2° C., at least 4° C., at least 6° C., at least 8° C., at least 10° C., at least 12° C., at least 14° C., at least 16° C., at least 18° C., at least 20° C., at least 22° C., at least 25° C., at least 30° C., or at least 37° C. In some embodiments, capsid disassembly is conducted at a temperature of at most 2° C., at most 4° C., at most 6° C., at most 8° C., at most 10° C., at most 12° C., at most 14° C., at most 16° C., at most 18° C., at most 20° C., at most 22° C., at most 25° C., at most 30° C., at most 37° C., at most 40° C. or at most 45° C. In some embodiments, capsid disassembly is conducted at a temperature of about −20° C., about −10° C., about 0° C., about 2° C., about 4° C., about 6° C., about 8° C., about 10° C., about 12° C., about 14° C., about 16° C., about 18° C., about 20° C., about 22° C., about 25° C., about 30° C., or about 37° C. In some embodiments, capsid disassembly is conducted at a temperature of about 2° C. to about 40° C., 2° C. to about 37° C., 2° C. to about 25° C., 2° C. to about 20° C., 4° C. to about 40° C., 4° C. to about 37° C., 4° C. to about 25° C., 4° C. to about 20° C., 2° C. to about 8° C., about 2° C. to about 4° C., about 20° C. to about 37° C., about 25° C. to about 37° C., about 20° C. to about 30° C., about 25° C. to about 30° C., or about 30° C. to about 37° C.

In some embodiments, capsid disassembly comprises incubating in a disassembly buffer for at least about 5 minutes, at least about 10 minutes, at least about 20 minutes, at least about 30 minutes, at least about 1 hour, at least about 2 hours, at least about 4 hours, at least about 6 hours, at least about 8 hours, at least about 10 hours, at least about 12 hours, at least about 16 hours, at least about 20 hours, at least about 24 hours, or more.

In some embodiments, a disassembly buffer comprises a pH of about 3, about 4, about 5, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 10, or about 11. In some embodiments, a disassembly buffer comprises a pH of at least about 3, at least about 4, at least about 5, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, or at least about 8. In some embodiments, a disassembly buffer comprises a pH of at most about 7, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 10, or at most about 11. In some embodiments, a disassembly buffer comprises a pH of about 5-9, about 5-8, about 5-7.8, about 5-7.6, about 5-7.5, about 5-7.4, about 6-8, about 6-7.8, about 6-7.6, about 6-7.5, about 6-7.4, about 7-8, about 7-7.8, about 7-7.6, or about 7-7.5.

In some embodiments, a reassembly buffer comprises one or more monovalent salts, for example, NaCl, KCl, LiCl, NaBr, NaF, or a combination thereof. In some embodiments, a reassembly buffer comprises one or more osmolytes, for example, Glycerol, Ethylene glycol, Mannitol, Glycine, Glycine Betaine, Trehalose, Sorbitol, or a combination thereof.

In some embodiments, a reassembly buffer does not contain or substantially lacks a chaotropic agent. In some embodiments, a reassembly buffer does not contain or substantially lacks a reducing agent.

A reassembly buffer can comprise a component disclosed herein at a concentration of at least 1 μm, at least 10 pM, at least 100 pM, at least 1 nM, at least 10 nM, at least 100 nM, at least 1 μM, at least 10 μM, at least 100 μM, at least 1 mM, at least 10 mM, at least 100 mM, at least 1 M, or at least 5 M. A reassembly buffer can comprise a component disclosed herein at a concentration of at most 1 μm, at most 10 pM, at most 100 pM, at most 1 nM, at most 10 nM, at most 100 nM, at most 1 μM, at most 10 μM, at most 100 μM, at most 1 mM, at most 10 mM, at most 100 mM, at most 1 M, or at most 5 M.

In some embodiments, a reassembly buffer comprises a physiological buffer. In some embodiments, a reassembly buffer comprises a balanced salt solution. In some embodiments, a reassembly buffer is or comprises a saline, e.g., a buffered saline. In some embodiments, a reassembly buffer is or comprises PBS. In some embodiments, a reassembly buffer comprises sodium phosphate. In some embodiments, a reassembly buffer is or comprises HBSS. In some embodiments, a reassembly buffer comprises is or ringer's solution.

In some embodiments, a PNMA2 or endo-Gag polypeptide of the disclosure exhibits favorable reassembly properties in buffers with low or physiological levels of salt.

In some embodiments, a reassembly buffer comprises less than 2000, less than 1000, less than 900, less than 800, less than 700, less than 600, less than 500, less than 400, less than 300, or less than 200 mOsm/kg of salt. In some embodiments, a reassembly buffer is not a balanced salt solution.

In some embodiments, a reassembly buffer comprises high salt. In some embodiments, a reassembly buffer comprises more than 200 mOsm/kg of salt. In some embodiments, a reassembly buffer comprises at least 8000, at least 7000, at least 6000, at least 5000, at least 4000, at least 3000, at least 2000, at least 1000, at least 900, at least 800, at least 700, at least 600, at least 500, at least 400, at least 300, or at least 200 mOsm/kg of salt.

In some embodiments, a reassembly buffer comprises glycerol. One example of a reassembly buffer is 75 mM NaCl, 50 mM Tris pH 8.0, 10% Glycerol. An additional example of a reassembly buffer is PBS. A further example of a reassembly buffer is 20 mM Na₂HPO₄/NaH₂PO₄, pH 7.4, 0.5 M NaCl, 10% glycerol, 40 mM imidazole, 1 mM DTT.

In some embodiments, capsid reassembly is conducted at a temperature of at least −20° C., at least −10° C., at least 0° C., at least 2° C., at least 4° C., at least 6° C., at least 8° C., at least 10° C., at least 12° C., at least 14° C., at least 16° C., at least 18° C., at least 20° C., at least 22° C., at least 25° C., at least 30° C., or at least 37° C. In some embodiments, capsid reassembly is conducted at a temperature of at most 2° C., at most 4° C., at most 6° C., at most 8° C., at most 10° C., at most 12° C., at most 14° C., at most 16° C., at most 18° C., at most 20° C., at most 22° C., at most 25° C., at most 30° C., at most 37° C., at most 40° C. or at most 45° C. In some embodiments, capsid reassembly is conducted at a temperature of about −20° C., about −10° C., about 0° C., about 2° C., about 4° C., about 6° C., about 8° C., about 10° C., about 12° C., about 14° C., about 16° C., about 18° C., about 20° C., about 22° C., about 25° C., about 30° C., or about 37° C. In some embodiments, capsid reassembly is conducted at a temperature of about 2° C. to about 40° C., 2° C. to about 37° C., 2° C. to about 25° C., 2° C. to about 20° C., 4° C. to about 40° C., 4° C. to about 37° C., 4° C. to about 25° C., 4° C. to about 20° C., 2° C. to about 8° C., about 2° C. to about 4° C., about 20° C. to about 37° C., about 25° C. to about 37° C., about 20° C. to about 30° C., about 25° C. to about 30° C., or about 30° C. to about 37° C.

In some embodiments, capsid reassembly comprises incubating in a reassembly buffer for at least about 5 minutes, at least about 10 minutes, at least about 20 minutes, at least about 30 minutes, at least about 1 hour, at least about 2 hours, at least about 4 hours, at least about 6 hours, at least about 8 hours, at least about 10 hours, at least about 12 hours, at least about 16 hours, at least about 20 hours, at least about 24 hours, or more.

In some embodiments, a reassembly buffer comprises a pH of about 3, about 4, about 5, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 10, or about 11. In some embodiments, a reassembly buffer comprises a pH of at least about 3, at least about 4, at least about 5, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, or at least about 8. In some embodiments, a reassembly buffer comprises a pH of at most about 7, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 10, or at most about 11. In some embodiments, a reassembly buffer comprises a pH of about 5-9, 5-8, 5-7.8, 5-7.6, 5-7.5, 5-7.4, 6-8, 6-7.8, 6-7.6, 6-7.5, 6-7.4, 7-8, 7-7.8, 7-7.6, or 7-7.5.

In some embodiments, the method comprises incubating a plurality of PNMA2 or endo-Gag polypeptides, engineered PNMA2 or endo-Gag polypeptides, and/or recombinant PNMA2 or endo-Gag polypeptides with a heterologous cargo in a solution for a time sufficient to generate a loaded PNMA2-based capsid or endo-Gag-based capsid.

In some cases, the time sufficient to generate a loaded PNMA2-based capsid or endo-Gag-based capsid is at least about 5 minutes, at least about 10 minutes, at least about 20 minutes, at least about 30 minutes, at least about 1 hour, at least about 2 hours, at least about 4 hours, at least about 6 hours, at least about 8 hours, at least about 10 hours, at least about 12 hours, at least about 16 hours, at least about 20 hours, at least about 24 hours, or more. In some embodiments, the time sufficient to generate a loaded PNMA2-based capsid or endo-Gag-based capsid is at most about 5 minutes, at most about 10 minutes, at most about 20 minutes, at most about 30 minutes, at most about 1 hour, at most about 2 hours, at most about 4 hours, at most about 6 hours, at most about 8 hours, at most about 10 hours, at most about 12 hours, at most about 16 hours, at most about 20 hours, at most about 24 hours, or at most about 48 hours.

In some cases, the PNMA2-based capsid or endo-Gag-based capsid is assembled, disassembled, or reassembled at a temperature from about 2° C. to about 37° C. In some instances, the PNMA2-based capsid or endo-Gag-based capsid is prepared at a temperature from about 2° C. to about 8° C., about 2° C. to about 4° C., about 20° C. to about 37° C., about 25° C. to about 37° C., about 20° C. to about 30° C., about 25° C. to about 30° C., about 30° C. to about 37° C., from about 2° C. to about 40° C., 2° C. to about 25° C., 2° C. to about 20° C., 4° C. to about 40° C., 4° C. to about 37° C., 4° C. to about 25° C., or 4° C. to about 20° C. In some cases, the PNMA2-based capsid or endo-Gag-based capsid is assembled, disassembled, or reassembled at room temperature.

In some instances, the method comprises mixing a solution comprising a plurality of engineered and/or recombinant PNMA2 polypeptides with a plurality of non-PNMA2 capsid forming subunits prior to incubating with the cargo. In some cases, the plurality of non-PNMA2 capsid forming subunits are mixed with the plurality of engineered and/or recombinant PNMA2 polypeptides at a ratio of 1:1, 2:1, 3:1, 4:1, 5:1, 6:1, 7:1, 8:1, 9:1, or 10:1. In other cases, the plurality of non-PNMA2 capsid forming subunits are mixed with the plurality of engineered and/or recombinant PNMA2 polypeptides at a ratio of 1:2, 1:3, 1:4, 1:5, 1:6, 1:7, 1:8, 1:9, or 1:10.

In some instances, the PNMA2-based capsid or endo-Gag-based capsid is further formulated for systemic administration. In some instances, the PNMA2-based capsid or endo-Gag-based capsid is further formulated for local administration. In some instances, the PNMA2-based capsid or endo-Gag-based capsid is further formulated for parenteral (e.g., intra-arterial, intra-articular, intradermal, intralesional, intramuscular, intraocular, intraosseous infusion, intraperitoneal, intrathecal, intravenous, intravitreal, or subcutaneous) administration. In some instances, the PNMA2-based capsid or endo-Gag-based capsid is further formulated for topical administration. In some instances, the PNMA2-based capsid or endo-Gag-based capsid is further formulated for oral administration. In some instances, the PNMA2-based capsid or endo-Gag-based capsid is further formulated for sublingual administration. In some instances, the PNMA2-based capsid or endo-Gag-based capsid is further formulated for aerosol administration.

In certain embodiments, also described herein is a use of a PNMA2-based capsid or endo-Gag-based capsid for delivery of a cargo to a site of interest.

In some instances, methods of the disclosure comprise contacting a cell with a PNMA2-based capsid or endo-Gag-based capsid. The contacting can be in vitro, ex vivo, or in vivo. In some embodiments, the contacting is in vivo at a site of interest.

In some instances, the method comprises contacting a cell at the site of interest with a PNMA2-based capsid or endo-Gag-based capsid for a time sufficient to facilitate cellular uptake of the capsid. The time sufficient to facilitate cellular uptake of the capsid can be at least about 5 minutes, at least about 10 minutes, at least about 20 minutes, at least about 30 minutes, at least about 1 hour, at least about 2 hours, at least about 4 hours, at least about 6 hours, at least about 8 hours, at least about 10 hours, at least about 12 hours, at least about 16 hours, at least about 20 hours, at least about 24 hours, or more. In some embodiments, the time sufficient to facilitate cellular uptake of the capsid is at most about 5 minutes, at most about 10 minutes, at most about 20 minutes, at most about 30 minutes, at most about 1 hour, at most about 2 hours, at most about 4 hours, at most about 6 hours, at most about 8 hours, at most about 10 hours, at most about 12 hours, at most about 16 hours, at most about 20 hours, at most about 24 hours, or at most about 48 hours.

In some embodiments, the method comprises contacting a cell with a PNMA2-based capsid or endo-Gag-based capsid at a concentration sufficient to elicit a desired effect on the cell. The concentration sufficient to elicit the desired effect can be at least about 0.001 pg/mL, at least about 0.01 pg/mL, at least about 0.1 pg/mL, at least about 1 pg/mL, at least about 10 pg/mL, at least about 100 pg/mL, at least about 1 ng/mL, at least about 10 ng/mL, at least about 100 ng/mL, at least about 1 μg/mL, at least about 10 μg/mL, at least about 100 μg/mL, at least about 1 mg/mL, at least about 10 mg/mL, or at least about 100 mg/mL.

In some cases, the cell is a muscle cell, a skin cell, a blood cell, or an immune cell (e.g., a T cell or a B cell).

In some instances, the cell is a tumor cell, e.g., a solid tumor cell or a cell from a hematologic malignancy. In some cases, the solid tumor cell is a cell from a bladder cancer, breast cancer, brain cancer, colorectal cancer, kidney cancer, liver cancer, lung cancer, pancreatic cancer, prostate cancer, skin cancer, stomach cancer, or thyroid cancer. In some cases, the cell from a hematologic malignancy is from a B-cell malignancy or a T-cell malignancy. In some cases, the cell is from a leukemia, a lymphoma, a myeloma, chronic lymphocytic leukemia (CLL), small lymphocytic lymphoma (SLL), diffuse large B cell lymphoma (DLBCL), follicular lymphoma, mantle cell lymphoma, Burkitt lymphoma, cutaneous T-cell lymphoma, peripheral T cell lymphoma, multiple myeloma, plasmacytoma, acute lymphoblastic leukemia (ALL), acute myeloid leukemia (AML), or chronic myeloid leukemia (CML).

In some embodiments, the cell is a somatic cell. In some instances, the cell is a blood cell, a skin cell, a connective tissue cell, a bone cell, a muscle cell, or a cell from an organ.

In some embodiments, the cell is an epithelial cell, a connective tissue cell, a muscular cell, or a neuron.

In some instances, the cell is an endodermal cell, a mesodermal cell, or an ectodermal cell. In some instances, the endoderm comprises cells of the respiratory system, the intestine, the liver, the gallbladder, the pancreas, the islets of Langerhans, the thyroid, or the hindgut. In some cases, the mesoderm comprises osteochondroprogenitor cells, muscle cells, cells from the digestive system, renal stem cells, cells from the reproductive system, cells from the circulatory system (such as endothelial cells). Exemplary cells from the ectoderm comprise epithelial cells, cells of the anterior pituitary, cells of the peripheral nervous system, cells of the neuroendocrine system, cells of the eyes, cells of the central nervous system, cells of the ependymal, or cells of the pineal gland. In some cases, cells derived from the central and peripheral nervous system comprise neurons, Schwann cells, satellite glial cells, oligodendrocytes, or astrocytes. In some cases, neurons further comprise interneurons, pyramidal neurons, GABAergic neurons, dopaminergic neurons, serotoninergic neurons, glutamatergic neurons, motor neurons from the spinal cord, or inhibitory spinal neurons.

In some embodiments, the cell is a stem cell or a progenitor cell. In some cases, the cell is a mesenchymal stem or progenitor cell. In other cases, the cell is a hematopoietic stem or progenitor cell.

In some cases, a target protein is overexpressed or is depleted in the cell. In some cases, the target protein is overexpressed in the cell. In additional cases, the target protein is depleted in the cell.

In some cases, a target gene in the cell has one or more mutations. In some cases, the cell comprises an impaired splicing mechanism.

In some instances, the PNMA2-based capsid is administered systemically to a subject in need thereof. In other instances, the PNMA2-based capsid or endo-Gag-based capsid is administered locally to a subject in need thereof. In some embodiments, the PNMA2-based capsid or endo-Gag-based capsid is administered parenterally, orally, topically, via sublingual, or by aerosol to a subject in need thereof. In some cases, the PNMA2-based capsid or endo-Gag-based capsid is administered parenterally to a subject in need thereof. In other cases, the PNMA2-based capsid or endo-Gag-based capsid is administered orally to a subject in need thereof. In additional cases, the PNMA2-based capsid or endo-Gag-based capsid is administered topically, via sublingual, or by aerosol to a subject in need thereof.

In some embodiments, a delivery component is combined with a PNMA2-based capsid or endo-Gag-based capsid for a targeted delivery to a site of interest. In some instances, the delivery component comprises a carrier, e.g., an extracellular vesicle such as a micelle, a liposome, or a microvesicle; or a viral envelope.

In some instances, the delivery component serves as a primary delivery vehicle for a PNMA2-based capsid or endo-Gag-based capsid which does not comprise its own delivery component. In such cases, the delivery component directs the PNMA2-based capsid or endo-Gag-based capsid to a target site of interest and optionally facilitates intracellular uptake.

In other instances, the delivery component enhances target specificity and/or sensitivity of a PNMA2-based capsid. In such cases, the delivery component enhances the specificity and/or affinity of the PNMA2-based capsid or endo-Gag-based capsid to the target site. In some embodiments, the delivery components enhance the specificity and/or affinity by at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 50-fold, at least 100-fold, at least 200-fold, at least 500-fold, or more. In additional cases, the delivery components enhance the specificity and/or affinity by about 2-fold, 3-fold, 4-fold, 5-fold, 6-fold, 7-fold, 8-fold, 9-fold, 10-fold, 20-fold, 30-fold, 50-fold, 100-fold, 200-fold, 500-fold, or more. In further cases, the delivery components enhance the specificity and/or affinity by at least 10%, at least 20%, at least 30%, at least 40%, at least 50%, at least 60%, at least 70%, at least 80%, at least 90%, at least 100%, at least 200%, at least 500%, or more.

In some embodiments, the capsid has a low or reduced off-target effect. An off-target effect can be, for example, an effect that occurs when a cargo is delivered to an unintended tissue or cell type, or an effect that occurs when a cargo binds to an unintended interaction partner. In some cases, the off-target effect is less than 10%, less than 5%, less than 4%, less than 3%, less than 2%, less than 1%, or less than 0.5%. In some cases, the capsid does not have an off-target effect.

In some cases, the delivery components enhance the specificity and/or affinity by at least 10%, at least 20%, at least 30%, at least 40%, at least 50%, at least 60%, at least 70%, at least 80%, at least 90%, at least 100%, at least 200%, at least 500%, or more. In further cases, the delivery components enhance the specificity and/or affinity by about 10%, 20%, 30%, 40%, 50%, 60%, 70%, 80%, 90%, 100%, 200%, 500%, or more. Further still, the delivery component optionally reduces an off-target effect by at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 50-fold, at least 100-fold, at least 200-fold, at least 500-fold, or more. Further still, the delivery component optionally reduces off-target effect by at least 10%, at least 20%, at least 30%, at least 40%, at least 50%, at least 60%, at least 70%, at least 80%, at least 90%, at least 100%, at least 200%, at least 500%, or more.

In additional instances, the delivery component serves as a first vehicle that transports a PNMA2-based capsid to a general target region (e.g., a tumor microenvironment) and the PNMA2-based or endo-Gag-based capsid comprises a second delivery component (for example, an exogenous polypeptide sequence as disclosed herein) that directs the PNMA2-based capsid or endo-Gag-based capsid to a more specific target site and optionally facilitates intracellular uptake. In some embodiments, the delivery component (e.g., delivery component that serves as the first vehicle) minimizes off-target effect by at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 50-fold, at least 100-fold, at least 200-fold, at least 500-fold, or more. In some embodiments, the delivery component minimizes off-target effect by at least 10%, at least 20%, at least 30%, at least 40%, at least 50%, at least 60%, at least 70%, at least 80%, at least 90%, at least 100%, at least 200%, at least 500%, or more.

In further instances, the delivery component serves as a first vehicle that transports a PNMA2-based capsid to a target site of interest, and the PNMA2-based or endo-Gag-based capsid comprises a second delivery component that facilitates intracellular uptake, for example, an exogenous polypeptide sequence as disclosed herein.

In some embodiments, the delivery component comprises an extracellular vesicle. In some instances, the extracellular vesicle comprises a microvesicle, a liposome, or a micelle. In some instances, the extracellular vesicle has an average diameter of from about 10 nm to about 2000 nm, from about 10 nm to about 1000 nm, from about 10 nm to about 800 nm, from about 20 nm to about 600 nm, from about 30 nm to about 500 nm, from about 50 nm to about 200 nm, or from about 80 nm to about 100 nm.

In some embodiments, the delivery component comprises a microvesicle. Also known as circulating microvesicles or microparticles, microvesicles are membrane-bound vesicles that comprise phospholipids. In some instances, the microvesicle has an average diameter of from about 50 nm to about 1000 nm, from about 100 nm to about 800 nm, from about 200 nm to about 500 nm, or from about 50 nm to about 400 nm.

In some instances, the microvesicle is originated from cell membrane inversion, exocytosis, shedding, blebbing, or budding. In some instances, the microvesicles are generated from differentiated cells. In other instances, the microvesicles are generated from undifferentiated cells, e.g., by blast cells, progenitor cells, or stem cells.

In some embodiments, the delivery component comprises a liposome. In some instances, the liposome comprises a plurality of lipopeptides, which are presented on the surface of the liposome, for targeted delivery to a site or region of interest. In some cases, the liposomes fuse with the target cell, whereby the contents of the liposome are then emptied into the target cell. In some cases, a liposome is endocytosed by cells that are phagocytic. Endocytosis is then followed by intralysosomal degradation of liposomal lipids and release of the encapsulated agents.

Exemplary liposomes suitable for incorporation include, and are not limited to, multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). In some instances, a liposome comprises Amphipol (A8-35). Techniques for preparing liposomes are described in, for example, COLLOIDAL DRUG DELIVERY SYSTEMS, vol. 66 (J. Kreuter ed., Marcel Dekker, Inc. (1994)), which is incorporated herein by reference for such disclosure.

Depending on the method of preparation, liposomes are unilamellar or multilamellar, and vary in size with diameters ranging from about 20 nm to greater than about 1000 nm.

In some instances, liposomes provided herein also comprise carrier lipids. In some embodiments the carrier lipids are phospholipids. Carrier lipids capable of forming liposomes include, but are not limited to, dipalmitoylphosphatidylcholine (DPPC), phosphatidylcholine (PC; lecithin), phosphatidic acid (PA), phosphatidylglycerol (PG), phosphatidylethanolamine (PE), or phosphatidylserine (PS). Other suitable phospholipids further include di stearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE) and the like, or combinations thereof. In some embodiments, the liposomes further comprise a sterol (e.g., cholesterol) which modulates liposome formation. The carrier lipids are optionally any non-phosphate polar lipids. In some embodiments, a liposome comprises an electroneutral lipid.

In some embodiments, a liposome or a delivery component comprises a cationic lipid. Cationic lipids have a head group with permanent positive charges. Non-limiting examples of cationic lipids include 1,2-di-O-octadecenyl-3-trimethylammonium-propane (DOTMA), 1,2-dioleoyl-3-trimethylammonium-propane (DOTAP), Dimethyl dioctadecylammonium bromide (DDAB), and 2,3-dioleyloxy-N-[2-(sperminecarboxamido)ethyl]-N,N-dimethyl-1-propanaminium trifluoroacetate (DOSPA), and commercially available transfection reagents.

In some embodiments, the delivery component comprises a micelle. In some instances, the micelle has an average diameter from about 2 nm to about 250 nm, from about 20 nm to about 200 nm, from about 20 nm to about 100 nm, or from about 50 to about 100 nm.

In some instances, the micelle is a polymeric micelle, characterized by a core shell structure, in which the hydrophobic core is surrounded by a hydrophilic shell. In some cases, the hydrophilic shell further comprises a hydrophilic polymer or copolymer and a pH sensitive component.

Exemplary hydrophilic polymers or copolymers include, but are not limited to, poly(N-substituted acrylamides), poly(N-acryloyl pyrrolidine), poly(N-acryloyl piperidine), poly(N-acryl-L-amino acid amides), poly(ethyl oxazoline), methylcellulose, hydroxypropyl acrylate, hydroxyalkyl cellulose derivatives and poly(vinyl alcohol), poly(N-isopropylacrylamide), poly(N-vinyl-2-pyrrolidone), polyethyleneglycol derivatives, and combinations thereof.

The delivery component may comprise a pH-sensitive moiety, which can include, but is not limited to, an alkylacrylic acid such as methacrylic acid, ethylacrylic acid, propyl acrylic acid and butyl acrylic acid, or an amino acid such as glutamic acid.

In some instances, a hydrophobic moiety constitutes the core of the micelle and includes, for example, a single alkyl chain, such as octadecyl acrylate or a double chain alkyl compound such as phosphatidylethanolamine or dioctadecylamine. In some cases, the hydrophobic moiety is optionally a water insoluble polymer such as a poly(lactic acid) or a poly(e-caprolactone).

Polymeric micelles exhibiting pH-sensitive properties are also contemplated and are formed, e.g., by using pH-sensitive polymers including, but not limited to, copolymers from methacrylic acid, methacrylic acid esters and acrylic acid esters, polyvinyl acetate phthalate, hydroxypropyl methyl cellulose phthalate, cellulose acetate phthalate, or cellulose acetate trimellitate.

A Delivery component can comprise a cationic moiety, for example, a cationic lipid, a cationic peptide, or a cationic polymer. A cationic moiety (e.g., lipid, peptide, or moiety) can associate with a capsid (e.g., a negative spike portion thereof) via electrostatic interactions. The presence of a cationic lipid, polymer, or peptide on a capsid can enhance binding to the negatively charged surface of a cell, facilitating increased capsid uptake.

In some embodiments, the delivery component comprises a cationic peptide, e.g., a cationic cell-penetrating peptide disclosed herein.

In some embodiments, the delivery component comprises a cationic polymer. Non-limiting examples of cationic polymers include cationic peptides and their derivatives (e.g., polylysine, polyornithine), linear or branched synthetic polymers (e.g., polybrene, polyethyleneimine), polysaccharide-based delivery molecules (e.g., cyclodextrin, chitosan), natural polymers (e.g., histone, collagen), and activated and non-activated dendrimers. Cationic reagents can adhere to the cell membrane through electrostatic interactions and promote cellular uptake via endocytosis.

In some embodiments, the delivery component comprises a viral envelope or a component thereof. For example, viral envelopes comprise glycoproteins, phospholipids, and additional proteins obtained from a host, any of which a delivery component can comprise. In some instances, the viral envelope or component thereof is permissive to a wide range of target cells. In other instances, the viral envelope or component thereof is non-permissive and is specific to a target cell of interest. In some cases, the viral envelope or component thereof comprises a cell-specific binding protein and optionally a fusogenic molecule that aids in the fusion of the cargo into a target cell. In some cases, the viral envelope or component thereof comprises an endogenous viral envelope. In other cases, the viral envelope is a modified envelop, comprising one or more foreign proteins.

In some instances, the viral envelope or component thereof is derived from a DNA virus. Exemplary enveloped DNA viruses include viruses from the family of Herpesviridae, Poxviridae, and Hepadnaviridae. In other instances, the viral envelope or component thereof is derived from an RNA virus. Exemplary enveloped RNA viruses include viruses from the family of Bunyaviridae, Coronaviridae, Filoviridae, Flaviviridae, Orthomyxoviridae, Paramyxoviridae, Rhabdoviridae, and Togaviridae. In additional instances, the viral envelope or component thereof is derived from a virus from the family of Retroviridae.

In some embodiments, the viral envelope or component thereof is from an oncolytic virus, such as an oncolytic DNA virus from the family of Herpesviridae (for example, HSV1) or Poxviridae (for example, Vaccinia virus and myxoma virus); or an oncolytic RNA virus from the family of Rhabdoviridae (for example, VSV) or Paramyxoviridae (for example MV and NDV).

In some instances, the delivery component, viral envelope, or component thereof further comprises a foreign or engineered protein that binds to an antigen or a cell surface molecule. Exemplary antigens and cell surface molecules for targeting include, but are not limited to, P-glycoprotein, Her2/Neu, erythropoietin (EPO), epidermal growth factor receptor (EGFR), vascular endothelial growth factor receptor (VEGF-R), cadherin, carcinoembryonic antigen (CEA), CD4. CD8, CD19. CD20, CD33, CD34, CD45, CD117 (c-kit), CD133, HLA-A. HLA-B, HLA-C, chemokine receptor 5 (CCRS), stem cell marker ABCG2 transporter, ovarian cancer antigen CA125, immunoglobulins, integrins, prostate specific antigen (PSA), prostate stem cell antigen (PSCA), dendritic cell-specific intercellular adhesion molecule 3-grabbing nonintegrin (DC-SIGN), thyroglobulin, granulocyte-macrophage colony stimulating factor (GM-CSF), myogenic differentiation promoting factor-1 (MyoD-1), Leu-7 (CD57), LeuM-1, cell proliferation-associated human nuclear antigen defined by the monoclonal antibody Ki-67 (Ki-67), viral envelope proteins, HIV gp120, or transferrin receptor.

In some embodiments, the PNMA2-based capsid or endo-Gag-based capsid is for in vitro use.

In some instances, the PNMA2-based capsid or endo-Gag-based capsid is for ex vivo use.

In some cases, the PNMA2-based capsid or endo-Gag-based capsid is for in vivo use.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

For example, the container(s) can include a recombinant or engineered PNMA2 or endo-Gag polypeptide described above. Such kits optionally include an identifying description or label or instructions relating to its use in the methods described herein. For example, a kit typically includes labels listing contents and/or instructions for use, and package inserts with instructions for use. A set of instructions will also typically be included.

### Certain Terminologies

Unless defined otherwise, all technical and scientific terms used herein have the same meaning as is commonly understood. It is to be understood that the detailed description is exemplary and explanatory only and not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### Embodiments

Embodiment 1. A capsid comprising a recombinant PNMA2 polypeptide and a heterologous cargo.

Embodiment 2. The capsid of embodiment 1, wherein the heterologous cargo is a nucleic acid.

Embodiment 3. The capsid of any one of embodiments 1-2, wherein the heterologous cargo is a DNA.

Embodiment 4. The capsid of any one of embodiments 1-2, wherein the heterologous cargo is an RNA.

Embodiment 5. The capsid of any one of embodiments 1-4, wherein the heterologous cargo is a therapeutic agent.

Embodiment 6. The capsid of embodiment 1, wherein the heterologous cargo is a protein, a peptide, or an antibody or binding fragment thereof, a peptidomimetic, a nucleotidomimetic, a drug, a diagnostic tool, an imaging tool, or a small molecule.

Embodiment 7. The capsid of any one of embodiments 1-6, wherein the recombinant PNMA2 polypeptide is a mammalian PNMA2.

Embodiment 8. The capsid of any one of embodiments 1-6, wherein the recombinant PNMA2 polypeptide is a human PNMA2.

Embodiment 9. The capsid of any one of embodiments 1-8, wherein the recombinant PNMA2 polypeptide comprises an amino acid sequence with at least 90% sequence identity to SEQ ID NO: 7, SEQ ID NO: 8, or SEQ ID NO: 1.

Embodiment 10. The capsid of any one of embodiments 1-8, wherein the PNMA2 polypeptide comprises an amino acid sequence with at least 91%, at least 92%, at least 93%, at least 94%, at least 95%, at least 96%, at least 97%, at least 98%, at least 99%, or at least 99.5% sequence identity to SEQ ID NO: 7, SEQ ID NO: 8, or SEQ ID NO: 1.

Embodiment 11. The capsid of any one of embodiments 1-8, wherein the PNMA2 polypeptide comprises an amino acid sequence that is SEQ ID NO: 7, SEQ ID NO: 8, or SEQ ID NO: 1.

Embodiment 12. The capsid of any one of embodiments 1-10, wherein the recombinant PNMA2 polypeptide comprises a sequence modification relative to SEQ ID NO: 1.

Embodiment 13. The capsid of any one of embodiments 1-12, wherein the capsid comprises a disulfide bond.

Embodiment 14. The capsid of any one of embodiments 1-12, wherein the capsid comprises a disulfide bond that comprises a Cysteine at position 10, 136, 233, or 310 of the recombinant PNMA2 polypeptide relative to SEQ ID NO: 1.

Embodiment 15. The capsid of any one of embodiments 1-12, wherein the capsid comprises a disulfide bond that comprises a Cysteine at position 136 of the recombinant PNMA2 polypeptide relative to SEQ ID NO: 1.

Embodiment 16. The capsid of any one of embodiments 1-12, wherein the capsid comprises a disulfide bond that comprises a Cysteine at position 310 of the recombinant PNMA2 polypeptide relative to SEQ ID NO: 1.

Embodiment 17. A composition comprising a plurality of the capsid of any one of embodiments 1-16, wherein the capsids are at least about 5%, at least about 10%, at least about 20%, at least about 30%, at least about 40%, at least about 50%, at least about 60%, at least about 70%, at least about 80%, or at least about 90% pure as determined by SDS-PAGE.

Embodiment 18. A composition comprising a plurality of the capsid of any one of embodiments 1-16, wherein the capsids comprise at least about 5%, at least about 10%, at least about 20%, at least about 30%, at least about 40%, at least about 50%, at least about 60%, at least about 70%, at least about 80%, or at least about 90% particle homogeneity as determined by multi-angle dynamic light scattering.

Embodiment 19. An engineered PNMA2 polypeptide comprising a sequence modification relative to SEQ ID NO: 1.

Embodiment 20. The engineered PNMA2 polypeptide of embodiment 19, wherein the sequence modification comprises an amino acid deletion.

Embodiment 21. The engineered PNMA2 polypeptide of any one of embodiments 19-20, wherein the sequence modification comprises an amino acid insertion.

Embodiment 22. The engineered PNMA2 polypeptide of any one of embodiments 19-21, wherein the sequence modification comprises an amino acid substitution.

Embodiment 23. The engineered PNMA2 polypeptide of any one of embodiments 19-22, wherein the sequence modification comprises a cargo binding domain.

Embodiment 24. The engineered PNMA2 polypeptide of any one of embodiments 19-23, wherein the sequence modification comprises a nucleic acid binding domain.

Embodiment 25. The engineered PNMA2 polypeptide of any one of embodiments 19-24, wherein the sequence modification comprises a DNA binding domain.

Embodiment 26. The engineered PNMA2 polypeptide of any one of embodiments 19-25, wherein the sequence modification comprises an RNA binding domain.

Embodiment 27. The engineered PNMA2 polypeptide of any one of embodiments 19-26, wherein the sequence modification comprises a zinc finger domain.

Embodiment 28. The engineered PNMA2 polypeptide of any one of embodiments 19-27, wherein the sequence modification comprises a sub-cellular localization signal.

Embodiment 29. The engineered PNMA2 polypeptide of any one of embodiments 19-28, wherein the sequence modification comprises a nuclear localization signal (NLS).

Embodiment 30. The engineered PNMA2 polypeptide of any one of embodiments 19-29, wherein the sequence modification comprises an arginine-rich domain.

Embodiment 31. The engineered PNMA2 polypeptide of any one of embodiments 19-30, wherein the sequence modification is at an N-terminus of SEQ ID NO: 1.

Embodiment 32. The engineered PNMA2 polypeptide of any one of embodiments 19-30, wherein the sequence modification is at a C-terminus of SEQ ID NO: 1.

Embodiment 33. The engineered PNMA2 polypeptide of any one of embodiments 19-30, wherein the sequence modification is within SEQ ID NO: 1.

Embodiment 34. A nucleic acid encoding the PNMA2 polypeptide of any one of embodiments 19-33.

Embodiment 35. A vector comprising the nucleic acid of embodiment 34.

Embodiment 36. A cell comprising the nucleic acid of embodiment 34.

Embodiment 37. A method of delivering a heterologous cargo to a cell, comprising contacting the cell with the capsid of any one of embodiments 1-18.

Embodiment 38. The method of embodiment 37, wherein the cell is a eukaryotic cell.

Embodiment 39. The method of any one of embodiments 37-38, wherein the cell is a vertebrate cell.

Embodiment 40. The method of any one of embodiments 37-39, wherein the cell is a mammalian cell.

Embodiment 41. The method of any one of embodiments 37-40, wherein the cell is a human cell.

Embodiment 42. The method of any one of embodiments 37-41, wherein the cell is contacted with the capsid at a concentration of at least about 0.001 pg/mL, at least about 0.01 pg/mL, at least about 0.1 pg/mL, at least about 1 pg/mL, at least about 10 pg/mL, at least about 100 pg/mL, at least about 1 ng/mL, at least about 10 ng/mL, at least about 100 ng/mL, at least about 1 μg/mL, at least about 10 μg/mL, at least about 100 μg/mL, at least about 1 mg/mL, at least about 10 mg/mL, or at least about 100 mg/mL.

Embodiment 43. The method of any one of embodiments 37-42, wherein the contacting is in vivo.

Embodiment 44. The method of any one of embodiments 37-43, wherein the contacting is in vitro or ex vivo.

Embodiment 45. The method of any one of embodiments 37-44, wherein the heterologous cargo comprises a nucleic acid, wherein the cell expresses a gene encoded by the nucleic acid after the delivering.

Embodiment 46. The method of any one of embodiments 37-44, wherein the heterologous cargo is a protein, a peptide, or an antibody or binding fragment thereof, a peptidomimetic, a nucleotidomimetic, a drug, a diagnostic tool, an imaging tool, or a small molecule.

Embodiment 47. An engineered endogenous retroviral capsid polypeptide comprising a cysteine residue, wherein upon isolation the engineered endogenous retroviral capsid polypeptide is capable of assembling to form a capsid with an efficiency of at least 5%.

Embodiment 48. The engineered endogenous retroviral capsid polypeptide of embodiment 47, wherein upon isolation the engineered endogenous retroviral capsid polypeptide is capable of assembling to form a capsid with an efficiency of at least about 10%, at least about 20%, at least about 30%, at least about 40%, at least about 50%, at least about 60%, at least about 70%, at least about 80%, or at least about 90%.

Embodiment 49. The engineered endogenous retroviral capsid polypeptide of any one of embodiments 47-48, wherein the engineered endogenous retroviral capsid polypeptide is an endo-Gag polypeptide.

Embodiment 50. The engineered endogenous retroviral capsid polypeptide of any one of embodiments 47-49, wherein the engineered endogenous retroviral capsid polypeptide is an engineered PNMA protein.

Embodiment 51. The engineered endogenous retroviral capsid polypeptide of any one of embodiments 47-50, wherein the engineered endogenous retroviral capsid polypeptide is an engineered PNMA2 protein.

Embodiment 52. The engineered endogenous retroviral capsid polypeptide of any one of embodiments 47-51, wherein the assembling is in a buffer containing less than 2000, less than 1000, less than 900, less than 800, less than 700, less than 600, less than 500, less than 400, less than 300, or less than 200 mOsm/kg of salt.

Embodiment 53. A capsid comprising an engineered endogenous retroviral capsid polypeptide, wherein the capsid is capable of being disassembled into a non-capsid state with an efficiency of at least 1%, and re-assembled into a capsid state with an efficiency of at least 1%.

Embodiment 54. The capsid of embodiment 53, wherein the capsid is capable of being disassembled into a non-capsid state with an efficiency of at least about 5%, at least about 10%, at least about 20%, at least about 30%, at least about 40%, at least about 50%, at least about 60%, at least about 70%, at least about 80%, or at least about 90%.

Embodiment 55. The capsid of any one of embodiments 53-54, wherein when in the disassembled non-capsid state, the capsid is capable of being reassembled into the capsid state with an efficiency of at least about 5%, at least about 10%, at least about 20%, at least about 30%, at least about 40%, at least about 50%, at least about 60%, at least about 70%, at least about 80%, or at least about 90%.

Embodiment 56. The capsid of any one of embodiments 53-55, wherein the engineered endogenous retroviral capsid polypeptide is an endo-Gag polypeptide.

Embodiment 57. The capsid of any one of embodiments 53-56, wherein the engineered endogenous retroviral capsid polypeptide is an engineered PNMA protein.

Embodiment 58. The capsid of any one of embodiments 53-57, wherein the engineered endogenous retroviral capsid polypeptide is an engineered PNMA2 protein.

Embodiment 59. The capsid of any one of embodiments 53-58, wherein the reassembly efficiency is in determined in a buffer containing less than 2000, less than 1000, less than 900, less than 800, less than 700, less than 600, less than 500, less than 400, less than 300, or less than 200 mOsm/kg of salt.

Embodiment 60. A method of making a capsid, the method comprising: (a) expressing a PNMA2 polypeptide in a host cell or a cell-free expression system; and (b) isolating the PNMA2 polypeptide; wherein the isolated PNMA2 polypeptide assembles to form the capsid.

Embodiment 61. The method of any embodiment 60, wherein at least about 5% of the isolated PNMA2 polypeptide assembles to form the capsid.

Embodiment 62. The method of any one of embodiments 60-61, wherein at least about 5%, at least about 10%, at least about 20%, at least about 30%, at least about 40%, at least about 50%, at least about 60%, at least about 70%, at least about 80%, at least about 90%, or at least about 95% of the isolated PNMA2 polypeptide assembles to form the capsid.

Embodiment 63. The method of any one of embodiments 60-62, wherein the percent of the isolated PNMA2 polypeptide present in capsid form is determined by a multi-angle dynamic light scattering assay.

Embodiment 64. The method of any one of embodiments 60-63, wherein the method produces assembled capsids of at least about 5%, at least about 10%, at least about 20%, at least about 30%, at least about 40%, at least about 50%, at least about 60%, at least about 70%, at least about 80%, or at least about 90% purity as determined by SDS-PAGE.

Embodiment 65. The method of any one of embodiments 60-64, wherein the method produces assembled capsids with at least about 5%, at least about 10%, at least about 20%, at least about 30%, at least about 40%, at least about 50%, at least about 60%, at least about 70%, at least about 80%, or at least about 90% particle homogeneity as determined by multi-angle dynamic light scattering.

Embodiment 66. The method of any one of embodiments 60-65, wherein the isolated PNMA2 polypeptide is assembled in a buffer containing less than 2000, less than 1000, less than 900, less than 800, less than 700, less than 600, less than 500, less than 400, less than 300, or less than 200 mOsm/kg of salt.

Embodiment 67. A method of making the capsid of any one of embodiments 1-18 or 37-59, the method comprising: (a) expressing a PNMA2 polypeptide in a host cell or a cell-free expression system; (b) isolating the PNMA2 polypeptide; (c) treating the isolated PNMA2 polypeptide with a disassembly buffer, thereby generating PNMA2 polypeptide in a disassembled form; (d) incubating the disassembled PNMA2 polypeptide in a reassembly buffer with the heterologous cargo present; thereby generating the capsid comprising the heterologous cargo.

Embodiment 68. The method of embodiment 67, wherein the isolated PNMA2 polypeptide is at least partially present in a capsid form prior to step (c).

Embodiment 69. The method of any one of embodiments 60-68, wherein the PNMA2 polypeptide is the recombinant PNMA2 polypeptide of any one of the preceding embodiments.

Embodiment 70. The method of any one of embodiments 60-68, wherein the PNMA2 polypeptide is the engineered PNMA2 polypeptide of any one of the preceding embodiments.

Embodiment 71. The method of any one of embodiments 67-70, wherein at least about 5% of the PNMA2 polypeptide that is present in capsid form before step (c) is disassembled after step (c).

Embodiment 72. The method of any one of embodiments 67-71, wherein at least about 5% of the PNMA2 polypeptide that is present in disassembled form after step (c) is present in capsid form after step (d).

Embodiment 73. The method of any one of embodiments 71-72, wherein the percent of the PNMA2 polypeptide that is present in a capsid form or a disassembled form is determined by a multi-angle dynamic light scattering assay.

Embodiment 74. The method of any one of embodiments 67-73, wherein the disassembly buffer comprises a reducing agent.

Embodiment 75. The method of embodiment 74, wherein the reducing agent is GSH or TCEP.

Embodiment 76. The method of any one of embodiments 67-73, wherein the disassembly buffer comprises a non-denaturing detergent.

Embodiment 77. The method of embodiment 76, wherein the nondenaturing detergent is CHAPS.

Embodiment 78. The method of any one of embodiments 67-77, wherein the reassembly buffer contains less than 2000, less than 1000, less than 900, less than 800, less than 700, less than 600, less than 500, less than 400, less than 300, or less than 200 mOsm/kg of salt.

Embodiment 79. A method of loading PNMA capsids comprising: a) disassembling PNMA capsids into a composition comprising PNMA monomers; b) contacting a cargo with the composition comprising PNMA monomers; and c) reassembling the composition comprising PNMA monomers into PNMA capsids; thereby loading the cargo into an interior of the PNMA capsids.

Embodiment 80. The method embodiment 79, wherein the disassembling comprises reducing a disulfide bond.

Embodiment 81. The method of any one of embodiments 67-80, wherein the reassembling comprises incubating the cargo and the composition comprising PNMA monomers in a physiological buffer.

Embodiment 82. The method of any one of embodiments 67-80, wherein the reassembling comprises incubating the cargo and the composition comprising PNMA monomers in a buffer that comprises about 270-330 mOsm/kg of salt.

### EXAMPLES

These examples are provided for illustrative purposes only and not to limit the scope of the claims provided herein.

### Example 1—Design and Construction of DNA Vectors Encoding Recombinant PNMA Proteins

To construct recombinant DNA vectors for PNMA2 expression, full length cDNA open reading frames, excluding the initial methionine, were inserted into a cloning vector and subsequently transferred into an expression vector according to standard methods.

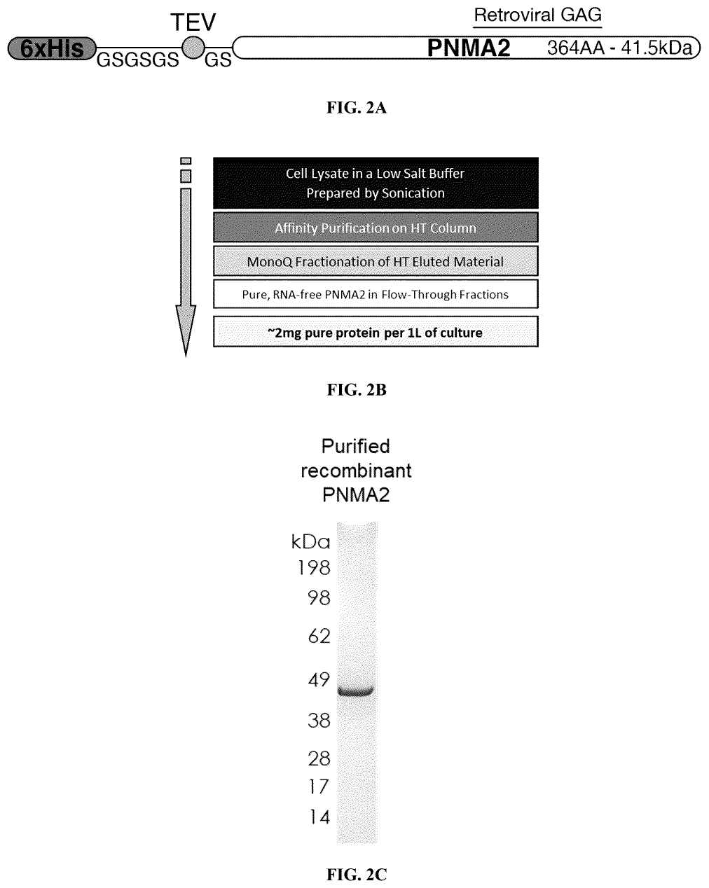

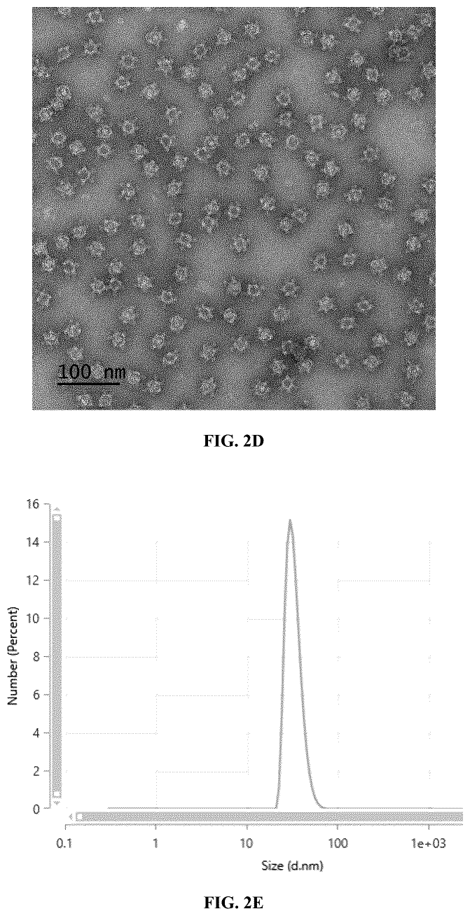

cDNA encoding PNMA2 was inserted into an expression vector derived from pET-41-a(+) (EMD Millipore (Novagen) Cat #70566). The entire cloning site of pET-41-a(+) was removed and replaced with the DNA having the nucleotide sequence of SEQ ID NO: 25, which encodes an N-terminal tag having the amino acid sequence of SEQ ID NO: 26, which comprises a 6×His tag (SEQ ID NO: 27), a 6 amino acid spacer (SEQ ID NO: 28), and a TEV cleavage site (SEQ ID NO: 29), in some instances followed by an additional SG linker. A PNMA2 open reading frame without the starting methionine codon (SEQ ID NO: 30, which is codon optimized and encodes SEQ ID NO: 7) was inserted after the TEV cleavage site by Gibson assembly. Gibson D G, Young L, Chuang R Y, Venter J C, Hutchison C A 3rd, Smith H O (2009), Enzymatic assembly of DNA molecules up to several hundred kilobases, Nature Methods. 6 (5): 343-345. FIG. 2A provides a schematic of the PNMA2 construct.

After expression and Tobacco Etch Virus (TEV) cleavage, the N-terminus of the resulting PNMA2 has a single residual Glycine from TEV cleavage (SEQ ID NO: 8). SEQ ID NOs: 31 and 1 provide nucleotide and amino acid sequences of PNMA2 including the N-terminal Methionine, respectively.

TABLE 2: Illustrative sequences.

TABLE 2

| SEQ | | |
|---|---|---|
| ID | | |
| NO: | Description | Sequence |
| 25 | Nucleotide | ATGCATCACCATCACCATCACGGCTCAGGGTCTGGTAGCGAAAAT |
| | sequence | CTGTACTTCCAGGGGTCAGGT |
| | encoding N- | |
| | terminal tag | |
| 26 | N-terminal | MHHHHHHGSGSGSENLYFQGSG |
| | tag amino | |
| | acid | |
| | sequence | |
| 37 | N-terminal | MHHHHHHGSGSGSENLYFQG |
| | amino acid | |
| | sequence | |
| | lacking SG | |
| | linker | |
| 27 | 6x His tag | HHHHHH |
| 28 | Spacer | GSGSGS |
| 29 | TEV | ENLYFQG |
| | cleavage site | |
| 30 | PNMA2- | GCGTTGGCACTGTTAGAAGATTGGTGCCGTATTATGTCTGTGGAC |
| | encoding | GAGCAGAAGTCTCTTATGGTTACTGGGATCCCGGCGGATTTCGAA |
| | NT seq, | GAAGCCGAAATCCAGGAAGTTCTGCAAGAAACATTAAAGAGCTT |
| | lacking N- | GGGCCGGTATCGGCTCTTAGGCAAAATTTTTCGGAAGCAAGAAAA |
| | terminal | TGCTAATGCGGTGTTACTCGAACTGCTCGAGGATACGGATGTTTC |
| | Met | AGCAATCCCATCTGAAGTGCAGGGCAAAGGTGGGGTGTGGAAAG |
| | | TTATCTTCAAGACGCCAAACCAGGATACTGAGTTCCTTGAACGCT |
| | | TGAATCTGTTCTTGGAGAAAGAAGGCCAGACCGTAAGCGGCATGT |
| | | TCCGGGCGCTTGGCCAAGAGGGGGTATCCCCGGCAACGGTACCTT |
| | | GTATTTCCCCTGAGTTGCTCGCGCACTTATTGGGTCAAGCCATGGC |
| | | ACACGCGCCTCAACCTCTCCTGCCGATGCGGTATCGCAAACTGCG |
| | | CGTATTTTCGGGTTCAGCGGTTCCGGCCCCTGAGGAAGAATCATT |
| | | TGAAGTTTGGCTGGAGCAGGCAACCGAGATCGTGAAGGAGTGGC |
| | | CGGTCACAGAGGCCGAAAAGAAACGGTGGTTAGCCGAGAGTCTT |
| | | CGTGGCCCTGCGCTTGACCTTATGCATATTGTACAGGCGGATAAC |
| | | CCTTCTATCTCGGTTGAAGAGTGTCTTGAGGCTTTCAAGCAAGTCT |
| | | TTGGTTCGCTGGAGTCCCGTCGTACTGCCCAGGTCCGGTATCTCAA |
| | | AACGTATCAGGAAGAGGGTGAGAAGGTCTCCGCATACGTCCTGC |
| | | GGTTGGAAACGCTTTTGCGCCGGGCAGTAGAAAAACGCGCAATTC |
| | | CTCGTCGCATTGCCGATCAAGTTCGTTTAGAGCAAGTGATGGCTG |
| | | GCGCAACTTTAAACCAAATGTTATGGTGCCGGTTACGTGAACTGA |
| | | AGGACCAGGGGCCTCCGCCATCTTTTCTTGAGCTGATGAAGGTGA |
| | | TCCGGGAAGAGGAGGAAGAAGAGGCGTCGTTCGAAAACGAGAGT |
| | | ATCGAAGAGCCGGAGGAACGTGATGGGTACGGTCGCTGGAACCA |
| | | CGAGGGCGACGAT |
| 7 | PNMA2 | ALALLEDWCRIMSVDEQKSLMVTGIPADFEEAEIQEVLQETLKSLGR |
| | amino acid | YRLLGKIFRKQENANAVLLELLEDTDVSAIPSEVQGKGGVWKVIFKT |
| | sequence | PNQDTEFLERLNLFLEKEGQTVSGMFRALGQEGVSPATVPCISPELLA |
| | lacking N- | HLLGQAMAHAPQPLLPMRYRKLRVFSGSAVPAPEEESFEVWLEQAT |
| | terminal | EIVKEWPVTEAEKKRWLAESLRGPALDLMHIVQADNPSISVEECLEA |
| | Met | FKQVFGSLESRRTAQVRYLKTYQEEGEKVSAYVLRLETLLRRAVEK |
| | | RAIPRRIADQVRLEQVMAGATLNQMLWCRLRELKDQGPPPSFLELM |
| | | KVIREEEEEEASFENESIEEPEERDGYGRWNHEGDD |
| 8 | PNMA2 | GSGALALLEDWCRIMSVDEQKSLMVTGIPADFEEAEIQEVLQETLKS |
| | amino acid | LGRYRLLGKIFRKQENANAVLLELLEDTDVSAIPSEVQGKGGVWKVI |
| | sequence | FKTPNQDTEFLERLNLFLEKEGQTVSGMFRALGQEGVSPATVPCISPE |
| | after | LLAHLLGQAMAHAPQPLLPMRYRKLRVFSGSAVPAPEEESFEVWLE |
| | expression | QATEIVKEWPVTEAEKKRWLAESLRGPALDLMHIVQADNPSISVEEC |
| | and | LEAFKQVFGSLESRRTAQVRYLKTYQEEGEKVSAYVLRLETLLRRAV |
| | cleavage | EKRAIPRRIADQVRLEQVMAGATLNQMLWCRLRELKDQGPPPSFLEL |
| | | MKVIREEEEEEASFENESIEEPEERDGYGRWNHEGDD |
| 38 | PNMA2 | GALALLEDWCRIMSVDEQKSLMVTGIPADFEEAEIQEVLQETLKSLG |
| | amino acid | RYRLLGKIFRKQENANAVLLELLEDTDVSAIPSEVQGKGGVWKVIFK |
| | sequence | TPNQDTEFLERLNLFLEKEGQTVSGMFRALGQEGVSPATVPCISPELL |
| | after | AHLLGQAMAHAPQPLLPMRYRKLRVFSGSAVPAPEEESFEVWLEQA |
| | expression | TEIVKEWPVTEAEKKRWLAESLRGPALDLMHIVQADNPSISVEECLE |
| | and | AFKQVFGSLESRRTAQVRYLKTYQEEGEKVSAYVLRLETLLRRAVE |
| | cleavage, | KRAIPRRIADQVRLEQVMAGATLNQMLWCRLRELKDQGPPPSFLEL |
| | without | MKVIREEEEEEASFENESIEEPEERDGYGRWNHEGDD |
| | additional | |
| | SG spacer | |
| 31 | PNMA2- | ATGGCGTTGGCACTGTTAGAAGATTGGTGCCGTATTATGTCTGTG |
| | encoding | GACGAGCAGAAGTCTCTTATGGTTACTGGGATCCCGGCGGATTTC |
| | NT seq | GAAGAAGCCGAAATCCAGGAAGTTCTGCAAGAAACATTAAAGAG |
| | | CTTGGGCCGGTATCGGCTCTTAGGCAAAATTTTTCGGAAGCAAGA |
| | | AAATGCTAATGCGGTGTTACTCGAACTGCTCGAGGATACGGATGT |
| | | TTCAGCAATCCCATCTGAAGTGCAGGGCAAAGGTGGGGTGTGGA |
| | | AAGTTATCTTCAAGACGCCAAACCAGGATACTGAGTTCCTTGAAC |
| | | GCTTGAATCTGTTCTTGGAGAAAGAAGGCCAGACCGTAAGCGGCA |
| | | TGTTCCGGGCGCTTGGCCAAGAGGGGGTATCCCCGGCAACGGTAC |
| | | CTTGTATTTCCCCTGAGTTGCTCGCGCACTTATTGGGTCAAGCCAT |
| | | GGCACACGCGCCTCAACCTCTCCTGCCGATGCGGTATCGCAAACT |
| | | GCGCGTATTTTCGGGTTCAGCGGTTCCGGCCCCTGAGGAAGAATC |
| | | ATTTGAAGTTTGGCTGGAGCAGGCAACCGAGATCGTGAAGGAGT |
| | | GGCCGGTCACAGAGGCCGAAAAGAAACGGTGGTTAGCCGAGAGT |
| | | CTTCGTGGCCCTGCGCTTGACCTTATGCATATTGTACAGGCGGATA |
| | | ACCCTTCTATCTCGGTTGAAGAGTGTCTTGAGGCTTTCAAGCAAGT |
| | | CTTTGGTTCGCTGGAGTCCCGTCGTACTGCCCAGGTCCGGTATCTC |
| | | AAAACGTATCAGGAAGAGGGTGAGAAGGTCTCCGCATACGTCCT |
| | | GCGGTTGGAAACGCTTTTGCGCCGGGCAGTAGAAAAACGCGCAA |
| | | TTCCTCGTCGCATTGCCGATCAAGTTCGTTTAGAGCAAGTGATGG |
| | | CTGGCGCAACTTTAAACCAAATGTTATGGTGCCGGTTACGTGAAC |
| | | TGAAGGACCAGGGGCCTCCGCCATCTTTTCTTGAGCTGATGAAGG |
| | | TGATCCGGGAAGAGGAGGAAGAAGAGGCGTCGTTCGAAAACGAG |
| | | AGTATCGAAGAGCCGGAGGAACGTGATGGGTACGGTCGCTGGAA |
| | | CCACGAGGGCGACGAT |
| 1 | PNMA2 | MALALLEDWCRIMSVDEQKSLMVTGIPADFEEAEIQEVLQETLKSLG |
| | amino acid | RYRLLGKIFRKQENANAVLLELLEDTDVSAIPSEVQGKGGVWKVIFK |
| | sequence | TPNQDTEFLERLNLFLEKEGQTVSGMFRALGQEGVSPATVPCISPELL |
| | | AHLLGQAMAHAPQPLLPMRYRKLRVFSGSAVPAPEEESFEVWLEQA |
| | | TEIVKEWPVTEAEKKRWLAESLRGPALDLMHIVQADNPSISVEECLE |
| | | AFKQVFGSLESRRTAQVRYLKTYQEEGEKVSAYVLRLETLLRRAVE |
| | | KRAIPRRIADQVRLEQVMAGATLNQMLWCRLRELKDQGPPPSFLEL |
| | | MKVIREEEEEEASFENESIEEPEERDGYGRWNHEGDD |

### Example 2—Expression and Purification of His-Tagged PNMA2 Protein

Expression vector constructs comprising His-tagged PNMA2 open reading frames were transformed into the Rosetta 2 (DE3) E. coli strain (Millipore Sigma, Cat #71400). PNMA2 expression was induced with 0.1 mM IPTG followed by a 16-hour incubation at 16° C. PNMA2 protein was purified as outlined in FIG. 2B. Cell pellets were lysed by sonication in 20 mM sodium phosphate pH 7.4, 0.1 M NaCl, 40 mM imidazole, 1 mM DTT, 2 mM MgCl₂, 1 mM ATP, and 10% glycerol. The lysate was treated with excess TURBO DNase (Thermo Fisher Scientific, Cat #AM2238), RNase Cocktail (Thermo Fisher Scientific, Cat #AM2286), and Benzonase Nuclease (Millipore Sigma, Cat #71205) to eliminate nucleic acids. The lysate was cleared of cell debris by centrifugation followed by filtration. 6×His-tagged recombinant protein was loaded onto a HisTrap HP column (Cytiva, Cat #17-5247-01), washed with HisTrap Buffer A (HTA) (20 mM sodium phosphate pH 7.4, 0.5 M NaCl, 40 mM imidazole, and 10% glycerol), and eluted with a linear gradient of HisTrap Buffer B (HTB) (20 mM sodium phosphate pH 7.4, 0.5 M NaCl, 500 mM imidazole, and 10% glycerol) (“6×His tag” disclosed as SEQ ID NO: 27). The peak of the eluted protein was collected, diluted 5 times with MonoQ dilution buffer (25 mM Tris HCl, pH 8.0, 10% glycerol) and loaded onto a MonoQ 5/50 GL anion exchange column (Cytiva, Cat #17516601) pre-equilibrated with MonoQ dilution buffer A supplemented with 100 mM NaCl. PNMA2 protein was eluted using a linear gradient of MonoQ elution buffer (20 mM Tris-HCl pH 8.0, 500 mM NaCl, and 10% glycerol). The resulting PNMA2 protein was generally more than 95% pure as revealed by SDS-PAGE analysis, with a yield of up to 2 mg per 1 L of bacterial culture.

Purified PNMA2 in the peak fractions was concentrated using a 10 kDa MWCO PES concentrator. The N-terminal 6×His tag and spacer were then removed by treating with 10% v/v of TEV Protease (10 units/mL). The cleavage efficiency was above 99% as revealed by SDS-PAGE assay. The protein was then diluted into HTA and cleaned by running over HisTrap HP resin to remove 6×His peptides, residual uncleaved PNMA2 containing the 6×His tag, and His-tagged TEV protease. The resulting purified PNMA2 has an N-terminal Glycine residue (SEQ ID NO: 38) and in some instances an uncleaved serine and glycine linker (SEQ ID NO: 8), and does not contain the initial methionine.

An illustrative image of protein gel stained with Coomassie R-250 showing purified recombinant PNMA2 is provided in FIG. 2C.

### Example 3—Capsid Detection after PNMA2 Purification

Purified PNMA2 was subjected to transmission electron microscopy (TEM) and multi-angle dynamic light scattering (MADLS) using a Malvern Zetasizer Ultra, which confirmed that the protocol for PNMA2 purification produces assembled capsids at greater than 90% purity as assessed by SDS-PAGE, and greater than 90% particle homogeneity as determined by MADLS.

Capsid structure and integrity were assessed by TEM. EM grids (Carbon Support Film, Square Grid, 400 mesh, 3-4 nm, Copper, CF400-Cu-UL) were prepared by glow discharge. A 5 sample of purified PNMA2 capsids was applied to the grid for 30 seconds and then wicked away using filter paper. The grid was then washed twice with MilliQ H₂O. MilliQ H₂O was immediately wicked away using filter paper after each wash. The grid was then washed twice with 5 μL of 1% Uranyl Acetate in H₂O. Uranyl Acetate was immediately wicked away using filter paper after each wash. The grid was then stained using 54, of 1% Uranyl Acetate in H₂O for 3 minutes and air dried for 1 minute. Images of PNMA2 capsids were acquired using a FEI Talos L120C TEM equipped with a Gatan 4k×4k OneView camera. FIG. 2D shows concentrated human PNMA2 capsids.

Capsids were also detected and characterized by MADLS. FIG. 2E shows a MADLS profile of purified PNMA2 capsids.

### Example 4—Design, Vector Construction, Expression, and Purification of Untagged PNMA2 Protein

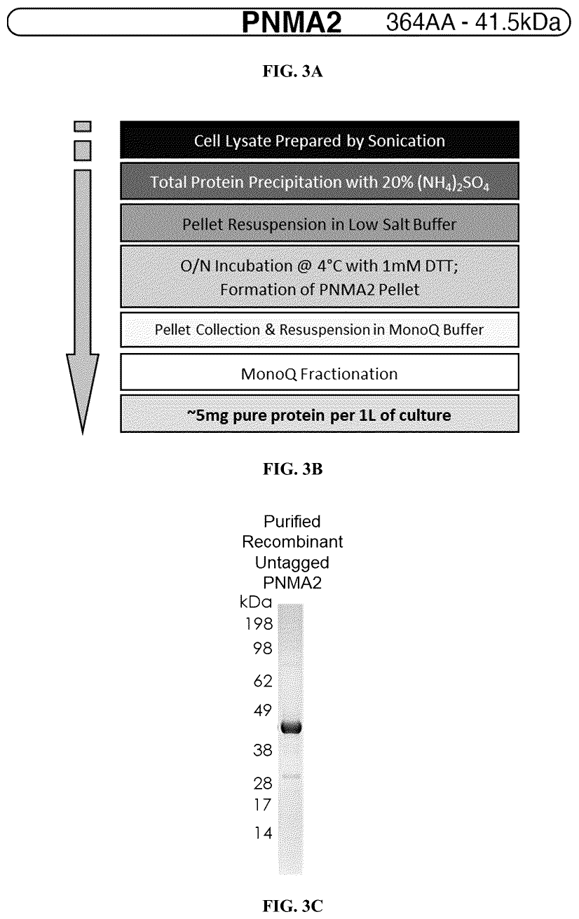

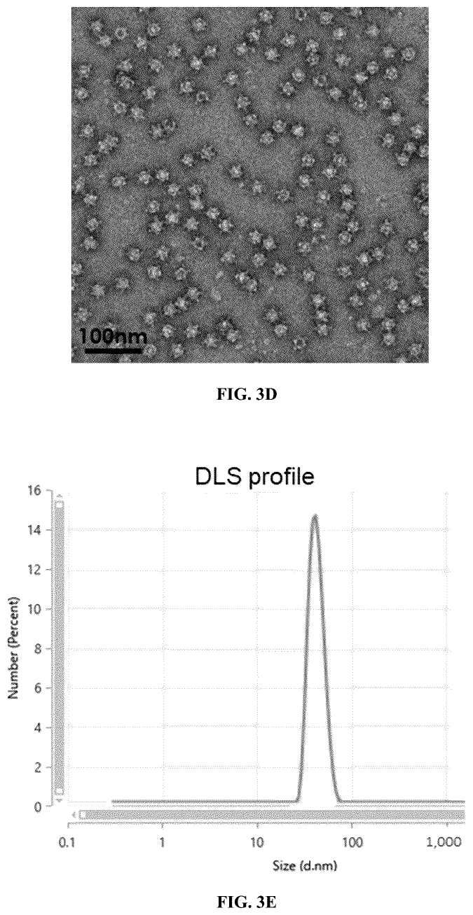

To construct a recombinant DNA vector for expression of untagged wild type PNMA2, a full-length cDNA open reading frame was inserted into a cloning vector and subsequently transferred into an expression vector derived from pET-41-a(+) (EMD Millipore (Novagen) Cat #70566). The entire cloning site of pET-41-a(+) was removed and replaced with DNA having the nucleotide sequence of SEQ ID NO: 31, which is a codon-optimized open reading frame that encodes PNMA2 (SEQ ID NO: 1). The procedure was performed according to Gibson et al., (2009), Enzymatic assembly of DNA molecules up to several hundred kilobases, Nature Methods 6 (5): 343-345. FIG. 3A provides a schematic of the untagged wild type PNMA2 construct.

The expression vector construct comprising a PNMA2 open reading frame was transformed into the Rosetta 2 (DE3) E. coli strain (Millipore Sigma, Cat #71400). PNMA2 expression was induced with 0.1 mM IPTG followed by a 16-hour incubation at 16° C. A purification procedure for untagged PNMA2 protein is shown in FIG. 3B. Cell pellets were lysed by sonication in 50 mM Tris HCl pH 8.0, 0.15 M NaCl, 2 mM EDTA supplemented with a protease inhibitors cocktail (Roche, EASYpack Protease Inhibitor Cocktail). Following centrifugation and filtration to remove cellular debris, the lysate was slowly supplemented with ammonium sulfate powder up to 20% w/v under constant stirring and incubated for 30 min at 21° C. These conditions promote preferential (e.g., exclusive) precipitation of PNMA2 from the lysate. The pellet was collected by centrifugation, resuspended in 50 mM Tris HCl pH 8.0, 2 mM EDTA, 1 mM DTT (in a volume equal to the initial lysate volume), and incubated under constant shaking at +4° C. overnight. The next morning, the material was centrifuged to remove insoluble material. The clarified supernatant was diluted 1:1 with a solution of 100 mM NaCl, 20% glycerol and loaded onto an MonoQ 4.6/100 PE anion exchange column (Cytiva, 17517901). The column was washed with MonoQ buffer A (20 mM Tris HCl, pH 8.0, 100 mM NaCl, 10% glycerol), and untagged PNMA2 was eluted with a linear gradient of MonoQ buffer B (20 mM Tris HCl, pH 8.0, 500 mM NaCl, 10% glycerol). The resulting PNMA2 protein was generally more than 90% pure as revealed by SDS-PAGE analysis, with a yield of up to 5 mg per 1 L of bacterial culture. An illustrative gel image showing purified recombinant PNMA2 is provided in FIG. 3C.

Purified PNMA2 in the peak fraction was dialyzed against PBS buffer using a 20 kDa MWCO Pierce dialysis cassette. Purified dialyzed untagged PNMA2 was subjected to TEM (FIG. 3D) and MADLS (FIG. 3E) analysis that confirmed it was present in a predominantly or entirely capsid form.

### Example 5—Expression, Purification, and Characterization of Recombinant Engineered PNMA Proteins

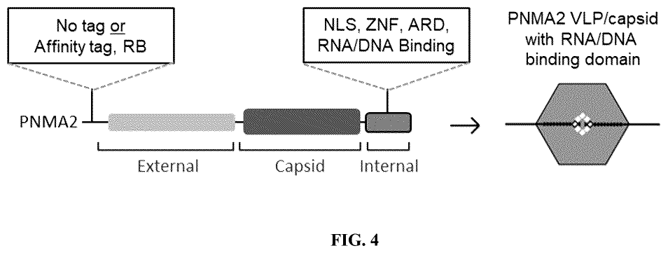

cDNAs encoding recombinant and/or engineered PNMA2 proteins of the disclosure are generated. In some instances, cDNAs can encode modified PNMA2 functional domains, and/or functional domains from other proteins on the N-terminus, C-terminus or within the PNMA2 sequence. For example, an affinity tag, receptor binding sequence (RB), RNA binding domain (RBD), or DNA binding domain (DBD) can be inserted and/or modified, as illustrated in FIG. 4. The sequence of PNMA2 can be modified to contain, e.g., a nuclear localization signal (NLS), Arginine rich domain (ARD), and/or zinc finger domain (ZNF), as illustrated in FIG. 4, or the PNMA2 sequence can be modified by any combination of these or other approaches exemplified herein.

Recombinant cDNA vectors for PNMA2 expression are generated, e.g., using similar techniques to those disclosed in EXAMPLE 1 and EXAMPLE 4.

The recombinant/engineered PNMA2 proteins are expressed and purified, e.g., using similar techniques to those disclosed in EXAMPLES 2, 4, 6, 7. Capsid formation by the purified PNMA2 proteins is evaluated, e.g., by MADLS and TEM, using similar techniques to those disclosed in EXAMPLE 3. The PNMA2 proteins are characterized for their ability to be disassembled, reassembled, and package cargo, e.g., as in EXAMPLES 9 and 11-13. Mutants are generated to test the contribution of disulfide bonds to capsid formation, e.g., as in EXAMPLE 8 and EXAMPLE 15. Cellular uptake of the capsids and/or delivery of cargo is evaluated, e.g., as in EXAMPLES 14-24.

Illustrative expression and purification details for recombinant/engineered PNMA2 proteins with high affinity toward RNA and ssDNA are disclosed in the EXAMPLES 6 and 7.

### Example 6—Expression and Purification of Tagged PNMA2 Engineered to have High Affinity Toward RNA and DNA Cargo

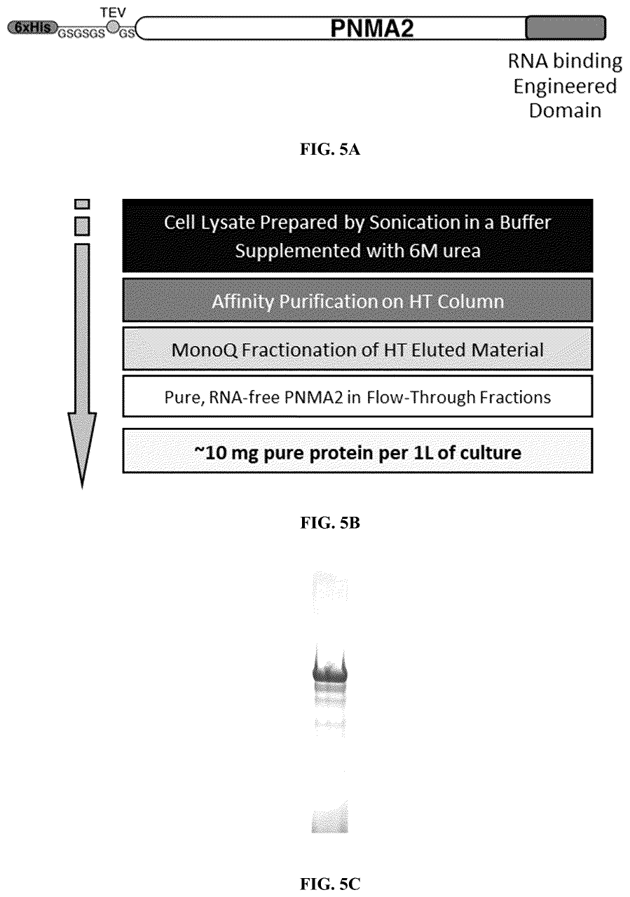

PNMA2 was engineered to have high affinity for RNA and DNA cargo (FIG. 5A) via introduction of a synthetic nucleic acid binding domain (SEQ ID NO: 39). The synthetic nucleic acid binding domain was designed using predicted secondary structure to extend the final C-terminal alpha helix of PNMA2 and turn the nucleic acid binding domain towards the interior of an assembled capsid. The synthetic nucleic acid binding domain binds to RNA (e.g., mRNA and hairpin RNA) and single stranded DNA (ssDNA).

Deletion analysis of PNMA2 indicated that the C-terminal 25 amino acids could be deleted without substantially inhibiting capsid assembly. The C-terminal 25 amino acids of SEQ ID NO: 7 were replaced with SEQ ID NO: 39 to provide an engineered PNMA2 polypeptide comprising SEQ ID NO: 40. The engineered PNMA2 polypeptide was cloned together with an N-terminal 6×His tag, GS linker, and TEV cleavage site as in EXAMPLE 1 to provide SEQ ID NO: 41. An untagged version of the engineered PNMA2 polypeptide that comprises an N-terminal methionine is provided in SEQ ID NO: 55.

TABLE 3: Illustrative sequences of PNMA2 engineered to have high affinity toward RNA.

TABLE 3

| SEQ | | |
|---|---|---|
| ID | | |
| NO: | Description | Sequence |
| 39 | Synthetic | EMRRARKRKPRRRQRRKKRGSGQP |
| | nucleic acid | |
| | binding | |
| | domain | |
| 40 | Engineered | ALALLEDWCRIMSVDEQKSLMVTGIPADFEEAEIQEVLQETLKSLGR |
| | PNMA2 | YRLLGKIFRKQENANAVLLELLEDTDVSAIPSEVQGKGGVWKVIFKT |
| | with C- | PNQDTEFLERLNLFLEKEGQTVSGMFRALGQEGVSPATVPCISPELLA |
| | terminal | HLLGQAMAHAPQPLLPMRYRKLRVFSGSAVPAPEEESFEVWLEQAT |
| | RNA | EIVKEWPVTEAEKKRWLAESLRGPALDLMHIVQADNPSISVEECLEA |
| | binding | FKQVFGSLESRRTAQVRYLKTYQEEGEKVSAYVLRLETLLRRAVEK |
| | domain | RAIPRRIADQVRLEQVMAGATLNQMLWCRLRELKDQGPPPSFLELM |
| | (RBD) | KVIREEEEEEAEMRRARKRKPRRRQRRKKRGSGQP |
| 41 | Engineered | MHHHHHHGSGSGSENLYFQGALALLEDWCRIMSVDEQKSLMVTGIP |
| | PNMA2 | ADFEEAEIQEVLQETLKSLGRYRLLGKIFRKQENANAVLLELLEDTD |
| | with C- | VSAIPSEVQGKGGVWKVIFKTPNQDTEFLERLNLFLEKEGQTVSGMF |
| | terminal | RALGQEGVSPATVPCISPELLAHLLGQAMAHAPQPLLPMRYRKLRVF |
| | RBD and N- | SGSAVPAPEEESFEVWLEQATEIVKEWPVTEAEKKRWLAESLRGPAL |
| | terminal | DLMHIVQADNPSISVEECLEAFKQVFGSLESRRTAQVRYLKTYQEEG |
| | 6xHis tag | EKVSAYVLRLETLLRRAVEKRAIPRRIADQVRLEQVMAGATLNQML |
| | and | WCRLRELKDQGPPPSFLELMKVIREEEEEEAEMRRARKRKPRRRQRR |
| | cleavage | KKRGSGQP |
| | site | |
| 55 | Engineered | MALALLEDWCRIMSVDEQKSLMVTGIPADFEEAEIQEVLQETLKSLG |
| | PNMA2 | RYRLLGKIFRKQENANAVLLELLEDTDVSAIPSEVQGKGGVWKVIFK |
| | with C- | TPNQDTEFLERLNLFLEKEGQTVSGMFRALGQEGVSPATVPCISPELL |
| | terminal | AHLLGQAMAHAPQPLLPMRYRKLRVFSGSAVPAPEEESFEVWLEQA |
| | RNA | TEIVKEWPVTEAEKKRWLAESLRGPALDLMHIVQADNPSISVEECLE |
| | binding | AFKQVFGSLESRRTAQVRYLKTYQEEGEKVSAYVLRLETLLRRAVE |
| | domain | KRAIPRRIADQVRLEQVMAGATLNQMLWCRLRELKDQGPPPSFLEL |
| | (RBD) and | MKVIREEEEEEAEMRRARKRKPRRRQRRKKRGSGQP |
| | N-terminal | |
| | Met | |

Expression constructs comprising the PNMA2 open reading frame with an N-terminal 6×His tag were transformed into the Rosetta 2 (DE3) E. coli strain (Millipore Sigma, Cat #71400).

FIG. 5B outlines the purification procedure. PNMA2 expression was induced at an optical density A600 of 0.8-0.9 with 0.1 mM IPTG followed by a 16-hour incubation at 16° C. Cell pellets were lysed by sonication in 20 mM sodium phosphate pH 7.4, 0.1 M NaCl, 40 mM imidazole, 10% glycerol, and 6 M urea. The lysate was clarified by centrifugation, filtered and loaded onto a HisTrap HP column (Cytiva, Cat #17-5247-01) equilibrated with HisTrap buffer A (HTA) (20 mM sodium phosphate pH 7.4, 0.5 M NaCl, 40 mM imidazole, 10% glycerol, and 6 M urea). The protein was eluted with a linear gradient of HisTrap buffer B (HTB) (20 mM sodium phosphate pH 7.4, 0.5 M NaCl, 500 mM imidazole, 10% glycerol, and 6 M urea). The peak of the eluted protein was collected, diluted 5 times with dilution buffer (25 mM Tris HCl, pH 8.0, 10% glycerol, and 6 M urea) and loaded onto an MonoQ 5/50 GL anion exchange column (Cytiva, Cat #17516601) pre-equilibrated with MonoQ buffer A (20 mM Tris HCl, pH 8.0, 100 mM NaCl, 10% glycerol, and 6 M urea). Under these conditions, PNMA2 did not bind the column and was collected in a flow-through fraction. The resulting PNMA2 protein was generally more than 90% pure as revealed by SDS-PAGE analysis (FIG. 5C), with a yield of up to 10 mg per 1 L of bacterial culture and 260/280 ratio around 0.6.

### Example 7—Expression and Purification of Untagged PNMA2 Engineered to have High Affinity Toward RNA and DNA Cargo

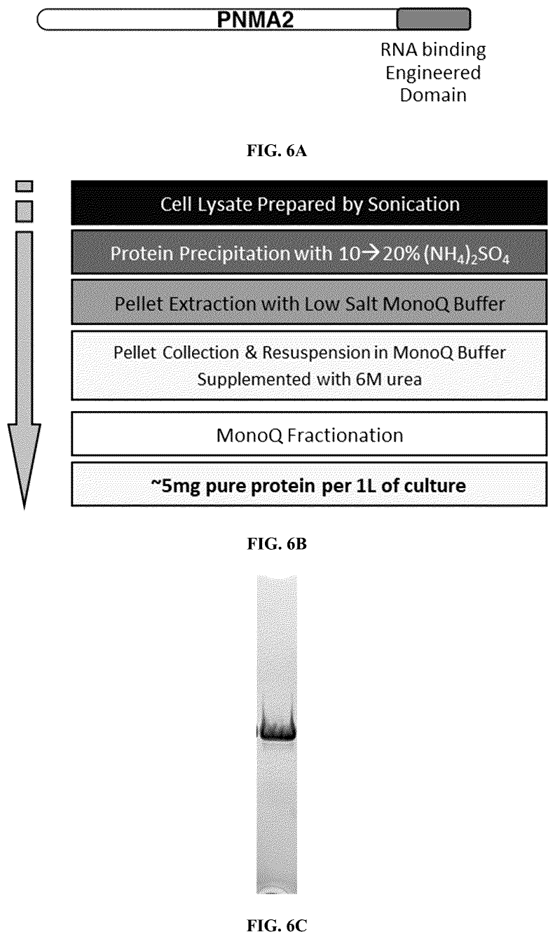

PNMA2 was engineered to have high affinity for RNA and DNA cargo (FIG. 6A) via introduction of the synthetic nucleic acid binding domain described in EXAMPLE 6. The C-terminal 25 amino acids of SEQ ID NO: 7 were replaced with SEQ ID NO: 39 to provide an engineered PNMA2 polypeptide comprising SEQ ID NO: 55.

Expression vector constructs comprising PNMA2 open reading frames (without an N-terminal 6×His tag) were transformed into the Rosetta 2 (DE3) E. coli strain (Millipore Sigma, Cat #71400). PNMA2 expression was induced at an optical density (A600) of 0.8-0.9 with 0.1 mM IPTG followed by a 16-hour incubation at 16° C. FIG. 6A provides a schematic of the PNMA2 construct, while FIG. 6B outlines the purification procedure. Until mentioned otherwise, all steps in the purification procedure were performed at room temperature. The cell pellet was rapidly lysed by sonication on ice in 750 mM sodium phosphate pH 7.2 supplemented with 10% glycerol. The lysate was subjected to centrifugation and filtration to remove cellular debris. Ammonium sulfate powder was added to the filtered lysate with constant mixing up to 10% w/v. The precipitate was removed by centrifugation and discarded. The concentration of ammonium sulfate in the supernatant was adjusted up to 20% w/v under constant mixing and the precipitated protein was collected by centrifugation. The pellet was completely resuspended in MonoQ loading buffer (20 mM Tris-HCl pH 8.0, 100 mM NaCl, and 10% glycerol) and centrifuged. The supernatant was discarded, and the pellet was redissolved in MonoQ loading buffer supplemented with 6 M urea. The redissolved pellet was diluted 5 times with MonoQ loading buffer supplemented with 6 M urea but without NaCl, filtered and loaded onto a MonoQ column equilibrated at +4° C. PNMA2 protein was eluted using a linear gradient of MonoQ elution buffer (20 mM Tris-HCl pH 8.0, 500 mM NaCl, 6 M urea, and 10% glycerol). PNMA2 eluted early in the gradient, while RNA, efficiently separated from PNMA2, eluted at 500 mM NaCl. The resulting PNMA2 protein was generally more than 95% pure as revealed by SDS-PAGE analysis (FIG. 6C) with a 260/280 nm ratio of −0.6. The yield of PNMA2 was 5 mg per 1 L of bacterial culture.

### Example 8—Disulfide Bonds Stabilize PNMA2 Multimers and Capsids

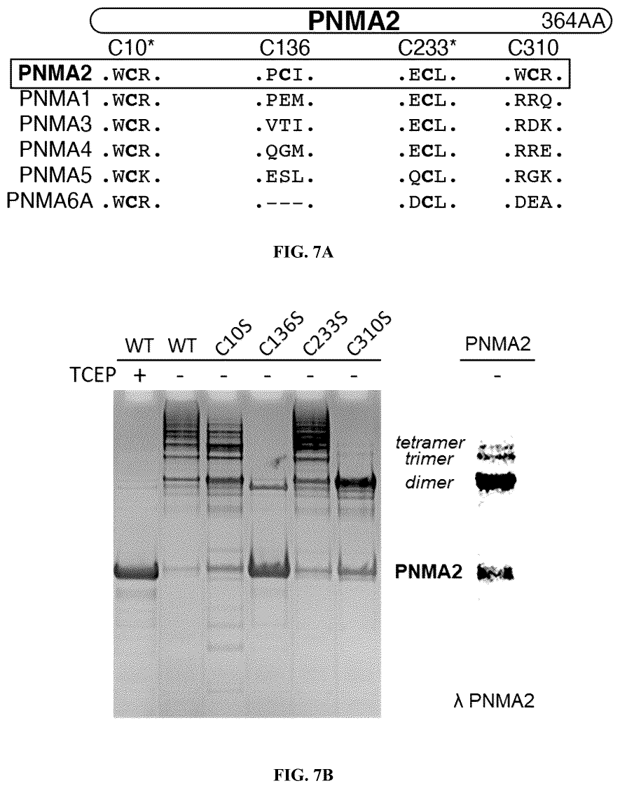

To evaluate the contribution of cysteine residues and disulfide bonds to PNMA2 multimerization and capsid formation, mutants were generated in which each of the four cysteine residues of SEQ ID NO: 1 at positions 10, 136, 233, and 310 were separately mutated to serine. FIG. 7A shows an alignment of the cysteine residues of PNMA2 to equivalent residues in PNMA1, PNMA3, PNMA4, PNMA5, and PNMA6A, showing that some cysteines are conserved between PNMA family members (e.g., C10, C233), while other are not (e.g., C136, C310).

The C10S, C136S, C233S, and C310S mutant versions of PNMA2 were expressed, and the resulting products compared to wild type PNMA2 (with or without TCEP treatment) by SDS-PAGE (FIG. 7B) and confirming protein identity by western blot (shown in the right side of FIG. 7B).

As shown in FIG. 7B, dimers, trimers, tetramers, and many higher-order interactions are observed for wild type PNMA2. These multimers were disrupted when disulfide bonds were reduced by TCEP treatment. The mutants exhibited reduction/loss of bands corresponding to PNMA2 multimers and/or increased abundance of PNMA2 monomers, suggesting that the disulfide bonds contribute to PNMA2 multimerization and renders the interaction between PNMA2 subunits resistant to disassembly in a denaturing SDS gel.

### Example 9—Capsid Disassembly and Reassembly

Recently purified wild type tagged/untagged PNMA2 protein (2 mg/mL in HTB or PBS) was diluted to 0.5 mg/mL in disassembly buffer containing 5.33 M Urea, 66.67 mM DTT, 500 mM NaCl, 20 mM NaP pH 7.4, 10% glycerol resulting in a final concentration of 4 M Urea, 50 mM DTT, 500 mM NaCl, 20 mM NaP pH 7.4, 10% glycerol. The solution was incubated on ice for 30 minutes. Following the incubation, disassembly was confirmed by i) MADLS and ii) TEM.

Recently purified PNMA2 protein with a C-terminal RNA binding domain (0.5-4 mg/mL in 6 M Urea HTB) was diluted to 0.5 mg/mL using a disassembly buffer: 4 M Urea, 50 mM DTT, 500 mM NaCl, 20 mM NaP pH 7.4, 10% glycerol giving a final concentration of 4-6 M Urea, 30-50 mM DTT, 500 mM NaCl, 10% glycerol. The solution was incubated on ice for 30 minutes. Following the incubation, disassembly was confirmed by i) MADLS and ii) TEM.

In similar assays, PNMA2 capsid disassembly and reassembly was evaluated by using TCEP, β-ME and reduced Glutathione (GSH) as reducing agents. All the reducing agents were able to facilitate capsid disassembly in a concentration-dependent manner. In a separate experiment, urea was replaced with 10% CHAPS or other solubilizing agents, which also resulted in capsid disassembly. PNMA2 capsid disassembly was confirmed by i) MADLS and ii) TEM.

Recently purified tagged/untagged PNMA2 was reassembled into capsids by overnight dialysis of the disassembled material against reassembly buffer (RB: 20 mM Na₂HPO₄/NaH₂PO₄, pH 7.4, 0.5 M NaCl, 10% glycerol, 40 mM imidazole, 1 mM DTT) at +4° C. in a dialysis cassette with a 3,000-10,000 Da molecular weight cutoff.

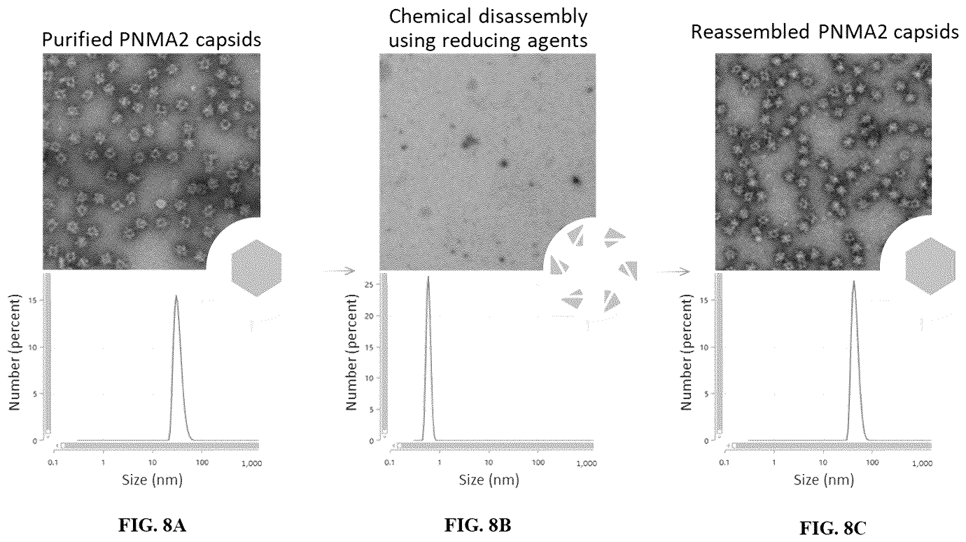

The quality and integrity of the soluble re-assembled PNMA2 capsids was analyzed by i) MADLS and ii) TEM. FIGS. 8A, 8B and 8C show purified intact PNMA2 capsids (before disassembly), chemically disassembled PNMA2 capsids, and reassembled PNMA2 capsids, respectively, as demonstrated by TEM (FIG. 8A-8C, upper panels) and MADLS (FIG. 8A-8C, bottom panels).

### Example 10—RNA is Tightly Associated with PNMA2 Capsids

The association of RNA with PNMA2 capsids was tested by anion exchange chromatography.

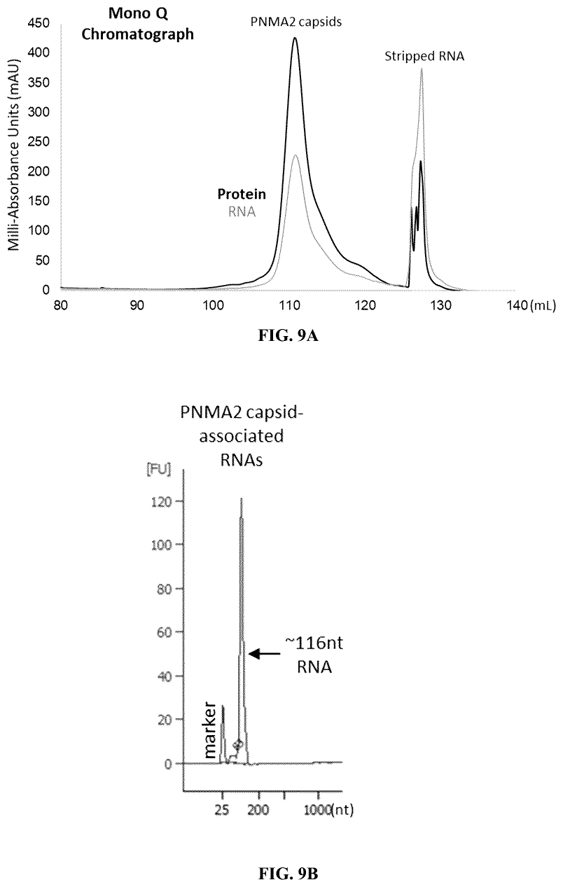

Nucleic acids not associated with purified PNMA2 were first removed by anion exchange chromatography on a Mono Q 4.6/100 PE column (GE Healthcare, Cat #17517901). Before loading on the column, recombinant protein was 5 times diluted with Mono Q dilution buffer (25 mM Tris-HCl pH 8.0 and 10% glycerol). After loading, the Mono Q resin was washed with 2 ml of buffer C (20 mM Tris-HCl pH 8.0, 100 mM NaCl, and 10% glycerol). PNMA2 protein was eluted using a linear gradient of buffer D (20 mM Tris-HCl pH 8.0, 500 mM NaCl, and 10% glycerol). Unbound RNA efficiently separated from PNMA2 capsids (VLPs) and eluted at 600 mM NaCl (FIG. 9A). FIG. 9B quantifies RNA extracted from the PNMA2 capsid (VLP) peak, demonstrating tight association of an approximately 116nt RNA with the capsids.

### Example 11—Nucleic Acid Cargo Loading During PNMA2 Capsid Reassembly

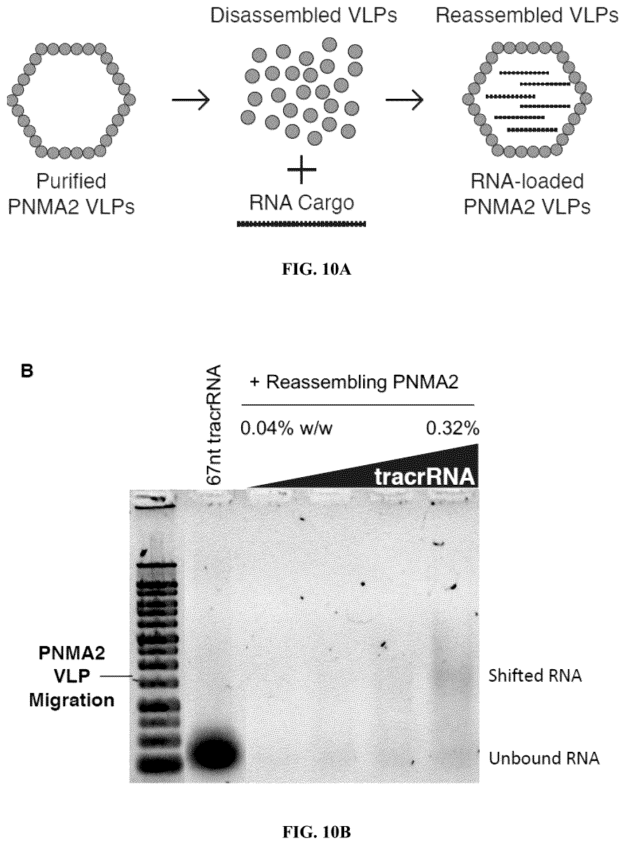

This example demonstrates packaging of an RNA cargo in PNMA2 capsids during reassembly, as illustrated in FIG. 10A.

Disassembled PNMA2 was incubated with a fixed amount of an illustrative non-specific cargo (Alt-R® tracrRNA, a 67nt universal tracrRNA that contains chemical modifications conferring increased nuclease resistance, IDT cat #1072532) at 0.04-0.32% w/w ratios in Disassembly Buffer for 30 minutes on ice. Disassembled PNMA2+RNA was then loaded into a 10 kDa MWCO and dialyzed overnight into Assembly Buffer (75 mM NaCl, 50 mM Tris pH 8.0, 10% Glycerol) at 4° C. The following day assembly was confirmed by i) MADLS and ii) TEM.

Loading of the RNA into capsids was evaluated by a gel shift assay. Assembled PNMA2+RNA complexes were adjusted to 0.5 M NaCl. A 1×TAE, 1% agarose gel was loaded with 300 ng RNA and subjected to electrophoresis at 70V for 1.25 hours. The gel was stained with 100 mL of 1×TAE buffer supplemented with 104, of 10000×SBYR II green RNA dye for 30 minutes with shaking at room temperature. The gel was then destained with three 10-minute washes in 100 mL H₂O. An upshift of RNA was observed for samples with PNMA2 and RNA, indicating that the RNA was packaged in the reassembled PNMA2 capsids (FIG. 10B).

### Example 12—Nucleic Acid Cargo Loading During Engineered PNMA2 Capsids Reassembly

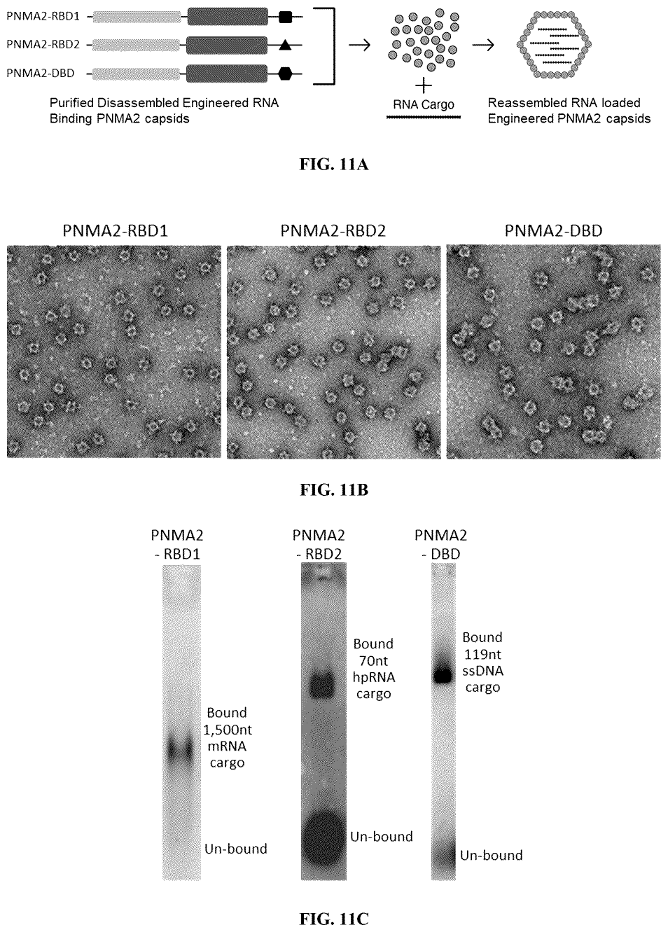

This example demonstrates packaging of RNA or DNA cargo in engineered PNMA2 capsids during reassembly, as illustrated in FIG. 11A.

A first engineered PNMA2 polypeptide, PNMA2-DBD was generated as described in EXAMPLE 6 with introduction of the synthetic RNA binding domain provided in SEQ ID NO: 39. The engineered PNMA2 polypeptide was cloned together with an N-terminal 6×His tag, GS linker, and TEV cleavage site as in EXAMPLE 1 to provide SEQ ID NO: 41.

A second engineered PNMA2 polypeptide, PNMA2-RBD1 was engineered to have high affinity for RNA and ssDNA cargo with introduction of amino acids R381-R403 (SEQ ID NO: 42) from human paraneoplastic antigen Ma3 isoform 2 NP 001269464.1. SEQ ID NO: 42 can bind RNA and ssDNA and can exhibit a preference for longer RNAs. The C-terminal 25 amino acids of SEQ ID NO: 7 were replaced with SEQ ID NO: 42 to provide an engineered PNMA2 polypeptide comprising SEQ ID NO: 43. The engineered PNMA2 polypeptide was cloned together with an N-terminal 6×His tag, GS linker, and TEV cleavage site as in EXAMPLE 1 to provide SEQ ID NO: 44.

A third engineered PNMA2 polypeptide, PNMA2-RBD2 was engineered to have a nucleic acid binding domain derived from the TAT peptide of HIV-1. The nucleic acid binding domain (SEQ ID NO: 45) used was an inverted TAT sequence that binds nucleic acids with a preference towards RNA, and high binding affinity towards RNA containing a bulge hairpin structural motif. SEQ ID NO: 45 was appended to the C-terminus of SEQ ID NO: 7 to provide an engineered PNMA2 polypeptide comprising SEQ ID NO: 46. The engineered PNMA2 polypeptide was cloned together with an N-terminal 6×His tag, GS linker, and TEV cleavage site as in EXAMPLE 1 to provide SEQ ID NO: 47.

TABLE 4: Illustrative sequences of PNMA2 engineered to have high affinity toward RNA or DNA.

TABLE 4

| SEQ | | |
|---|---|---|
| ID | | |
| NO: | Description | Sequence |
| 42 | Nucleic acid | RRRRGRGQHRRGGVARAGSR |
| | binding domain | |
| | from PNMA3 | |
| 43 | Engineered | ALALLEDWCRIMSVDEQKSLMVTGIPADFEEAEIQEVLQETLKS |
| | PNMA2 with C- | LGRYRLLGKIFRKQENANAVLLELLEDTDVSAIPSEVQGKGGV |
| | terminal nucleic | WKVIFKTPNQDTEFLERLNLFLEKEGQTVSGMFRALGQEGVSPA |
| | acid binding | TVPCISPELLAHLLGQAMAHAPQPLLPMRYRKLRVFSGSAVPAP |
| | domain | EEESFEVWLEQATEIVKEWPVTEAEKKRWLAESLRGPALDLMH |
| | | IVQADNPSISVEECLEAFKQVFGSLESRRTAQVRYLKTYQEEGE |
| | | KVSAYVLRLETLLRRAVEKRAIPRRIADQVRLEQVMAGATLNQ |
| | | MLWCRLRELKDQGPPPSFLELMKVIREEEEEEARRRRGRGQHR |
| | | RGGVARAGSR |
| 44 | Engineered | MHHHHHHGSGSGSENLYFQGALALLEDWCRIMSVDEQKSLMV |
| | PNMA2 with C- | TGIPADFEEAEIQEVLQETLKSLGRYRLLGKIFRKQENANAVLLE |
| | terminal nucleic | LLEDTDVSAIPSEVQGKGGVWKVIFKTPNQDTEFLERLNLFLEK |
| | acid binding | EGQTVSGMFRALGQEGVSPATVPCISPELLAHLLGQAMAHAPQ |
| | domain and N- | PLLPMRYRKLRVFSGSAVPAPEEESFEVWLEQATEIVKEWPVTE |
| | terminal 6xHis | AEKKRWLAESLRGPALDLMHIVQADNPSISVEECLEAFKQVFGS |
| | tag and cleavage | LESRRTAQVRYLKTYQEEGEKVSAYVLRLETLLRRAVEKRAIPR |
| | site | RIADQVRLEQVMAGATLNQMLWCRLRELKDQGPPPSFLELMK |
| | | VIREEEEEEARRRRGRGQHRRGGVARAGSR |
| 45 | Inverted TAT | PRRRQRRKKRGSGQP |
| | nucleic acid | |
| | binding domain | |
| 46 | Engineered | ALALLEDWCRIMSVDEQKSLMVTGIPADFEEAEIQEVLQETLKS |
| | PNMA2 with C- | LGRYRLLGKIFRKQENANAVLLELLEDTDVSAIPSEVQGKGGV |
| | terminal nucleic | WKVIFKTPNQDTEFLERLNLFLEKEGQTVSGMFRALGQEGVSPA |
| | acid binding | TVPCISPELLAHLLGQAMAHAPQPLLPMRYRKLRVFSGSAVPAP |
| | domain | EEESFEVWLEQATEIVKEWPVTEAEKKRWLAESLRGPALDLMH |
| | | IVQADNPSISVEECLEAFKQVFGSLESRRTAQVRYLKTYQEEGE |
| | | KVSAYVLRLETLLRRAVEKRAIPRRIADQVRLEQVMAGATLNQ |
| | | MLWCRLRELKDQGPPPSFLELMKVIREEEEEEASFENESIEEPEE |
| | | RDGYGRWNHEGDDPRRRQRRKKRGSGQP |
| 47 | Engineered | MHHHHHHGSGSGSENLYFQGALALLEDWCRIMSVDEQKSLMV |
| | PNMA2 with C- | TGIPADFEEAEIQEVLQETLKSLGRYRLLGKIFRKQENANAVLLE |
| | terminal nucleic | LLEDTDVSAIPSEVQGKGGVWKVIFKTPNQDTEFLERLNLFLEK |
| | acid binding | EGQTVSGMFRALGQEGVSPATVPCISPELLAHLLGQAMAHAPQ |
| | domain and N- | PLLPMRYRKLRVFSGSAVPAPEEESFEVWLEQATEIVKEWPVTE |
| | terminal 6xHis | AEKKRWLAESLRGPALDLMHIVQADNPSISVEECLEAFKQVFGS |
| | tag and cleavage | LESRRTAQVRYLKTYQEEGEKVSAYVLRLETLLRRAVEKRAIPR |
| | site | RIADQVRLEQVMAGATLNQMLWCRLRELKDQGPPPSFLELMK |
| | | VIREEEEEEASFENESIEEPEERDGYGRWNHEGDDPRRRQRRKK |
| | | RGSGQP |

The engineered PNMA2 polypeptides were expressed and purified. Disassembled PNMA2 with C-terminal RNA-binding domains was incubated with 0.5-5% w/v nucleic acid cargo for 30 minutes on ice. Engineered PNMA2-RBD1 was loaded with an approximately 1500 nucleotide mRNA cargo. Engineered PNMA2-RBD2 was loaded with an approximately 70 nucleotide hairpin RNA cargo. Engineered PNMA2-DBD was loaded with an approximately 119 nucleotide ssDNA cargo. The mixture was then loaded into a 10 kDa MWCO dialysis cassette and dialyzed overnight into Reassembly Buffer (RB: 20 mM Na₂HPO₄/NaH₂PO₄, pH 7.4, 0.5 M NaCl, 10% glycerol, 40 mM imidazole, 1 mM DTT) at +4° C. In some experiments, an extra dialysis step against standard PBS buffer (pH 7.2-7.5) was performed. The following day assembly was confirmed by TEM (FIG. 11B). RNA or DNA association with reassembled capsids was evaluated by gel shift analysis (FIG. 11C).

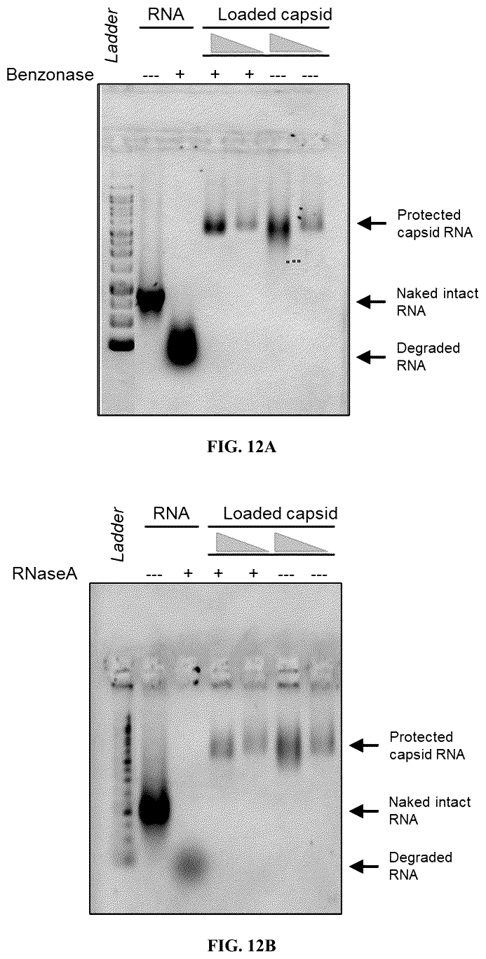

RNA encapsulation was confirmed with a nuclease protection assay. The nuclease protection assay was performed by incubating either naked cargo RNA or reassembled RNA cargo-loaded PNMA2 capsids (comprising SEQ ID NO: 41) with either benzonase (FIG. 12A) or RNaseA (FIG. 12B) nucleases. Protected RNA was visualized by staining the gel with SBYR II green.

### Example 13—Hybrid PNMA2 Capsids

This example demonstrates formation of hybrid PNMA2 capsids with a heterogeneous mixture of (i) subunits with an N-terminal His-tag (as an illustrative exogenous polypeptide sequence), and (ii) subunits that lack the His-tag sequence.

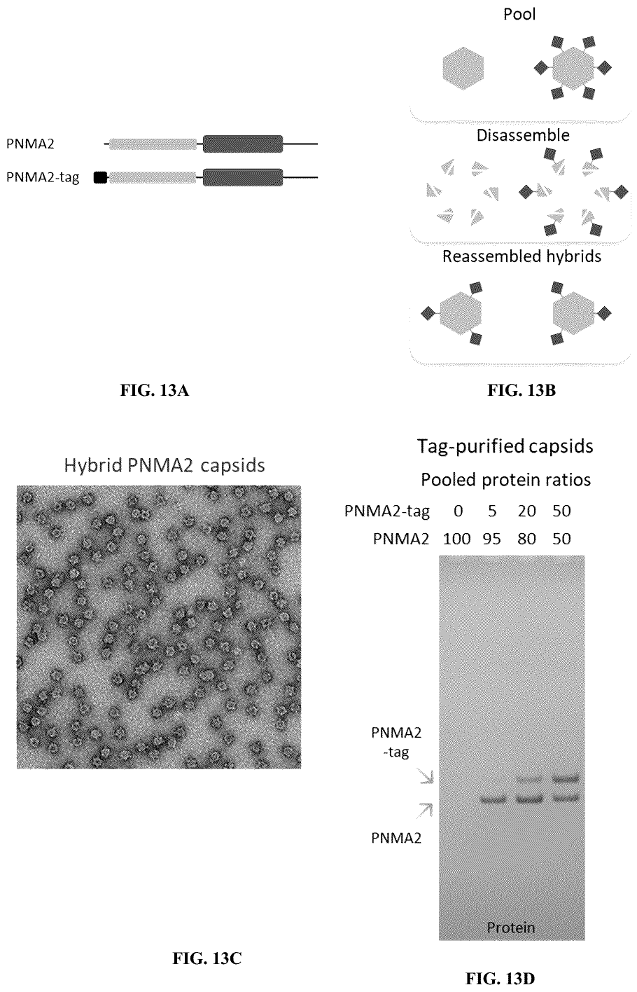

PNMA2 polypeptides that either contained an N-terminal 6×His tag or lacked the 6×His tag (FIG. 13A) were expressed and purified.

Following chemical disassembly, the two types of engineered PNMA2 polypeptides were premixed and reassembled into hybrid PNMA2 capsids (FIG. 13B). Transmission electron microscopy analysis confirmed the formation of hybrid capsids (FIG. 13C). Adjusting relative amounts of the individual engineered PNMA2 polypeptides allowed for control of the mass/molar ratio of individual subunits (e.g., with and without the exogenous polypeptide sequence) within a given reassembled hybrid capsid.

FIG. 13D provides an illustrative image of a denaturing polyacrylamide gel with Coomassie R-250 stained untagged and tagged PNMA2 proteins following hybrid capsid reassembly and affinity purification on Ni-NTA agarose.

### Example 14—Selective Cellular Internalization of PNMA2 Capsids

Primary neurons were grown from E18 Sprague Dawley rat cortex (Brain Bits, LLC) that was enzymatically dissociated with papain and plated in poly-D-lysine coated 8-well chamber slides at a density of 23,000 cells/cm². Cultures were grown for 14 days prior to capsid administration with one half volume media replacements every 3 days. For treatments, 10% of the primary culture media was removed and replaced with PNMA2 capsids (1.2 mg/ml) for a final treatment concentration of 0.12 mg/ml. Treatments proceeded for 7 hours at 37° C., followed by 7 iterative one-half media replacements to remove capsids from solution. After treatments, cells were washed with phosphate buffered saline, fixed with 4% formaldehyde and permeabilized with 0.2% Triton-X. PNMA2 was visualized with immunofluorescence using a PNMA2 primary antibody (ProteinTech 16445-1-AP 1:1000) followed by goat anti rabbit AlexaFluor 647 secondary (Abcam, ab150083 1:500).

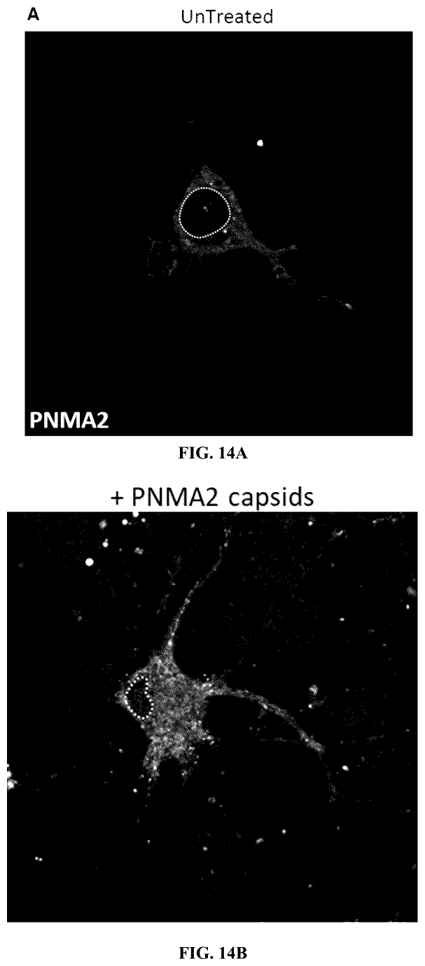

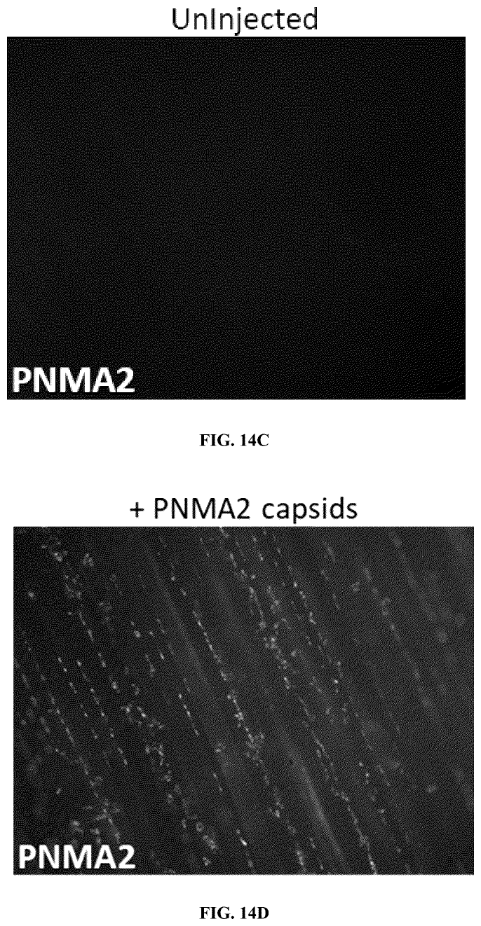

Fluorescence microscopy revealed a punctate staining pattern, suggesting that the PNMA2 capsids were internalized by the neurons (FIG. 14B). Little or no intracellular staining was observed in mock-treated cells (FIG. 14A).

To evaluate internalization of PNMA2 capsids in vivo, C57BL/6 mice were injected intramuscularly in the hind quarter with 10 μg of PNMA2 capsids. Four hours after capsid administration, animals were sacrificed and muscle tissue from both the injected side, and the contralateral un-injected side were collected and washed in ice cold PBS. Tissues were fixed with −20° C. methanol for 15 minutes, then blocked and permeabilized with BSA blocker and 1% Triton-X100. Capsids were stained with anti-PNMA2 primary antibody (Sigma HPA001936 1:500) followed by goat anti rabbit AlexaFluor 647 secondary (Abcam, ab150083 1:500). FIG. 14D provides an epifluorescence microcopy image of tissue from the injected side, demonstrating the presence of capsids in muscle fibers. FIG. 14C shows a control image from the un-injected tissue.

### Example 15—Functional Elements can be Attached to PNMA2 Capsids

Chemical attachment of various fluorescent dyes to the engineered PNMA2 polypeptides allows for convenient and sensitive tracing of the resulting PNMA2 capsids in various biological systems (e.g., from individual cells to organs within a living organism) in vivo and in vitro without compromising or substantially without compromising their structure-functional properties.

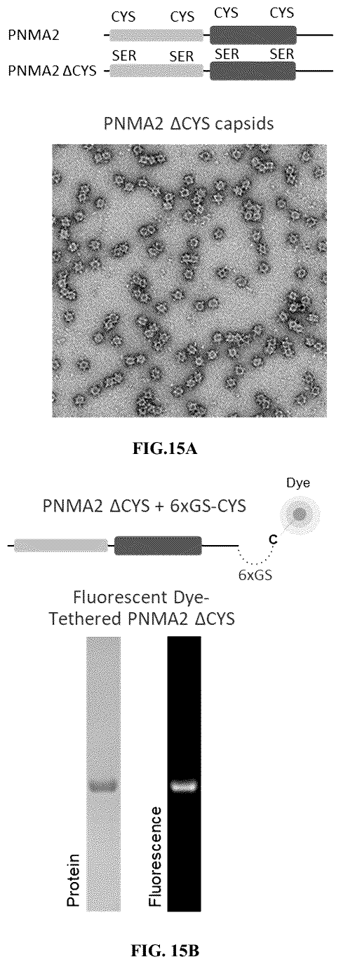

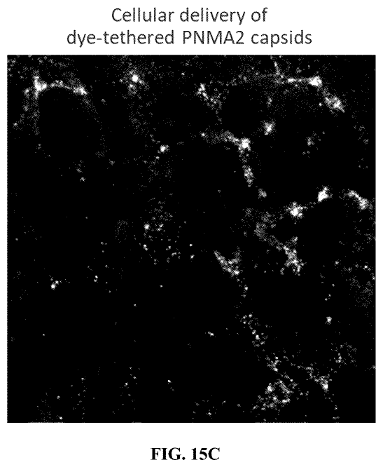

In this example, efficient chemical attachment of Alexa 647 fluorescent dye is demonstrated. The fluorescent dye was attached to an engineered quadruple cysteine mutant PNMA2 (ΔCYS: C10S/C136S/C233S/C310S, FIG. 15A, top panel; SEQ ID NO: 48) that carries a flexible cysteine residue appended to the extreme C-terminus of the polypeptide via a GS linker (SEQ ID NO: 49).

Table 5: sequences of quadruple cysteine mutant PNMA2 (ΔCYS) with and without C-terminal cysteine

TABLE 5

| SEQ | | |
|---|---|---|
| ID | | |
| NO: | Description | Sequence |
| 48 | PNMA2 | ALALLEDWSRIMSVDEQKSLMVTGIPADFEEAEIQEVLQETLKSLGR |
| | C10S/C136S/ | YRLLGKIFRKQENANAVLLELLEDTDVSAIPSEVQGKGGVWKVIFKT |
| | C233S/C310S | PNQDTEFLERLNLFLEKEGQTVSGMFRALGQEGVSPATVPSISPELLA |
| | | HLLGQAMAHAPQPLLPMRYRKLRVFSGSAVPAPEEESFEVWLEQAT |
| | | EIVKEWPVTEAEKKRWLAESLRGPALDLMHIVQADNPSISVEESLEA |
| | | FKQVFGSLESRRTAQVRYLKTYQEEGEKVSAYVLRLETLLRRAVEK |
| | | RAIPRRIADQVRLEQVMAGATLNQMLWSRLRELKDQGPPPSFLELM |
| | | KVIREEEEEEASFENESIEEPEERDGYGRWNHEGDD |
| 49 | C10S/C136S/ | ALALLEDWSRIMSVDEQKSLMVTGIPADFEEAEIQEVLQETLKSLGR |
| | C233S/C310S | YRLLGKIFRKQENANAVLLELLEDTDVSAIPSEVQGKGGVWKVIFKT |
| | with C- | PNQDTEFLERLNLFLEKEGQTVSGMFRALGQEGVSPATVPSISPELLA |
| | terminal | HLLGQAMAHAPQPLLPMRYRKLRVFSGSAVPAPEEESFEVWLEQAT |
| | cysteine | EIVKEWPVTEAEKKRWLAESLRGPALDLMHIVQADNPSISVEESLEA |
| | attached via | FKQVFGSLESRRTAQVRYLKTYQEEGEKVSAYVLRLETLLRRAVEK |
| | flexible | RAIPRRIADQVRLEQVMAGATLNQMLWSRLRELKDQGPPPSFLELM |
| | linker | KVIREEEEEEASFENESIEEPEERDGYGRWNHEGDDGSGSGSGSGSGS |
| | | C |

Similar to its wild type counterpart, the quadruple cysteine PNMA2 mutant was able to form capsids upon reassembly, as shown by transmission electron microscopy analysis (FIG. 15A, bottom panel).

The fluorescent dye was conjugated to the introduced C-terminal cysteine via maleimide chemistry (FIG. 15B, upper panel) and its association was confirmed by biochemical analysis and fluorescence detection (FIG. 15B). Alexa 647 labeled PNMA2 ΔCYS+6×GS-CYS protein was reassembled into capsids and incubated with colorectal carcinoma HCT116 cells (“6×GS-CYS” disclosed as SEQ ID NO: 59). FIG. 15C demonstrates intracellular detection of Alexa 647 labeled PNMA2 capsids by fluorescent microscopy.

### Example 16—Cellular-Targeting Elements can be Engineered into PNMA2 Capsids

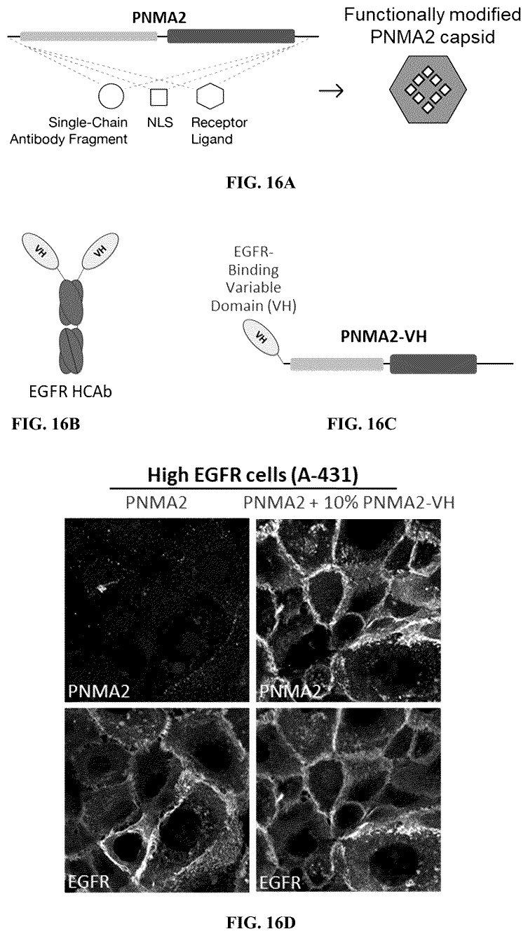

PNMA2 can be engineered to include various moieties to increase cellular uptake or modify intracellular localization upon uptake into cells (FIG. 16A). For example, single chain antibody fragments targeting specific receptors on cells can be engineered onto the N or C terminus of the protein. Receptor ligands and subcellular localization signals such as a nuclear localization signal (NLS) can also be attached or conjugated to PNMA2 or incorporated into PNMA2 fusion proteins. Single chain antibodies (e.g., single chain variable fragments (scFv's)) or single domain antibody fragments from heavy chain-only antibodies (HCAb), such as VHH domains from camelid antibodies (FIG. 16B), are also suitable for targeting because of their relatively small size (e.g., 12-15 kD) and high affinities for their targets.

In this example, the variable domain from a HCAb that binds EGFR (SEQ ID NO: 50) was engineered into the N terminus of PNMA2 to generate PNMA2-VH (FIG. 16C; SEQ ID NO: 52). A flexible 5×SG linker (SEQ ID NO: 57) was used to join the anti-EGFR domain to PNMA2, and the construct was expressed with an N-terminal 6×His tag and TEV cleavage site (SEQ ID NO: 53) Table 6: illustrative engineered PNMA2 with a cellular-targeting element

TABLE 6

| SEQ | | |
|---|---|---|
| ID | | |
| NO: | Description | Sequence |
| 50 | Anti-EGFR VHH | AAQVKLEESGGGSVQTGGSLRLTCAASGRTSRSYGMGWFRQA |
| | domain | PGKEREFVSGISWRGDSTGYADSVKGRFTISRDNAKNTVDLQM |
| | | NSLKPEDTAIYYCAAAAGSAWYGTLYEYDYWGQGTQVTVSS |
| 51 | Anti-EGFR VHH | MHHHHHHGSGSGSENLYFQGAAQVKLEESGGGSVQTGGSLRL |
| | domain with N- | TCAASGRTSRSYGMGWFRQAPGKEREFVSGISWRGDSTGYADS |
| | terminal 6x-His | VKGRFTISRDNAKNTVDLQMNSLKPEDTAIYYCAAAAGSAWY |
| | tag and cleavage | GTLYEYDYWGQGTQVTVSSSGSGSGSGSG |
| | site and C- | |
| | terminal linker | |
| 52 | PNMA2-VH | AAQVKLEESGGGSVQTGGSLRLTCAASGRTSRSYGMGWFRQA |
| | | PGKEREFVSGISWRGDSTGYADSVKGRFTISRDNAKNTVDLQM |
| | | NSLKPEDTAIYYCAAAAGSAWYGTLYEYDYWGQGTQVTVSSS |
| | | GSGSGSGSGALALLEDWCRIMSVDEQKSLMVTGIPADFEEAEIQ |
| | | EVLQETLKSLGRYRLLGKIFRKQENANAVLLELLEDTDVSAIPSE |
| | | VQGKGGVWKVIFKTPNQDTEFLERLNLFLEKEGQTVSGMFRAL |
| | | GQEGVSPATVPCISPELLAHLLGQAMAHAPQPLLPMRYRKLRVF |
| | | SGSAVPAPEEESFEVWLEQATEIVKEWPVTEAEKKRWLAESLR |
| | | GPALDLMHIVQADNPSISVEECLEAFKQVFGSLESRRTAQVRYL |
| | | KTYQEEGEKVSAYVLRLETLLRRAVEKRAIPRRIADQVRLEQV |
| | | MAGATLNQMLWCRLRELKDQGPPPSFLELMKVIREEEEEEASF |
| | | ENESIEEPEERDGYGRWNHEGDD |
| 53 | PNMA2-VH with | MHHHHHHGSGSGSENLYFQGAAQVKLEESGGGSVQTGGSLRL |
| | N-terminal tag | TCAASGRTSRSYGMGWFRQAPGKEREFVSGISWRGDSTGYADS |
| | | VKGRFTISRDNAKNTVDLQMNSLKPEDTAIYYCAAAAGSAWY |
| | | GTLYEYDYWGQGTQVTVSSSGSGSGSGSGALALLEDWCRIMSV |
| | | DEQKSLMVTGIPADFEEAEIQEVLQETLKSLGRYRLLGKIFRKQE |
| | | NANAVLLELLEDTDVSAIPSEVQGKGGVWKVIFKTPNQDTEFLE |
| | | RLNLFLEKEGQTVSGMFRALGQEGVSPATVPCISPELLAHLLGQ |
| | | AMAHAPQPLLPMRYRKLRVFSGSAVPAPEEESFEVWLEQATEIV |
| | | KEWPVTEAEKKRWLAESLRGPALDLMHIVQADNPSISVEECLE |
| | | AFKQVFGSLESRRTAQVRYLKTYQEEGEKVSAYVLRLETLLRR |
| | | AVEKRAIPRRIADQVRLEQVMAGATLNQMLWCRLRELKDQGP |
| | | PPSFLELMKVIREEEEEEASFENESIEEPEERDGYGRWNHEGDD |

Capsids were generated with either 100% PNMA2 or 90% PNMA2 and 10% PNMA2-VH. A431 cells (a cancer cell line with high EGFR expression) were treated with the capsids at 0.5 mg/ml in PBS (supplemented with 10% FBS) for 4 hours at 37° C. The cells were then washed three times for 10 minutes each with complete media, followed by a quick PBS wash and fixation with 4% paraformaldehyde (PFA) in PBS. Fixed cells were then permeabilized with 0.2% Triton-X100 and blocked with BSA blocker and 5% goat serum prior to overnight incubation with a rabbit monoclonal antibody against PNMA2 and a chicken antibody against EGFR. For immunofluorescent staining a goat anti-rabbit Alexa647 secondary antibody was used to detect the anti-PNMA2 antibodies (FIG. 16D top panels) and a goat anti-chicken Alexa 488 secondary was used to detect the anti EGFR antibodies (FIG. 16D bottom panels). Immunofluorescence imaging demonstrated that significantly more of the PNMA2 capsids with the anti-EGFR antibody domain were delivered to A431 cells, and that the PNMA2-VH capsids were colocalizing with EGFR.

### Example 17—Peptides (Engineered or Added in Trans) can Enhance PNMA2 Capsid Uptake

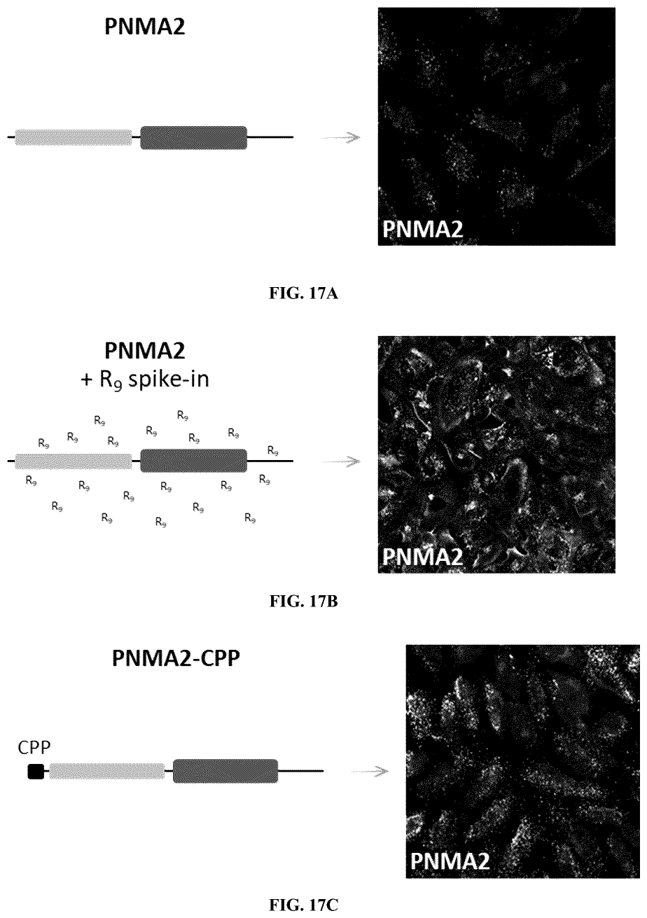

Short cationic peptides have been used to enhance delivery of proteins and nucleic acids to cells. This example demonstrates enhanced uptake of PNMA2 capsids when short cationic peptides are either added in trans or engineered into the PNMA2 protein scaffold. FIG. 17A shows PNMA2 capsid uptake in HeLa cells when treated at 0.5 mg/ml for 4 hours at 37° C. Immunofluorescence was used as detailed in EXAMPLE 16 to visualize PNMA2 protein in cells after treatment. Separately, PNMA2 capsids were mixed with 20 μM R9 peptide (a positivity charged peptide comprised of 9 arginine residues; Anaspec; SEQ ID NO: 56) prior to treatment of cells in the same manner as described above, resulting in a significant increase in PNMA2 uptake compared to capsid alone (FIG. 17B). In a third experimental condition, a short cationic peptide was engineered into the N terminus of the PNMA2 protein (PNMA2-CPP) to generated cell penetrating PNMA2 capsids. PNMA-CPP was generated with the TAT sequence (YGRKKRRQRRR; SEQ ID NO: 54) added to the PNMA2 protein N-terminus, then hybrid capsids were generated as detailed in EXAMPLE 13, with 50% PNMA2 and 50% PNMA2-CPP. Hybrid CPP-PNMA2 capsids were added to HeLa cells at 0.5 mg/ml for 4 hours at 37° C. Immunofluorescent staining for PNMA2 was used to visualize cellular uptake. The addition of the cell penetrating peptide increased uptake of the PNMA2 capsid.

### Example 18—PNMA2 Capsids Deliver Heterologous and Functional RNA

This example demonstrates delivery of heterologous and functional RNA to cells using PNMA2 capsids of the disclosure.

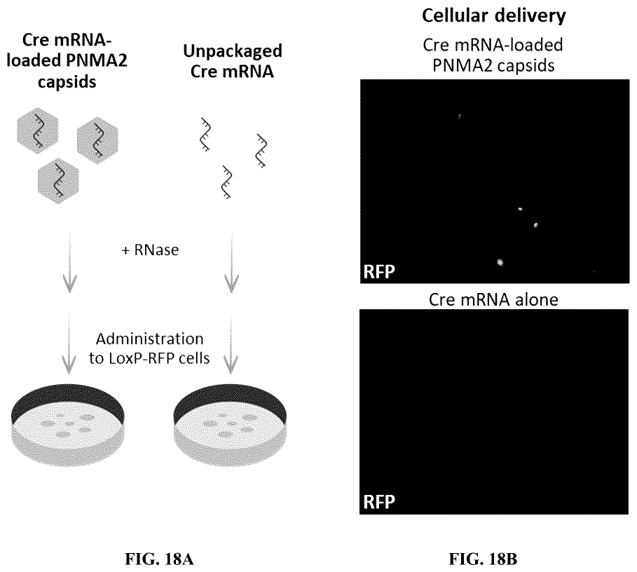

PNMA2 capsids were loaded with Cre mRNA, and the capsids were used to deliver the Cre mRNA to GFP/RFP switch reporter cells comprising a GFP-loxP-Stop-loxP-RFP construct. Upon exposure to Cre recombinase, the Stop codon between the two loxP sites is excised, allowing read-through expression of RFP. For this assay, Cre mRNA-loaded PNMA2 capsids or Cre mRNA alone were formulated in PBS with 2.5 mM MgCl and then subjected to nuclease digestion for 15-30 minutes at room temperature, followed by addition onto GFP/RFP reporter cells (FIG. 18A). Specifically, U87 MG glioma reporter cells were plated at a density of 12,000 cells/well (100 μL volume) in a 96 well format. Twenty-four hours later, 50 μL of nuclease treated Cre loaded PNMA2 capsids (0.25-0.5 mg/ml), or an equivalent amount of unpackaged Cre mRNA, was added to the 100 μL of tissue culture media. 48-72 hours post treatment, cells were imaged to visualize RFP expression.

RFP fluorescence was observed for cells treated with the PNMA2 capsids containing Cre mRNA as a cargo, but not for unpackaged Cre mRNA, demonstrating that: (i) the capsids protected the Cre mRNA from degradation by RNase, and (ii) the PNMA2 capsids delivered functional Cre mRNA that was translated to Cre recombinase, which induced RFP expression (FIG. 18B).

### Example 19—Engineered PNMA2 (PNMA2-RBD) Capsids Deliver Functional RNA

As described in EXAMPLE 12, PNMA2 can be engineered to include an RNA binding domain (RBD) that facilitates loading of RNA during capsid assembly or reassembly. In this example, PNMA2-RBD (SEQ ID NO: 55) was used to generate capsids loaded with Cre mRNA.

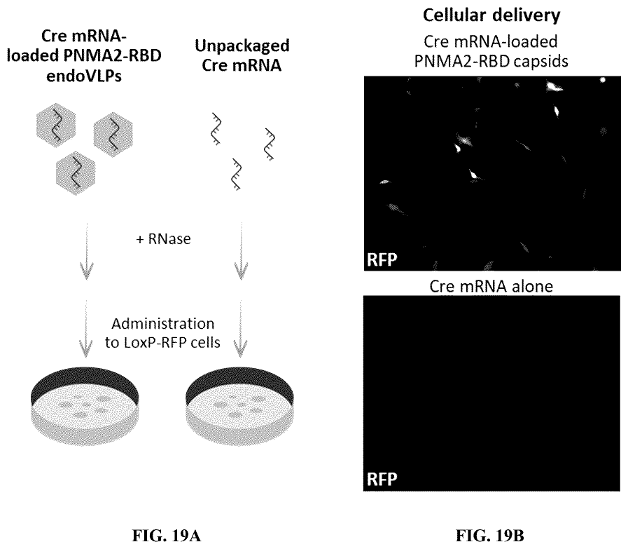

These capsids (0.25-0.5 mg/ml), and an equivalent amount of Cre mRNA alone, were then subject to nuclease digestion for 15 min at room temperature prior to being administered onto GFP/RFP switch cells (FIG. 19A). The Cre mRNA alone was a control to ensure full nuclease digestion of any unloaded Cre mRNA. Forty-eight hours after treatments, cells were then imaged with an epi fluorescence microscope to visualize RFP expression.

RFP fluorescence was observed for cells treated with the PNMA2-RBD capsids containing Cre mRNA as a cargo, but not for unpackaged Cre mRNA, demonstrating that: (i) the capsids protected the Cre mRNA from degradation by RNase, and (ii) the PNMA2 capsids delivered functional Cre mRNA that was translated to Cre recombinase, which induced RFP expression (FIG. 19B).

### Example 20—Lipids Enhance PNMA2 Capsid Functional mRNA Delivery

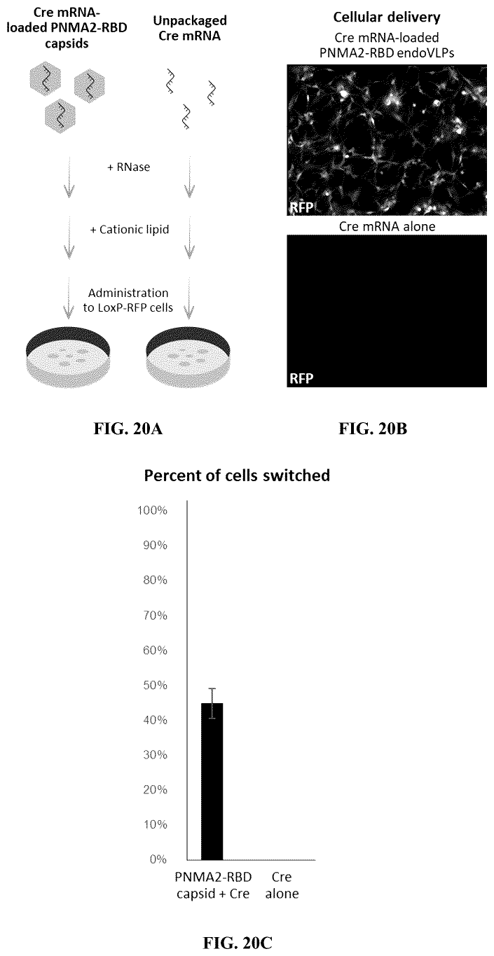

To enhance transduction of Cre mRNA loaded PNMA2-RBD capsids, the addition of cationic lipids was investigated. In this example, capsids were formed as disclosed in EXAMPLE 12 and subject to nuclease digestion. Prior to administration onto cells, a cationic lipid (jetMESSENGER, or analogous lipid agents) was added at 0.5% volume to volume and allowed to incubate for 10 minutes at room temperature (FIG. 20A). An equivalent amount of free Cre mRNA was subject to the same treatment as a control for nuclease activity. The cationic lipid can associate with the capsid (e.g., a negative spike portion thereof) via electrostatic interactions. The presence of the cationic lipid on a capsid can enhance binding to the negatively charged surface of a cell, facilitating increased capsid uptake. U87 MG GFP/RFP switch cells were imaged 48-72 hours after treatments to visualize RFP expression

RFP fluorescence was observed for cells treated with the PNMA2-RBD capsids containing Cre mRNA as a cargo and incubated with the cationic lipid, but not for unpackaged Cre mRNA, demonstrating that: (i) the capsids protected the Cre mRNA from degradation by RNase, and (ii) the PNMA2 capsids delivered functional Cre mRNA that was translated to Cre recombinase, which induced RFP expression (FIG. 20B).

To quantify the efficiency of mRNA delivery, masks were generated in Image J to locate all GFP+ pixels in the green channel of a treatment image. For the same field of view, that mask was applied to the red channel and the percent of GFP+ pixels that were also positive for RFP was determined. This measurement was used to estimate the number of GFP+ cells that were successfully transduced with Cre mRNA, as demonstrated by RFP expression. This treatment was done in quadruplicate, and Cre loaded PNMA2-RBD capsids induced RFP expression in approximately 45% of treated cells, whereas unprotected RNA did not induce any RFP expression (FIG. 20C).

### Example 21—Peptides Enhance PNMA2 Capsid Functional mRNA Delivery

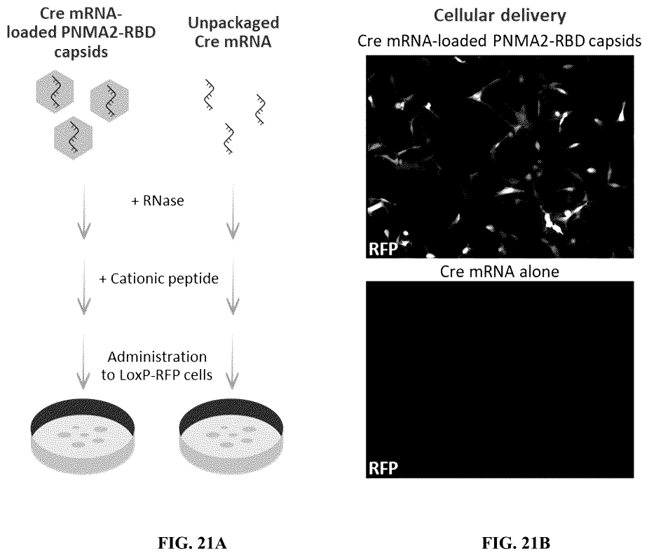

This example demonstrates that cationic peptides can enhance delivery of functional RNA via loaded PNMA2-RBD capsids. A cationic peptide can associate with the capsid (e.g., a negative spike portion thereof) via electrostatic interactions. The presence of the cationic peptide on a capsid can enhance binding to the negatively charged surface of a cell, facilitating increased capsid uptake. Capsids were generated as disclosed in EXAMPLE 12, subject to nuclease digestion, and then cationic peptides were added in trans. In this example, vectofusion-1 peptide was added to capsids at a concentration of 30 μg/ml) and allowed to incubate for 10 minutes prior to administration into cells (FIG. 21A). For cell treatments, 50 μL of capsid/peptide cocktail was spiked into the 100 μL of tissue culture media. At least 48 hours after treatments, cells were imaged to visualized RFP expression.

RFP fluorescence was observed for cells treated with the PNMA2-RBD capsids containing Cre mRNA as a cargo and incubated with the cationic peptide, but not for unpackaged Cre mRNA, demonstrating that: (i) the capsids protected the Cre mRNA from degradation by RNase, and (ii) the PNMA2 capsids delivered functional Cre mRNA that was translated to Cre recombinase, which induced RFP expression (FIG. 21B)

### Example 22—PNMA2 Capsid Delivery of Functional hpRNA with or without Delivery-Enhancing Agents

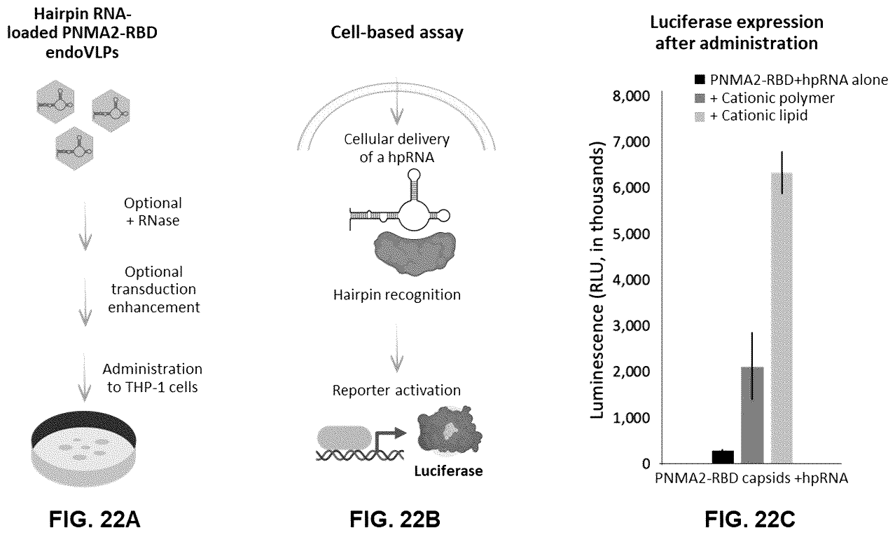

PNMA2-RBD capsids were formed as disclosed in EXAMPLE 12 in the presence of a hairpin RNA (hpRNA). hpRNA loaded capsids were subjected to nuclease digestion. In certain experimental conditions, additional transduction enhancers were included (cationic polymer or cationic lipid), followed by administration of the capsids onto THP-1 reporter cells (FIG. 22A). The THP-1 monocytes have stable integration of an inducible reporter and secrete luciferase under the control of an ISG54 (interferon-stimulated gene) minimal promoter in conjunction with multiple interferon (IFN)-stimulated response elements. The structure of the hpRNA is similar to that of viral RNA and is designed to induce activation of the interferon regulatory pathway upon delivery to the cell (FIG. 22B).

THP-1 cells were plated at a density of 1×10\hat{ }5 cells/well (180 μL) in a 96 well plate. Capsid treatments were conducted as described in FIG. 22A, with the addition of 20 μL of hpRNA loaded PNMA2-RBD capsid. Capsids were administered alone, with the addition of polybrene (a cationic polymer) at 60 μg/ml, or with the addition of jet messenger (cationic lipid) at 0.5% v/v. The cationic lipid or polymer can associate with the capsid (e.g., negative spike portion thereof) via electrostatic interactions. The presence of a cationic lipid or polymer on a capsid can enhance binding to the negatively charged surface of a cell, facilitating increased capsid uptake. Twenty-four hours after capsid treatments (done in triplicate), 20 μL of cell culture supernatant was transferred to a fresh 96 well plate and 50 μL of luciferase substrate was added. Luminescence measurements were taken immediately post addition of substrate with an integration time of 0.1 ms.

Luminescence data showed that PNMA2-RBD capsids packaged with hpRNA induced expression of the reporter gene, and expression was higher for conditions that included the cationic polymer or cationic lipid as a delivery-enhancing agent (FIG. 22C).

### Example 23—Loaded and Protected RNA is the Functional Agent

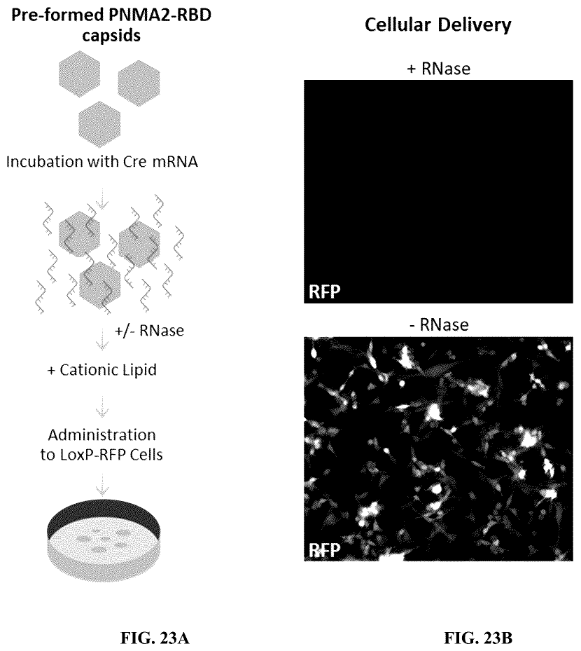

PNMA2-RBD capsids were formed in the absence of nucleic acids and then mixed with Cre mRNA at a ratio of 4 μg RNA per 50 μg Protein. This capsid/RNA mixture was then treated with a nuclease in the same manner as EXAMPLES 18-21 with addition of cationic lipid as in EXAMPLE 20, prior to being administered to U87 MG GFP/RFP switch cells (FIG. 23A).

Empty PNMA2-RBD capsids mixed with Cre mRNA followed by nuclease addition did not induce any RFP expression by the cells (FIG. 23B, top panel). Treatment in the absence of nuclease was included as a control (FIG. 23B, bottom panel) to verify the functionality of the RNA prior to nuclease digestion.

### Example 24—In Vivo Loading and Delivery of Functional RNAs

A reporter mouse was used to demonstrate in vivo delivery of a functional RNA using a PNMA2 capsid of the disclosure. The Ai14 mouse strain was utilized, which is genetically modified to express robust tdTomato fluorescence following Cre-mediated recombination.

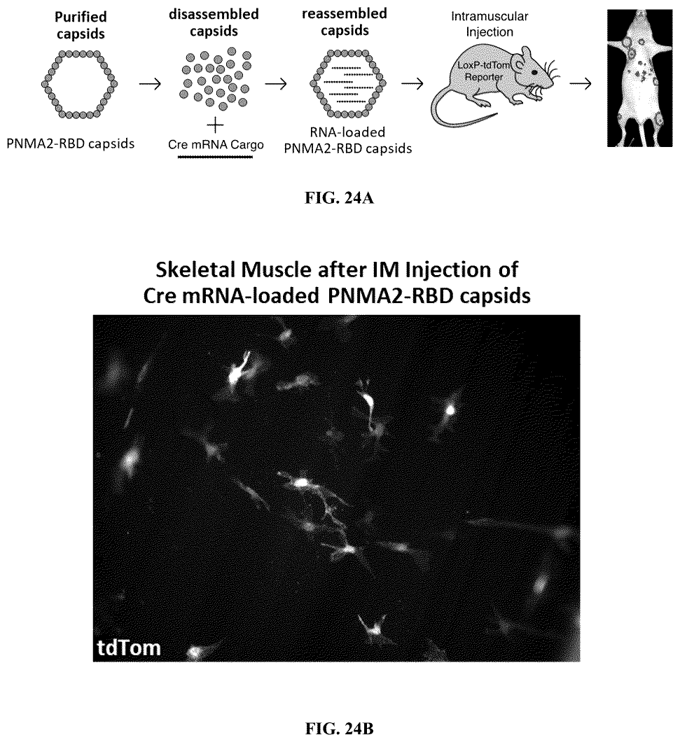

Purified PNMA2 capsids were disassembled and then reassembled in the presence of Cre mRNA to load the functional cargo into the capsid. These capsids were then sterile filtered and injected into the hind quarter of a reporter mouse (FIG. 24A). Seven days after in intramuscular (IM) injection of 40 μL of Cre mRNA loaded PNMA2-RBD capsid (at 0.3 mg/ml), animals were sacrificed and the muscle tissue of the hind limbs was collected and processed for fluorescent imaging. tdTomato expression was detected in the treated muscle tissue (FIG. 24B) demonstrating delivery of functional RNA in vivo.

### Example 25—Comparison of Biochemical Properties of PNMA2 Capsids to Arc Capsids

Human PNMA2 protein was expressed, purified, and characterized as disclosed herein.

Human Arc was expressed, purified, and characterized to compare properties of PNMA2 capsids to Arc capsids, e.g., capsid formation efficiency, capsid disassembly efficiency, capsid reassembly efficiency, and nucleic acid binding abilities.

To construct recombinant DNA vectors for Arc expression, full length cDNA open reading frames, excluding the initial methionine, were inserted into a cloning vector and subsequently transferred into an expression vector.

cDNA encoding Arc was inserted into an expression vector derived from pET-41-a(+) (EMD Millipore (Novagen) Cat #70566). The entire cloning site of pET-41-a(+) was removed and replaced with DNA having the nucleotide sequence of SEQ ID NO: 32, which encodes an N-terminal tag having the amino acid sequence of SEQ ID NO: 33, comprising a 6×His tag (SEQ ID NO: 27), a 6 amino acid spacer (SEQ ID NO: 28), and a TEV cleavage site (SEQ ID NO: 29). The Arc open reading frame without the N-terminal methionine codon (SEQ ID NO: 34, encoding SEQ ID NO: 35) was inserted after the TEV cleavage site by Gibson assembly. After expression and TEV cleavage, the N-terminus of the resulting Arc protein has a single residual Glycine from the TEV cleavage site (SEQ ID NO: 36). Table 7 provides sequences related to the Arc polypeptides of this example.

TABLE 7

| SEQ | | |
|---|---|---|
| ID | | |
| NO: | Description | Sequence |
| 32 | Nucleotide | ATGCATCACCATCACCATCACGGCTCAGGGTCTGGTAGCGAAAAT |
| | sequence | CTGTACTTCCAGGGG |
| | encoding N- | |
| | terminal tag | |
| 33 | N-terminal | MHHHHHHGSGSGSENLYFQG |
| | tag amino | |
| | acid | |
| | sequence | |
| 34 | Arc | GGGGAGCTGGACCACCGGACCAGCGGCGGGCTCCACGCCTACCC |
| | nucleotide | CGGGCCGCGGGGGGGGCAGGTGGCCAAGCCCAACGTGATCCTGC |
| | sequence, | AGATCGGGAAGTGCCGGGCCGAGATGCTGGAGCACGTGCGGCGG |
| | lacking N- | ACGCACCGGCACCTGCTGGCCGAGGTGTCCAAGCAGGTGGAGCG |
| | terminal | CGAGCTGAAGGGGCTGCACCGGTCGGTCGGGAAGCTGGAGAGCA |
| | Met | ACCTGGACGGCTACGTGCCCACGAGCGACTCGCAGCGCTGGAAG |
| | | AAGTCCATCAAGGCCTGCCTGTGCCGCTGCCAGGAGACCATCGCC |
| | | AACCTGGAGCGCTGGGTCAAGCGCGAGATGCACGTGTGGCGCGA |
| | | GGTGTTCTACCGCCTGGAGCGCTGGGCCGACCGCCTGGAGTCCAC |
| | | GGGCGGCAAGTACCCGGTGGGCAGCGAGTCAGCCCGCCACACCG |
| | | TTTCCGTGGGCGTGGGGGGTCCCGAGAGCTACTGCCACGAGGCAG |
| | | ACGGCTACGACTACACCGTCAGCCCCTACGCCATCACCCCGCCCC |
| | | CAGCCGCTGGCGAGCTGCCCGGGCAGGAGCCCGCCGAGGCCCAG |
| | | CAGTACCAGCCGTGGGTCCCCGGCGAGGACGGGCAGCCCAGCCC |
| | | CGGCGTGGACACGCAGATCTTCGAGGACCCTCGAGAGTTCCTGAG |
| | | CCACCTAGAGGAGTACTTGCGGCAGGTGGGCGGCTCTGAGGAGT |
| | | ACTGGCTGTCCCAGATCCAGAATCACATGAACGGGCCGGCCAAG |
| | | AAGTGGTGGGAGTTCAAGCAGGGCTCCGTGAAGAACTGGGTGGA |
| | | GTTCAAGAAGGAGTTCCTGCAGTACAGCGAGGGCACGCTGTCCCG |
| | | AGAGGCCATCCAGCGCGAGCTGGACCTGCCGCAGAAGCAGGGCG |
| | | AGCCGCTGGACCAGTTCCTGTGGCGCAAGCGGGACCTGTACCAGA |
| | | CGCTCTACGTGGACGCGGACGAGGAGGAGATCATCCAGTACGTG |
| | | GTGGGCACCCTGCAGCCCAAGCTCAAGCGTTTCCTGCGCCACCCC |
| | | CTGCCCAAGACCCTGGAGCAGCTCATCCAGAGGGGCATGGAGGT |
| | | GCAGGATGACCTGGAGCAGGCGGCCGAGCCGGCCGGCCCCCACC |
| | | TCCCGGTGGAGGATGAGGCGGAGACCCTCACGCCCGCCCCCAAC |
| | | AGCGAGTCCGTGGCCAGTGACCGGACCCAGCCCGAG |
| 35 | Arc amino | ELDHRTSGGLHAYPGPRGGQVAKPNVILQIGKCRAEMLEHVRRTHR |
| | acid | HLLAEVSKQVERELKGLHRSVGKLESNLDGYVPTSDSQRWKKSIKA |
| | sequence, | CLCRCQETIANLERWVKREMHVWREVFYRLERWADRLESTGGKYP |
| | lacking N- | VGSESARHTVSVGVGGPESYCHEADGYDYTVSPYAITPPPAAGELPG |
| | terminal | QEPAEAQQYQPWVPGEDGQPSPGVDTQIFEDPREFLSHLEEYLRQVG |
| | Met | GSEEYWLSQIQNHMNGPAKKWWEFKQGSVKNWVEFKKEFLQYSEG |
| | | TLSREAIQRELDLPQKQGEPLDQFLWRKRDLYQTLYVDADEEEIIQY |
| | | VVGTLQPKLKRFLRHPLPKTLEQLIQRGMEVQDDLEQAAEPAGPHLP |
| | | VEDEAETLTPAPNSESVASDRTQPE |
| 36 | Arc amino | GELDHRTSGGLHAYPGPRGGQVAKPNVILQIGKCRAEMLEHVRRTH |
| | acid | RHLLAEVSKQVERELKGLHRSVGKLESNLDGYVPTSDSQRWKKSIK |
| | sequence | ACLCRCQETIANLERWVKREMHVWREVFYRLERWADRLESTGGKY |
| | after | PVGSESARHTVSVGVGGPESYCHEADGYDYTVSPYAITPPPAAGELP |
| | expression | GQEPAEAQQYQPWVPGEDGQPSPGVDTQIFEDPREFLSHLEEYLRQV |
| | and | GGSEEYWLSQIQNHMNGPAKKWWEFKQGSVKNWVEFKKEFLQYSE |
| | cleavage | GTLSREAIQRELDLPQKQGEPLDQFLWRKRDLYQTLYVDADEEEIIQ |
| | | YVVGTLQPKLKRFLRHPLPKTLEQLIQRGMEVQDDLEQAAEPAGPHL |
| | | PVEDEAETLTPAPNSESVASDRTQPE |

An expression vector construct comprising Arc open reading frame was transformed into the Rosetta 2 (DE3)pLysS E. coli strain (Millipore Sigma, Cat #71403). Arc expression was induced with 0.1 mM IPTG followed by a 16-hour incubation at 16° C. Cell pellets were lysed by sonication in 20 mM sodium phosphate pH 7.4, 0.1 M NaCl, 40 mM imidazole, 1 mM DTT, 2 mM MgCl₂, 1 mM ATP, and 10% glycerol. The lysate was treated with excess TURBO DNase (Thermo Fisher Scientific, Cat #AM2238), RNase Cocktail (Thermo Fisher Scientific, Cat #AM2286), and Benzonase Nuclease (Millipore Sigma, Cat #71205) to eliminate nucleic acids. NaCl was added to lysate in order to adjust the NaCl concentration to 0.5 M followed by centrifugation and filtration to remove cellular debris. 6×His-tagged recombinant protein was loaded onto a HisTrap HP column (GE Healthcare, Cat #17-5247-01), washed with HisTrap buffer A (HTA) (20 mM sodium phosphate pH 7.4, 0.5 M NaCl, 40 mM imidazole, and 10% glycerol), and eluted with a linear gradient of HisTrap buffer B (HTB) (20 mM sodium phosphate pH 7.4, 0.5 M NaCl, 500 mM imidazole, and 10% glycerol). Collection tubes were supplemented in advance with 1011.1 of 0.5 M EDTA pH 8.0 per 1 ml eluate. The resulting Arc protein was more than 95% pure as revealed by SDS-PAGE analysis, with a yield of up to 50 mg per 1 L of bacterial culture.

Residual nucleic acid was removed by anion exchange chromatography on a mono Q 5/50 GL column (GE Healthcare, Cat #17516601). Before loading to the column, recombinant protein was buffer exchanged to buffer C (20 mM Tris-HCl pH 8.0, 100 mM NaCl, and 10% glycerol) using “Pierce Protein Concentrator PES, 10K MWCO, 5-20 ml” (Thermo Scientific, Cat #88528) according to the manufacturer's protocol. After loading, the Mono Q resin was washed with 2 ml of buffer C. Arc protein was eluted using a linear gradient of buffer D (20 mM Tris-HCl pH 8.0, 500 mM NaCl, and 10% glycerol). RNA efficiently separated from Arc and eluted at 600 mM NaCl.

The N-terminal 6×His tag and spacer was removed from concentrating peak fractions of the Mono Q purified Arc using a 10 kDa MWCO PES concentrator and then treating with 10% v/v of TEV (10 units/mL). The cleavage efficiency was above 99% as revealed by SDS-PAGE assay. The protein was then diluted into HTA2 and cleaned with HisTrap HP resin. The resulting purified Arc has an N-terminal Glycine residue and does not contain the initial methionine.

Cleaved Arc protein (1 mg/mL) was loaded into a 20 kDa MWCO dialysis cassette and dialyzed overnight in 1 M sodium phosphate (pH 7.5) at room temperature. The following day, the solution was removed from the cassette, transferred to microcentrifuge tubes, and spun at max speed for 5 minutes in a tabletop centrifuge. The supernatant was transferred to a 100 kDa MWCO Regenerated Cellulose Amicon Ultrafiltration Centrifugal concentrator. The buffer was exchanged to PBS pH 7.5 and the volume was reduced 20-fold to concentrate the capsids.

Following Arc purification, capsid assembly was assayed by transmission electron microscopy. EM grids (Carbon Support Film, Square Grid, 400 mesh, 3-4 nm, Copper, CF400-Cu-UL) were prepared by glow discharge. A 5 μL sample of purified Arc was applied to the grid for 30 seconds and then wicked away using filter paper. The grid was then washed twice with MilliQ H₂O. MilliQ H₂O was immediately wicked away using filter paper after each wash. The grid was then washed twice with 5 μL of 1% Uranyl Acetate in H₂O. Uranyl Acetate was immediately wicked away using filter paper after each wash. The grid was then stained using 54, of 1% Uranyl Acetate in H₂O for 3 minutes and air dried for 1 minute. Images of Arc capsids were acquired using a FEI Talos L120C TEM equipped with a Gatan 4k×4k OneView camera. The Arc capsids were also subjected to multi-angle dynamic light scattering (MARLS) using a Malvern Zetasizer Ultra. Arc capsid disassembly and reassembly properties were assessed using similar techniques to Example 4. In contrast to PNMA2, no disassembly or reassembly of Arc capsids was observed in any of the tested conditions.

To calculate PNMA2 and Arc capsid disassembly and reassembly efficiency, the amount of protein loss was measured, and particle sizes and concentrations were calculated using multi-angle dynamic light scattering, with reassembly confirmed by TEM. As shown in TABLE 8, PNMA2 exhibited surprisingly and unexpectedly superior capsid formation, disassembly, and reassembly properties compared to Arc.

TABLE 8

| | Arc | PNMA2 |
|---|---|---|
| Protein yield per L of E. coli culture | ~40 mg | 5-20 mg |
| Capsid formation efficiency | <5% | ≥90% |
| Capsid disassembly efficiency | <5% | ≥90% |
| Capsid reassembly efficiency | <5% | ≥90% |
| Nucleic acid binding ability | + | + |

An example of a technique for assessing in vitro capsid formation assembly efficiency is provided in Gross et al., (1997) In vitro assembly properties of purified bacterially expressed capsid proteins of human immunodeficiency virus. European Journal of Biochemistry, 249(2), 592-600, which is incorporated herein by reference for such disclosure. For example, the size and/or number of assembled capsid particles per EM mesh grid can be counted as a measure of assembly efficiency.

### Example 26—Capsid Disassembly and Reassembly

Recently purified PNMA2 protein (1 mg/mL in HTB) was added 1:1 dropwise into 2× disassembly buffer A (10% CHAPS, 20 mM TCEP, 100 mM Tris pH 8.0) and incubated on ice for 30 minutes. Following the incubation, disassembly was confirmed by i) MADLS and ii) TEM.

In a separate assay, PNMA2 protein was disassembled by loading PNMA2 (0.2 mg/mL) into a 10 kDaMWCO dialysis cassette and dialyzing overnight into disassembly buffer B (10 mM TCEP, 10 mM MgCl₂, 50 mM Tris pH 8.0) at 4° C. Disassembly was confirmed by i) MADLS and ii) TEM.

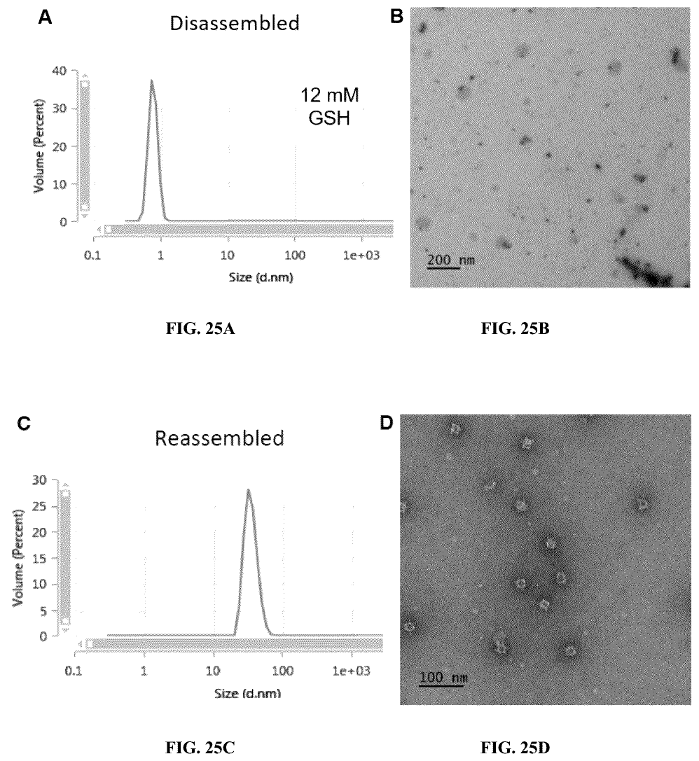

In an additional assay, PNMA2 capsids were evaluated by a disassembly and reassembly method using Glutathione (GSH) as a reducing agent. HisTrap-eluted, PBS-dialyzed PNMA2 capsids (100 μl at 1 mg/mL) were incubated for 1 h on ice in the presence of 12 mM freshly prepared GSH (reduced glutathione). PNMA2 capsid disassembly was confirmed by i) MADLS and ii) TEM. Following incubation, the reduced disassembled PNMA2 was collected by 10 min centrifugation at 11,000 rpm at 4° C. The supernatant fraction was completely removed and the visible pellet was reconstituted in 150 μl of 1×PBS buffer (pH 7.5) by mixing and vortexing. The reconstituted material was incubated overnight at 4° C. and centrifuged as above to remove irreversibly aggregated material (no more than 20% of input). The quality and integrity of the soluble re-assembled PNMA2 capsids was analyzed by i) MADLS and ii) TEM. FIGS. 25A and 25B show disassembly of PNMA2 capsids as demonstrated by MADLS (FIG. 25A) and TEM (FIG. 25B). FIGS. 25C and 25D show reassembly of PNMA2 capsids as demonstrated by MADLS (FIG. 25C) and TEM (FIG. 25D).

### Example 27—Delivery of Heterologous RNA to Cells by Capsids

Human PNMA2 protein is expressed and purified, e.g., utilizing techniques as disclosed in examples 1-2. Human PNMA2 capsids are loaded with Cre RNA by spiking in excess RNA during capsid reassembly (by dialysis into 1×PBS). Cre RNA-loaded capsids are administered to HeLa cells in biological triplicate at a final capsid concentration of 0.05 mg/ml for 4-hours at 37° C. The cells are then washed 3-times with ice-cold 1×PBS prior to RNA extraction (Invitrogen™ TRIzol™ Reagent #15596026). Purified cell-associated RNA is quantified by qPCR in technical triplicate, normalizing values to cellular GAPDH-levels, and comparing to Escherichia coli rrsA mRNA and PNMA2 RNA that could have carried over from protein purification. The amount of cell-associated Cre RNA is higher for PNMA2 capsids of the disclosure compared to control capsids not loaded with Cre RNA, demonstrating that capsids of the disclosure can effectively deliver cargo to cells.

Additionally or alternatively, delivery of a heterologous RNA to a cell can be evaluated as illustrated in FIG. 24A. 6×His-tagged PNMA2 genes are expressed in a host cell. The resulting PNMA2 monomers are mixed with translatable Cre recombinase-encoding mRNA under capsid forming conditions to form Cre mRNA-loaded capsids. Cre-loaded capsids are then administered to LoxP-luciferase reporter mice. Upon successful delivery of Cre mRNA into mouse cells and subsequent translation of Cre recombinase protein, LoxP sites of the reporter are recombined, leading to luciferase expression, which is optionally detected by bioluminescence imaging upon administration of luciferin. This method is used to test the delivery potential of candidate PNMA2 genes. A positive luciferase signal indicates that the candidate PNMA2 gene encodes a protein capable of assembling into capsids that incorporate a heterologous cargo and deliver that cargo to a target cell.

### Example 28—Efficiency of Capsid Assembly, Disassembly, and Reassembly

This example provides illustrative protocols that can be used to calculate the efficiency of assembly, disassembly, and reassembly of capsids disclosed herein that comprise endogenous Gag polypeptides (e.g., native or engineered endogenous Gag polypeptides, such as native or engineered PNMA2 polypeptides) and the recovery of assembled, disassembled, and reassembled endogenous Gag polypeptides. Also provided are methods to purify assembled and reassembled capsids.

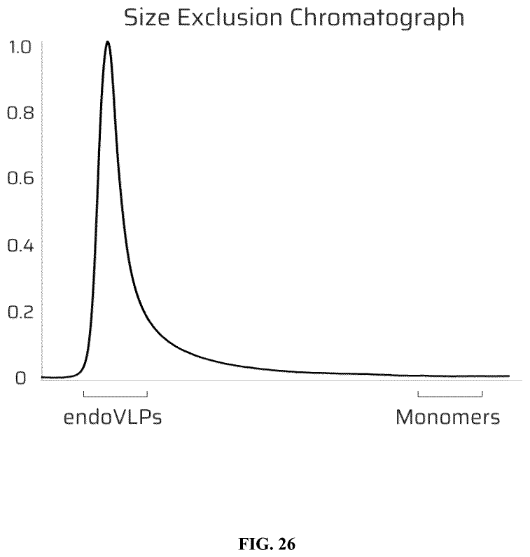

Isolated capsids are disassembled into monomers and other non-capsid forms by incubation in a disassembly buffer that may contain a chaotropic agent (e.g. urea) and/or a reducing agent (e.g. DTT). The efficiency of disassembly can be determined by MADLS or size exclusion chromatography (SEC). MADLS shows a monomer peak at about 1 nm and a capsid peak at about 30 nm. SEC separates capsids in the void volume from monomers that enter the column matrix and are eluted according to their size. For SEC, samples are loaded onto a Superose 6 Increase 10/300 GL column (GE Healthcare, Cat #29091596) in PBS-500 buffer (1×PBS pH 7.5, 363 mM NaCl). 1 Column Volume of PB S-500 buffer is run through the column and capsids elute in the column Void Volume. Monomers and other non-capsid forms are eluted from the matrix later, after approximately 15 mL of PB S-500 has eluted. Results from an SEC analysis of isolated PNMA2 capsids are shown in FIG. 26. For MADLS or SEC, the efficiency of disassembly is determined by quantifying the percent of total protein in the monomer peak. When the efficiency of disassembly is greater than about 95%, recovery of disassembled endo-Gag polypeptides is determined by comparing the amount of protein in solution after disassembly to the amount of protein in solution before disassembly.

Disassembled endo-Gag monomers are reassembled into capsids by incubation in a reassembly buffer that may contain a heterologous cargo and high salt. The reassembly buffer may not contain a chaotropic agent or a reducing agent. After reassembly, aggregates are removed by centrifugation at 15,000×g for 5 min at +4° C. The efficiency of reassembly can be determined by comparing the amounts of endo-Gag polypeptides in the monomer and capsid peaks as assayed by MADLS or SEC. Reassembled capsid compositions where at least 95% of the endo-Gag polypeptides are incorporated into capsids can be used directly for cargo delivery to cells or can be further purified. Further purification can be recommended when reassembly is less than 95% efficient. Reassembled capsids can be purified by SEC or Ion-exchange chromatography (IEC). The recovery of reassembled capsids is determined by comparing the amount of endo-Gag polypeptides in reassembled capsids (with or without further purification) to the input amount of endo-Gag monomer polypeptides.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A capsid comprising an engineered endogenous retroviral capsid polypeptide, wherein the capsid is capable of disassembling into a non-capsid state with an efficiency of at least 1% and reassembling into a capsid state with an efficiency of at least 1%.

2. The capsid of claim 1, wherein the efficiency of the disassembling is determined by quantifying an amount of the endogenous retroviral capsid polypeptide in solution after treating purified capsids with a disassembly buffer.

3. The capsid of claim 1 or claim 2, wherein the efficiency of the reassembling is determined by comparing an amount of engineered endogenous retroviral capsid polypeptide in a capsid peak from an amount of engineered endogenous retroviral capsid polypeptide in a monomer peak, and wherein the capsid peak and the monomer peak are identified by multi-angle dynamic light scattering or by size exclusion chromatography.

4. The capsid of any one of claims 1-3, wherein the capsid is capable of disassembling into the non-capsid state with an efficiency of at least about 20%.

5. The capsid of any one of claims 1-4, wherein the capsid is capable of reassembling into the capsid state with an efficiency of at least about 20%.

6. The capsid of any one of claims 1-5, wherein the engineered endogenous retroviral capsid polypeptide is an engineered endo-Gag polypeptide.

7. The capsid of any one of claims 1-6, wherein the engineered endogenous retroviral capsid polypeptide is an engineered PNMA protein.

8. The capsid of claim 7, wherein the engineered endogenous retroviral capsid polypeptide is an engineered PNMA2 protein.

9. The capsid of any one of claims 1-8, wherein the reassembling occurs in a buffer containing less than 500 mOsm/kg of salt.

10. The capsid of any one of claims 1-9, further comprising a heterologous cargo that is not associated the endogenous retroviral capsid polypeptide in nature.

11. The capsid of claim 10, wherein the heterologous cargo comprises a nucleic acid.

12. The capsid of claim 10, wherein the heterologous cargo comprises an RNA.

13. The capsid of claim 10, wherein the heterologous cargo comprises a DNA.

14. The capsid of any one of claims 10-13, wherein the heterologous cargo comprises or encodes a gene editing system or a component thereof.

15. The capsid of any one of claims 10-13, wherein the heterologous cargo comprises or encodes a CRISPR/Cas system or a component thereof.

16. The capsid of any one of claims 10-13, wherein the heterologous cargo comprises or encodes a zinc finger nuclease system or a component thereof.

17. The capsid of any one of claims 10-13, wherein the heterologous cargo comprises or encodes a TALEN system or a component thereof.

18. The capsid of any one of claims 10-17, wherein the heterologous cargo comprises a therapeutic agent.

19. The capsid of any one of claims 10-18, wherein the heterologous cargo comprises a polypeptide.

20. The capsid of any one of claims 10-19, wherein the heterologous cargo comprises an antibody or antigen-binding fragment thereof, a peptidomimetic, a nucleotidomimetic, a drug, a diagnostic tool, an imaging tool, a small molecule, or a combination thereof.

21. The capsid of any one of claims 10-20, wherein at least 50% of the heterologous cargo is in an interior of the capsids.

22. The capsid of any one of claims 10-21, wherein at least 1% of the heterologous cargo is on an exterior of the capsids.

23. The capsid of any one of claims 8-22, wherein the engineered PNMA2 polypeptide comprises an amino acid sequence of a mammalian PNMA2.

24. The capsid of any one of claims 8-23, wherein the engineered PNMA2 polypeptide comprises an amino acid sequence of a human PNMA2.

25. The capsid of any one of claims 8-24, wherein the engineered PNMA2 polypeptide comprises an amino acid sequence with at least 90% sequence identity to a sequence comprising at least 100 consecutive amino acids of SEQ ID NO: 7, SEQ ID NO: 8, or SEQ ID NO: 1.

26. The capsid of any one of claims 8-24, wherein the PNMA2 polypeptide comprises an amino acid sequence with at least 90% sequence identity to a sequence comprising at least 250 consecutive amino acids of SEQ ID NO: 7, SEQ ID NO: 8, or SEQ ID NO: 1.

27. The capsid of any one of claims 8-24, wherein the engineered PNMA2 polypeptide comprises an amino acid sequence with at least 90% sequence identity to SEQ ID NO: 7, SEQ ID NO: 8, or SEQ ID NO: 1.

28. The capsid of any one of claims 8-24, wherein the engineered PNMA2 polypeptide comprises an amino acid sequence that is SEQ ID NO: 7, SEQ ID NO: 8, or SEQ ID NO: 1.

29. An isolated engineered endogenous retroviral capsid polypeptide that is capable of assembling to form a capsid with an efficiency of at least 5%.

30. The isolated engineered endogenous retroviral capsid polypeptide of claim 29, wherein the efficiency of the assembling is determined by size exclusion chromatography assay to quantify a percentage of endogenous retroviral capsid polypeptide present in a capsid state.

31. The isolated engineered endogenous retroviral capsid polypeptide of claim 29 or claim 30, wherein upon isolation the engineered endogenous retroviral capsid polypeptide is capable of assembling to form a capsid with an efficiency of at least about 20%.

32. The isolated engineered endogenous retroviral capsid polypeptide of any one of claims 29-31, wherein upon isolation the engineered endogenous retroviral capsid polypeptide is capable of assembling to form a capsid with an efficiency of at least about 50%.

33. The isolated engineered endogenous retroviral capsid polypeptide of any one of claims 29-32, wherein the assembling occurs in a buffer containing less than 500 mOsm/kg of salt.

34. The isolated engineered endogenous retroviral capsid polypeptide of any one of claims 29-33, wherein the engineered endogenous retroviral capsid polypeptide comprises a sequence of an endo-Gag polypeptide.

35. The isolated engineered endogenous retroviral capsid polypeptide of any one of claims 29-34, wherein the engineered endogenous retroviral capsid polypeptide is an engineered PNMA protein.

36. The isolated engineered endogenous retroviral capsid polypeptide of any one of claims 39-35, wherein the engineered endogenous retroviral capsid polypeptide is an engineered PNMA2 protein.

37. The isolated engineered endogenous retroviral capsid polypeptide of claim 36, wherein the engineered PNMA2 protein comprises a sequence modification relative to SEQ ID NO: 1 or SEQ ID NO: 7.

38. The isolated engineered endogenous retroviral capsid polypeptide of claim 37, wherein the sequence modification comprises an amino acid insertion.

39. The isolated engineered endogenous retroviral capsid polypeptide of claim 37 or claim 38, wherein the sequence modification comprises an amino acid deletion.

40. The isolated engineered endogenous retroviral capsid polypeptide of any one of claims 37-39, wherein the sequence modification comprises an amino acid substitution

41. The isolated engineered endogenous retroviral capsid polypeptide of any one of claims 37-40, wherein the sequence modification comprises a cargo binding domain.

42. The isolated engineered endogenous retroviral capsid polypeptide of any one of claims 37-41, wherein the sequence modification comprises a nucleic acid binding domain.

43. The isolated engineered endogenous retroviral capsid polypeptide of any one of claims 37-42, wherein the sequence modification comprises an RNA binding domain.

44. The isolated engineered endogenous retroviral capsid polypeptide of any one of claims 37-43, wherein the sequence modification comprises a DNA binding domain.

45. The isolated engineered endogenous retroviral capsid polypeptide of any one of claims 37-44, wherein the sequence modification comprises a zinc finger domain.

46. The isolated engineered endogenous retroviral capsid polypeptide of any one of claims 37-45, wherein the sequence modification comprises a sub-cellular localization signal.

47. The isolated engineered endogenous retroviral capsid polypeptide of any one of claims 37-46, wherein the sequence modification comprises a nuclear localization signal (NLS).

48. The isolated engineered endogenous retroviral capsid polypeptide of any one of claims 37-47, wherein the sequence modification comprises an antibody or an antigen-binding fragment thereof.

49. The isolated engineered endogenous retroviral capsid polypeptide of claim 48, wherein the antibody or antigen-binding fragment comprises a single chain variable fragment (scFv) or a single domain antibody.

50. The isolated engineered endogenous retroviral capsid polypeptide of any one of claims 37-49, wherein the sequence modification comprises a domain that binds to a cell surface molecule.

51. The isolated engineered endogenous retroviral capsid polypeptide of any one of claims 37-50, wherein the sequence modification comprises an arginine-rich domain.

52. The isolated engineered endogenous retroviral capsid polypeptide of any one of claims 37-51, wherein the sequence modification is at an N-terminus of the engineered PNMA2 polypeptide.

53. The isolated engineered endogenous retroviral capsid polypeptide of any one of claims 37-51, wherein the sequence modification is at a C-terminus of the engineered PNMA2 polypeptide.

54. The isolated engineered endogenous retroviral capsid polypeptide of any one of claims 37-51, wherein the sequence modification is within the PNMA2 polypeptide.

55. The isolated engineered endogenous retroviral capsid polypeptide of any one of claims 37-54, wherein the sequence modification comprises addition of a cysteine residue that is not present in native PNMA2 or elimination of a cysteine residue that is present in native PNMA2.

56. A method of making a capsid, the method comprising:
(a) expressing an endogenous retroviral capsid polypeptide in a host cell or a cell-free expression system; and
(b) isolating the endogenous retroviral capsid polypeptide;
wherein at least about 5% of the isolated endogenous retroviral capsid polypeptide assembles to form the capsid.

57. The method of claim 56, wherein the percentage of the isolated endogenous retroviral capsid that assembles to form the capsid is determined by size exclusion chromatography.

58. The method of claim 56 or claim 57, wherein at least 20% of the isolated endogenous retroviral capsid polypeptide assembles to form the capsid.

59. A method of making a capsid comprising a cargo, the method comprising:
(a) expressing an endogenous retroviral capsid polypeptide in a host cell or a cell-free expression system;
(b) isolating a capsid comprising the endogenous retroviral capsid polypeptide;
(c) disassembling the capsid, thereby generating endogenous retroviral capsid polypeptide in a disassembled state;
(d) contacting the disassembled endogenous retroviral capsid polypeptide to a cargo,
(e) reassembling the disassembled endogenous retroviral capsid polypeptide; thereby generating the capsid comprising the cargo.

60. The method of claim 59, wherein the isolated PNMA2 polypeptide is at least partially present in a capsid form prior to step (c).

61. The method of claim 59 or claim 60, wherein at least about 5% of the PNMA2 polypeptide that is present in capsid form before step (c) is disassembled after step (c).

62. The method of any one of claims 59-61, wherein at least about 5% of the PNMA2 polypeptide that is present in disassembled form after step (c) is present in capsid form after step (d).

63. A method of loading endogenous retroviral capsids, the method comprising:
a) disassembling endogenous retroviral capsids into a composition comprising endogenous retroviral capsid monomers;
b) contacting a cargo with the composition comprising endogenous retroviral capsid monomers; and
c) reassembling the composition comprising endogenous retroviral capsid monomers into endogenous retroviral capsids;
thereby loading the cargo into an interior of the endogenous retroviral capsids.

64. The method of any one of claims 56-63, wherein the endogenous retroviral capsid polypeptide is a native endo-Gag polypeptide or an engineered endo-Gag polypeptide.

65. The method of any one of claims 56-64, wherein the endogenous retroviral capsid polypeptide is a native PNMA protein or an engineered PNMA protein.

66. The method of any one of claims 56-65, wherein the endogenous retroviral capsid polypeptide is a native PNMA2 protein or an engineered PNMA2 protein.

67. The method of any one of claims 56-66, wherein the method produces assembled capsids of at least about 50% purity as determined by SDS-PAGE.

68. The method of any one of claims 56-67, wherein the method produces assembled capsids with at least about 50% particle homogeneity as determined by multi-angle dynamic light scattering.

69. The method of any one of claims 59-62, wherein the disassembling occurs in a disassembly buffer comprising a reducing agent.

70. The method of claim 69, wherein the reducing agent comprises reduced glutathione (GSH), beta mercaptoethanol (β-ME), Dithiothreitol (DTT), or tris(2-carboxyethyl)phosphine (TCEP).

71. The method of any one of claims 59-62 and 69-70, wherein the disassembling occurs in a disassembly buffer comprising a non-denaturing detergent.

72. The method of claim 71, wherein the non-denaturing detergent is CHAPS.

73. The method of any one of claims 59-72, wherein the reassembling occurs in a reassembly buffer containing less than 500 mOsm/kg of salt.

74. The method of any one of claims 59-72, wherein the reassembling occurs in a reassembly buffer containing about 270-330 mOsm/kg of salt.

75. An engineered PNMA2 polypeptide that comprises a sequence modification relative to SEQ ID NO: 1 or SEQ ID NO: 7.

76. The engineered PNMA2 polypeptide of claim 75, wherein the sequence modification comprises an amino acid insertion.

77. The engineered PNMA2 polypeptide of claim 75 or claim 76, wherein the sequence modification comprises an amino acid deletion.

78. The engineered PNMA2 polypeptide of any one of claims 75-77, wherein the sequence modification comprises an amino acid substitution

79. The engineered PNMA2 polypeptide of any one of claims 75-78, wherein the sequence modification comprises a cargo binding domain.

80. The engineered PNMA2 polypeptide of any one of claims 75-79, wherein the sequence modification comprises a nucleic acid binding domain.

81. The engineered PNMA2 polypeptide of any one of claims 75-80, wherein the sequence modification comprises an RNA binding domain.

82. The engineered PNMA2 polypeptide of any one of claims 75-81, wherein the sequence modification comprises a DNA binding domain.

83. The engineered PNMA2 polypeptide of any one of claims 75-82, wherein the sequence modification comprises a zinc finger domain.

84. The engineered PNMA2 polypeptide of any one of claims 75-83, wherein the sequence modification comprises a sub-cellular localization signal.

85. The engineered PNMA2 polypeptide of any one of claims 75-84, wherein the sequence modification comprises a nuclear localization signal (NLS).

86. The engineered PNMA2 polypeptide of any one of claims 75-85, wherein the sequence modification comprises an antibody or an antigen-binding fragment thereof.

87. The engineered PNMA2 polypeptide of claim 86, wherein the antibody or antigen-binding fragment comprises a single chain variable fragment (scFv) or a single domain antibody.

88. The engineered PNMA2 polypeptide of any one of claims 75-85, wherein the sequence modification comprises a domain that binds to a cell surface molecule.

89. The engineered PNMA2 polypeptide of any one of claims 75-88, wherein the sequence modification comprises an arginine-rich domain.

90. The engineered PNMA2 polypeptide of any one of claims 75-89, wherein the sequence modification is at an N-terminus of the engineered PNMA2 polypeptide.

91. The engineered PNMA2 polypeptide of any one of claims 75-89, wherein the sequence modification is at a C-terminus of the engineered PNMA2 polypeptide.

92. The engineered PNMA2 polypeptide of any one of claims 75-91, wherein the sequence modification is within the PNMA2 polypeptide.

93. The engineered PNMA2 polypeptide of any one of claims 75-92, wherein the sequence modification comprises a cysteine residue that is not present in native PNMA2.

94. A capsid comprising a PNMA2 polypeptide and a heterologous cargo that is not associated with a capsid comprising PNMA2 in nature, wherein the PNMA2 polypeptide comprises an amino acid sequence having at least 90% sequence identity to SEQ ID NO: 7, SEQ ID NO: 8, or SEQ ID NO: 1.

95. A capsid comprising the engineered PNMA2 polypeptide of any one of claims 75-93.

96. A capsid comprising: (a) a first endogenous retroviral capsid polypeptide that comprises an amino acid sequence of a native PNMA2 polypeptide or an engineered PNMA2 polypeptide; and (b) a second endogenous retroviral capsid polypeptide; wherein the amino acid sequence of the first endogenous retroviral capsid polypeptide is not identical to the amino acid sequence of the second endogenous retroviral capsid polypeptide.

97. A capsid comprising: (a) a first endogenous retroviral capsid polypeptide that is the engineered PNMA2 polypeptide of any one of claims 75-93; and (b) a second endogenous retroviral capsid polypeptide; wherein the amino acid sequence of the first endogenous retroviral capsid polypeptide is not identical to the amino acid sequence of the second endogenous retroviral capsid polypeptide.

98. The capsid of claim 96 or claim 97, wherein the second endogenous retroviral capsid polypeptide comprises an amino acid sequence of a native PNMA polypeptide or an engineered PNMA polypeptide.

99. The capsid of any one of claims 96-98, wherein the second endogenous retroviral capsid polypeptide comprises an amino acid sequence of a native PNMA2 polypeptide or an engineered PNMA2 polypeptide.

100. The capsid of any one of claims 96-98, wherein the second endogenous retroviral capsid polypeptide is not a PNMA2 polypeptide.

101. The capsid of any one of claims 94-100, further comprising a heterologous cargo that is not associated with a capsid comprising PNMA2 in nature.

102. The capsid of claim 101, wherein the heterologous cargo comprises a nucleic acid.

103. The capsid of claim 101 or claim 102, wherein the heterologous cargo comprises an RNA.

104. The capsid of any one of claims 101-103, wherein the heterologous cargo comprises a DNA.

105. The capsid of any one of claims 101-104, wherein the heterologous cargo comprises or encodes a gene editing system or a component thereof.

106. The capsid of any one of claims 101-104, wherein the heterologous cargo comprises or encodes a CRISPR/Cas system or a component thereof.

107. The capsid of any one of claims 101-104, wherein the heterologous cargo comprises or encodes a zinc finger nuclease system or a component thereof.

108. The capsid of any one of claims 101-104, wherein the heterologous cargo comprises or encodes a TALEN system or a component thereof.

109. The capsid of any one of claims 101-108, wherein the heterologous cargo comprises a therapeutic agent.

110. The capsid of any one of claims 101-109, wherein the heterologous cargo comprises a polypeptide.

111. The capsid of any one of claims 101-110, wherein the heterologous cargo comprises an antibody or antigen-binding fragment thereof, a peptidomimetic, a nucleotidomimetic, a drug, a diagnostic tool, an imaging tool, a small molecule, or a combination thereof.

112. A plurality of the capsid of any one of claims 101-111, wherein at least 50% of the heterologous cargo is in an interior of the capsids.

113. A plurality of the capsid of any one of claims 101-111, wherein at least 1% of the heterologous cargo is on an exterior of the capsids.

114. The capsid of any one of claims 94-111, wherein the PNMA2 polypeptide comprises an amino acid sequence of a mammalian PNMA2.

115. The capsid of any one of claims 94-111, wherein the PNMA2 polypeptide comprises an amino acid sequence of a human PNMA2.

116. The capsid of any one of claims 94-111, wherein the PNMA2 polypeptide comprises an amino acid sequence with at least 90% sequence identity to a sequence comprising at least 100 consecutive amino acids of SEQ ID NO: 7, SEQ ID NO: 8, or SEQ ID NO: 1.

117. The capsid of any one of claims 94-111, wherein the PNMA2 polypeptide comprises an amino acid sequence with at least 90% sequence identity to a sequence comprising at least 250 consecutive amino acids of SEQ ID NO: 7, SEQ ID NO: 8, or SEQ ID NO: 1.

118. The capsid of any one of claims 94-111, wherein the PNMA2 polypeptide comprises an amino acid sequence with at least 90% sequence identity to SEQ ID NO: 7, SEQ ID NO: 8, or SEQ ID NO: 1.

119. The capsid of any one of claims 94-111, wherein the PNMA2 polypeptide comprises an amino acid sequence that is SEQ ID NO: 7, SEQ ID NO: 8, or SEQ ID NO: 1.

120. The capsid of any one of claims 94-111, wherein the capsid comprises a disulfide bond.

121. A composition comprising a plurality of capsids that comprise a PNMA2 polypeptide, wherein the capsids are at least 50% pure as determined by SDS-PAGE.

122. A composition comprising a plurality of the capsid of any one of claims 1-28 and 94-111, wherein the capsids are at least 50% pure as determined by SDS-PAGE.

123. A composition comprising a plurality of capsids that comprise a PNMA2 polypeptide, wherein the composition comprises at least 50% particle homogeneity as determined by multi-angle dynamic light scattering.

124. A composition comprising a plurality of capsids of any one of claims 1-28 and 94-111, wherein the composition comprises at least 50% particle homogeneity as determined by multi-angle dynamic light scattering.

125. A composition comprising the composition of any one of claims 121-124 or the capsid of any one of claim 1-28 or 94-120, further comprising a delivery component.

126. The composition of claim 125, wherein the delivery component comprises a lipid.

127. The composition of claim 125 or claim 126, wherein the delivery component comprises a polypeptide.

128. The composition of any one of claims 125-127, wherein the delivery component comprises a polymer.

129. The composition of any one of claims 125-128, wherein the delivery component comprises a cationic lipid.

130. The composition of any one of claims 125-129, wherein the delivery component comprises a cationic peptide.

131. The composition of any one of claims 125-130, wherein the delivery component comprises a cationic polymer.

132. The composition of any one of claims 125-131, wherein the delivery component comprises a cell-penetrating peptide.

133. The composition of any one of claims 125-131, wherein the delivery component comprises a fusogenic protein.

134. The composition of any one of claims 125-131, wherein the delivery component comprises an endogenous retroviral envelope protein.

135. The composition of any one of claims 125-132, wherein the delivery component comprises a liposome.

136. A nucleic acid encoding the engineered PNMA2 polypeptide of any one of claims 75-93.

137. A vector comprising the nucleic acid of claim 136.

138. A cell comprising the nucleic acid of claim 137.

139. A method of delivering a heterologous cargo to a cell, comprising contacting the cell with the composition of any one of claims 121-135 or the capsid of any one of claims 1-28 and 94-111.

140. A method of delivering a cargo to a cell, the method comprising contacting the cell with a composition comprising a plurality of isolated capsids, wherein each capsid of the plurality of isolated capsids comprises a polypeptide comprising a sequence of PNMA2 and the cargo.

141. A method of delivering a cargo to a cell, the method comprising contacting the cell with at least about 0.001 pg/mL of a capsid that comprises a capsid polypeptide comprising at least 100 consecutive amino acids of SEQ ID NO: 1 and a heterologous cargo that is not associated with the polypeptide comprising the at least 100 consecutive amino acids of SEQ ID NO: 1 in nature.

142. The method of any one of claims 139-141, wherein the cell is a mammalian cell.

143. The method of any one of claims 139-142, wherein the cell is a human cell.

144. The method of any one of claims 139-143, wherein the cell is contacted with the capsid at a concentration of at least about at least about 0.01 pg/mL, at least about 0.1 pg/mL, at least about 1 pg/mL, at least about 10 pg/mL, at least about 100 pg/mL, at least about 1 ng/mL, at least about 10 ng/mL, at least about 100 ng/mL, at least about 1 μg/mL, at least about 10 μg/mL, at least about 100 μg/mL, at least about 1 mg/mL, at least about 10 mg/mL, or at least about 100 mg/mL.

145. The method of any one of claims 139-144, wherein the contacting is in vivo.

146. The method of any one of claims 139-144, wherein the contacting is in vitro or ex vivo.

147. The method of any one of claims 139-146, wherein the heterologous cargo comprises a nucleic acid, wherein the cell expresses a gene encoded by the nucleic acid after the delivering.

148. The method of any one of claims 139-147, wherein the heterologous cargo comprises a protein, a peptide, or an antibody or binding fragment thereof, a peptidomimetic, a nucleotidomimetic, a drug, a diagnostic tool, an imaging tool, a small molecule, or a combination thereof.

149. The method of any one of claims 139-148, wherein the heterologous cargo comprises or encodes a gene editing system or a component thereof.

150. The method of any one of claims 139-148, wherein the heterologous cargo comprises a CRISPR/Cas system or a component thereof.

151. The method of any one of claims 139-148, wherein the heterologous cargo comprises a zinc finger nuclease system or a component thereof.

152. The method of any one of claims 139-148, wherein the heterologous cargo comprises a TALEN system or a component thereof.


SEQUENCE LISTING (59 sequences)

SEQ ID NO: 1 - protein, 364 aa, Homo sapiens
MALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 2 - protein, 353 aa, Unknown
  Description of Unknown PNMA 1 sequence
MAMTLLEDWCRGMDVNSQRALLVWGIPVNCDEAEIEETLQAAMPQVSYRMLGRMFWREENAKAALLELTGAVDYAAIPREMPGKGGVWKVLFKPPTSDAEFLERLHLFLAREGWTVQDVARVLGFQNPTPTPGPEMPAEMLNYILDNVIQPLVESIWYKRLTLFSGRDIPGPGEETFDPWLEHTNEVLEEWQVSDVEKRRRLMESLRGPAADVIRILKSNNPAITTAECLKALEQVFGSVESSRDAQIKFLNTYQNPGEKLSAYVIRLEPLLQKVVEKGAIDKDNVNQARLEQVIAGANHSGAIRRQLWLTGAGEGPAPNLFQLLVQIREEEAKEEEEEAEATLLQLGLEGHF

SEQ ID NO: 3 - protein, 463 aa, Unknown
  Description of Unknown PNMA 3 sequence
MPLTLLQDWCRGEHLNTRRCMLILGIPEDCGEDEFEETLQEACRHLGRYRVIGRMFRREENAQAILLELAQDIDYALLPREIPGKGGPWEVIVKPRNSDGEFLNRLNRFLEEERRTVSDMNRVLGSDTNCSAPRVTISPEFWTWAQTLGAAVQPLLEQMLYRELRVFSGNTISIPGALAFDAWLEHTTEMLQMWQVPEGEKRRRLMECLRGPALQVVSGLRASNASITVEECLAALQQVFGPVESHKIAQVKLCKAYQEAGEKVSSFVLRLEPLLQRAVENNVVSRRNVNQTRLKRVLSGATLPDKLRDKLKLMKQRRKPPGFLALVKLLREEEEWEATLGPDRESLEGLEVAPRPPARITGVGAVPLPASGNSFDARPSQGYRRRRGRGQHRRGGVARAGSRGSRKRKRHTFCYSCGEDGHIRVQCINPSNLLLVKQKKQAAVESGNGNWAWDKSHPKSKAK

SEQ ID NO: 4 - protein, 351 aa, Unknown
  Description of Unknown PNMA4 (MOAP1) sequence
MTLRLLEDWCRGMDMNPRKALLIAGISQSCSVAEIEEALQAGLAPLGEYRLLGRMFRRDENRKVALVGLTAETSHALVPKEIPGKGGIWRVIFKPPDPDNTFLSRLNEFLAGEGMTVGELSRALGHENGSLDPEQGMIPEMWAPMLAQALEALQPALQCLKYKKLRVFSGRESPEPGEEEFGRWMFHTTQMIKAWQVPDVEKRRRLLESLRGPALDVIRVLKINNPLITVDECLQALEEVFGVTDNPRELQVKYLTTYQKDEEKLSAYVLRLEPLLQKLVQRGAIERDAVNQARLDQVIAGAVHKTIRRELNLPEDGPAPGFLQLLVLIKDYEAAEEEEALLQAILEGNFT

SEQ ID NO: 5 - protein, 448 aa, Unknown
  Description of Unknown PNMA 5 sequence
MALTLLEDWCKGMDMDPRKALLIVGIPMECSEVEIQDTVKAGLQPLCAYRVLGRMFRREDNAKAVFIELADTVNYTTLPSHIPGKGGSWEVVVKPRNPDDEFLSRLNYFLKDEGRSMTDVARALGCCSLPAESLDAEVMPQVRSPPLEPPKESMWYRKLKVFSGTASPSPGEETFEDWLEQVTEIMPIWQVSEVEKRRRLLESLRGPALSIMRVLQANNDSITVEQCLDALKQIFGDKEDFRASQFRFLQTSPKIGEKVSTFLLRLEPLLQKAVHKSPLSVRSTDMIRLKHLLARVAMTPALRGKLELLDQRGCPPNFLELMKLIRDEEEWENTEAVMKNKEKPSGRGRGASGRQARAEASVSAPQATVQARSFSDSSPQTIQGGLPPLVKRRRLLGSESTRGEDHGQATYPKAENQTPGREGPQAAGEELGNEAGAGAMSHPKPWET

SEQ ID NO: 6 - protein, 399 aa, Unknown
  Description of Unknown PNMA 6A sequnece
MAVTMLQDWCRWMGVNARRGLLILGIPEDCDDAEFQESLEAALRPMGHFTVLGKAFREEDNATAALVELDREVNYALVPREIPGTGGPWNVVFVPRCSGEEFLGLGRVFHFPEQEGQMVESVAGALGVGLRRVCWLRSIGQAVQPWVEAVRCQSLGVFSGRDQPAPGEESFEVWLDHTTEMLHVWQGVSERERRRRLLEGLRGTALQLVHALLAENPARTAQDCLAALAQVFGDNESQATIRVKCLTAQQQSGERLSAFVLRLEVLLQKAMEKEALARASADRVRLRQMLTRAHLTEPLDEALRKLRMAGRSPSFLEMLGLVRESEAWEASLARSVRAQTQEGAGARAGAQAVARASTKVEAVPGGPGREPEGLLQAGGQEAEELLQEGLKPVLEECDN

SEQ ID NO: 7 - protein, 363 aa, Artificial Sequence
  Description of Artificial Sequence Synthetic polypeptide
ALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 8 - protein, 366 aa, Artificial Sequence
  Description of Artificial Sequence Synthetic polypeptide
GSGALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 9 - protein, 25 aa, Artificial Sequence
  Description of Artificial Sequence Synthetic peptide
  SITE (1)..(25): This sequence may encompass 1-5 “Glu Ala Ala Ala Lys” repeating units
  See specification as filed for detailed description of substitutions and preferred embodiments
EAAAKEAAAKEAAAKEAAAKEAAAK

SEQ ID NO: 10 - protein, 25 aa, Artificial Sequence
  Description of Artificial Sequence Synthetic peptide
  SITE (1)..(25): This sequence may encompass 1-5 “Glu Ala Ala Ala Arg” repeating units
  See specification as filed for detailed description of substitutions and preferred embodiments
EAAAREAAAREAAAREAAAREAAAR

SEQ ID NO: 11 - protein, 50 aa, Artificial Sequence
  Description of Artificial Sequence Synthetic polypeptide
  SITE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS

SEQ ID NO: 12 - protein, 40 aa, Artificial Sequence
  Description of Artificial Sequence Synthetic polypeptide
  SITE (1)..(40): This sequence may encompass 1-10 “Gly Gly Gly Ser” repeating units
GGGSGGGSGGGSGGGSGGGSGGGSGGGSGGGSGGGSGGGS

SEQ ID NO: 13 - protein, 18 aa, Artificial Sequence
  Description of Artificial Sequence Synthetic peptide
KESGSVSSEQLAQFRSLD

SEQ ID NO: 14 - protein, 14 aa, Artificial Sequence
  Description of Artificial Sequence Synthetic peptide
EGKSSGSGSESKST

SEQ ID NO: 15 - protein, 10 aa, Artificial Sequence
  Description of Artificial Sequence Synthetic peptide
GGAANLVRGG

SEQ ID NO: 16 - protein, 10 aa, Artificial Sequence
  Description of Artificial Sequence Synthetic peptide
SGRIGFLRTA

SEQ ID NO: 17 - protein, 5 aa, Artificial Sequence
  Description of Artificial Sequence Synthetic peptide
SGRSA

SEQ ID NO: 18 - protein, 4 aa, Artificial Sequence
  Description of Artificial Sequence Synthetic peptide
GFLG

SEQ ID NO: 19 - protein, 4 aa, Artificial Sequence
  Description of Artificial Sequence Synthetic peptide
ALAL

SEQ ID NO: 20 - protein, 5 aa, Artificial Sequence
  Description of Artificial Sequence Synthetic peptide
  MOD_RES (3)..(3): S-ethylcysteine
PICFF

SEQ ID NO: 21 - protein, 5 aa, Artificial Sequence
  Description of Artificial Sequence Synthetic peptide
  MOD_RES (3)..(3): S or T
  MOD_RES (4)..(4): L or I
  MOD_RES (5)..(5): S or T
PRXXX

SEQ ID NO: 22 - protein, 4 aa, Artificial Sequence
  Description of Artificial Sequence Synthetic peptide
DEVD

SEQ ID NO: 23 - protein, 6 aa, Artificial Sequence
  Description of Artificial Sequence Synthetic peptide
GWEHDG

SEQ ID NO: 24 - protein, 8 aa, Artificial Sequence
  Description of Artificial Sequence Synthetic peptide
RPLALWRS

SEQ ID NO: 25 - DNA, 66 bp, Artificial Sequence
  Description of Artificial Sequence Synthetic oligonucleotide
atgcatcaccatcaccatcacggctcagggtctggtagcgaaaatctgtacttccaggggtcaggt

SEQ ID NO: 26 - protein, 22 aa, Artificial Sequence
  Description of Artificial Sequence Synthetic peptide
MHHHHHHGSGSGSENLYFQGSG

SEQ ID NO: 27 - protein, 6 aa, Artificial Sequence
  Description of Artificial Sequence Synthetic 6xHis tag
HHHHHH

SEQ ID NO: 28 - protein, 6 aa, Artificial Sequence
  Description of Artificial Sequence Synthetic peptide
GSGSGS

SEQ ID NO: 29 - protein, 7 aa, Tobacco etch virus
ENLYFQG

SEQ ID NO: 30 - DNA, 1089 bp, Artificial Sequence
  Description of Artificial Sequence Synthetic polynucleotide
gcgttggcactgttagaagattggtgccgtattatgtctgtggacgagcagaagtctcttatggttactgggatcccggcggatttcgaagaagccgaaatccaggaagttctgcaagaaacattaaagagcttgggccggtatcggctcttaggcaaaatttttcggaagcaagaaaatgctaatgcggtgttactcgaactgctcgaggatacggatgtttcagcaatcccatctgaagtgcagggcaaaggtggggtgtggaaagttatcttcaagacgccaaaccaggatactgagttccttgaacgcttgaatctgttcttggagaaagaaggccagaccgtaagcggcatgttccgggcgcttggccaagagggggtatccccggcaacggtaccttgtatttcccctgagttgctcgcgcacttattgggtcaagccatggcacacgcgcctcaacctctcctgccgatgcggtatcgcaaactgcgcgtattttcgggttcagcggttccggcccctgaggaagaatcatttgaagtttggctggagcaggcaaccgagatcgtgaaggagtggccggtcacagaggccgaaaagaaacggtggttagccgagagtcttcgtggccctgcgcttgaccttatgcatattgtacaggcggataacccttctatctcggttgaagagtgtcttgaggctttcaagcaagtctttggttcgctggagtcccgtcgtactgcccaggtccggtatctcaaaacgtatcaggaagagggtgagaaggtctccgcatacgtcctgcggttggaaacgcttttgcgccgggcagtagaaaaacgcgcaattcctcgtcgcattgccgatcaagttcgtttagagcaagtgatggctggcgcaactttaaaccaaatgttatggtgccggttacgtgaactgaaggaccaggggcctccgccatcttttcttgagctgatgaaggtgatccgggaagaggaggaagaagaggcgtcgttcgaaaacgagagtatcgaagagccggaggaacgtgatgggtacggtcgctggaaccacgagggcgacgat

SEQ ID NO: 31 - DNA, 1092 bp, Artificial Sequence
  Description of Artificial Sequence Synthetic polynucleotide
atggcgttggcactgttagaagattggtgccgtattatgtctgtggacgagcagaagtctcttatggttactgggatcccggcggatttcgaagaagccgaaatccaggaagttctgcaagaaacattaaagagcttgggccggtatcggctcttaggcaaaatttttcggaagcaagaaaatgctaatgcggtgttactcgaactgctcgaggatacggatgtttcagcaatcccatctgaagtgcagggcaaaggtggggtgtggaaagttatcttcaagacgccaaaccaggatactgagttccttgaacgcttgaatctgttcttggagaaagaaggccagaccgtaagcggcatgttccgggcgcttggccaagagggggtatccccggcaacggtaccttgtatttcccctgagttgctcgcgcacttattgggtcaagccatggcacacgcgcctcaacctctcctgccgatgcggtatcgcaaactgcgcgtattttcgggttcagcggttccggcccctgaggaagaatcatttgaagtttggctggagcaggcaaccgagatcgtgaaggagtggccggtcacagaggccgaaaagaaacggtggttagccgagagtcttcgtggccctgcgcttgaccttatgcatattgtacaggcggataacccttctatctcggttgaagagtgtcttgaggctttcaagcaagtctttggttcgctggagtcccgtcgtactgcccaggtccggtatctcaaaacgtatcaggaagagggtgagaaggtctccgcatacgtcctgcggttggaaacgcttttgcgccgggcagtagaaaaacgcgcaattcctcgtcgcattgccgatcaagttcgtttagagcaagtgatggctggcgcaactttaaaccaaatgttatggtgccggttacgtgaactgaaggaccaggggcctccgccatcttttcttgagctgatgaaggtgatccgggaagaggaggaagaagaggcgtcgttcgaaaacgagagtatcgaagagccggaggaacgtgatgggtacggtcgctggaaccacgagggcgacgat

SEQ ID NO: 32 - DNA, 60 bp, Artificial Sequence
  Description of Artificial Sequence Synthetic oligonucleotide
atgcatcaccatcaccatcacggctcagggtctggtagcgaaaatctgtacttccagggg

SEQ ID NO: 33 - protein, 20 aa, Artificial Sequence
  Description of Artificial Sequence Synthetic peptide
MHHHHHHGSGSGSENLYFQG

SEQ ID NO: 34 - DNA, 1188 bp, Artificial Sequence
  Description of Artificial Sequence Synthetic polynucleotide
ggggagctggaccaccggaccagcggcgggctccacgcctaccccgggccgcggggcgggcaggtggccaagcccaacgtgatcctgcagatcgggaagtgccgggccgagatgctggagcacgtgcggcggacgcaccggcacctgctggccgaggtgtccaagcaggtggagcgcgagctgaaggggctgcaccggtcggtcgggaagctggagagcaacctggacggctacgtgcccacgagcgactcgcagcgctggaagaagtccatcaaggcctgcctgtgccgctgccaggagaccatcgccaacctggagcgctgggtcaagcgcgagatgcacgtgtggcgcgaggtgttctaccgcctggagcgctgggccgaccgcctggagtccacgggcggcaagtacccggtgggcagcgagtcagcccgccacaccgtttccgtgggcgtggggggtcccgagagctactgccacgaggcagacggctacgactacaccgtcagcccctacgccatcaccccgcccccagccgctggcgagctgcccgggcaggagcccgccgaggcccagcagtaccagccgtgggtccccggcgaggacgggcagcccagccccggcgtggacacgcagatcttcgaggaccctcgagagttcctgagccacctagaggagtacttgcggcaggtgggcggctctgaggagtactggctgtcccagatccagaatcacatgaacgggccggccaagaagtggtgggagttcaagcagggctccgtgaagaactgggtggagttcaagaaggagttcctgcagtacagcgagggcacgctgtcccgagaggccatccagcgcgagctggacctgccgcagaagcagggcgagccgctggaccagttcctgtggcgcaagcgggacctgtaccagacgctctacgtggacgcggacgaggaggagatcatccagtacgtggtgggcaccctgcagcccaagctcaagcgtttcctgcgccaccccctgcccaagaccctggagcagctcatccagaggggcatggaggtgcaggatgacctggagcaggcggccgagccggccggcccccacctcccggtggaggatgaggcggagaccctcacgcccgcccccaacagcgagtccgtggccagtgaccggacccagcccgag

SEQ ID NO: 35 - protein, 395 aa, Artificial Sequence
  Description of Artificial Sequence Synthetic polypeptide
ELDHRTSGGLHAYPGPRGGQVAKPNVILQIGKCRAEMLEHVRRTHRHLLAEVSKQVERELKGLHRSVGKLESNLDGYVPTSDSQRWKKSIKACLCRCQETIANLERWVKREMHVWREVFYRLERWADRLESTGGKYPVGSESARHTVSVGVGGPESYCHEADGYDYTVSPYAITPPPAAGELPGQEPAEAQQYQPWVPGEDGQPSPGVDTQIFEDPREFLSHLEEYLRQVGGSEEYWLSQIQNHMNGPAKKWWEFKQGSVKNWVEFKKEFLQYSEGTLSREAIQRELDLPQKQGEPLDQFLWRKRDLYQTLYVDADEEEIIQYVVGTLQPKLKRFLRHPLPKTLEQLIQRGMEVQDDLEQAAEPAGPHLPVEDEAETLTPAPNSESVASDRTQPE

SEQ ID NO: 36 - protein, 396 aa, Artificial Sequence
  Description of Artificial Sequence Synthetic polypeptide
GELDHRTSGGLHAYPGPRGGQVAKPNVILQIGKCRAEMLEHVRRTHRHLLAEVSKQVERELKGLHRSVGKLESNLDGYVPTSDSQRWKKSIKACLCRCQETIANLERWVKREMHVWREVFYRLERWADRLESTGGKYPVGSESARHTVSVGVGGPESYCHEADGYDYTVSPYAITPPPAAGELPGQEPAEAQQYQPWVPGEDGQPSPGVDTQIFEDPREFLSHLEEYLRQVGGSEEYWLSQIQNHMNGPAKKWWEFKQGSVKNWVEFKKEFLQYSEGTLSREAIQRELDLPQKQGEPLDQFLWRKRDLYQTLYVDADEEEIIQYVVGTLQPKLKRFLRHPLPKTLEQLIQRGMEVQDDLEQAAEPAGPHLPVEDEAETLTPAPNSESVASDRTQPE

SEQ ID NO: 37 - protein, 20 aa, Artificial Sequence
  Description of Artificial Sequence Synthetic peptide
MHHHHHHGSGSGSENLYFQG

SEQ ID NO: 38 - protein, 364 aa, Artificial Sequence
  Description of Artificial Sequence Synthetic polypeptide
GALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 39 - protein, 24 aa, Artificial Sequence
  Description of Artificial Sequence Synthetic peptide
EMRRARKRKPRRRQRRKKRGSGQP

SEQ ID NO: 40 - protein, 362 aa, Artificial Sequence
  Description of Artificial Sequence Synthetic polypeptide
ALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEAEMRRARKRKPRRRQRRKKRGSGQP

SEQ ID NO: 41 - protein, 382 aa, Artificial Sequence
  Description of Artificial Sequence Synthetic polypeptide
MHHHHHHGSGSGSENLYFQGALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEAEMRRARKRKPRRRQRRKKRGSGQP

SEQ ID NO: 42 - protein, 20 aa, Artificial Sequence
  Description of Artificial Sequence Synthetic peptide
RRRRGRGQHRRGGVARAGSR

SEQ ID NO: 43 - protein, 358 aa, Artificial Sequence
  Description of Artificial Sequence Synthetic polypeptide
ALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEARRRRGRGQHRRGGVARAGSR

SEQ ID NO: 44 - protein, 378 aa, Artificial Sequence
  Description of Artificial Sequence Synthetic polypeptide
MHHHHHHGSGSGSENLYFQGALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEARRRRGRGQHRRGGVARAGSR

SEQ ID NO: 45 - protein, 15 aa, Artificial Sequence
  Description of Artificial Sequence Synthetic peptide
PRRRQRRKKRGSGQP

SEQ ID NO: 46 - protein, 378 aa, Artificial Sequence
  Description of Artificial Sequence Synthetic polypeptide
ALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDDPRRRQRRKKRGSGQP

SEQ ID NO: 47 - protein, 398 aa, Artificial Sequence
  Description of Artificial Sequence Synthetic polypeptide
MHHHHHHGSGSGSENLYFQGALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDDPRRRQRRKKRGSGQP

SEQ ID NO: 48 - protein, 363 aa, Artificial Sequence
  Description of Artificial Sequence Synthetic polypeptide
ALALLEDWSRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPSISPELLAHLLGQAMAHAPQPLLPMRYRKLRVFSGSAVPAPEEESFEVWLEQATEIVKEWPVTEAEKKRWLAESLRGPALDLMHIVQADNPSISVEESLEAFKQVFGSLESRRTAQVRYLKTYQEEGEKVSAYVLRLETLLRRAVEKRAIPRRIADQVRLEQVMAGATLNQMLWSRLRELKDQGPPPSFLELMKVIREEEEEEASFENESIEEPEERDGYGRWNHEGDD

SEQ ID NO: 49 - protein, 376 aa, Artificial Sequence
  Description of Artificial Sequence Synthetic polypeptide
ALALLEDWSRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPSISPELLAHLLGQAMAHAPQPLLPMRYRKLRVFSGSAVPAPEEESFEVWLEQATEIVKEWPVTEAEKKRWLAESLRGPALDLMHIVQADNPSISVEESLEAFKQVFGSLESRRTAQVRYLKTYQEEGEKVSAYVLRLETLLRRAVEKRAIPRRIADQVRLEQVMAGATLNQMLWSRLRELKDQGPPPSFLELMKVIREEEEEEASFENESIEEPEERDGYGRWNHEGDDGSGSGSGSGSGSC

SEQ ID NO: 50 - protein, 126 aa, Artificial Sequence
  Description of Artificial Sequence Synthetic polypeptide
AAQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 51 - protein, 156 aa, Artificial Sequence
  Description of Artificial Sequence Synthetic polypeptide
MHHHHHHGSGSGSENLYFQGAAQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGSGSGSGSG

SEQ ID NO: 52 - protein, 499 aa, Artificial Sequence
  Description of Artificial Sequence Synthetic polypeptide
AAQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGSGSGSGSGALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 53 - protein, 519 aa, Artificial Sequence
  Description of Artificial Sequence Synthetic polypeptide
MHHHHHHGSGSGSENLYFQGAAQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGSGSGSGSGALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 54 - protein, 11 aa, Artificial Sequence
  Description of Artificial Sequence Synthetic peptide
YGRKKRRQRRR

SEQ ID NO: 55 - protein, 363 aa, Artificial Sequence
  Description of Artificial Sequence Synthetic polypeptide
MALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEAEMRRARKRKPRRRQRRKKRGSGQP

SEQ ID NO: 56 - protein, 9 aa, Artificial Sequence
  Description of Artificial Sequence Synthetic peptide
RRRRRRRRR

SEQ ID NO: 57 - protein, 10 aa, Artificial Sequence
  Description of Artificial Sequence Synthetic peptide
SGSGSGSGSG

SEQ ID NO: 58 - protein, 6 aa, Artificial Sequence
  Description of Artificial Sequence Synthetic peptide
GSGSGS

SEQ ID NO: 59 - protein, 13 aa, Artificial Sequence
  Description of Artificial Sequence Synthetic peptide
GSGSGSGSGSGSC